SEQUENCE LISTING (83 sequences)

SEQ ID NO: 1 - DNA, 26 bp, synthetic construct
  source 1..26: mol_type = other DNA
tgacatcaagaaggtggtgaagcagg

SEQ ID NO: 2 - DNA, 23 bp, synthetic construct
  source 1..23: mol_type = other DNA
gcgtcaaaggtggaggagtgggt

SEQ ID NO: 3 - DNA, 20 bp, synthetic construct
  source 1..20: mol_type = other DNA
ctgggttgatcctcggacct

SEQ ID NO: 4 - DNA, 19 bp, synthetic construct
  source 1..19: mol_type = other DNA
ccatcggagttgctctcca

SEQ ID NO: 5 - DNA, 20 bp, synthetic construct
  source 1..20: mol_type = other DNA
gatttgtgggcctgaagaaa

SEQ ID NO: 6 - DNA, 20 bp, synthetic construct
  source 1..20: mol_type = other DNA
cagatccatggaggaaggaa

SEQ ID NO: 7 - DNA, 26 bp, synthetic construct
  source 1..26: mol_type = other DNA
ttgcgtgagtgtggatgggattggtg

SEQ ID NO: 8 - DNA, 26 bp, synthetic construct
  source 1..26: mol_type = other DNA
gggaaatgggaggggtgcaaaagagg

SEQ ID NO: 9 - DNA, 22 bp, synthetic construct
  source 1..22: mol_type = other DNA
cgaagaagagccggcctgtacc

SEQ ID NO: 10 - DNA, 22 bp, synthetic construct
  source 1..22: mol_type = other DNA
cggaagcccatgcaacgatctc

SEQ ID NO: 11 - DNA, 22 bp, synthetic construct
  source 1..22: mol_type = other DNA
gctaacatctgccaccccacca

SEQ ID NO: 12 - DNA, 22 bp, synthetic construct
  source 1..22: mol_type = other DNA
ggattctgcggtgctgctgaca

SEQ ID NO: 13 - DNA, 21 bp, synthetic construct
  source 1..21: mol_type = other DNA
cgccgctacaagttccttaaa

SEQ ID NO: 14 - DNA, 19 bp, synthetic construct
  source 1..19: mol_type = other DNA
ggatctgctcgtcgaaggg

SEQ ID NO: 15 - DNA, 21 bp, synthetic construct
  source 1..21: mol_type = other DNA
agaaacgggcaaagacaagac

SEQ ID NO: 16 - DNA, 21 bp, synthetic construct
  source 1..21: mol_type = other DNA
gctgacaggttctatttccgc

SEQ ID NO: 17 - DNA, 23 bp, synthetic construct
  source 1..23: mol_type = other DNA
ttgaggaagagaatcagatccag

SEQ ID NO: 18 - DNA, 20 bp, synthetic construct
  source 1..20: mol_type = other DNA
cgttctgtacggggtcactc

SEQ ID NO: 19 - DNA, 19 bp, synthetic construct
  source 1..19: mol_type = other DNA
caggagctgtcgtgcaagt

SEQ ID NO: 20 - DNA, 21 bp, synthetic construct
  source 1..21: mol_type = other DNA
agtagcagacgtggttgttct

SEQ ID NO: 21 - DNA, 21 bp, synthetic construct
  source 1..21: mol_type = other DNA
tttccctcattcctgaactgc

SEQ ID NO: 22 - DNA, 25 bp, synthetic construct
  source 1..25: mol_type = other DNA
cagcaaaatcaaccatcaaataaac

SEQ ID NO: 23 - DNA, 20 bp, synthetic construct
  source 1..20: mol_type = other DNA
agcacatcaactcggaggag

SEQ ID NO: 24 - DNA, 21 bp, synthetic construct
  source 1..21: mol_type = other DNA
cattccctgggtggttcactg

SEQ ID NO: 25 - DNA, 22 bp, synthetic construct
  source 1..22: mol_type = other DNA
cagaagtcctacaaggtgtcca

SEQ ID NO: 26 - DNA, 21 bp, synthetic construct
  source 1..21: mol_type = other DNA
ctctggttgaccgtaactgcg

SEQ ID NO: 27 - DNA, 22 bp, synthetic construct
  source 1..22: mol_type = other DNA
tcgcaaatactgtggacaatgc

SEQ ID NO: 28 - DNA, 21 bp, synthetic construct
  source 1..21: mol_type = other DNA
gcagtcgtgtgatattggtgt

SEQ ID NO: 29 - DNA, 21 bp, synthetic construct
  source 1..21: mol_type = other DNA
accaagtttgagacggaacag

SEQ ID NO: 30 - DNA, 21 bp, synthetic construct
  source 1..21: mol_type = other DNA
ccctcagcgtactgatttcct

SEQ ID NO: 31 - DNA, 24 bp, synthetic construct
  source 1..24: mol_type = other DNA
aggaaatcctcagactcctgggtt

SEQ ID NO: 32 - DNA, 24 bp, synthetic construct
  source 1..24: mol_type = other DNA
cccaaactgttcaagtggcagaca

SEQ ID NO: 33 - DNA, 20 bp, synthetic construct
  source 1..20: mol_type = other DNA
ccctgggatcgagtgaagga

SEQ ID NO: 34 - DNA, 21 bp, synthetic construct
  source 1..21: mol_type = other DNA
ctgggacacatagtctctgcc

SEQ ID NO: 35 - DNA, 21 bp, synthetic construct
  source 1..21: mol_type = other DNA
ctgtgctactcctcaccatct

SEQ ID NO: 36 - DNA, 21 bp, synthetic construct
  source 1..21: mol_type = other DNA
ctctccacacatggctccttt

SEQ ID NO: 37 - DNA, 21 bp, synthetic construct
  source 1..21: mol_type = other DNA
cctacgaacaggtgatgcact

SEQ ID NO: 38 - DNA, 21 bp, synthetic construct
  source 1..21: mol_type = other DNA
gatttcttctcccttgcgtct

SEQ ID NO: 39 - DNA, 21 bp, synthetic construct
  source 1..21: mol_type = other DNA
ctcagttcctacgcttcgcat

SEQ ID NO: 40 - DNA, 20 bp, synthetic construct
  source 1..20: mol_type = other DNA
gtcgaggtcagtgaacagca

SEQ ID NO: 41 - DNA, 20 bp, synthetic construct
  source 1..20: mol_type = other DNA
gcacctctcccagcatctca

SEQ ID NO: 42 - DNA, 20 bp, synthetic construct
  source 1..20: mol_type = other DNA
gtcggaggatctctcgttgc

SEQ ID NO: 43 - DNA, 22 bp, synthetic construct
  source 1..22: mol_type = other DNA
aaaattacacgccagatttgcc

SEQ ID NO: 44 - DNA, 23 bp, synthetic construct
  source 1..23: mol_type = other DNA
ggtgtgacattactccagagttg

SEQ ID NO: 45 - DNA, 19 bp, synthetic construct
  source 1..19: mol_type = other DNA
aagtagccatggcgctgta

SEQ ID NO: 46 - DNA, 20 bp, synthetic construct
  source 1..20: mol_type = other DNA
gagacgtgactgctgtggag

SEQ ID NO: 47 - DNA, 21 bp, synthetic construct
  source 1..21: mol_type = other DNA
ctgaggatctctggttgtggt

SEQ ID NO: 48 - DNA, 22 bp, synthetic construct
  source 1..22: mol_type = other DNA
cgaactggacacacatacagtg

SEQ ID NO: 49 - skipped

SEQ ID NO: 50 - DNA, 18 bp, synthetic construct
  source 1..18: mol_type = other DNA
cgggagtccgcagtctta

SEQ ID NO: 51 - DNA, 20 bp, synthetic construct
  source 1..20: mol_type = other DNA
gcttgagggtctgaatcttg

SEQ ID NO: 52 - DNA, 20 bp, synthetic construct
  source 1..20: mol_type = other DNA
cttatgtttggggggaggtt

SEQ ID NO: 53 - DNA, 20 bp, synthetic construct
  source 1..20: mol_type = other DNA
tagccaagcaatcacggaga

SEQ ID NO: 54 - DNA, 21 bp, synthetic construct
  source 1..21: mol_type = other DNA
ctttgtcgttggttagctggt

SEQ ID NO: 55 - DNA, 20 bp, synthetic construct
  source 1..20: mol_type = other DNA
gacgccatcaacaccgagtt

SEQ ID NO: 56 - DNA, 22 bp, synthetic construct
  source 1..22: mol_type = other DNA
atcagcctaaccatacaactct

SEQ ID NO: 57 - DNA, 19 bp, synthetic construct
  source 1..19: mol_type = other DNA
caggaacaaattggcacaa

SEQ ID NO: 58 - DNA, 22 bp, synthetic construct
  source 1..22: mol_type = other DNA
gggttctttcatttgttttggt

SEQ ID NO: 59 - DNA, 23 bp, synthetic construct
  source 1..23: mol_type = other DNA
actccaaacagcttctcttctga

SEQ ID NO: 60 - DNA, 21 bp, synthetic construct
  source 1..21: mol_type = other DNA
cagatcacgtcatcgcacaac

SEQ ID NO: 61 - DNA, 21 bp, synthetic construct
  source 1..21: mol_type = other DNA
gagggccaagacgaagacatc

SEQ ID NO: 62 - DNA, 19 bp, synthetic construct
  source 1..19: mol_type = other DNA
gcccggatgtcgcttacag

SEQ ID NO: 63 - DNA, 21 bp, synthetic construct
  source 1..21: mol_type = other DNA
aaatgcagacgcagggtacag

SEQ ID NO: 64 - DNA, 20 bp, synthetic construct
  source 1..20: mol_type = other DNA
gactccaccgagatcgacca

SEQ ID NO: 65 - DNA, 21 bp, synthetic construct
  source 1..21: mol_type = other DNA
cttgtagcactctccgtaggc

SEQ ID NO: 66 - DNA, 22 bp, synthetic construct
  source 1..22: mol_type = other DNA
gcacagaatccttggtgaacag

SEQ ID NO: 67 - DNA, 22 bp, synthetic construct
  source 1..22: mol_type = other DNA
atggaaggtgaatgtttcagca

SEQ ID NO: 68 - DNA, 21 bp, synthetic construct
  source 1..21: mol_type = other DNA
cagtggaccccatcatggaga

SEQ ID NO: 69 - DNA, 21 bp, synthetic construct
  source 1..21: mol_type = other DNA
ggctttgctgccttgaacatc

SEQ ID NO: 70 - DNA, 21 bp, synthetic construct
  source 1..21: mol_type = other DNA
cttggggccaaaaaggtgttc

SEQ ID NO: 71 - DNA, 21 bp, synthetic construct
  source 1..21: mol_type = other DNA
gaggtagcggtcctcatacag

SEQ ID NO: 72 - DNA, 22 bp, synthetic construct
  source 1..22: mol_type = other DNA
aatgaggtgggcttaaagcaag

SEQ ID NO: 73 - DNA, 21 bp, synthetic construct
  source 1..21: mol_type = other DNA
agttccactccacagttcaga

SEQ ID NO: 74 - DNA, 20 bp, synthetic construct
  source 1..20: mol_type = other DNA
cggcccgtgtattgtctagc

SEQ ID NO: 75 - DNA, 21 bp, synthetic construct
  source 1..21: mol_type = other DNA
tagccagggtgtccaaatctg

SEQ ID NO: 76 - DNA, 21 bp, synthetic construct
  source 1..21: mol_type = other DNA
ggtggtgaatgaaacgctcag

SEQ ID NO: 77 - DNA, 21 bp, synthetic construct
  source 1..21: mol_type = other DNA
acccctttgggaatgaacatc

SEQ ID NO: 78 - DNA, 20 bp, synthetic construct
  source 1..20: mol_type = other DNA
cactggtcgccatctttgta

SEQ ID NO: 79 - DNA, 20 bp, synthetic construct
  source 1..20: mol_type = other DNA
agtgcatggaagagacctgc

SEQ ID NO: 80 - DNA, 20 bp, synthetic construct
  source 1..20: mol_type = other DNA
acagggctctgaacatgcac

SEQ ID NO: 81 - DNA, 20 bp, synthetic construct
  source 1..20: mol_type = other DNA
ggcattgaaaaactcccgta

SEQ ID NO: 82 - DNA, 21 bp, synthetic construct
  source 1..21: mol_type = other DNA
actacatcaacgaccgccagt

SEQ ID NO: 83 - DNA, 21 bp, synthetic construct
  source 1..21: mol_type = other DNA
atctgctcgatcatctgccag

(19) United States
(12) Patent Application Publication
(10) Pub. No.: US 2025/0340848 A1
(43) Pub. Date: Nov. 6, 2025

(54) METHODS FOR CHEMICAL REPROGRAMMING AND PLURIPOTENT STEM CELLS

(71) Applicants: Peking University, Beijing (CN); BeiCell Therapeutics Inc., Beijing (CN)

(72) Inventors: Hongkui Deng, Beijing (CN); Jinlin Wang, Beijing (CN); Jingyang Guan, Beijing (CN); Guan Wang, Beijing (CN); Shijia Liuyang, Beijing (CN); Yanglu Wang, Beijing (CN); Huanjing He, Beijing (CN); Jiacong Guo, Beijing (CN)

(73) Assignees: Peking University, Beijing (CN); BeiCell Therapeutics Inc., Beijing (CN)

(21) Appl. No.: 19/174,084

(22) Filed: Apr. 9, 2025

Related U.S. Application Data
(63) Continuation-in-part of application No. PCT/CN2023/113082, filed on Aug. 15, 2023.
Application No. PCT/CN2023/113082 is a continuation of application No. PCT/CN2022/124960, filed on Oct. 12, 2022.

(51) Int. Cl. C12N 5/074

(52) U.S. Cl. CPC C12N 5/0696; C12N 2500/02; C12N 2500/38; C12N 2501/15; C12N 2501/155; C12N 2501/385; C12N 2501/727; C12N 2501/73; C12N 2506/1384; C12N 2509/00

(57) ABSTRACT

Provided herein, in some aspects, are methods and compositions for cell conversion. The methods may convert a cell population comprising one cell type to another cell population comprising another cell type. The converted cell types may have increased cell differentiation potential. The converted cell types can comprise pluripotent stem cells. The compositions provided herein may comprise chemical reprogramming factors for converting cells. The compositions provided herein may comprise chemical reprogramming factors and cells. Also provided herein are reagents for carrying out the methods for converting cells. Additionally, provided herein are methods and compositions for using various cell types obtained by the methods and/or compositions provided herein.

### BACKGROUND

This application is a bypass continuation-in-part application of the national phase filing under 35 U.S.C. § 371 of International Application No. PCT/CN2023/113082, filed on Aug. 15, 2023, which claims priority to International Application No. PCT/CN2022/124960, filed on Oct. 12, 2022, the entire contents of which is/are hereby incorporated by reference herein.

### REFERENCE TO SEQUENCE LISTING

This application contains one or more sequence listings in computer readable form, which are incorporated herein by reference in their entireties. This application contains a sequence listing which has been submitted electronically in ST.26 (xml) format and is hereby incorporated by reference in its entirety. Said ST.26 copy, created on 9 Apr. 2025, is named “246047-30001 Seq Listing.xml” and is 72 kilobytes in size.

### FIELD

This application relates to compositions, kits, methods for cell dedifferentiation and reprogramming, their applications, and cells generated therefrom.

### BACKGROUND

There remains a significant need for cell resources for applications in basic research, therapeutics, agriculture, and food industry. Improved methods for industrial scale production of stem cells, as well as cells of various differentiation states and cell types, remain to be developed.

Cell identity can be established during development to acquire and maintain specialized cellular functions in somatic cells. Cellular reprogramming can manipulate cell identity, thereby enabling the generation of desired cell types that provide broad applications in disease modelling, drug discovery and regenerative medicine. Using cellular factors, including oocyte components and transcription factors, mouse and human somatic cells can be reprogrammed into pluripotent stem cells. Alternatively, chemical reprogramming can be utilized to induce somatic cells into pluripotent stem cells by simple exposure to small molecules. However, efficiency and kinetics of human chemical reprogramming system needs to be improved to robustly induce pluripotent stem cells from human somatic cells.

A substantial hurdle of human chemically induced pluripotent stem cells (hCiPSCs) generation, which take nearly two month to achieve a high yield, is its slow kinetics. Additionally, some somatic cell lines from different donors are relatively refractory to the current chemical conditions, corresponding to their lower reprogramming efficiency. The inventors of the present invention discovered that for the refractory cell lines, the major rate-limiting event occurs at the early stage of reprogramming, in which a prolonged duration was required to erase the somatic cell identity. These findings strongly imply the existence of additional intrinsic barriers that preserve somatic cell fate, thereby impeding the chemical reprogramming of human somatic cells. Consequently, there is a pressing need for a fast and more robust chemical reprogramming system to produce hCiPSCs, which would greatly promote the application of this regenerative medicine approach for timely and personalized clinical applications.

### SUMMARY

Provided herein, in some aspects, are methods and compositions for cell conversion using chemical reprogramming factors. The methods and compositions may generate cells with enhanced differentiation potentials. The methods and compositions may convert a cell population comprising at least a cell type to another cell population comprising another cell type that exhibits increased differentiation potentials. Various cells generated by the methods and/or compositions may comprise pluripotent stem cells or other intermediate cells with increased potential to become pluripotent stem cells relative to the cell that is not converted. These intermediate cells may comprise epithelial-like cells, intermediate plastic state cells, progenies thereof, or derivatives thereof.

The methods and compositions may bypass using genetic modification to generate cells with enhanced differentiation potentials. In some cases, not utilizing genetic modification to generate stem cells or cells of various differentiation states may decrease the negative impact of the genetic modification. Such negative impact may comprise accidental induction of mutations in the stem cells. In some cases, such negative impacts may also be generated by exogenous nucleic acid molecules or sequences used in the genetic modification of the cells. Cells comprising the accidental mutation(s) may have various undesirable properties, including but limited to, enhanced or unregulated cell proliferation potentials (that can lead to neoplastic diseases such as cancers), unforeseen differentiation properties, and/or undesirable cell senescence stages. Utilizing chemical reprogramming factors, the methods and compositions provided herein can eliminate these negative impacts induced by the genetic modification. Furthermore, wherein when genetic modification needs to be utilized in downstream purposes after generation of converted cells, the methods and compositions provided herein can reduce the negative impacts described herein.

Using the chemical programming factors described herein, the methods and compositions can also increase the scalability for producing stem cells as well as cells of various differentiation states. These methods and compositions can generate stem cells or cells of various differentiation states within a shorter period of time, compared to existing methods. Hence, the methods and compositions can satisfy the need for cell resources for applications in basic research, therapeutics, agriculture, and food industry.

Provided herein, in some aspects, are methods for producing pluripotent stem cells. In an aspect, a method for producing pluripotent stem cells comprises: (a) obtaining epithelial-like cells that express LIN28A; (b) converting the epithelial-like cells or progenies thereof into intermediate plastic state cells that express LIN28A and SALL4, and one or more of MSX2, NMYC, SDC1, WNT4, FGF19, or TOP2A; and (c) converting the intermediate plastic state cells or progenies thereof into pluripotent stem cells.

In some embodiments, the converting the epithelial-like cells or progenies thereof comprises contacting the epithelial-like cells with a composition comprising a glycogen synthase kinase 3 (GSK-3) inhibitor, a transforming growth factor-beta (TGFβ) receptor inhibitor, and a c-Jun kinase inhibitor. In some embodiments, the composition further comprises a CBP/p300 bromodomain inhibitor or an adenosine kinase inhibitor. In some embodiments, the composition further comprises an S-adenosyl-L-homocysteine (SAH) hydrolase inhibitor or an adenosine kinase inhibitor.

Provided herein, in some aspects, are methods for producing pluripotent stem cells. In an aspect, a method for producing pluripotent stem cells, comprising: (a) obtaining epithelial-like cells that express LIN28A; (b) contacting the epithelial-like cells or progenies thereof with: (i) a CBP/p300 bromodomain inhibitor or an adenosine kinase inhibitor; (ii) a glycogen synthase kinase 3 (GSK-3); (iii) a transforming growth factor-beta (TGFβ) receptor inhibitor; and (iv) a c-Jun kinase inhibitor, thereby converting the epithelial-like cells or the progenies thereof into intermediate plastic state cells that express LIN28A and SALL4, and one or more of MSX2, NMYC, WNT4, FGF19, or TOP2A; and (c) converting the intermediate plastic state cells or progenies thereof into pluripotent stem cells.

In some embodiments, the epithelial-like cells express one or more of KRT18, KRT19, WT1, NMYC, WNT2B, PAX8, SMAD3, GLI3, or TBX2. In some embodiments, the epithelial-like cells express one or more of NMYC, WNT2B, PAX8, SMAD3, or GLI3. In some embodiments, the epithelial-like cells further express one or more of KRT18, KRT19, WT1, or TBX2. In some embodiments, the intermediate plastic state cells express one or more of MSX1, HOXB9, WT1, GATA2, HMGA2, LEF1, FGF9, HOXA9, HOXA1, PTCH1, HOXA5, CCND2, SDC1, TBX3, BMP4, or IGF2. In some embodiments, the intermediate plastic state cells express one or more of MSX1, HOXB9, WT1, GATA2, HMGA2, or LEF1. In some embodiments, the intermediate plastic state cells further express one or more of FGF9, HOXA9, HOXA1, PTCH1, HOXA5, CCND2, SDC1, TBX3, BMP4, or IGF2. In some embodiments, the pluripotent stem cells express one or more of OCT4, SOX2, or NANOG. In some embodiments, the pluripotent cells express one or more of FGF4, ZFP57, REX1, DPPA4, TDGF1, TRA-1-60, TRA-1-81, SSEA4, KLF4, KLF17, DPPA3, DPPA5, DNMT3L, REX1, or UTF1. In some embodiments, the pluripotent cells express one or more of FGF4, ZFP57, DPPA5, or REX1. In some embodiments, the pluripotent cells further express one or more of DPPA4, TDGF1, TRA-1-60, TRA-1-81, SSEA4, KLF4, KLF17, DPPA3, DNMT3L, or UTF1.

In some embodiments, the method further comprises treating a population of somatic cells, thereby converting at least a subset of the somatic cells in the population into the epithelial-like cells. In some embodiments, the somatic cells comprise primary human adult adipose derived mesenchymal stromal cells (hADSCs). In some embodiments, the somatic cells comprise fibroblasts. In some embodiments, the method converts the somatic cells into the pluripotent stem cells within less about 50 days. In some embodiments, the method converts the somatic cells into the pluripotent stem cells within at most about 32 days. In some embodiments, the method converts the somatic cells into the pluripotent stem cells within at most about 24 days. In some embodiments, the method results in generation of one pluripotent stem cell per at most 1,000 somatic cells in the population of somatic cells. In some embodiments, the method results in generation of one pluripotent stem cell per at most 200 somatic cells in the population of somatic cells. In some embodiments, the method results in generation of one pluripotent stem cell per at most 50 somatic cells in the population of somatic cells. In some embodiments, the method further comprises plating the somatic cells at a density of at most about 1×10\hat{ }6 cells per square centimeter (cm\hat{ }2) of cell growth area. In some embodiments, the somatic cells are plated at a density of at most about 5×10\hat{ }5 cells per cm\hat{ }2 of cell growth area. In some embodiments, the somatic cells are plated at a density of at most about 2.5×10\hat{ }5 cells per cm\hat{ }2 of cell growth area.

Provided herein, in some aspects, are methods for producing pluripotent stem cells. In an aspect, method for producing pluripotent stem cells, comprising: (a) obtaining a first cell population that comprises epithelial-like cells that express LIN28A; (b) contacting the first cell population with a second composition comprising: (i) a glycogen synthase kinase 3 (GSK-3); (ii) a transforming growth factor-beta (TGFβ) receptor inhibitor; and (iii) a c-Jun kinase inhibitor, thereby obtaining a second cell population; and (c) contacting the second cell population with a third composition comprising: (i) a MEK inhibitor; (ii) a B-Raf inhibitor; and (iii) a histone deacetylase inhibitor, thereby obtaining a third cell population comprising pluripotent stem cells.

In some embodiments, the glycogen synthase kinase 3 (GSK-3) comprises CHIR99021 or CHIR98014. In some embodiments, the glycogen synthase kinase 3 (GSK-3) comprises CHIR99021. In some embodiments, CHIR99021 is present at about 0.5 micromolar (μM) to about 50 μM within the second composition. In some embodiments, CHIR99021 is present at about 1 μM to about 25 μM within the second composition. In some embodiments, CHIR99021 is present at about 2 μM to about 12.5 μM within the second composition. In some embodiments, CHIR99021 is present at about 5 μM within the second composition. In some embodiments, the transforming growth factor-beta (TGFβ) receptor inhibitor is an ALK5 inhibitor. In some embodiments, the transforming growth factor-beta (TGFβ) receptor inhibitor comprises E-616452, A 83-01, SB431542, SB 505124, GW 788388, or SB 525334, optionally wherein the transforming growth factor-beta (TGFβ) receptor inhibitor comprises E-616452. In some embodiments, E-616452 is present at about 1 μM to about 100 μM within the second composition. In some embodiments, E-616452 is present at about 2 μM to about 50 μM within the second composition. In some embodiments, E-616452 is present at about 4 μM to about 25 μM within the second composition. In some embodiments, E-616452 is present at about 10 μM within the second composition. In some embodiments, the c-Jun kinase inhibitor comprises JNKIN8, JNKIN7, JNKIN5, or JNKIN12. In some embodiments, the c-Jun kinase inhibitor comprises JNKIN8. In some embodiments, JNKIN8 is present at about 0.05 μM to about 5 μM within the second composition. In some embodiments, JNKIN8 is present at about 0.1 μM to about 2.5 μM within the second composition. In some embodiments, JNKIN8 is present at about 0.2 μM to about 1.25 μM within the second composition. In some embodiments, JNKIN8 is present at about 0.5 μM within the second composition. In some embodiments, the MEK inhibitor comprises PD0325901, AZD8330, or TAK-733. In some embodiments, the MEK inhibitor comprises PD0325901. In some embodiments, PD0325901 is present at about 0.1 μM to about 10 μM with the third composition. In some embodiments, PD0325901 is present at about 0.2 μM to about 5 μM with the third composition. In some embodiments, PD0325901 is present at about 0.4 μM to about 2.5 μM with the third composition. In some embodiments, PD0325901 is present at about 1 μM with the third composition. In some embodiments, the B-Raf inhibitor comprises SB590885, Vemurafenib, RAF265, or PLX4720. In some embodiments, the B-Raf inhibitor comprises SB590885. In some embodiments, SB590885 is present at about 0.05 μM to about 5 μM with the third composition. In some embodiments, SB590885 is present at about 0.1 μM to about 2.5 μM with the third composition. In some embodiments, SB590885 is present at about 0.2 μM to about 1.25 μM with the third composition. In some embodiments, SB590885 is present at about 0.5 μM with the third composition. In some embodiments, the histone deacetylase inhibitor comprises valproic acid (VPA), LMK235, MS275, or HDACi I. In some embodiments, the histone deacetylase inhibitor comprises VPA. In some embodiments, VPA is present at about 0.1 millimolar (mM) to about 10 mM within the third composition. In some embodiments, VPA is present at about 0.2 mM to about 5 mM within the third composition. In some embodiments, VPA is present at about 0.4 mM to about 2.5 mM within the third composition. In some embodiments, VPA is present at about 1 mM within the third composition. In some embodiments, the second composition further comprises a CBP/p300 bromodomain inhibitor or an adenosine kinase inhibitor. In some embodiments, the second composition further comprises: (a) a retinoic acid receptor (RAR) agonist; (b) a CBP/p300 bromodomain inhibitor; and (c) a S-adenosyl-L-homocysteine (SAH) hydrolase inhibitor or an adenosine kinase inhibitor. In some embodiments, the retinoic acid receptor (RAR) agonist comprises TTNPB, Ch55, or AM580. In some embodiments, the retinoic acid receptor (RAR) agonist comprises TTNPB. In some embodiments, TTNPB is present at about 0.2 μM to about 20 μM within the composition. In some embodiments, TTNPB is present at about 0.4 μM to about 10 μM within the composition. In some embodiments, TTNPB is present at about 0.8 μM to about 5 μM within the composition. In some embodiments, TTNPB is present at about 2 μM within the second composition. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.02 μM to about 2 μM within the second composition. In some embodiments, DZNep is present at about 0.04 μM to about 1 μM within the second composition. In some embodiments, DZNep is present at about 0.08 μM to about 0.5 μM within the second composition. In some embodiments, DZNep is present at about 0.2 μM with the second composition. In some embodiments, the CBP/p300 bromodomain inhibitors comprises SGC-CBP30, I-CBP112, or GNE27. In some embodiments, the CBP/p300 bromodomain inhibitor comprises SGC-CBP30. In some embodiments, SGC-CBP30 is present at about 0.2 μM to about 20 μM within the second composition. In some embodiments, SGC-CBP30 is present at about 0.4 μM to about 10 μM within the second composition. In some embodiments, SGC-CBP30 is present at about 0.8 μM to about 5 μM within the second composition. In some embodiments, SGC-CBP30 is present at about 2 μM within the second composition. In some embodiments, the adenosine kinase inhibitor comprises 5-Iodotubercidin (5-ITU) or ABT 702. In some embodiments, the adenosine kinase inhibitor 5-ITU. In some embodiments, 5-ITU is present at about 0.05 μM to about 5 μM within the composition. In some embodiments, 5-ITU is present at about 0.1 micromolar μM to about 2.5 μM within the composition. In some embodiments, 5-ITU is present at about 0.2 μM to about 1 μM within the composition. In some embodiments, the 5-ITU is present at about 0.5 μM within the composition.

In some embodiments, the method comprises culturing the first cell population in the second composition for at most about 20 days. In some embodiments, the method comprises culturing the first cell population in the second composition for at most about 16 days. In some embodiments, the method comprises culturing the first cell population in the second composition from about 4 days to 16 days. In some embodiments, the method further comprises removing the second composition from the second cell population. In some embodiments, the method comprises culturing the second cell population in the third composition for at most about 20 days. In some embodiments, the method comprises culturing the second cell population in the third composition for at most about 12 days. In some embodiments, the method comprises culturing the second cell population in the third composition from about 4 days to 12 days. In some embodiments, the epithelial-like cells comprise or progenies thereof a genetic modification. In some embodiments, the pluripotent stem cells or progenies thereof comprise the genetic modification. In some embodiments, the genetic modification comprises an exogenous nucleic acid sequence. In some embodiments, the exogenous nucleic acid sequence encodes a polypeptide. In some embodiments, the exogenous nucleic acid sequence comprises a sequence of a non-coding nucleic acid molecule. In some embodiments, the genetic modification comprises alteration of a genomic sequence. In some embodiments, the genetic modification reduces immunogenicity of the pluripotent stem cells or the progenies thereof.

Provided herein, in some aspects, are methods for reprogramming epithelial-like cells that express LIN28A. In an aspect, a method for reprogramming epithelial-like cells that express LIN28A comprises contacting a population of cells comprising the epithelial-like cells or progenies thereof with a composition comprising: (a) a S-adenosyl-L-homocysteine (SAH) hydrolase inhibitor or an adenosine kinase inhibitor; (b) a glycogen synthase kinase 3 (GSK-3); (c) a transforming growth factor-beta (TGFβ) receptor inhibitor; and (d) a c-Jun kinase inhibitor.

In some embodiments, during the contacting, the population of cells are incubated with about 21% atmospheric oxygen. In some embodiments, the composition comprises the adenosine kinase inhibitor. In some embodiments, the adenosine kinase inhibitor comprises 5-Iodotubercidin (5-ITU) or ABT 702. In some embodiments, the adenosine kinase inhibitor 5-ITU. In some embodiments, 5-ITU is present at about 0.05 micromolar (μM) to about 5 μM within the composition. In some embodiments, 5-ITU is present at about 0.1 μM to about 2.5 μM within the composition. In some embodiments, 5-ITU is present at about 0.2 μM to about 1 μM within the composition. In some embodiments, 5-ITU is present at about 0.5 μM within the composition. In some embodiments, the composition comprises the S-adenosyl-L-homocysteine (SAH) hydrolase inhibitor. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.02 μM to about 2 μM within the second composition. In some embodiments, DZNep is present at about 0.04 μM to about 1 μM within the second composition. In some embodiments, DZNep is present at about 0.08 μM to about 0.5 μM within the second composition. In some embodiments, DZNep is present at about 0.2 micromolar (μM) within the composition. In some embodiments, the glycogen synthase kinase 3 (GSK-3) comprises CHIR99021 or CHIR98014. In some embodiments, the glycogen synthase kinase 3 (GSK-3) comprises CHIR99021. In some embodiments, the glycogen synthase kinase 3 (GSK-3) comprises CHIR99021. In some embodiments, CHIR99021 is present at about 0.5 micromolar (μM) to about 50 μM within the composition. In some embodiments, CHIR99021 is present at about 1 μM to about 25 μM within the composition. In some embodiments, CHIR99021 is present at about 2 μM to about 12.5 μM within the composition. In some embodiments, CHIR99021 is present at about 5 μM within the composition. In some embodiments, the transforming growth factor-beta (TGFβ) receptor inhibitor is an ALK5 inhibitor. In some embodiments, the transforming growth factor-beta (TGFβ) receptor inhibitor comprises E-616452, A 83-01, SB431542, SB 505124, GW 788388, or SB 525334, optionally wherein the transforming growth factor-beta (TGFβ) receptor inhibitor comprises E-616452. In some embodiments, E-616452 is present at about 1 micromolar (μM) to about 100 μM within the composition. In some embodiments, E-616452 is present at about 2 μM to about 50 μM within the composition. In some embodiments, E-616452 is present at about 4 μM to about 25 μM within the composition. In some embodiments, E-616452 is present at about 10 μM within the composition. In some embodiments, the c-Jun kinase inhibitor comprises JNKIN8, JNKIN7, JNKIN5, or JNKIN12. In some embodiments, the c-Jun kinase inhibitor comprises JNKIN8. In some embodiments, JNKIN8 is present at about 0.05 μM to about 50 μM within the composition. In some embodiments, JNKIN8 is present at about 0.1 μM to about 2.5 μM within the composition. In some embodiments, JNKIN8 is present at about 0.2 μM to about 1.25 μM within the composition. In some embodiments, JNKIN8 is present at about 0.5 μM within the composition. In some embodiments, the composition further comprises a CBP/p300 bromodomain inhibitor. In some embodiments, the CBP/p300 bromodomain inhibitors comprises SGC-CBP30, I-CBP112, GNE272, or GNE409. In some embodiments, the CBP/p300 bromodomain inhibitor comprises SGC-CBP30. In some embodiments, SGC-CBP30 is present at about 0.2 μM to about 20 μM within the composition. In some embodiments, SGC-CBP30 is present at about 0.4 μM to about 10 μM within the composition. In some embodiments, SGC-CBP30 is present at about 0.8 μM to about 5 μM within the composition. In some embodiments, SGC-CBP30 is present at about 2 μM within the composition. In some embodiments, the composition further comprises one or more of a SET domain containing 2 (SETD2) inhibitor, an Akt inhibitor, or a casein kinase 2 inhibitor. In some embodiments, the SET domain containing 2 (SETD2) inhibitor comprises SETD2-IN-1, EPZ-719, or MMSET-IN-1. In some embodiments, the SET domain containing 2 (SETD2) inhibitor comprises the SETD2-IN-1. In some embodiments, SETD2-IN-1 is present at about 0.05 μM to about 5 μM within the composition. In some embodiments, SETD2-IN-1 is present at about 0.1 μM to about 2.5 μM within the composition. In some embodiments, SETD2-IN-1 is present at about 0.2 μM to about 1.25 μM within the composition. In some embodiments, SETD2-IN-1 is present at about 0.4 μM within the composition. In some embodiments, the Akt inhibitor comprises AKT Kinase Inhibitor. In some embodiments, AKT Kinase Inhibitor is present at about 0.1 μM to about 10 μM within the composition. In some embodiments, AKT Kinase Inhibitor is present at about 0.2 μM to about 5 μM within the composition. In some embodiments, AKT Kinase Inhibitor is present at about 0.4 μM to about 2.5 μM within the composition. In some embodiments, AKT Kinase Inhibitor is present at about 1 μM within the composition. In some embodiments, the casein kinase 2 inhibitor comprises CX-4945, TPP 22, or Ellagic acid. In some embodiments, the casein kinase 2 inhibitor comprises the CX-4945. In some embodiments, CX-4945 is present at about 0.08 μM to about 8 μM within the composition. In some embodiments, CX-4945 is present at about 0.16 μM to about 4 μM within the composition. In some embodiments, CX-4945 is present at about 0.32 μM to about 2 μM within the composition. In some embodiments, CX-4945 is present at about 0.8 μM within the composition. In some embodiments, the composition further comprises one or more of a Menin-MLL interaction inhibitor, an agonist for the G protein-coupled receptor Smoothened, a ROCK inhibitor, or a BMP receptor/AMPK inhibitor. In some embodiments, the Menin-MLL interaction inhibitor comprises VTP50469, MI3454, or WDR5-IN-4. In some embodiments, the Menin-MLL interaction inhibitor comprises the VTP50469. In some embodiments, VTP50469 is present at about 0.05 μM to about 5 μM within the composition. In some embodiments, VTP50469 is present at about 0.1 μM to about 2.5 μM within the composition. In some embodiments, VTP50469 is present at about 0.2 μM to about 1.25 μM within the composition. In some embodiments, VTP50469 is present at about 0.5 μM within the composition. In some embodiments, the agonist for the G protein-coupled receptor Smoothened comprises SAG, Purmorphamine, Hh-Ag1.5, or human SHH. In some embodiments, the agonist for the G protein-coupled receptor Smoothened comprises SAG. In some embodiments, SAG is present at about 0.05 μM to about 5 μM within the composition. In some embodiments, SAG is present at about 0.1 μM to about 2.5 μM within the composition. In some embodiments, SAG is present at about 0.2 μM to about 1.25 μM within the composition. In some embodiments, SAG is present at about 0.5 μM within the composition. In some embodiments, the ROCK inhibitor comprises Y-27632 or thiazovivin. In some embodiments, the ROCK inhibitor comprises Y-27632. In some embodiments, Y-27632 is present at about 1 μM to about 100 μM within the composition. In some embodiments, Y-27632 is present at about 2 μM to about 50 μM within the composition. In some embodiments, Y-27632 is present at about 4 μM to about 25 μM within the composition. In some embodiments, Y-27632 is present at about 10 μM within the composition. In some embodiments, the BMP receptor/AMPK inhibitor comprises Dorsomorphin. In some embodiments, Dorsomorphin is present at about 0.05 μM to about 5 μM within the composition. In some embodiments, Dorsomorphin is present at about 0.1 μM to about 2.5 μM within the composition. In some embodiments, Dorsomorphin is present at about 0.2 μM to about 1.25 μM within the composition. In some embodiments, Dorsomorphin is present at about 0.5 μM within the composition. In some embodiments, the composition further comprises a retinoic acid receptor (RAR) agonist. In some embodiments, the retinoic acid receptor (RAR) agonist comprises TTNPB, Ch55, or AM580. In some embodiments, the retinoic acid receptor (RAR) agonist comprises TTNPB. In some embodiments, TTNPB is present at about 0.2 μM to about 20 μM within the composition. In some embodiments, TTNPB is present at about 0.4 μM to about 10 μM within the composition. In some embodiments, TTNPB is present at about 0.8 μM to about 5 μM within the composition. In some embodiments, TTNPB is present at about 2 μM within the composition. In some embodiments, the composition further comprises one or more of a Dot1L inhibitor, a Jak1/Jak2 inhibitor, or a p38 MAPK inhibitor. In some embodiments, the Dot1L inhibitor comprises EPZ004777 or EPZ5676. In some embodiments, the Dot1L inhibitor comprises EPZ5676. In some embodiments, EPZ5676 is present at about 0.2 μM to about 20 μM within the composition. In some embodiments, EPZ5676 is present at about 0.4 μM to about 10 μM within the composition. In some embodiments, EPZ5676 is present at about 0.8 μM to about 5 μM within the composition. In some embodiments, EPZ5676 is present at about 2 μM within the composition. In some embodiments, the Jak1/Jak2 inhibitor comprises Ruxolitinib, Tofacitinib, AZD1480, Baricitinib, S-Ruxolitinib, or Fedratinib. In some embodiments, the Jak1/Jak2 inhibitor comprises Ruxolitinib. In some embodiments, Ruxolitinib is present at about 0.1 μM to about 10 μM within the composition. In some embodiments, Ruxolitinib is present at about 0.2 μM to about 5 μM within the composition. In some embodiments, Ruxolitinib is present at about 0.4 μM to about 2.5 μM within the composition. In some embodiments, Ruxolitinib is present at about 1 μM within the composition. In some embodiments, the p38 MAPK inhibitor comprises BIRB796, SB203580, or SB202190. In some embodiments, the p38 MAPK inhibitor comprises BIRB796. In some embodiments, BIRB796 is present at about 0.2 μM to about 20 μM within the composition. In some embodiments, BIRB796 is present at about 0.4 μM to about 10 μM within the composition. In some embodiments, BIRB796 is present at about 0.8 μM to about 5 μM within the composition. In some embodiments, BIRB796 is present at about 2 μM within the composition.

In some embodiments, the method comprises culturing the population of cells in the composition for at most about 20 days. In some embodiments, the method comprises culturing the population of cells in the composition for at most about 16 days. In some embodiments, the method comprises culturing the population of cells in the composition from about 4 days to about 16 days.

In some embodiments, the method leads to conversion of the epithelial-like cells into intermediate plastic state cells that express LIN28A and SALL4, and one or more of MSX2, NMYC, SDC1, WNT4, FGF19, or TOP2A. In some embodiments, the intermediate plastic state cells express one or more of MSX1, HOXB9, WT1, GATA2, HMGA2, LEF1, FGF9, HOXA9, HOXA1, PTCH1, HOXA5, CCND2, SDC1, TBX3, BMP4, or IGF2. In some embodiments, the intermediate plastic state cells express one or more of MSX1, HOXB9, WT1, GATA2, HMGA2, or LEF1. In some embodiments, the intermediate plastic state cells further express one or more of FGF9, HOXA9, HOXA1, PTCH1, HOXA5, CCND2, SDC1, TBX3, BMP4, or IGF2.

In some embodiments, the intermediate plastic state cells or progenies thereof comprise a genetic modification. In some embodiments, the genetic modification comprises an exogenous nucleic acid sequence. In some embodiments, the exogenous nucleic acid sequence encodes a polypeptide. In some embodiments, the exogenous nucleic acid sequence comprises a sequence of a non-coding nucleic acid molecule. In some embodiments, the genetic modification comprises alteration of a genomic sequence. In some embodiments, the genetic modification reduces immunogenicity of the intermediate plastic state cells or the progenies thereof.

Provided herein, in some aspects, are methods for reprogramming somatic cells. In an aspect, a method for reprogramming somatic cells comprises contacting a population of cells comprising the somatic cells with a composition comprising: one or more of (a) a glycogen synthase kinase 3 (GSK-3); (b) a transforming growth factor-beta (TGFβ) receptor inhibitor; (c) a retinoic acid receptor (RAR) agonist; and (d) an Akt inhibitor or a SET domain containing 2 (SETD2) inhibitor.

In some embodiments, the somatic cells comprise primary human adult adipose derived mesenchymal stromal cells (hADSCs). In some embodiments, the somatic cells comprise fibroblasts. In some embodiments, during the contacting, the population of cells are incubated with at most about 21% atmospheric oxygen. In some embodiments, during the contacting, the population of cells are incubated with at most about 5% atmospheric oxygen. In some embodiments, the glycogen synthase kinase 3 (GSK-3) comprises CHIR99021. In some embodiments, CHIR99021 is present at about 0.5 μM to about 50 μM within the composition. In some embodiments, CHIR99021 is present at about 1 μM to about 25 μM within the composition. In some embodiments, CHIR99021 is present at about 2 μM to about 12.5 μM within the composition. In some embodiments, CHIR99021 is present at about 5 μM within the composition. In some embodiments, the transforming growth factor-beta (TGFβ) receptor inhibitor is an ALK5 inhibitor. In some embodiments, the transforming growth factor-beta (TGFβ) receptor inhibitor comprises E-616452, A 83-01, SB431542, SB 505124, GW 788388, or SB 525334, optionally wherein the transforming growth factor-beta (TGFβ) receptor inhibitor comprises E-616452. In some embodiments, E-616452 is present at about 1 μM to about 100 μM within the composition. In some embodiments, E-616452 is present at about 2 μM to about 50 μM within the composition. In some embodiments, E-616452 is present at about 4 μM to about 25 μM within the composition. In some embodiments, E-616452 is present at about 10 μM within the composition. In some embodiments, the retinoic acid receptor (RAR) agonist comprises TTNPB, Ch55, or AM580. In some embodiments, the retinoic acid receptor (RAR) agonist comprises TTNPB. In some embodiments, TTNPB is present at about 0.2 μM to about 20 μM within the composition. In some embodiments, TTNPB is present at about 0.4 μM to about 10 μM within the composition. In some embodiments, TTNPB is present at about 0.8 μM to about 5 μM within the composition. In some embodiments, TTNPB is present at about 2 μM within the composition. In some embodiments, the composition comprises the Akt inhibitor. In some embodiments, the Akt inhibitor comprises AKT Kinase Inhibitor. In some embodiments, AKT Kinase Inhibitor is present at about 0.1 μM to about 10 μM within the composition. In some embodiments, AKT Kinase Inhibitor is present at about 0.2 μM to about 5 μM within the composition. In some embodiments, AKT Kinase Inhibitor is present at about 0.4 μM to about 2.5 μM within the composition. In some embodiments, AKT Kinase Inhibitor is present at about 1 μM within the composition. In some embodiments, the composition comprises the SET domain containing 2 (SETD2) inhibitor. In some embodiments, the SET domain containing 2 (SETD2) inhibitor comprises SETD2-IN-1, EPZ-719, or MMSET-IN-1. In some embodiments, the SET domain containing 2 (SETD2) inhibitor comprises SETD2-IN-1. In some embodiments, SETD2-IN-1 is present at about 0.05 μM to about 5 μM within the composition. In some embodiments, SETD2-IN-1 is present at about 0.1 μM to about 2.5 μM within the composition. In some embodiments, SETD2-IN-1 is present at about 0.2 μM to about 1.25 μM within the composition. In some embodiments, SETD2-IN-1 is present at about 0.4 μM within the composition. In some embodiments, the composition is serum free. In some embodiments, the composition is feeder-cell free. In some embodiments, the composition further comprises an agonist for the G protein-coupled receptor Smoothened or a Menin-MLL interaction inhibitor. In some embodiments, the agonist for the G protein-coupled receptor Smoothened comprises SAG, Purmorphamine, Hh-Ag1.5, or human SHH. In some embodiments, the agonist for the G protein-coupled receptor Smoothened comprises SAG. In some embodiments, SAG is present at about 0.05 μM to about 5 μM within the composition. In some embodiments, SAG is present at about 0.1 μM to about 2.5 μM within the composition. In some embodiments, SAG is present at about 0.2 μM to about 1.25 μM within the composition. In some embodiments, SAG is present at about 0.5 μM within the composition. In some embodiments, the Menin-MLL interaction inhibitor comprises VTP50469, MI3454, or WDR5-IN-4. In some embodiments, the Menin-MLL interaction inhibitor comprises VTP50469. In some embodiments, VTP50469 is present at about 0.05 μM to about 5 μM within the composition. In some embodiments, VTP50469 is present at about 0.1 μM to about 2.5 μM within the composition. In some embodiments, VTP50469 is present at about 0.2 μM to about 1.25 μM within the composition. In some embodiments, VTP50469 is present at about 0.5 μM within the composition. In some embodiments, the composition further comprises a Jak1/Jak2 inhibitor, an S-adenosyl-L-homocysteine (SAH) hydrolase inhibitor, or a Dot1L inhibitor. In some embodiments, the Jak1/Jak2 inhibitor comprises Ruxolitinib, Tofacitinib, AZD1480, Baricitinib, S-Ruxolitinib, or Fedratinib. In some embodiments, the Jak1/Jak2 inhibitor comprises Ruxolitinib. In some embodiments, Ruxolitinib is present at about 0.1 μM to about 10 μM within the composition. In some embodiments, Ruxolitinib is present at about 0.2 μM to about 5 μM within the composition. In some embodiments, Ruxolitinib is present at about 0.4 μM to about 2.5 μM within the composition. In some embodiments, Ruxolitinib is present at about 1 μM within the composition. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.02 μM to about 2 μM within the second composition. In some embodiments, DZNep is present at about 0.04 μM to about 1 μM within the second composition. In some embodiments, DZNep is present at about 0.08 μM to about 0.5 μM within the second composition. In some embodiments, DZNep is present at about 0.2 μM with the second composition. In some embodiments, the Dot1L inhibitor comprises EPZ004777 or EPZ5676. In some embodiments, the Dot1L inhibitor comprises the EPZ5676. In some embodiments, EPZ5676 is present at about 0.2 μM to about 20 μM within the composition. In some embodiments, EPZ5676 is present at about 0.4 μM to about 10 μM within the composition. In some embodiments, EPZ5676 is present at about 0.8 μM to about 5 μM within the composition. In some embodiments, EPZ5676 is present at about 2 μM within the composition.

In some embodiments, the method comprises culturing the population of cells in the composition for at most about 20 days. In some embodiments, the method comprises culturing the population of cells in the composition for at most about 12 days. In some embodiments, the method comprises culturing the population of cells in the composition from about 4 days to about 12 days. In some embodiments, the method leads to conversion of the somatic cells into epithelial-like cells that express LIN28A. In some embodiments, the epithelial-like cells express one or more of KRT18, KRT19, WT1, NMYC, WNT2B, PAX8, SMAD3, GLI3, or TBX2. In some embodiments, the epithelial-like cells express one or more of NMYC, WNT2B, PAX8, SMAD3, or GLI3. In some embodiments, the epithelial-like cells further express one or more of KRT18, KRT19, WT1, or TBX2. In some embodiments, the epithelial-like cells or progenies thereof comprise a genetic modification. In some embodiments, the genetic modification comprises an exogenous nucleic acid sequence. In some embodiments, the exogenous nucleic acid sequence encodes a polypeptide. In some embodiments, the exogenous nucleic acid sequence comprises a sequence of a non-coding nucleic acid molecule. In some embodiments, the genetic modification comprises alteration of a genomic sequence. In some embodiments, the genetic modification reduces immunogenicity of the epithelial-like cells or the progenies thereof.

In some aspects, the method further includes the step of exposing the somatic cells to one or more histone acetyltransferase inhibitors under conditions that enable the somatic cells to form a cell population with increased differentiation potential compared to the somatic cells, and the step of further dedifferentiating the cell population into pluripotent stem cells. In some embodiments, the histone acetyltransferase inhibitor used in the method disclosed herein is a KAT3A/KAT3B (also known as CBP/P300) inhibitor. In further embodiments, the KAT3A/KAT3B inhibitor comprises one or more of A485, ICBP112, GEN049, CBP/P300 IN 12, or SGC/CBP30. In some further embodiments, the KAT3A/KAT3B inhibitor comprises A485. In another embodiments, the histone acetyltransferase inhibitor used in the method disclosed herein is a KAT6A (also known as MOZ) inhibitor, the KAT6A inhibitor comprises one or more of CBP/P300 IN 8, WM8014, or WM1119. In some embodiments, the KAT6A inhibitor comprises WM8014. In some other embodiments, the histone acetyltransferase inhibitor used in the method disclosed herein is a combination of a KAT3A/KAT3B inhibitor and a KAT6A inhibitor. The combination can be selected from the combinations of A485 and WM8014, ICBP112 and WM8014, GEN049 and WM8014, CBP/P300 IN 12 and WM8014, SGC/CBP30 and WM8014, A485 and WM1119, ICBP112 and WM1119, GEN049 and WM1119, CBP/P300 IN 12 and WM1119, SGC/CBP30 and WM1119, A485 and CBP/P300 IN 8, ICBP112 and CBP/P300 IN 8, GEN049 and CBP/P300 IN 8, CBP/P300 IN 12 and CBP/P300 IN 8, or SGC/CBP30 and CBP/P300 IN 8. In certain embodiments, the combination is of A485 and WM8014.

In some embodiments, the concentration of the histone acetyltransferase inhibitor used in the method disclosed herein is 0.01 to 10 μM. In further embodiments, the concentration of the histone acetyltransferase inhibitor used is 0.05 to 5 μM. In some further embodiment, the concentration of the histone acetyltransferase inhibitor used is 0.1 to 5 μM. In another embodiments, the concentration of the histone acetyltransferase inhibitor used is 0.1 to 2 μM. In some embodiments, the chemical reprogramming method disclosed herein uses A485 at a concentration of 0.1 to 2 μM or WM8014 at a concentration of 0.1 to 5 μM or a combination of both. In further embodiments, the chemical reprogramming method disclosed herein uses A485 at a concentration of 0.5 to 1 μM or WM8014 at a concentration of 0.5 to 1 μM or a combination of both. In some further embodiments, the chemical reprogramming method disclosed herein uses A485 at a concentration of 0.5 μM or WM8014 at a concentration of 1 μM or a combination of both.

In some embodiments, the chemical reprogramming method disclosed herein exposes the somatic cells to the histone acetyltransferase inhibitor for less than 8 days. In further embodiments, the chemical reprogramming method disclosed herein exposes the somatic cells to the histone acetyltransferase inhibitor for 5 to 8 days. In some further embodiment, the chemical reprogramming method disclosed herein exposes the somatic cells to the histone acetyltransferase inhibitor for 8 days, 7 days, 6 days, or 5 days.

The chemical reprogramming method disclosed herein shortens the time required for somatic cells to become a cell population with increased differentiation potential. In some embodiments, compared with the case where somatic cells are not exposed to the histone acetyltransferase inhibitor, the method disclosed herein can shorten the time required for somatic cells to become a cell population with increased differentiation potential by at least 8 days, preferably 8 to 12 days. In some embodiments, in the chemical reprogramming method disclosed herein, the conditions for enabling the somatic cells to form a cell population with increased differentiation potential compared to the somatic cells include contacting the somatic cells with a composition capable of increasing the differentiation potential of the cells, and the composition comprises a glycogen synthase kinase 3 (GSK-3) inhibitor, a transforming growth factor-beta (TGFβ) receptor inhibitor, and a retinoic acid receptor (RAR) agonist. In some embodiments, the composition comprises a glycogen synthase kinase (GSK) 33 inhibitor, a transforming growth factor-beta (TGFβ) receptor inhibitor, a retinoic acid receptor (RAR) agonist, and a serine-threonine kinase (Akt) inhibitor. In some embodiments, the composition comprises a glycogen synthase kinase 3 (GSK-3) inhibitor, a transforming growth factor-beta (TGFβ) receptor inhibitor, a retinoic acid receptor (RAR) agonist, a serine-threonine kinase (Akt) inhibitor, and any one selected from a G protein-coupled receptor Smoothened agonist, a Dot1L inhibitor, a Menin-MLL interaction inhibitor, a S-adenosyl-L-homocysteine (SAH) hydrolase inhibitor, a Jak1/Jak2 inhibitor, or any combination thereof. For example, in certain embodiments, the composition comprises a glycogen synthase kinase 3 (GSK-3) inhibitor, a transforming growth factor-beta (TGFβ) receptor inhibitor, a retinoic acid receptor (RAR) agonist, a serine-threonine kinase (Akt) inhibitor, a G protein-coupled receptor Smoothened agonist, and a Dot1L inhibitor. In some other embodiments, the composition comprises a glycogen synthase kinase 3 (GSK-3) inhibitor, a transforming growth factor-beta (TGFβ) receptor inhibitor, a retinoic acid receptor (RAR) agonist, a serine-threonine kinase (Akt) inhibitor, a Dot1L inhibitor, and a Jak1/Jak2 inhibitor. In other embodiments, the composition comprises a glycogen synthase kinase 3 (GSK-3) inhibitor, a transforming growth factor-beta (TGFβ) receptor inhibitor, a retinoic acid receptor (RAR) agonist, a serine-threonine kinase (Akt) inhibitor, a Dot1L inhibitor, and a S-adenosyl-L-homocysteine (SAH) hydrolase inhibitor. In another embodiments, the composition comprises a glycogen synthase kinase 3 (GSK-3) inhibitor, a transforming growth factor-beta (TGFβ) receptor inhibitor, a retinoic acid receptor (RAR) agonist, a serine-threonine kinase (Akt) inhibitor, a Jak1/Jak2 inhibitor, and a Menin-MLL interaction inhibitor. In further embodiment, the composition further comprises anyone selected from a c-Jun kinase inhibitor, a histone methyltransferase inhibitor, or an LPA1 receptor antagonist, or any combination thereof. In some further embodiment, the composition comprises a glycogen synthase kinase 3 (GSK-3) inhibitor, a transforming growth factor-beta (TGFβ) receptor inhibitor, a retinoic acid receptor (RAR) agonist, a G protein-coupled receptor Smoothened agonist, a Dot1L inhibitor, a Menin-MLL interaction inhibitor, a S-adenosyl-L-homocysteine (SAH) hydrolase inhibitor, a Jak1/Jak2 inhibitor, a c-Jun kinase inhibitor, a serine-threonine kinase (Akt) inhibitor, a histone methyltransferase inhibitor, and an LPA1 receptor antagonist. In some embodiments, the histone methyltransferase inhibitor disclosed herein is selected from SETD2-IN-1, EPZ-719, MMSET-IN-1, or any combination thereof. In some embodiments, the histone methyltransferase inhibitor disclosed herein is SETD2-IN-1. In some embodiments, the concentration of the histone methyltransferase inhibitor disclosed herein is 0.01-2 μM. In further embodiments, the concentration of the histone methyltransferase inhibitor disclosed herein is 0.2 μM. In some embodiment, the LPA1 receptor antagonist contained in the composition is selected from AM095, AM966, Ki16425, or any combination thereof. In some embodiments, the LPA1 receptor antagonist contained in the composition is AM095. In some embodiments, the concentration of the LPA1 receptor antagonist contained in the composition is 0.1 to 5 μM. In some embodiments, the concentration of the LPA1 receptor antagonist contained in the composition is 0.5 to 1 μM. In further embodiments, the LPA1 receptor antagonist contained in the composition is AM095 at a concentration of 0.5 to 1 μM.

Provided herein, in some aspects, are methods for generating pluripotent stem cells In an aspect, a method for generating pluripotent stem cells comprises contacting a population of cells comprising intermediate plastic state cells or progenies thereof with a composition comprising: (a) a MEK inhibitor; (b) a B-Raf inhibitor; and (c) a histone deacetylase inhibitor; thereby generating the pluripotent stem cells, wherein the intermediate plastic state cells express LIN28A and SALL4, and one or more of MSX2, NMYC, WNT4, FGF19, or TOP2A.

In some embodiments, during the contacting, the population of cells are incubated with about 21% atmospheric oxygen. In some embodiments, the MEK inhibitor comprises PD0325901, AZD8330, or TAK-733. In some embodiments, the MEK inhibitor comprises PD0325901. In some embodiments, PD0325901 is present at about 0.1 μM to about 10 μM with the third composition. In some embodiments, PD0325901 is present at about 0.2 μM to about 5 μM with the third composition. In some embodiments, PD0325901 is present at about 0.4 μM to about 2.5 μM with the third composition. In some embodiments, PD0325901 is present at about 1 μM with the third composition. In some embodiments, the B-Raf inhibitor comprises SB590885, Vemurafenib, RAF265, or PLX4720. In some embodiments, the B-Raf inhibitor comprises SB590885. In some embodiments, SB590885 is present at about 0.05 μM to about 5 μM with the composition. In some embodiments, SB590885 is present at about 0.1 μM to about 2.5 μM with the composition. In some embodiments, SB590885 is present at about 0.2 μM to about 1.25 μM with the composition. In some embodiments, SB590885 is present at about 0.5 μM within the composition. In some embodiments, the histone deacetylase inhibitor comprises valproic acid (VPA), LMK235, MS275, or HDACi I. In some embodiments, the histone deacetylase inhibitor comprises VPA. In some embodiments, VPA is present at about 0.1 mM to 10 mM within the composition. In some embodiments, VPA is present at about 0.2 mM to 5 mM within the composition. In some embodiments, VPA is present at about 0.4 mM to 2.5 mM within the composition. In some embodiments, VPA is present at about 1 mM within the composition. In some embodiments, the composition further comprises one or more of a Wnt inhibitor, a glycogen synthase kinase 3 (GSK-3) inhibitor, or a ROCK inhibitor. In some embodiments, the composition further comprises the Wnt inhibitor. In some embodiments, the Wnt inhibitor comprises IWR-1 or IWP-2. In some embodiments, the Wnt inhibitor comprises IWR-1. In some embodiments, the Wnt inhibitor comprises IWP-2. In some embodiments, IWP-2 is present at about 0.2 μM to about 20 μM within the composition. In some embodiments, IWP-2 is present at about 0.4 μM to about 10 μM within the composition. In some embodiments, IWP-2 is present at about 0.8 μM to about 5 μM within the composition. In some embodiments, IWP-2 is present at about 2 μM within the composition. In some embodiments, the composition further comprises the glycogen synthase kinase 3 (GSK-3) inhibitor. In some embodiments, the glycogen synthase kinase 3 (GSK-3) inhibitor comprises CHIR99021 or CHIR98014. In some embodiments, the glycogen synthase kinase 3 (GSK-3) inhibitor comprises CHIR99021. In some embodiments, CHIR99021 is present at about 0.1 μM to about 10 μM within the composition. In some embodiments, CHIR99021 is present at about 0.2 μM to about 5 μM within the composition. In some embodiments, CHIR99021 is present at about 0.4 μM to about 2.5 μM within the composition. In some embodiments, CHIR99021 is present at about 1 μM within the composition. In some embodiments, the composition further comprises the ROCK inhibitor. In some embodiments, the ROCK inhibitor comprises Y-27632 or thiazovivin. In some embodiments, the ROCK inhibitor comprises Y-27632. In some embodiments, Y-27632 is present at about 1 μM to about 100 μM within the composition. In some embodiments, Y-27632 is present at about 2 μM to about 50 μM within the composition. In some embodiments, Y-27632 is present at about 4 μM to about 25 μM within the composition. In some embodiments, Y-27632 is present at about 10 μM within the composition. In some embodiments, the composition further comprises one or more of an inhibitor of histone demethylation, a Dot1L inhibitor, or a S-adenosyl-L-homocysteine (SAH) hydrolase inhibitor. In some embodiments, the composition further comprises the inhibitor of histone demethylation. In some embodiments, the inhibitor of histone demethylation comprises Tranylcypromine. In some embodiments, Tranylcypromine is present at about 1 μM to about 100 μM within the composition. In some embodiments, Tranylcypromine is present at about 2 μM to about 50 μM within the composition. In some embodiments, Tranylcypromine is present at about 4 μM to about 25 μM within the composition. In some embodiments, Tranylcypromine is present at about 10 μM within the composition. In some embodiments, the composition further comprises the Dot1L inhibitor. In some embodiments, the Dot1L inhibitor comprises EPZ004777 or EPZ5676. In some embodiments, the Dot1L inhibitor comprises EPZ5676. In some embodiments, EPZ5676 is present at about 0.2 μM to about 20 μM within the composition. In some embodiments, EPZ5676 is present at about 0.4 μM to about 10 μM within the composition. In some embodiments, EPZ5676 is present at about 0.8 μM to about 5 μM within the composition. In some embodiments, EPZ5676 is present at about 2 μM within the composition. In some embodiments, the composition further comprises the S-adenosyl-L-homocysteine (SAH) hydrolase inhibitor. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.02 μM to about 20 μM within the composition. In some embodiments, DZNep is present at about 0.04 μM to about 10 μM within the composition. In some embodiments, DZNep is present at about 0.08 μM to about 5 μM within the composition. In some embodiments, DZNep is present at about 0.2 μM within the composition. In some embodiments, the contacting comprises culturing the population of cells in the composition. In some embodiments, the method further comprises, after the culturing for about 5 days, replacing the composition with a second composition for culturing. In some embodiments, the second composition comprises the histone deacetylase inhibitor. In some embodiments, a concentration of the histone deacetylase inhibitor within the second composition is about 50% of a concentration of the histone deacetylase inhibitor within the composition. In some embodiments, the method further comprises, after the culturing in the second composition for about 5 days, replacing the second composition with a third composition for culturing, In some embodiments, the third composition does not comprise the histone deacetylase inhibitor, the inhibitor of histone demethylation, the Dot1L inhibitor, or the S-adenosyl-L-homocysteine (SAH) hydrolase inhibitor.

In some embodiments, the method comprises culturing the population of cells in the composition for at most about 20 days. In some embodiments, the method comprises culturing the population of cells in the composition for at most about 12 days. In some embodiments, the method comprises culturing the population of cells in the composition from about 4 days to about 12 days. In some embodiments, the method leads to conversion of the intermediate plastic state cells or progenies thereof into pluripotent stem cells. In some embodiments, the pluripotent stem cells express one or more of OCT4, SOX2, or NANOG. In some embodiments, the pluripotent cells express one or more of FGF4, ZFP57, REX1, DPPA4, TDGF1, TRA-1-60, TRA-1-81, SSEA4, KLF4, KLF17, DPPA3, DPPA5, DNMT3L, REX1, or UTF1. In some embodiments, the pluripotent cells express one or more of FGF4, ZFP57, DPPA5, or REX1. In some embodiments, the pluripotent cells further express one or more of DPPA4, TDGF1, TRA-1-60, TRA-1-81, SSEA4, KLF4, KLF17, DPPA3, DPPA5, DNMT3L, REX1, or UTF1. In some embodiments, the chemical reprogramming method disclosed herein may further include the step of purifying pluripotent stem cells. In some embodiments, pluripotent stem cells are purified according to pluripotency markers, and the pluripotency markers are selected from OCT4, SOX2, NANOG, FGF4, ZFP57, DPPA5, REX1, DPPA4, TDGF1, TRA-1-60, TRA-1-81, SSEA4, KLF4, KLF17, DPPA3, DNMT3L, UTF, or any combination thereof. In some embodiments, based on the pluripotency markers used for purification, the purity of the obtained pluripotent stem cell population is above about 50%, above about 55%, above about 60%, above about 65%, above about 70%, above about 75%, above about 80%, above about 85%, above about 90%, above about 91%, above about 92%, above about 93%, above about 94%, above about 95%, above about 96%, above about 97%, above about 98%, above about 99%, or about 100%.

In some embodiments, the pluripotent stem cells or progenies thereof comprise a genetic modification. In some embodiments, the genetic modification comprises an exogenous nucleic acid sequence. In some embodiments, the exogenous nucleic acid sequence encodes a polypeptide. In some embodiments, the exogenous nucleic acid sequence comprises a sequence of a non-coding nucleic acid molecule. In some embodiments, the genetic modification comprises alteration of a genomic sequence. In some embodiments, the genetic modification reduces immunogenicity of the pluripotent stem cells or the progenies thereof.

Provided herein, in some aspects, are isolated populations of cells. In an aspect, an isolated population of cells comprises intermediate plastic state cells that express: (a) LIN28A and SALL4; (b) one or more of MSX2, NMYC, WNT4, FGF19, or TOP2A; and (c) one or more of MSX1, HOXB9, WT1, GATA2, HMGA2, or LEF1.

In some embodiments, the intermediate plastic state cells further express one or more of FGF9, HOXA9, HOXA1, PTCH1, HOXA5, CCND2, SDC1, TBX3, BMP4, OR IGF2. In some embodiments, the intermediate plastic state cells comprise a genetic modification. In some embodiments, the genetic modification comprises an exogenous nucleic acid sequence. In some embodiments, the exogenous nucleic acid sequence encodes a polypeptide. In some embodiments, the exogenous nucleic acid sequence comprises a sequence of a non-coding nucleic acid molecule. In some embodiments, the genetic modification comprises alteration of a genomic sequence. In some embodiments, the genetic modification reduces immunogenicity of the intermediate plastic state cells.

Provided herein, in some aspects, are compositions. In some aspects, a composition provided herein is a medium for culturing cells. In an aspect, a composition provided herein comprises: intermediate plastic state cells that express LIN28A, SALL4, and one or more of MSX2, NMYC, WNT4, FGF19, or TOP2A; and one or more of a glycogen synthase kinase 3 (GSK-3), a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor or an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, a casein kinase 2 inhibitor.

In some embodiments, the glycogen synthase kinase 3 (GSK-3) inhibitor comprises CHIR99021 or CHIR98014. In some embodiments, the glycogen synthase kinase 3 (GSK-3) inhibitor comprises CHIR99021. In some embodiments, the transforming growth factor-beta (TGFβ) receptor inhibitor comprises E-616452, A 83-01, SB431542, SB 505124, GW 788388, or SB 525334. In some embodiments, the transforming growth factor-beta (TGFβ) receptor inhibitor comprises E-616452. In some embodiments, the retinoic acid receptor (RAR) agonist comprises TTNPB, Ch55, or AM580. In some embodiments, the retinoic acid receptor (RAR) agonist comprises TTNPB. In some embodiments, the c-Jun kinase inhibitor comprises, JNKIN7, JNKIN5, or JNKIN12. In some embodiments, the c-Jun kinase inhibitor comprises JNKIN8. In some embodiments, the CBP/p300 bromodomain inhibitors comprises SGC-CBP30, I-CBP112, GNE272, or GNE409. In some embodiments, the CBP/p300 bromodomain inhibitor comprises SGC-CBP30. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, the adenosine kinase inhibitor comprises 5-Iodotubercidin or ABT 702. In some embodiments, the adenosine kinase inhibitor comprises 5-Iodotubercidin (5-ITU). In some embodiments, the Dot1L inhibitor comprises EPZ004777 or EPZ5676. In some embodiments, the Dot1L inhibitor comprises EPZ5676. In some embodiments, the Menin-MLL interaction inhibitor comprises VTP50469, MI3454, or WDR5-IN-4. In some embodiments, the Menin-MLL interaction inhibitor comprises VTP50469. In some embodiments, the SET domain containing 2 (SETD2) inhibitor comprises SETD2-IN-1, EPZ-719, or MMSET-IN-1. In some embodiments, the SET domain containing 2 (SETD2) inhibitor comprises SETD2-IN-1. In some embodiments, the agonist for the G protein-coupled receptor Smoothened comprises SAG, Purmorphamine, Hh-Ag1.5, or human SHH. In some embodiments, the agonist for the G protein-coupled receptor Smoothened comprises SAG. In some embodiments, the ROCK inhibitor comprises Y-27632 or thiazovivin. In some embodiments, the ROCK inhibitor comprises Y-27632. In some embodiments, the BMP receptor/AMPK inhibitor comprises Dorsomorphin. In some embodiments, the Jak1/Jak2 inhibitor comprises Ruxolitinib, Tofacitinib, AZD1480, Baricitinib, S-Ruxolitinib, or Fedratinib. In some embodiments, the Jak1/Jak2 inhibitor comprises Ruxolitinib. In some embodiments, the p38 MAPK inhibitor comprises BIRB796, SB203580, or SB202190. In some embodiments, the p38 MAPK inhibitor comprises BIRB796. In some embodiments, the Akt inhibitor comprises AKT Kinase Inhibitor. In some embodiments, the casein kinase 2 inhibitor comprises CX-4945, TPP 22, or Ellagic acid. In some embodiments, the casein kinase 2 inhibitor comprises CX-4945.

In an aspect, a composition provided herein comprises: epithelial-like cells that express LIN28A; and (a) a S-adenosyl-L-homocysteine (SAH) hydrolase inhibitor or an adenosine kinase inhibitor; (b) a glycogen synthase kinase 3 (GSK-3); (c) a transforming growth factor-beta (TGFβ) receptor inhibitor; and (d) a c-Jun kinase inhibitor.

In some embodiments, the composition further comprises a CBP/p300 bromodomain inhibitor.

In an aspect, a composition provided herein comprises: (a) a S-adenosyl-L-homocysteine (SAH) hydrolase inhibitor or an adenosine kinase inhibitor; (b) a glycogen synthase kinase 3 (GSK-3); (c) a transforming growth factor-beta (TGFβ) receptor inhibitor; (d) a c-Jun kinase inhibitor; and (e) a CBP/p300 bromodomain inhibitor.

In some embodiments, the composition comprises the adenosine kinase inhibitor. In some embodiments, the adenosine kinase inhibitor comprises 5-Iodotubercidin or ABT 702. In some embodiments, the adenosine kinase inhibitor 5-Iodotubercidin (5-ITU). In some embodiments, 5-ITU is present at about 0.05 μM to about 5 μM within the composition. In some embodiments, 5-ITU is present at about 0.1 micromolar μM to about 2.5 μM within the composition. In some embodiments, 5-ITU is present at about 0.2 micromolar μM to about 1 μM within the composition. In some embodiments, 5-ITU is present at about 0.5 μM within the composition. In some embodiments, the composition comprises the S-adenosyl-L-homocysteine (SAH) hydrolase inhibitor. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.02 μM to about 20 μM within the composition. In some embodiments, DZNep is present at about 0.04 μM to about 10 μM within the composition. In some embodiments, DZNep is present at about 0.08 μM to about 5 μM within the composition. In some embodiments, DZNep is present at about 0.2 μM within the composition. In some embodiments, the glycogen synthase kinase 3 (GSK-3) comprises CHIR99021 or CHIR98014. In some embodiments, the glycogen synthase kinase 3 (GSK-3) comprises CHIR99021. In some embodiments, CHIR99021 is present at about 0.5 μM to about 50 μM within the composition. In some embodiments, CHIR99021 is present at about 1 μM to about 25 μM within the composition. In some embodiments, CHIR99021 is present at about 2 μM to about 12.5 μM within the composition. In some embodiments, CHIR99021 is present at about 5 μM within the composition. In some embodiments, the transforming growth factor-beta (TGFβ) receptor inhibitor comprises E-616452, A 83-01, SB431542, SB 505124, GW 788388, or SB 525334. In some embodiments, the transforming growth factor-beta (TGFβ) receptor inhibitor comprises E-616452. In some embodiments, E-616452 is present at about 1 μM to about 100 μM within the composition. In some embodiments, E-616452 is present at about 2 μM to about 50 μM within the composition. In some embodiments, E-616452 is present at about 4 μM to about 25 μM within the composition. In some embodiments, E-616452 is present at about 10 μM within the composition. In some embodiments, the c-Jun kinase inhibitor comprises JNKIN8, JNKIN7, JNKIN5, or JNKIN12. In some embodiments, the c-Jun kinase inhibitor comprises JNKIN8. In some embodiments, JNKIN is present at about 0.05 μM to about 50 μM within the composition. In some embodiments, JNKIN is present at about 0.1 μM to about 2.5 μM within the composition. In some embodiments, JNKIN is present at about 0.2 μM to about 1.25 μM within the composition. In some embodiments, JNKIN is present at about 0.5 μM within the composition. In some embodiments, the CBP/p300 bromodomain inhibitors comprises SGC-CBP30, I-CBP112, GNE272, or GNE409. In some embodiments, the CBP/p300 bromodomain inhibitor comprises SGC-CBP30. In some embodiments, SGC-CBP30 is present at about 0.2 μM to about 20 μM within the composition. In some embodiments, SGC-CBP30 is present at about 0.4 μM to about 10 μM within the composition. In some embodiments, SGC-CBP30 is present at about 0.8 μM to about 5 μM within the composition. In some embodiments, SGC-CBP30 is present at about 2 μM within the composition. In some embodiments, the composition further comprises one or more of a SET domain containing 2 (SETD2) inhibitor, an Akt inhibitor, or a casein kinase 2 inhibitor. In some embodiments, the SET domain containing 2 (SETD2) inhibitor comprises SETD2-IN-1, EPZ-719, or MMSET-IN-1. In some embodiments, the SET domain containing 2 (SETD2) inhibitor comprises SETD2-IN-1. In some embodiments, SETD2-IN-1 is present at about 0.05 μM to about 5 μM within the composition. In some embodiments, SETD2-IN-1 is present at about 0.1 μM to about 2.5 μM within the composition. In some embodiments, SETD2-IN-1 is present at about 0.2 μM to about 1.25 μM within the composition. In some embodiments, SETD2-IN-1 is present at about 0.4 μM within the composition. In some embodiments, the Akt inhibitor comprises AKT Kinase Inhibitor. In some embodiments, AKT Kinase Inhibitor is present at about 0.1 μM to about 10 μM within the composition. In some embodiments, AKT Kinase Inhibitor is present at about 0.2 μM to about 5 μM within the composition. In some embodiments, AKT Kinase Inhibitor is present at about 0.4 μM to about 2.5 μM within the composition. In some embodiments, AKT Kinase Inhibitor is present at about 1 μM within the composition. In some embodiments, the casein kinase 2 inhibitor comprises CX-4945, TPP 22, or Ellagic acid. In some embodiments, the casein kinase 2 inhibitor comprises CX-4945. In some embodiments, CX-4945 is present at about 0.08 μM to about 8 μM within the composition. In some embodiments, CX-4945 is present at about 0.16 μM to about 4 μM within the composition. In some embodiments, CX-4945 is present at about 0.32 μM to about 2 μM within the composition. In some embodiments, CX-4945 is present at about 0.8 μM within the composition. In some embodiments, the composition further comprises one or more of a Menin-MLL interaction inhibitor, an agonist for the G protein-coupled receptor Smoothened, a ROCK inhibitor, or a BMP receptor/AMPK inhibitor. In some embodiments, the Menin-MLL interaction inhibitor comprises VTP50469, MI3454, or WDR5-IN-4. In some embodiments, the Menin-MLL interaction inhibitor comprises VTP50469. In some embodiments, VTP50469 is present at about 0.05 μM to about 5 μM within the composition. In some embodiments, VTP50469 is present at about 0.1 μM to about 2.5 μM within the composition. In some embodiments, VTP50469 is present at about 0.2 μM to about 1.25 μM within the composition. In some embodiments, VTP50469 is present at about 0.5 μM within the composition. In some embodiments, the agonist for the G protein-coupled receptor Smoothened comprises SAG, Purmorphamine, Hh-Ag1.5, or human SHH. In some embodiments, the agonist for the G protein-coupled receptor Smoothened comprises SAG. In some embodiments, SAG is present at about 0.05 μM to about 5 μM within the composition. In some embodiments, SAG is present at about 0.1 μM to about 2.5 μM within the composition. In some embodiments, SAG is present at about 0.2 μM to about 1.25 μM within the composition. In some embodiments, SAG is present at about 0.5 μM within the composition. In some embodiments, the ROCK inhibitor comprises Y-27632 or thiazovivin. In some embodiments, the ROCK inhibitor comprises Y-27632. In some embodiments, Y27632 is present at about 1 μM to about 100 μM within the composition. In some embodiments, Y27632 is present at about 2 μM to about 50 μM within the composition. In some embodiments, Y27632 is present at about 4 μM to about 25 μM within the composition. In some embodiments, Y27632 is present at about 10 μM within the composition. In some embodiments, the BMP receptor/AMPK inhibitor comprises Dorsomorphin. In some embodiments, Dorsomorphin is present at about 0.05 μM to about 5 μM within the composition. In some embodiments, Dorsomorphin is present at about 0.1 μM to about 2.5 μM within the composition. In some embodiments, Dorsomorphin is present at about 0.2 μM to about 1.25 μM within the composition. In some embodiments, Dorsomorphin is present at about 0.5 μM within the composition. In some embodiments, the method further comprises one or more of a Dot1L inhibitor, a Jak1/Jak2 inhibitor, or a p38 MAPK inhibitor. In some embodiments, the Dot1L inhibitor comprises EPZ004777 or EPZ5676. In some embodiments, the Dot1L inhibitor comprises the EPZ5676. In some embodiments, EPZ5676 is present at about 0.2 μM to about 20 μM within the composition. In some embodiments, EPZ5676 is present at about 0.4 μM to about 10 μM within the composition. In some embodiments, EPZ5676 is present at about 0.8 μM to about 5 μM within the composition. In some embodiments, EPZ5676 is present at about 2 μM within the composition. In some embodiments, the Jak1/Jak2 inhibitor comprises Ruxolitinib, Tofacitinib, AZD1480, Baricitinib, S-Ruxolitinib, or Fedratinib. In some embodiments, the Jak1/Jak2 inhibitor comprises Ruxolitinib. In some embodiments, Ruxolitinib is present at about 0.1 μM to about 10 μM within the composition. In some embodiments, Ruxolitinib is present at about 0.2 μM to about 5 μM within the composition. In some embodiments, Ruxolitinib is present at about 0.4 μM to about 2.5 μM within the composition. In some embodiments, Ruxolitinib is present at about 1 μM within the composition. In some embodiments, the p38 MAPK inhibitor comprises BIRB796, SB203580, or SB202190. In some embodiments, the p38 MAPK inhibitor comprises BIRB796. In some embodiments, BIRB796 is present at about 0.2 μM to about 20 μM within the composition. In some embodiments, BIRB796 is present at about 0.4 μM to about 10 μM within the composition. In some embodiments, BIRB796 is present at about 0.8 μM to about 5 μM within the composition. In some embodiments, BIRB796 is present at about 2 μM within the composition. In some embodiments, the composition further comprises a retinoic acid receptor (RAR) agonist. In some embodiments, the retinoic acid receptor (RAR) agonist comprises TTNPB, Ch55, or AM580. In some embodiments, the retinoic acid receptor (RAR) agonist comprises TTNPB. In some embodiments, TTNPB is present at about 0.2 μM to about 20 μM within the composition. In some embodiments, TTNPB is present at about 0.4 μM to about 10 μM within the composition. In some embodiments, TTNPB is present at about 0.8 μM to about 5 μM within the composition. In some embodiments, TTNPB is present at about 2 μM within the composition.

Provided herein, in some aspects, are isolated populations of cells. In an aspect, an isolated population of cells comprises epithelial-like cells that express LIN28A and one or more of NMYC, WNT2B, PAX8, SMAD3, or GLI3.

In some embodiments, the isolated population of cells comprising epithelial-like cells expresses one or more of KRT18, KRT19, WT1, or TBX2. In some embodiments, the isolated population of cells comprising epithelial-like cells does not express any one of MMP1, ZEB1, VIM, COL1A1, COL5A1, COL6A2, PRRX1, SNAI2, TWIST1, or TWIST2.

In an aspect, a composition provided herein comprises epithelial-like cells that express LIN28A; and (a) a glycogen synthase kinase 3 (GSK-3), (b) a transforming growth factor-beta (TGFβ) receptor inhibitor, (c) a retinoic acid receptor (RAR) agonist, and (d) an Akt inhibitor or a SET domain containing 2 (SETD2) inhibitor.

In some embodiments, the composition comprises the Akt inhibitor. In some embodiments, the Akt inhibitor comprises AKT Kinase Inhibitor. In some embodiments, the composition comprises the SET domain containing 2 (SETD2) inhibitor. In some embodiments, the SET domain containing 2 (SETD2) inhibitor comprises SETD2-IN-1, EPZ-719, or MMSET-IN-1. In some embodiments, the SET domain containing 2 (SETD2) inhibitor comprises SETD2-IN-1.

In an aspect, a composition provided herein comprises epithelial-like cells that express LIN28A and one or more of NMYC, WNT2B, PAX8, SMAD3, or GLI3; and (a) a glycogen synthase kinase 3 (GSK-3), (b) a transforming growth factor-beta (TGFβ) receptor inhibitor, and (c) a retinoic acid receptor (RAR) agonist.

In some embodiments, the composition comprises an Akt inhibitor or a SET domain containing 2 (SETD2) inhibitor. In some embodiments, the composition comprises the Akt inhibitor. In some embodiments, the Akt inhibitor comprises AKT Kinase Inhibitor. In some embodiments, the composition comprises the SET domain containing 2 (SETD2) inhibitor. In some embodiments, the SET domain containing 2 (SETD2) inhibitor comprises SETD2-IN-1, EPZ-719, or MMSET-IN-1. In some embodiments, the SET domain containing 2 (SETD2) inhibitor comprises SETD2-IN-1. In some embodiments, the composition is serum free. In some embodiments, the composition is feeder-cell free. In some embodiments, the glycogen synthase kinase 3 (GSK-3) comprises CHIR99021 or CHIR98014. In some embodiments, the glycogen synthase kinase 3 (GSK-3) comprises CHIR99021. In some embodiments, the transforming growth factor-beta (TGFβ) receptor inhibitor comprises E-616452, A 83-01, SB431542, SB 505124, GW 788388, dorsomorphine, or SB 525334. In some embodiments, the transforming growth factor-beta (TGFβ) receptor inhibitor comprises E-616452. In some embodiments, the retinoic acid receptor (RAR) agonist comprises TTNPB, Ch55, or AM580. In some embodiments, the retinoic acid receptor (RAR) agonist comprises TTNPB. In some embodiments, the composition further comprises an agonist for the G protein-coupled receptor Smoothened or a Menin-MLL interaction inhibitor. In some embodiments, the agonist for the G protein-coupled receptor Smoothened comprises SAG, Purmorphamine, Hh-Ag1.5, or human SHH. In some embodiments, the agonist for the G protein-coupled receptor Smoothened comprises SAG. In some embodiments, the Menin-MLL interaction inhibitor comprises VTP50469, MI3454, or WDR5-IN-4. In some embodiments, the Menin-MLL interaction inhibitor comprises VTP50469. In some embodiments, the composition further comprises a Jak1/Jak2 inhibitor, an S-adenosyl-L-homocysteine (SAH) hydrolase inhibitor, a Dot1L inhibitor. In some embodiments, the Jak1/Jak2 inhibitor comprises Ruxolitinib, Tofacitinib, AZD1480, Baricitinib, S-Ruxolitinib, or Fedratinib. In some embodiments, the Jak1/Jak2 inhibitor comprises Ruxolitinib. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, the Dot1L inhibitor comprises EPZ004777 or EPZ5676. In some embodiments, the Dot1L inhibitor comprises EPZ5676.

In an aspect, a composition provided herein comprises: (a) a glycogen synthase kinase 3 (GSK-3), (b) a transforming growth factor-beta (TGFβ) receptor inhibitor, (c) a retinoic acid receptor (RAR) agonist, and (d) an Akt inhibitor or a SET domain containing 2 (SETD2) inhibitor.

In some embodiments, the composition further comprises somatic cells. In some embodiments, the somatic cells comprise primary human adult adipose derived mesenchymal stromal cells (hADSCs). In some embodiments, the somatic cells comprise fibroblasts. In some embodiments, the composition is serum free. In some embodiments, the composition is feeder-cell free. In some embodiments, the glycogen synthase kinase 3 (GSK-3) comprises CHIR99021 or CHIR98014. In some embodiments, the glycogen synthase kinase 3 (GSK-3) comprises CHIR99021. In some embodiments, CHIR99021 is present at about 0.5 μM to about 50 μM within the composition. In some embodiments, CHIR99021 is present at about 1 μM to about 25 μM within the composition. In some embodiments, CHIR99021 is present at about 2 μM to about 12.5 μM within the composition. In some embodiments, CHIR99021 is present at about 5 μM within the composition. In some embodiments, the transforming growth factor-beta (TGFβ) receptor inhibitor is an ALK5 inhibitor. In some embodiments, the transforming growth factor-beta (TGFβ) receptor inhibitor comprises E-616452, A 83-01, SB431542, SB 505124, GW 788388, or SB 525334, optionally wherein the transforming growth factor-beta (TGFβ) receptor inhibitor comprises E-616452. In some embodiments, the E-616452 is present at about 1 μM to about 100 μM within the composition. In some embodiments, the E-616452 is present at about 2 μM to about 50 μM within the composition. In some embodiments, the E-616452 is present at about 4 μM to about 25 μM within the composition. In some embodiments, the E-616452 is present at about 10 μM within the composition. In some embodiments, the retinoic acid receptor (RAR) agonist comprises TTNPB, Ch55, or AM580. In some embodiments, the retinoic acid receptor (RAR) agonist comprises TTNPB. In some embodiments, TTNPB is present at about 0.2 μM to about 20 μM within the composition. In some embodiments, TTNPB is present at about 0.4 μM to about 10 μM within the composition. In some embodiments, TTNPB is present at about 0.8 μM to about 5 μM within the composition. In some embodiments, TTNPB is present at about 2 μM within the composition. In some embodiments, the composition comprises the Akt inhibitor. In some embodiments, the Akt inhibitor comprises AKT Kinase Inhibitor. In some embodiments, AKT Kinase Inhibitor is present at about 0.1 μM to about 10 μM within the composition. In some embodiments, AKT Kinase Inhibitor is present at about 0.2 μM to about 5 μM within the composition. In some embodiments, AKT Kinase Inhibitor is present at about 0.4 μM to about 2.5 μM within the composition. In some embodiments, AKT Kinase Inhibitor is present at about 1 μM within the composition. In some embodiments, the composition comprises the SET domain containing 2 (SETD2) inhibitor. In some embodiments, the SET domain containing 2 (SETD2) inhibitor comprises SETD2-IN-1, EPZ-719, or MMSET-IN-1. In some embodiments, the SET domain containing 2 (SETD2) inhibitor comprises SETD2-IN-1. In some embodiments, SETD2-IN-1 is present at about 0.05 μM to about 5 μM within the composition. In some embodiments, SETD2-IN-1 is present at about 0.1 μM to about 2.5 μM within the composition. In some embodiments, SETD2-IN-1 is present at about 0.2 μM to about 1.25 μM within the composition. In some embodiments, SETD2-IN-1 is present at about 0.4 μM within the composition. In some embodiments, the composition further comprises an agonist for the G protein-coupled receptor Smoothened or a Menin-MLL interaction inhibitor. In some embodiments, the agonist for the G protein-coupled receptor Smoothened comprises SAG, Purmorphamine, Hh-Ag1.5, or human SHH. In some embodiments, the agonist for the G protein-coupled receptor Smoothened comprises SAG. In some embodiments, SAG is present at about 0.05 μM to about 5 μM within the composition. In some embodiments, SAG is present at about 0.1 μM to about 2.5 μM within the composition. In some embodiments, SAG is present at about 0.2 μM to about 1.25 μM within the composition. In some embodiments, SAG is present at about 0.5 μM within the composition. In some embodiments, the Menin-MLL interaction inhibitor comprises VTP50469, MI3454, or WDR5-IN-4. In some embodiments, the Menin-MLL interaction inhibitor comprises the VTP50469. In some embodiments, VTP50469 is present at about 0.05 μM to about 5 μM within the composition. In some embodiments, VTP50469 is present at about 0.1 μM to about 2.5 μM within the composition. In some embodiments, VTP50469 is present at about 0.2 μM to about 1.25 μM within the composition. In some embodiments, VTP50469 is present at about 0.5 μM within the composition. In some embodiments, the composition further comprises a Jak1/Jak2 inhibitor, an S-adenosyl-L-homocysteine (SAH) hydrolase inhibitor, a Dot1L inhibitor. In some embodiments, the Jak1/Jak2 inhibitor comprises Ruxolitinib, Tofacitinib, AZD1480, Baricitinib, S-Ruxolitinib, or Fedratinib. In some embodiments, the Jak1/Jak2 inhibitor comprises Ruxolitinib. In some embodiments, Ruxolitinib is present at about 0.1 μM to about 10 μM within the composition. In some embodiments, Ruxolitinib is present at about 0.2 μM to about 5 μM within the composition. In some embodiments, Ruxolitinib is present at about 0.4 μM to about 2.5 μM within the composition. In some embodiments, Ruxolitinib is present at about 1 μM within the composition. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.002 μM to about 0.2 μM within the composition. In some embodiments, DZNep is present at about 0.004 μM to about 0.1 μM within the composition. In some embodiments, DZNep is present at about 0.008 μM to about 0.05 μM within the composition. In some embodiments, DZNep is present at about 0.02 μM within the composition. In some embodiments, the Dot1L inhibitor comprises EPZ004777 or EPZ5676. In some embodiments, the Dot1L inhibitor comprises the EPZ5676. In some embodiments, EPZ5676 is present at about 0.2 μM to about 20 μM within the composition. In some embodiments, EPZ5676 is present at about 0.4 μM to about 10 μM within the composition. In some embodiments, EPZ5676 is present at about 0.8 μM to about 5 μM within the composition. In some embodiments, EPZ5676 is present at about 2 μM within the composition.

In an aspect, a composition provided herein comprises: intermediate plastic state cells that express LIN28A and SALL4, and one or more of MSX2, NMYC, WNT4, FGF19, or TOP2A; and one or more of a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor.

In some embodiments, the intermediate plastic state cells express one or more of FGF9, HOXA9, HOXA1, PTCH1, HOXA5, CCND2, SDC1, TBX3, BMP4, OR IGF2. In some embodiments, the MEK inhibitor comprises PD0325901, AZD8330, or TAK-733. In some embodiments, the MEK inhibitor comprises PD0325901. In some embodiments, PD0325901 is present at about 0.1 μM to about 10 μM with the third composition. In some embodiments, PD0325901 is present at about 0.2 μM to about 5 μM with the third composition. In some embodiments, PD0325901 is present at about 0.4 μM to about 2.5 μM with the third composition. In some embodiments, PD0325901 is present at about 1 μM with the third composition. In some embodiments, the B-Raf inhibitor comprises SB590885, Vemurafenib, RAF265, or PLX4720. In some embodiments, the B-Raf inhibitor comprises SB590885. In some embodiments, SB590885 is present at about 0.05 μM to about 5 μM with the composition. In some embodiments, SB590885 is present at about 0.1 μM to about 2.5 μM with the composition. In some embodiments, SB590885 is present at about 0.2 μM to about 1.25 μM with the composition. In some embodiments, SB590885 is present at about 0.5 μM within the composition. In some embodiments, the histone deacetylase inhibitor comprises valproic acid (VPA), LMK235, MS275, or HDACi IV. In some embodiments, the histone deacetylase inhibitor comprises valproic acid (VPA). In some embodiments, VPA is present at about 0.1 mM to 10 mM within the composition. In some embodiments, VPA is present at about 0.2 mM to 5 mM within the composition. In some embodiments, VPA is present at about 0.4 mM to 2.5 mM within the composition. In some embodiments, VPA is present at about 1 mM within the composition. In some embodiments, the inhibitor of histone demethylation comprises Tranylcypromine. In some embodiments, Tranylcypromine is present at about 1 μM to about 100 μM within the composition. In some embodiments, Tranylcypromine is present at about 2 μM to about 50 μM within the composition. In some embodiments, Tranylcypromine is present at about 4 μM to about 25 μM within the composition. In some embodiments, Tranylcypromine is present at about 10 μM within the composition. In some embodiments, the Dot1L inhibitor comprises EPZ004777 or EPZ5676. In some embodiments, the Dot1L inhibitor comprises EPZ5676. In some embodiments, EPZ5676 is present at about 0.2 μM to about 20 μM within the composition. In some embodiments, EPZ5676 is present at about 0.4 μM to about 10 μM within the composition. In some embodiments, EPZ5676 is present at about 0.8 μM to about 5 μM within the composition. In some embodiments, EPZ5676 is present at about 2 μM within the composition. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.02 μM to about 20 μM within the composition. In some embodiments, DZNep is present at about 0.04 μM to about 10 μM within the composition. In some embodiments, DZNep is present at about 0.08 μM to about 5 μM within the composition. In some embodiments, DZNep is present at about 0.2 μM within the composition. In some embodiments, the Wnt inhibitor comprises IWR-1 or IWP-2. In some embodiments, the Wnt inhibitor comprises IWR-1. In some embodiments, the Wnt inhibitor comprises IWP-2. In some embodiments, IWP-2 is present at about 0.2 μM to about 20 μM within the composition. In some embodiments, IWP-2 is present at about 0.4 μM to about 10 μM within the composition. In some embodiments, IWP-2 is present at about 0.8 μM to about 5 μM within the composition. In some embodiments, IWP-2 is present at about 2 μM within the composition. In some embodiments, the glycogen synthase kinase 3 (GSK-3) inhibitor comprises CHIR99021 or CHIR98014. In some embodiments, the glycogen synthase kinase 3 (GSK-3) inhibitor comprises CHIR99021. In some embodiments, CHIR99021 is present at about 0.1 micromolar (μM) to about 10 μM within the composition. In some embodiments, CHIR99021 is present at about 0.2 μM to about 5 μM within the composition. In some embodiments, CHIR99021 is present at about 0.4 μM to about 2.5 μM within the composition. In some embodiments, CHIR99021 is present at about 1 μM within the composition. In some embodiments, the ROCK inhibitor comprises Y-27632 or thiazovivin. In some embodiments, the ROCK inhibitor comprises Y-27632. In some embodiments, Y-27632 is present at about 1 μM to about 100 μM within the composition. In some embodiments, Y-27632 is present at about 2 μM to about 50 μM within the composition. In some embodiments, Y-27632 is present at about 4 μM to about 25 μM within the composition. In some embodiments, Y-27632 is present at about 10 μM within the composition.

In an aspect, a composition provided herein comprises: (a) a MEK inhibitor, a B-Raf inhibitor, and a histone deacetylase inhibitor; and (b) an inhibitor of histone demethylation, a Dot1L inhibitor, or a S-adenosyl-L-homocysteine (SAH) hydrolase inhibitor.

In some embodiments, the composition further comprises pluripotent stem cells that express OCT4, SOX2, and NANOG. In some embodiments, the pluripotent cells express one or more of FGF4, ZFP57, DPPA5, or REX1. In some embodiments, the pluripotent cells express one or more of DPPA4, TDGF1, TRA-1-60, TRA-1-81, SSEA4, KLF4, KLF17, DPPA3, DNMT3L, or UTF1.

In some embodiments, the MEK inhibitor comprises PD0325901, AZD8330, or TAK-733. In some embodiments, the MEK inhibitor comprises PD0325901. In some embodiments, the B-Raf inhibitor comprises SB590885, Vemurafenib, RAF265, or PLX4720. In some embodiments, the B-Raf inhibitor comprises SB590885. In some embodiments, the histone deacetylase inhibitor comprises valproic acid (VPA), LMK235, MS275, or HDACi IV. In some embodiments, the histone deacetylase inhibitor comprises VPA. In some embodiments, the composition comprises the inhibitor of histone demethylation. In some embodiments, the inhibitor of histone demethylation comprises Tranylcypromine. In some embodiments, the composition comprises the Dot1L inhibitor. In some embodiments, the Dot1L inhibitor comprises EPZ004777 or EPZ5676. In some embodiments, the Dot1L inhibitor comprises EPZ5676. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, the composition further comprises a Wnt inhibitor, a glycogen synthase kinase 3 (GSK-3), or a ROCK inhibitor. In some embodiments, the composition comprises the Wnt inhibitor. In some embodiments, the Wnt inhibitor comprises IWR-1 or IWP-2. In some embodiments, the Wnt inhibitor comprises IWR-1. In some embodiments, the Wnt inhibitor comprises IWP-2. In some embodiments, the composition comprises the glycogen synthase kinase 3 (GSK-3). In some embodiments, the glycogen synthase kinase 3 (GSK-3) comprises CHIR99021 or CHIR98014. In some embodiments, the glycogen synthase kinase 3 (GSK-3) comprises CHIR99021. In some embodiments, the glycogen synthase kinase 3 (GSK-3) comprises CHIR98014. In some embodiments, the composition comprises the ROCK inhibitor. In some embodiments, the ROCK inhibitor comprises Y-27632 or thiazovivin. In some embodiments, the ROCK inhibitor comprises Y-27632. In some embodiments, the pluripotent stem cells comprise the genetic modification. In some embodiments, the genetic modification comprises an exogenous nucleic acid sequence. In some embodiments, the exogenous nucleic acid sequence encodes a polypeptide. In some embodiments, the exogenous nucleic acid sequence comprises a sequence of a non-coding nucleic acid molecule. In some embodiments, the genetic modification comprises alteration of a genomic sequence. In some embodiments, the genetic modification reduces immunogenicity of the pluripotent stem cells. In some embodiments, the MEK inhibitor comprises PD0325901, AZD8330, or TAK-733. In some embodiments, the MEK inhibitor comprises PD0325901. In some embodiments, PD0325901 is present at about 0.1 μM to about 10 μM with the third composition. In some embodiments, PD0325901 is present at about 0.2 μM to about 5 μM with the third composition. In some embodiments, PD0325901 is present at about 0.4 μM to about 2.5 μM with the third composition. In some embodiments, PD0325901 is present at about 1 μM with the third composition. In some embodiments, the B-Raf inhibitor comprises SB590885, Vemurafenib, RAF265, or PLX4720. In some embodiments, the B-Raf inhibitor comprises SB590885. In some embodiments, SB590885 is present at about 0.05 μM to about 5 μM with the composition. In some embodiments, SB590885 is present at about 0.1 μM to about 2.5 μM with the composition. In some embodiments, SB590885 is present at about 0.2 μM to about 1.25 μM with the composition. In some embodiments, SB590885 is present at about 0.5 μM within the composition. In some embodiments, the histone deacetylase inhibitor comprises valproic acid (VPA), LMK235, MS275, or HDACi IV. In some embodiments, the histone deacetylase inhibitor comprises valproic acid (VPA). In some embodiments, VPA is present at about 0.1 mM to 10 mM within the composition. In some embodiments, VPA is present at about 0.2 mM to 5 mM within the composition. In some embodiments, VPA is present at about 0.4 mM to 2.5 mM within the composition. In some embodiments, VPA is present at about 1 mM within the composition. In some embodiments, the inhibitor of histone demethylation comprises Tranylcypromine. In some embodiments, Tranylcypromine is present at about 1 μM to about 100 μM within the composition. In some embodiments, Tranylcypromine is present at about 2 μM to about 50 μM within the composition. In some embodiments, Tranylcypromine is present at about 4 μM to about 25 μM within the composition. In some embodiments, Tranylcypromine is present at about 10 μM within the composition. In some embodiments, the Dot1L inhibitor comprises EPZ004777 or EPZ5676. In some embodiments, the Dot1L inhibitor comprises EPZ5676. In some embodiments, EPZ5676 is present at about 0.2 μM to about 20 μM within the composition. In some embodiments, EPZ5676 is present at about 0.4 μM to about 10 μM within the composition. In some embodiments, EPZ5676 is present at about 0.8 μM to about 5 μM within the composition. In some embodiments, EPZ5676 is present at about 2 μM within the composition. In some embodiments, the S-adenosyl-L-homocysteine (SAH) hydrolase inhibitor comprises DZNep, NepA, Adox, or DZA. In some embodiments, the S-adenosyl-L-homocysteine (SAH) hydrolase inhibitor comprises DZNep. In some embodiments, DZNep is present at about 0.02 μM to about 20 μM within the composition. In some embodiments, DZNep is present at about 0.04 μM to about 10 μM within the composition. In some embodiments, DZNep is present at about 0.08 μM to about 5 μM within the composition. In some embodiments, DZNep is present at about 0.2 μM within the composition. In some embodiments, the Wnt inhibitor comprises IWR-1 or IWP-2. In some embodiments, the Wnt inhibitor comprises IWR-1. In some embodiments, the Wnt inhibitor comprises IWP-2. In some embodiments, IWP-2 is present at about 0.2 μM to about 20 μM within the composition. In some embodiments, IWP-2 is present at about 0.4 μM to about 10 μM within the composition. In some embodiments, IWP-2 is present at about 0.8 μM to about 5 μM within the composition. In some embodiments, IWP-2 is present at about 2 μM within the composition. In some embodiments, the glycogen synthase kinase 3 (GSK-3) comprises CHIR99021 or CHIR98014. In some embodiments, the glycogen synthase kinase 3 (GSK-3) comprises CHIR99021. In some embodiments, CHIR99021 is present at about 0.1 μM to about 10 μM within the composition. In some embodiments, CHIR99021 is present at about 0.2 μM to about 5 μM within the composition. In some embodiments, CHIR99021 is present at about 0.4 μM to about 2.5 μM within the composition. In some embodiments, CHIR99021 is present at about 1 μM within the composition. In some embodiments, the ROCK inhibitor comprises Y-27632 or thiazovivin. In some embodiments, the ROCK inhibitor comprises Y-27632. In some embodiments, Y-27632 is present at about 1 μM to about 100 μM within the composition. In some embodiments, Y-27632 is present at about 2 μM to about 50 μM within the composition. In some embodiments, Y-27632 is present at about 4 μM to about 25 μM within the composition. In some embodiments, Y-27632 is present at about 10 μM within the composition.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “FIG.” or “FIGs.” herein), of which:

FIG. 1 depicts a schematic for converting somatic cells to human chemically induced pluripotent stem cells (hCiPSCs).

FIG. 2 depicts another schematic for converting somatic cells to hCiPSCs.

FIG. 3 depicts representative images of cells at the end of stage 1 using various media for stage 1 conversion process.

FIG. 4 depicts the numbers of the hCiPSC colonies generated using various media for stage 1 conversion process.

FIG. 5 depicts the numbers of the hCiPSC colonies generated using another set of various media for stage 1 conversion process.

FIG. 6 depicts the numbers of the hCiPSC colonies generated using stage 1 conversion media with or without a SET domain containing 2 (SETD2) inhibitor.

FIG. 7A depicts the numbers of the hCiPSC colonies generated using stage 2 conversion media with or without an adenosine kinase inhibitor.

FIG. 7B depicts the numbers of the hCiPSC colonies generated using stage 2 conversion media with or without a Menin-MLL interaction inhibitor.

FIG. 8 depicts representative images of immunofluorescence analysis of a set of pluripotency markers in hCiPSCs derived from human adipose derived stromal cells (hADSCs).

FIG. 9 depicts representative images of morphological analysis of hCiPSCs derived from hADSCs.

FIG. 10 depicts the reprogramming efficiencies to generate hCiPSCs from different donors.

FIG. 11 depicts a comparison of the numbers of the hCiPSC colonies generated using a method described herein and another method.

FIG. 12 depicts representative images of immunofluorescence analysis of another set of pluripotency markers in hCiPSCs derived from hADSCs.

FIG. 13 depicts the Reverse transcription (RT)-quantitative PCR (qPCR) analysis of pluripotency markers in the indicated hCiPSCs and control.

FIG. 14 depicts e representative images of haematoxylin and eosin staining of endoderm (respiratory epithelium), mesoderm (cartilage) and ectoderm (pigmented retinal epithelium and neural tissue) from a single teratoma of the indicated hCiPSC clones.

FIG. 15A depicts immunofluorescence analysis of the regeneration-related genes at the end of stage 1 and stage 2. FIG. 15B depicts the analysis of the expression of somatic cells related genes and regeneration related genes in hADSCs and cells at the end of stage 2.

FIG. 16 depicts GO term enrichment analyses of the upregulated genes in hADSCs and in the cells at the end of stage 2.

FIG. 17 depicts the numbers of the hCiPSC colonies using various stage 2 conversion media.

FIG. 18 depicts the numbers of the hCiPSC colonies generated using stage 2 conversion media with or without a serine-threonine kinase Akt inhibitor and a casein kinase 2 inhibitor.

FIG. 19 depicts the numbers of the hCiPSC colonies using various stage 3 conversion media.

FIG. 20 depicts exemplary epithelial-like cells that express LIN28A in stage 1 or stage 2 shown in a heatmap.

FIG. 21 depicts exemplary compositions comprising chemical reprogramming factors and optional cells in stage 1.

FIG. 22 depicts gene expression profile of exemplary intermediate plastic state cells in stage 2.

FIG. 23 depicts exemplary compositions comprising chemical reprogramming factors and optional cells in stage 2.

FIG. 24 depicts exemplary hCiPSCs that express OCT4, SOX2, and NANOG shown in a heatmap.

FIG. 25 depicts exemplary compositions comprising chemical reprogramming factors and optional cells in stage 3.

FIG. 26 shows the results of the Gene Ontology (GO) analysis of the transcriptomes of cell lines with different chemical reprogramming conversion efficiencies in Example 6A. FIG. 26A shows the GO analysis results of 7 cell lines with different conversion efficiencies. FIG. 26B shows the types of genes that are enriched and expressed in the cell lines with low conversion efficiency. FIG. 26C is a schematic diagram of the screening process of the candidate small molecule library.

FIG. 27 shows the changes in the number of hCiPSC clones obtained by adding different histone acetyltransferase inhibitors in the early stage of chemical-induced reprogramming. FIG. 27A and FIG. 27B are the induction results of the “8+4+8” method and the “6+4+8” method in Example 6B respectively. “NC” represents the control group without the addition of any HATi, and each test group added HATi to a final concentration of 0.5 μM.

FIG. 28 shows the reprogramming results of Example 6C. “SI+WM8014” represents the group that additionally added 1 μM WM8014 alone to the early induction medium; “SI+A485” represents the group that additionally added 0.5 μM A485 alone to the early induction medium; “SI+A485+WM8014” represents the group that additionally added a combination of 1 μM WM8014 and 0.5 μM A485 to the early induction medium.

FIG. 29 shows the reprogramming results of Example 6D. “SII+RA” represents the group that additionally added 5 μM RA alone to the Stage II induction medium; “SII+GSK3689032” represents the group that additionally added 0.02 μM GSK3689032 alone to the Stage II induction medium; “SI+GSK3689032+RA” represents the combination of additionally adding 0.02 μM GSK3689032 and 5 μM RA to the Stage II induction medium.

FIG. 30 shows the reprogramming results of Example 6E. “SIII+PY60” represents the group that additionally added 10 μM PY60 alone to the Stage III induction medium; “SIII+PY60, VPA200-500” represents the group that additionally added 10 μM PY60 to the Stage III induction medium and reduced the concentration of VPA to 200 μM in the first three days of incubation with the Stage III induction medium; “SIII+PY60, VPA200-200” represents the group that additionally added 2 μM PY60 to the Stage III induction medium and reduced the concentration of VPA to 200 μM in the first six days of incubation with the Stage III induction medium.

FIG. 31 shows the number of hCiPSC clones obtained by the exemplary method in Example 6F under different induction duration combinations.

FIG. 32 shows the number of hCiPSC clones obtained by the exemplary method in Example 6F when incubated with the Stage II induction medium for different durations.

FIG. 33 shows the number of hCiPSC clones obtained by the exemplary method in Example 6F when incubated with the Stage III induction medium for different durations.

FIG. 34 shows the results of immunofluorescence staining of OCT4 and SOX2 on the hCiPSC cell population obtained by the exemplary method in Example 6G.

FIG. 35 shows the counting results of the hCiPSC clones obtained by the exemplary method in Example 6H.

FIG. 36 shows the conversion curve when inducing the reprogramming of hADSC cells using the exemplary method in Example 6H. The X-axis represents the number of days since the start of induction, and the Y-axis represents the number of generated hCiPSC clones.

FIG. 37 shows the conversion curve when inducing the reprogramming of HD cells using the exemplary method in Example 6H. The X-axis represents the number of days since the start of induction, and the Y-axis represents the number of generated hCiPSC clones.

FIG. 38 shows the number of hCiPSC clones obtained by two different hADSC cell lines through the exemplary method in Example 6F under different induction duration combinations.

FIG. 39 shows screening results of chemical libraries targeting histone modifications during chemical reprogramming stage 1 in Example 7. The KAT3A/KAT3B catalytic inhibitor A-485 was the top candidate in the 0.5 μM screening batch.

FIG. 40 shows another screening results of chemical libraries targeting histone modifications during chemical reprogramming stage 1 in Example 7. The KAT6A inhibitor WM-8014 was the top candidate in the 1 μM screening batch.

FIG. 41 shows the results from inhibition with A-485 and WM-8014. (A) The inhibitory activity of A-485 and WM-8014 on KAT3B. Error bars indicate mean±SD of 3 biological replicates. (A) The inhibitory activity of A-485 and WM-8014 on KAT6A. Error bars indicate mean±SD of 3 biological replicates. (C) Western blot showed the level of H3K27ac after treatment by the indicated conditions. Representative of two independent experiments.

FIG. 42 shows the number of hCiPSC colonies generated under the original stage 1 condition (control), stage 1 condition plus A-485 or WM-8014 or a combination of the two (n=8). Error bars indicate mean±s.d. of eight biological replicates.

FIG. 43 shows the relative reprogramming efficiency of hCiPSCs induced from hADSCs and hADSCs with KAT3A, KAT3B and KAT6A knockdown (n=4). Error bars indicate mean±s.d. of four biological replicates.

FIG. 44 shows (A) an expression analysis by RT-qPCR for KAT3A, KAT3B, and KAT6A in control and knockdown cells (n=3, technical replicates). (B) the relative reprogramming efficiency of hCiPSCs induced from refractory hADSCs with KAT3A, KAT3B, and KAT6A knockdown (n=4). Error bars indicate the mean±SDs of 4 biological replicates.

FIG. 45 shows (A) the number of hCiPSC colonies generated from hADSCs isolated from the indicated donors in 16 days using the original or fast protocol (n=8). Error bars indicate mean±s.d. of eight biological replicates, and (B) the number of hCiPSC colonies generated from hASFs isolated from the indicated donors in 20 days (hASFs) using the original or the fast protocol (n=8). Error bars indicate mean±s.d. of eight biological replicates.

FIG. 46 shows a summary of reprogramming efficiencies of hADSCs and hASFs isolated from different donors in 16 days (hADSCs) or 20 days (hASFs) using the original or the fast protocol reported in this study.

FIG. 47 show (A) the number of hCiPS cell colonies generated from hADSCs (line 1013) and hASFs (line 38040), along with the associated reprogramming kinetics, using the original or fast protocol (n=8 biological replicates), and (B) immunofluorescence images of primary hCiPSC colonies in the wells of 24-well plates at different induction times using the original protocol or the fast protocol; ‘+2 days’ indicates that the duration of stage 3 was extended by 2 days, allowing the primary colonies to grow larger for improved imaging. Data are representative of more than three independent experiments: scale bar, 100 μm.

FIG. 48 shows the number of hCiPSC colonies generated from hADSCs-0618 and hADSCs-0809, along with the associated reprogramming kinetics, using the original or the fast protocol (n=8, biological replicates).

FIG. 49 shows (A) the number of hCiPSC colonies generated using the fast protocol from 10 to 14 days (n=8). Error bars indicate mean±s.d. of the eight biological replicates. and (B) the immunofluorescence of pluripotency markers in primary hCiPSC colonies generated in 10 days using the fast protocol. Data are representative of more than three independents experiments: scale bar, 100 μm.

### DETAILED DESCRIPTION

### Definitions

The term “converting” or “reprogramming,” and their grammatical equivalents as used herein when referring to cells refers to a process that alters or reverses the differentiation state of a cell (e.g., a somatic cell). As used herein, a conversion process, when referring to conversion of a cell of a first cell type into a cell of a second cell type, refer to a process where the cell of the first cell type is converted into a cell of the second cell type, or a process where one or more progenies of the cell of the first cell type is converted into a cell of the second cell type.

The term “differentiation” and its grammatical equivalents as used herein can refer to the process by which a less specialized cell (e.g., a more naive cell with a higher cell potency) becomes a more specialized cell type (e.g., a less naive cell with a lower cell potency); and that the term “de-differentiation” can refer to the process by which a more specialized cell becomes a less specialized cell type (e.g., a more naive cell with a higher cell potency).

As used herein, the term “cell potency” can refer to the ability of a cell to differentiate into cells of different lineages. For example, without wishing to be bound by a certain theory, a pluripotent cell (e.g., a stem cell) has the potential to differentiate into cells of, or derived from, any of the three germ layers, that is, endoderm (e.g., interior stomach lining, gastrointestinal tract, the lungs), mesoderm (e.g., muscle, bone, blood, urogenital), or ectoderm (e.g., epidermal tissues and nervous system), and accordingly has high cell potency; a multipotent cell (e.g., a stem cell of a certain type, such as hematopoietic stem cells, cardiac stem cells, or neural stem cells, etc.) has the ability to give rise to cells from a multiple, but limited, number of lineages (such as blood cell lineage, cardiac cell lineage, neural cell lineage) comparatively has a lower cell potency than pluripotent cells. Cells that are committed to a particular lineage or are terminally differentiated can have yet a lower cell potency.

The term “isolated population of cells” or “isolated cell population” as used herein refers to a group of non-naturally occurring cells.

The term “population of cells,” “cell population,” or grammatically equivalent thereof, as used herein refers to a group of cells, their progenies or progenies thereof, and/or the cells derived from thereof. For example, a population of first cells may comprise the first cells or the progenies of the first cells.

The term “progenies,” when used herein with reference toa cell, can refer to any of the daughter cells derived from mitotic division of the cell or mitotic division of any of the progenies of the cell.

Whenever the term “at least,” “greater than,” or “greater than or equal to” precedes the first numerical value in a series of two or more numerical values, the term “at least” or “greater than” applies to each one of the numerical values in that series of numerical values.

Whenever the term “at most,” “no more than,” “less than,” or “less than or equal to” precedes the first numerical value in a series of two or more numerical values, the term “no more than” or “less than” applies to each one of the numerical values in that series of numerical values.

As used herein, the singular forms “a”, “an”, and “the” include plural references unless the context clearly dictates otherwise.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

### Methods

Provided herein, in some aspects, are methods and compositions for cell conversions. In some instances, a method may comprise contacting a first population cells with a composition comprising one or more reprogramming factors. In some case, the contacting may comprise incubating a first population of cells with a composition comprising one or more reprogramming factors for a period of time. Subsequent to the contacting, at least a subset of the first population of cells may be converted into different cells. The population of cells subsequent to the conversion may comprise a second population of cells. During or subsequent to the conversion, the first population and second population of cells may have different biological properties. The different biological properties may comprise different expressions of genes, different expression of proteins, different cell proliferation properties (e.g., cell divisions or cell growth/increase of cell masses), different sizes of cells, different numbers of cells, different modifications of the genomes of the cells (e.g., epigenetic modifications), different cell cycle stages, different senescence stages, or different differentiation stages or types, or any combination thereof. The first population and second population of cells may differ by having at least two cells that have at least a different cellular activity. The first population and second population of cells may differ by having different cell types. The different cell types may have different cellular activities described herein.

Provided herein, in some aspects, are methods and compositions for converting cells in a plurality of stages. The methods and compositions for converting cells may comprise at least 2, 3, 4, 5, or more stages. The methods and compositions for converting cells may comprise at most 2, 3, 4, or 5 more stages. The methods and compositions for converting cells may comprise at most 2 stages. The methods and compositions for converting cells may comprise at most 3 stages. The methods and compositions for converting cells may comprise at 3 stages. The 3 stages may comprise stage 1 (or stage I), stage 2 (or stage II), and/or stage 3 (or stage III). The methods and compositions described herein may comprise stage 1 that involves conversion of a somatic cell into a cell with a higher cell potency (e.g., less specialized cell), such as an epithelial-like cell. The methods and compositions described herein may comprise stage 2 that involves conversion of an epithelial-like cell into a cell with a higher cell potency (e.g., less specialized cell), such as intermediate plastic state cell. The methods and compositions described herein may comprise stage 3 that involves conversion of an intermediate plastic state cell into a cell with a higher cell potency (e.g., less specialized cell), such as a pluripotent stem cell. The methods and compositions described herein may comprise stages 1, 2, and 3. A stage of the plurality of stages may comprise contacting a first population of cells with a first composition and converting at least a subset of the first population of cells into different cells. The population of cells subsequent to the conversion may comprise a second population of cells.

In some cases, a method may comprise (1) contacting a first population of cells with a first composition; (2) converting at least a subset of the first population of cells into different cells and generating a second population of cells comprising the converted cells; (3) contacting the second population of cells with a second composition; (4) converting at least a subset of the second population of cells into different cells and generating a third population of cells comprising the converted cells; (5) contacting the third population of cells with a third composition; (6) converting at least a subset of the third population of cells into different cells and generating a fourth population of cells comprising the converted cells. The first, second, and third compositions may each comprise one or more reprogramming factors. Any of the first, second, or third composition may comprise one or more reprogramming factors. The first, second, and third compositions may be different. The first, second, third, and fourth populations of cells may be different. In some cases, prior to, during, or subsequent to each contacting, at least a cell of the population of cells being contacted with a composition may be cultured. In some cases, prior to, during, or subsequent to each converting, the population of cells being contacted with a composition may be cultured. When the cells are cultured, the cells may undergo cell proliferation.

It will be appreciated that the ordinal numbering of a populations of cells or composition relative to other populations of cells or compositions may not limit the populations of cells or composition to specific populations of cells or composition. In some cases, the ordinal numbering of a populations of cells or composition should be understood to distinguish a population of cells or compositions from another population(s) of cells or composition(s).

In some cases, a method may comprise (1) contacting a first population of cells comprising somatic cells with a first composition; (2) converting at least a subset of the somatic cells or the progenies thereof into different cells and generating a second population of cells comprising the converted cells comprising epithelial-like cells; (3) contacting the second population of cells comprising epithelial-like cells with a second composition; (4) converting at least a subset of the second population of cells into different cells and generating a third population of cells comprising the converted cells comprising intermediate plastic state cells; (5) contacting the third population of cells comprising intermediate plastic state cells with a third composition; (6) converting at least a subset of the third population of cells comprising intermediate plastic state cells into different cells and generating a fourth population of cells comprising the converted cells comprising pluripotent stem cells.

In some cases, a method may convert a population of cells comprising somatic cells into a different population of cells comprising pluripotent stem cells within a pluripotent stem cell conversion time period. The pluripotent stem cell conversion time period may comprise the time period from contacting the population of cells comprising somatic cells to the time when at least a pluripotent stem cell is generated. The pluripotent stem cell conversion time period may be less than about 100 days, 90 days, 80 days, 70 days, 60 days, 59 days, 58 days, 57 days, 56 days, 55 days, 54 days, 53 days, 52 days, 51 days, 50 days, 49 days, 48 days, 47 days, 46 days, 45 days, 44 days, 43 days, 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days or less. The pluripotent stem cell conversion time period may be less than about 55 days. The pluripotent stem cell conversion time period may be less than about 54 days. The pluripotent stem cell conversion time period may be less than about 53 days. The pluripotent stem cell conversion time period may be less than about 52 days. The pluripotent stem cell conversion time period may be less than about 51 days. The pluripotent stem cell conversion time period may be less than about 50 days. The pluripotent stem cell conversion time period may be less than about 49 days. The pluripotent stem cell conversion time period may be less than about 48 days. The pluripotent stem cell conversion time period may be less than about 47 days. The pluripotent stem cell conversion time period may be less than about 46 days. The pluripotent stem cell conversion time period may be less than about 45 days. The pluripotent stem cell conversion time period may be less than about 44 days. The pluripotent stem cell conversion time period may be less than about 43 days. The pluripotent stem cell conversion time period may be less than about 42 days. The pluripotent stem cell conversion time period may be less than about 41 days. The pluripotent stem cell conversion time period may be less than about 40 days. The pluripotent stem cell conversion time period may be less than about 39 days. The pluripotent stem cell conversion time period may be less than about 38 days. The pluripotent stem cell conversion time period may be less than about 37 days. The pluripotent stem cell conversion time period may be less than about 36 days. The pluripotent stem cell conversion time period may be less than about 35 days. The pluripotent stem cell conversion time period may be less than about 34 days. The pluripotent stem cell conversion time period may be less than about 33 days. The pluripotent stem cell conversion time period may be less than about 32 days. The pluripotent stem cell conversion time period may be less than about 31 days. The pluripotent stem cell conversion time period may be less than about 30 days. The pluripotent stem cell conversion time period may be less than about 29 days. The pluripotent stem cell conversion time period may be less than about 28 days. The pluripotent stem cell conversion time period may be less than about 27 days. The pluripotent stem cell conversion time period may be less than about 26 days. The pluripotent stem cell conversion time period may be less than about 25 days. The pluripotent stem cell conversion time period may be less than about 24 days. The pluripotent stem cell conversion time period may be less than about 23 days. The pluripotent stem cell conversion time period may be less than about 22 days. The pluripotent stem cell conversion time period may be less than about 21 days. The pluripotent stem cell conversion time period may be less than about 20 days. The pluripotent stem cell conversion time period may be less than about 19 days. The pluripotent stem cell conversion time period may be less than about 18 days. The pluripotent stem cell conversion time period may be less than about 17 days. The pluripotent stem cell conversion time period may be less than about 16 days. The pluripotent stem cell conversion time period may be less than about 15 days.

In some cases, a method may convert a population of cells comprising somatic cells into a different population of cells comprising pluripotent stem cells with a pluripotent stem cell conversion efficiency. The pluripotent stem cell conversion efficiency may be measured as a ratio (e.g., percentage) of the number of pluripotent stem cells generated relative to the number of somatic cells, to which the method disclosed herein is applied for generating the number of pluripotent stem cells. For example, if the method generates one pluripotent stem cell from 1000 somatic cells, the pluripotent stem cell conversion efficiency of the method is 0.1%. In some cases, the pluripotent stem cell conversion efficiency of the method may be at least about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.5% or more. In some cases, the pluripotent stem cell conversion efficiency of the method may be about 0.0001%, 0.0002%, 0.0003%, 0.0004%, 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, or 0.5%. The pluripotent stem cell conversion efficiency of the method may be about 0.0005%. The pluripotent stem cell conversion efficiency of the method may be about 0.0006%. The pluripotent stem cell conversion efficiency of the method may be about 0.0007%. The pluripotent stem cell conversion efficiency of the method may be about 0.0008%. The pluripotent stem cell conversion efficiency of the method may be about 0.0009%. The pluripotent stem cell conversion efficiency of the method may be about 0.001%. The pluripotent stem cell conversion efficiency of the method may be about 0.002%. The pluripotent stem cell conversion efficiency of the method may be about 0.003%. The pluripotent stem cell conversion efficiency of the method may be about 0.004%. The pluripotent stem cell conversion efficiency of the method may be about 0.005%. The pluripotent stem cell conversion efficiency of the method may be about 0.006%. The pluripotent stem cell conversion efficiency of the method may be about 0.007%. The pluripotent stem cell conversion efficiency of the method may be about 0.008%. The pluripotent stem cell conversion efficiency of the method may be about 0.009%. The pluripotent stem cell conversion efficiency of the method may be about 0.01%. The pluripotent stem cell conversion efficiency of the method may be about 0.02%. The pluripotent stem cell conversion efficiency of the method may be about 0.03%. The pluripotent stem cell conversion efficiency of the method may be about 0.04%. The pluripotent stem cell conversion efficiency of the method may be about 0.05%. The pluripotent stem cell conversion efficiency of the method may be about 0.06%. The pluripotent stem cell conversion efficiency of the method may be about 0.07%. The pluripotent stem cell conversion efficiency of the method may be about 0.08%. The pluripotent stem cell conversion efficiency of the method may be about 0.09%. The pluripotent stem cell conversion efficiency of the method may be about 0.1%.

In some cases, provided herein is a method for producing pluripotent stem cells. In some cases, the method comprises obtaining epithelial-like cells that express LIN28A for conversion. In some cases, the method comprises converting the epithelial-like cells or progenies thereof into intermediate plastic state cells for further conversion. In some cases, the intermediate plastic state cells express LIN28A and SALL4, and one or more of MSX2, NMYC, SDC1, WNT4, FGF19, or TOP2A. In some cases, the method comprises converting the intermediate plastic state cells into pluripotent stem cells. In some cases, conversion of the epithelial-like cells comprises contacting the epithelial-like cells with a composition comprising a glycogen synthase kinase 3 (GSK-3), a transforming growth factor-beta (TGFβ) receptor inhibitor; and a c-Jun kinase inhibitor. In some cases, the composition contacted to the epithelial-like cells further comprises a CBP/p300 bromodomain inhibitor or an adenosine kinase inhibitor. In some cases, the composition contacted to the epithelial-like cells further comprises a S-adenosyl-L-homocysteine (SAH) hydrolase inhibitor or an adenosine kinase inhibitor.

In some cases, the method for producing pluripotent stem comprises obtaining a first cell population that comprises epithelial-like cells that express LIN28A. In some cases, the method further comprises contacting the first cell population with a second composition, thereby obtaining a second cell population. The second composition may comprise (i) a glycogen synthase kinase 3 (GSK-3); (ii) a transforming growth factor-beta (TGFβ) receptor inhibitor; and (iii) a c-Jun kinase inhibitor. In some cases, the method further comprises contacting the second cell population with a third composition, thereby obtaining a third cell population comprising pluripotent stem cells. The third composition can comprise: (1) a MEK inhibitor; (2) a B-Raf inhibitor; and (3) a histone deacetylase inhibitor.

In some cases, the epithelial-like cells that express LIN28A are obtained by reprogramming somatic cells. In some cases, the method disclosed herein comprises contacting a population of cells comprising the somatic cells with a composition comprising one or more reprogramming factors. In some cases, the composition contacted to the somatic cells comprises one or more of a glycogen synthase kinase 3 (GSK-3), a transforming growth factor-beta (TGFβ) receptor inhibitor, and a retinoic acid receptor (RAR) agonist. In some cases, the composition contacted to the somatic cells further comprises an Akt inhibitor or a SET domain containing 2 (SETD2) inhibitor.

In some cases, the epithelial-like cells that express LIN28A are obtained by reprogramming a population of cells comprising epithelial cells. Epithelial cells can be converted into epithelial-like cells that express LIN28A by contacting epithelial cells with a composition comprising one or more reprogramming factors. In some cases, the composition contacted to the epithelial cells comprises one or more of a glycogen synthase kinase 3 (GSK-3), a transforming growth factor-beta (TGFβ) receptor inhibitor, and a retinoic acid receptor (RAR) agonist. In some cases, the composition contacted to the epithelial cells further comprises an Akt inhibitor or a SET domain containing 2 (SETD2) inhibitor. In other case, the composition contacted to the epithelial cells comprises one or more of a CBP/p300 bromodomain inhibitor, an adenosine kinase inhibitor, a glycogen synthase kinase 3 (GSK-3), a transforming growth factor-beta (TGFβ) receptor inhibitor, or a c-Jun kinase inhibitor. In some cases, the composition contacted to the epithelial cells comprises a CBP/p300 bromodomain inhibitor or an adenosine kinase inhibitor, a glycogen synthase kinase 3 (GSK-3), a transforming growth factor-beta (TGFβ) receptor inhibitor, and a c-Jun kinase inhibitor. In some cases, the composition contacted to the epithelial cells further comprises a retinoic acid receptor (RAR) agonist, a CBP/p300 bromodomain inhibitor, and a S-adenosyl-L-homocysteine (SAH) hydrolase inhibitor or an adenosine kinase inhibitor.

A cell in any populations of cells described herein may comprise a mammalian cell. A cell may comprise a mouse cell, a hamster cell, a rat cell, or a rodent cell. A cell may comprise a mouse cell. A cell may comprise a hamster cell. A cell may comprise a rat cell. A cell may comprise a rodent cell. In some cases, a cell may comprise a primate cell. A cell may comprise a strepsirrhine cell or a haplorrhine cell. A cell may comprise a monkey cell, an ape cell, or a human cell. In some cases, a cell may comprise a human cell. A cell may comprise a monkey cell. A cell may comprise an ape cell.

In some embodiments, the method disclosed herein is a method for obtaining induced pluripotent stem cells from somatic cells selected from fibroblasts, blood cells, epithelial cells, or endothelial cells, etc. In certain embodiment, the chemical reprogramming method disclosed herein is a method for obtaining induced pluripotent stem cells from fibroblasts. In further embodiments, the fibroblasts are selected from adipose fibroblasts, skin fibroblasts, or umbilical cord mesenchymal stem cells.

An object of the present invention is to provide the use of a histone acetyltransferase inhibitor in the chemical reprogramming of somatic cells into induced pluripotent stem cells, wherein the inhibitor is used before the somatic cells become a cell population with the following characteristics: the cell population mainly comprises epithelial-like cells positive for the pluripotency marker LIN28A, and compared with the somatic cells as the starting cells, it has epithelial cell surface markers with increased expression levels and somatic cell markers with decreased expression levels. In some embodiments, the epithelial cell surface markers are selected from one or more of KRT8, KRT18, or KRT19. In some embodiments, the somatic cell markers are selected from one or more of VIM, SNAI2, COL5A1, PRKX1, COL6A2, ZEB1, MMP1, TWIST1, COL1AL. In some embodiments, the histone acetyltransferase inhibitor is a KAT3A/KAT3B inhibitor. In some embodiments, the histone acetyltransferase inhibitor is KAT6A inhibitor. In some embodiment, the histone acetyltransferase inhibitor is a combination of a KAT3A/KAT3B inhibitor and a KAT6A inhibitor. In some embodiments, the histone acetyltransferase inhibitor is selected from A485, CBP/P300 IN 8, WM8014, GEN049, WM1119, CBP/P300 IN 12, SGCCBP30, ICBP112, or any combination thereof. In some embodiments, the histone acetyltransferase inhibitor is selected from WM8014, A485, or a combination of both.

In some embodiments, the present invention provides a chemical reprogramming method for obtaining induced pluripotent stem cells from somatic cells of primates. The method includes the step of exposing the somatic cells to the first composition disclosed herein, optionally for 5 to 8 days, to obtain a cell population with increased differentiation potential compared to the somatic cells, and the step of treating the cell population first with the second composition disclosed herein, optionally for 1 to 8 days, and then treating it with the third composition disclosed herein, optionally for 4 to 8 days, to obtain induced pluripotent stem cells.

In certain embodiment, the method disclosed herein includes exposing somatic cells to a composition containing 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, and 0.5 μM A-485; or a composition containing 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, and 1 μM WM8014; or a composition containing 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 0.5 μM A-485, and 1 μM WM8014; or a composition containing 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 0.5 μM A-485, and 0.5 μM AM095; or a composition containing 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 0.5 μM A-485, 1 μM WM8014, and 0.5 μM AM095; or a composition containing 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 1 μM WM8014, and 0.5 μM AM095. After 5 to 8 days, a cell population with increased differentiation potential compared to the somatic cells is obtained. The cell population is first treated with a composition containing 5 μM CHIR999021, 10 μM 616452, 2 μM TTNPB, 0.5 μM SAG, 10 μM Y-27632, 0.5 μM JNKIN8, 1 μM Ruxolitinib, 2 μM BIRB796, 2 μM SGC-CBP30, 2 μM EPZ5676, 0.2 μM DZNep, 0.5 μM VTP50469, 0.5 μM Iodotubercidin, 5 μM RA, 0.02 μM GSK3685032, 0.2 μM HY-10249A (CAS No. 842148-40-7), and 1 μM CX-4945 for 1 to 8 days, then treated with a composition containing 1 μM CHIR999021, 10 μM Y-27632, 1 μM PD0325901, 0.5 μM SB590885, 200 μM VPA, 10 μM Tranylcypromine, 2 μM EPZ5676, 0.2 μM DZNep, and 10 μM PY60 for 2 to 4 days, and then treated with a composition containing 1 μM CHIR999021, 10 μM Y-27632, 1 μM PD0325901, 0.5 μM SB590885, and 200 μM VPA for 2 to 4 days to obtain induced pluripotent stem cells.

In some embodiments, through the chemical reprogramming method disclosed herein, somatic cells can be converted into pluripotent stem cells within about 10 days to about 30 days. In some embodiments, through the chemical reprogramming method disclosed herein, somatic cells can be converted into pluripotent stem cells within about 10 days to about 24 days. In some embodiments, through the chemical reprogramming method disclosed herein, somatic cells can be converted into pluripotent stem cells within about 10 days to about 18 days. In some embodiments, through the chemical reprogramming method disclosed herein, somatic cells can be converted into pluripotent stem cells at about 10 days, about 11 days, about 12 days, about 13 days, about 14 days, about 15 days, about 16 days, about 17 days, or about 18 days.

In some embodiments, the chemical reprogramming method for obtaining induced pluripotent stem cells from somatic cells of primates disclosed herein includes the step of contacting the somatic cells with the following composition for 5 to 8 days (for example, contacting for 5 days, 6 days, 7 days, or 8 days) to obtain a cell population with increased differentiation potential compared to the somatic cells, and the step of further dedifferentiating the cell population into pluripotent stem cells, wherein the composition contains 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, and 0.5 μM A-485; or contains 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, and 1 μM WM8014; or contains 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 1 μM WM8014, and 0.5 μM A-485; or contains 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 0.5 μM A-485, and 0.5 μM AM095; or contains 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 1 μM WM8014, and 0.5 μM AM095; or contains 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 0.5 μM A-485, 1 μM WM8014, and 0.5 μM AM095.

### Stage 1

In some aspects, provided herein are methods and compositions for conversion of a somatic cell into a cell with a higher cell potency (e.g., less specialized cell), such as an epithelial-like cell—the conversion process referred herein also as “stage 1.” A stage 1 method may be part of a conversion process that reprograms somatic cells into pluripotent stem cells. A stage 1 method may be the first stage of a conversion process that reprograms somatic cells into pluripotent stem cells.

A stage 1 method may comprise contacting a first cell population with a first composition. A stage 1 method may comprise, subsequent to or during the contacting, converting a subset of the first cell population into different cells. The cell population comprising the different cells may comprise a second cell population. A stage 1 method may comprise incubating the first cell population with the first composition for a period of time. The subset of the first cell population may be converted into the different cells prior to, during, or subsequent to the incubating. In some cases, a stage 1 method may comprise removing the first composition from the second population of cells. In other cases, a stage 1 method may comprise removing the first composition from the first population of cells.

The first population of stage 1 cells may comprise somatic cells. A somatic cell may comprise a skin cell, a nerve cell, a muscle cell, or a blood cell. A somatic cell may not comprise a germ cell. A somatic cell may not comprise an undifferentiated cell. A somatic cell may not comprise a gamete (sperms or eggs). A somatic cell may not comprise a gametocyte. In some cases, a somatic cell may also comprise a muscle cell, a fat cell, a connective tissue cell, a vasculature cell, a neuron, a bone cell, or a skin cell. The first population of stage 1 cells may comprise fibroblasts, primary human adult adipose derived mesenchymal stromal cells (hADSCs), smooth muscle cells, cardiac muscle cells, skeletal muscle cells, neurons, red blood cells, white blood cells, platelets, osteoblasts, osteoclasts, squamous cells, basal cells, or melanocytes, or any combination thereof. The first population of stage 1 cells may comprise fibroblasts or hADSCs. The first population of stage 1 cells may comprise fibroblasts. The first population of stage 1 cells may comprise primary human adult adipose derived mesenchymal stromal cells (hADSCs). The first population of stage 1 cells may comprise smooth muscle cells. The first population of stage 1 cells may comprise cardiac muscle cells. The first population of stage 1 cells may comprise skeletal muscle cells. The first population of stage 1 cells may comprise neurons. The first population of stage 1 cells may comprise red blood cells. The first population of stage 1 cells may comprise white blood cells. The first population of stage 1 cells may comprise platelets. The first population of stage 1 cells may comprise osteoblasts. The first population of stage 1 cells may comprise osteoclasts. The first population of stage 1 cells may comprise squamous cells. The first population of stage 1 cells may comprise basal cells. The first population of stage 1 cells may comprise melanocytes. The first population of stage 1 cells may comprise cells from the intestinal epithelium. The first population of stage 1 cells may comprise neonatal (for example foreskin) or adult fibroblasts. The first population of stage 1 cells may comprise epithelial cells, endothelial cells, cells of mesenchymal origin, parenchymal cells (for example, hepatocytes), neurological cells, or connective tissue cells, or any combination thereof. The first population of stage 1 cells may not comprise cells that are not somatic cells. In some cases, the first population of stage 1 cells may comprise germ cells.

The first population of stage 1 cells may be isolated by disaggregating an appropriate organ or tissue. For example, the tissue or organ can be disaggregated mechanically and/or treated with digestive enzymes and/or chelating agents that weaken the connections between neighboring cells, so that the tissue can be dispersed to form a suspension of individual cells without appreciable cell breakage. Enzymatic dissociation can be accomplished by mincing the tissue and treating the minced tissue with one or more enzymes such as trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, pressure cells, or insonators.

The second population of stage 1 cells may comprise epithelial-like cells. An epithelial-like cell may not be a naturally occurring cell. An epithelial-like cell may express a combination of genes that are not expressed by a naturally occurring cell. An epithelial-like cell may express at least one gene at level at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 5-fold, 100-fold, or higher, relative to a naturally occurring cell. An epithelial-like cell may express at least one gene at level at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% lower, relative to a naturally occurring cell. The second population of stage 1 cells may comprise somatic or epithelial-like cells. The second population of stage 1 cells may comprise somatic and epithelial-like cells. The second population of stage 1 cells may not comprise somatic cells. In some cases, the second population of stage 1 cells may comprise fewer somatic cells than the first population of stage 1 cells. For example, the second population of stage 1 cells may have 0.001%, 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90% or 99% fewer somatic cells than the first population of stage 1 cells. In some cases, the second population of stage 1 cells may comprise more epithelial-like cells than the first population of stage 1 cells. For example, the second population of stage 1 cells may have 0.001%, 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 5-fold, 100-fold, or more epithelial-like cells than the first population of stage 1 cells.

An epithelial-like cell may express LIN28A, NMYC, WNT2B, PAX8, SMAD3, GLI3, KRT18, KRT19, WT1, or TBX2, or any combination thereof. An epithelial-like cell may express LIN28A. An epithelial-like cell may express NMYC. An epithelial-like cell may express WNT2B. An epithelial-like cell may express PAX8. An epithelial-like cell may express SMAD3. An epithelial-like cell may express GLI3. An epithelial-like cell may express KRT18. An epithelial-like cell may express KRT19. An epithelial-like cell may express WT1. An epithelial-like cell may express TBX2. An epithelial-like cell may express LIN28A and one or more of NMYC, WNT2B, PAX8, SMAD3, GLI3, KRT18, KRT19, WT1, or TBX2. An epithelial-like cell may express LIN28A and NMYC. An epithelial-like cell may express LIN28A and WNT2B. An epithelial-like cell may express LIN28A and PAX8. An epithelial-like cell may express LIN28A and SMAD3. An epithelial-like cell may express LIN28A and GLI3. An epithelial-like cell may express LIN28A and KRT18. An epithelial-like cell may express LIN28A and KRT19. An epithelial-like cell may express LIN28A and WT1. An epithelial-like cell may express LIN28A and TBX2. An epithelial-like cell may not express MMP1, ZEB1, VIM, COL1A1, COL5A1, COL6A2, PRRX1, SNAI2, TWIST1, or TWIST2, or any combination thereof. An epithelial-like cell may not express MMP1. An epithelial-like cell may not express ZEB1. An epithelial-like cell may not express VIM. An epithelial-like cell may not express COL1AL. An epithelial-like cell may not express COL5A1. An epithelial-like cell may not express COL6A2. An epithelial-like cell may not express PRRX1. An epithelial-like cell may not express SNAI2. An epithelial-like cell may not express TWIST1. An epithelial-like cell may not express TWIST2. An epithelial-like cell may express LIN28A but does not express MMP1, ZEB1, VIM, COL1A1, COL5A1, COL6A2, PRRX1, SNAI2, TWIST1, or TWIST2, or any combination thereof. An epithelial-like cell may express LIN28A but does not express VIM. An epithelial-like cell may express LIN28A but does not express COL1AL. An epithelial-like cell may express LIN28A but does not express COL5A1. An epithelial-like cell may express LIN28A but does not express COL6A2. An epithelial-like cell may express LIN28A but does not express PRRX1. An epithelial-like cell may express LIN28A but does not express SNAI2. An epithelial-like cell may express LIN28A but does not express TWIST1. An epithelial-like cell may express LIN28A but does not express TWIST2. An epithelial-like cell may express LIN28A; one or more of NMYC, WNT2B, PAX8, SMAD3, GLI3, KRT18, KRT19, WT1, or TBX2; but does not express MMP1, ZEB1, VIM, COL1A1, COL5A1, COL6A2, PRRX1, SNAI2, TWIST1, or TWIST2, or any combination thereof.

A first population of stage 1 cells may not comprise the epithelial-like cell described herein. A somatic cell of the first population of stage 1 cells may not express LIN28A, NMYC, WNT2B, PAX8, SMAD3, GLI3, KRT18, KRT19, WT1, or TBX2, or any combination thereof. The somatic cell may not express LIN28A. The somatic cell may not express NMYC. The somatic cell may not express WNT2B. The somatic cell may not express PAX8. The somatic cell may not express SMAD3. The somatic cell may not express GLI3. The somatic cell may not express KRT18. The somatic cell may not express KRT19. The somatic cell may not express WT1. The somatic cell may not express TBX2. The somatic cell may not express LIN28A and may not one or more of NMYC, WNT2B, PAX8, SMAD3, GLI3, KRT18, KRT19, WT1, or TBX2. The somatic cell may not express LIN28A or NMYC. The somatic cell may not express LIN28A or WNT2B. The somatic cell may not express LIN28A or PAX8. The somatic cell may not express LIN28A or SMAD3. The somatic cell may not express LIN28A or GLI3. The somatic cell may not express LIN28A or KRT18. The somatic cell may not express LIN28A or KRT19. The somatic cell may not express LIN28A or WT1. The somatic cell may not express LIN28A or TBX2. The somatic cell may express MMP1, ZEB1, VIM, COL1A1, COL5A1, COL6A2, PRRX1, SNAI2, TWIST1, or TWIST2 or any combination thereof. The somatic cell may express MMP1. The somatic cell may express ZEB1. The somatic cell may express VIM. The somatic cell may express COL1AL. The somatic cell may express COL5A1. The somatic cell may express COL6A2. The somatic cell may express PRRX1. The somatic cell may express SNAI2. The somatic cell may express TWIST1. The somatic cell may express TWIST2.

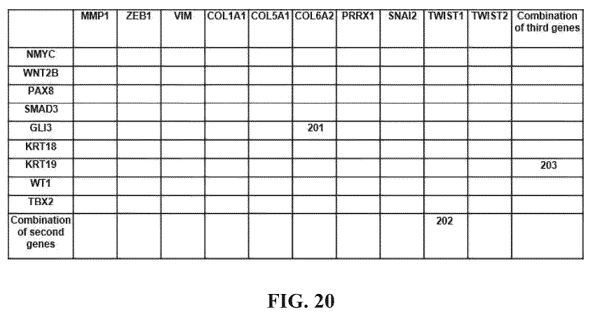

An epithelial-like cell may express LIN28A and a second gene but does not express a third gene. An epithelial-like cell may express LIN28A and one or more second genes but does not express one or more third genes. The second gene expressed by the epithelial-like cell may comprise NMYC, WNT2B, PAX8, SMAD3, GLI3, KRT18, KRT19, WT1, or TBX2, or any combination thereof. A somatic cell may not express LIN28A or a second gene but expresses a third gene. A somatic cell may not express LIN28A or one or more second genes but expresses one or more third genes. The second gene expressed by the epithelial-like cell but not by the somatic cell may comprise NMYC, WNT2B, PAX8, SMAD3, GLI3, KRT18, KRT19, WT1, or TBX2, or any combination thereof. The second gene thereof may comprise NMYC. The second gene thereof may comprise WNT2B. The second gene thereof may comprise PAX8. The second gene thereof may comprise SMAD3. The second gene thereof may comprise GLI3. The second gene thereof may comprise KRT18. The second gene thereof may comprise KRT19. The second gene thereof may comprise WT1. The second gene thereof may comprise TBX2. The third gene not expressed by the epithelial-like cell may comprise MMP1, ZEB1, VIM, COL1A1, COL5A1, COL6A2, PRRX1, SNAI2, TWIST1, or TWIST2, or any combination thereof. The third gene thereof may comprise MMP1. The third gene thereof may comprise ZEB1. The third gene thereof may comprise VIM. The third gene thereof may comprise COL1AL. The third gene thereof may comprise COL5A1. The third gene thereof may comprise COL6A2. The third gene thereof may comprise PRRX1. The third gene thereof may comprise SNAI2. The third gene thereof may comprise TWIST1. The third gene thereof may comprise TWIST2. FIG. 20 depicts exemplary epithelial-like cells that express LIN28A in stage 1 or stage 2 shown in a heatmap. The y-axis and x-axis of the heatmap show the second and the third genes, respectively. Each pixel of the heatmap represents one cell population. For example, cells 201 express LIN28A and GLI3 but not COL6A2. Cells 202 express LIN28A and any combinations of the second gene (e.g., NYMC and WNT2B) but not TWIST1. Cells 203 express LIN28A and KRT19 but not any combinations of the third genes (e.g., MMP1 and VIM).

A cell of the second population of stage 1 cells may express higher levels of LIN28A, NMYC, WNT2B, PAX8, SMAD3, GLI3, KRT18, KRT19, WT1, or TBX2, or any combination thereof, relative to a cell of the first population of stage 1 cells. The higher level of expression of any one of LIN28A, NMYC, WNT2B, PAX8, SMAD3, GLI3, KRT18, KRT19, WT1, or TBX2 in a cell of the second population of stage 1 cells may be at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 5-fold, 100-fold, or more, relative to a cell of the first population of stage 1 cells. A cell of the second population of stage 1 cells may express lower levels of any one of MMP1, ZEB1, VIM, COL1A1, COL5A1, COL6A2, PRRX1, SNAI2, TWIST1, or TWIST2, relative to a cell of the first population of stage 1 cells. The lower level of expression of any one of MMP1, ZEB1, VIM, COL1A1, COL5A1, COL6A2, PRRX1, SNAI2, TWIST1, or TWIST2 in a cell of the second population of stage 1 cells may be at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%, relative to a cell of the first population of stage 1 cells. The levels of expression can be measured by any methods described herein. For examples, gene expression can be measured by methods described in EXAMPLE 2. Gene expression can be measured by using any one of SEQ ID NO: 1-83 (including controls).

In some aspects, provided herein is a composition that comprises reprogramming factors for stage 1 conversion, or comprises cells of stage 1 (the first population of cells or the second population of cells), or comprises cells of stage 1 (the first population of cells or the second population of cells) and reprogramming factors for stage 1 conversion. In some cases, a composition comprises a culture medium comprising the reprogramming factors for stage 1 conversion.

In some cases, a composition may comprise an isolated population of the second population of stage 1 cells. In some cases, a composition may comprise an isolated population the first population of stage 1 cells. An isolated population of stage 1 cells may comprise at least about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more cells. An isolated population of stage 1 cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 cells. An isolated population of stage 1 cells may comprise at least one epithelial-like cell. In some cases, an isolated population of stage 1 cells may comprise at least about 1, 1×10\hat{ }1, 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more epithelial-like cells. An isolated population of cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 epithelial-like cells. In some cases, an isolated population of stage 1 cells may comprise at least about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more somatic cells. An isolated population of cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 somatic cells. In some cases, an isolated population of stage 1 cells may comprise at least about 1×10\hat{ }1, 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more somatic cells, epithelial-like cells, or a combination thereof. An isolated population of stage 1 cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 epithelial-like cells, somatic cells, or a combination thereof.

A composition may comprise a chemical reprogramming factor. A composition may comprise a plurality of chemical reprogramming factors. A composition may comprise at least 1, 2, 3, 4, 5, 6, 7, 8, 9, 10, 11, 12, 13, 14, 15, 16, 17, 18, 19, 20 or more chemical reprogramming factors. A composition may comprise at most 1, 2, 3, 4, 5, 6, 7, 8, 9, 10, 11, 12, 13, 14, 15, 16, 17, 18, 19, 20 or more chemical reprogramming factors. A composition may comprise 1 chemical reprogramming factors. A composition may comprise 2 chemical reprogramming factors. A composition may comprise 3 chemical reprogramming factors. A composition may comprise 4 chemical reprogramming factors. A composition may comprise 5 chemical reprogramming factors. A composition may comprise 6 chemical reprogramming factors. A composition may comprise 7 chemical reprogramming factors. A composition may comprise 8 chemical reprogramming factors. A composition may comprise 9 chemical reprogramming factors. A composition may comprise 10 chemical reprogramming factors. A composition may comprise 11 chemical reprogramming factors. A composition may comprise 12 chemical reprogramming factors. A composition may comprise 13 chemical reprogramming factors. A composition may comprise 14 chemical reprogramming factors. A composition may comprise 15 chemical reprogramming factors. A composition may comprise 16 chemical reprogramming factors. A composition may comprise 17 chemical reprogramming factors. A composition may comprise 18 chemical reprogramming factors. A composition may comprise 19 chemical reprogramming factors. A composition may comprise 20 chemical reprogramming factors. A chemical reprogramming factor in a composition may comprise any chemical reprogramming factors described here.

A composition may comprise a GSK-3 inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor, or any combination thereof. A composition may comprise at least 1, 2, 3, 4, 5, 6, 7, 8, 9, or 10 of a GSK-3 inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor. A composition may comprise at most 1, 2, 3, 4, 5, 6, 7, 8, 9, or 10 of a glycogen synthase kinase 3 (GSK-3) inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor. A composition may comprise a GSK-3 inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor.

A composition may comprise a GSK-3 inhibitor. A composition may comprise a transforming growth factor-beta (TGFβ) receptor inhibitor. A composition may comprise a retinoic acid receptor (RAR) agonist. A composition may comprise an Akt inhibitor. A composition may comprise a SET domain containing 2 (SETD2) inhibitor. A composition may comprise a Dot1L inhibitor. A composition may comprise an agonist for the G protein-coupled receptor Smoothened. A composition may comprise a Jak1/Jak2 inhibitor. A composition may comprise a S-adenosyl-L-homocysteine (SAH) hydrolase inhibitor. A composition may comprise a Menin-MLL interaction inhibitor. A composition may comprise a GSK-3 inhibitor and a transforming growth factor-beta (TGFβ) receptor inhibitor. A composition may comprise a transforming growth factor-beta (TGFβ) receptor inhibitor and a retinoic acid receptor (RAR) agonist. A composition may comprise a glycogen synthase kinase 3 (GSK-3) inhibitor and a retinoic acid receptor (RAR) agonist.

A composition may comprise a GSK-3 inhibitor, a transforming growth factor-beta (TGFβ) receptor inhibitor, or a retinoic acid receptor (RAR) agonist, or any combination thereof. A composition may comprise a GSK-3 inhibitor, a transforming growth factor-beta (TGFβ) receptor inhibitor, and a retinoic acid receptor (RAR) agonist. A composition may comprise a glycogen synthase kinase 3 (GSK-3) inhibitor, a Transforming growth factor-beta (TGFβ) receptor inhibitor, a retinoic acid receptor (RAR) agonist, an Akt inhibitor, or a SET domain containing 2 (SETD2) inhibitor, or any combination thereof. A composition may comprise a GSK-3 inhibitor, a transforming growth factor-beta (TGFβ) receptor inhibitor, a retinoic acid receptor (RAR) agonist, an Akt inhibitor, and a SET domain containing 2 (SETD2) inhibitor. A composition may comprise a glycogen synthase kinase 3 (GSK-3) inhibitor, a transforming growth factor-beta (TGFβ) receptor inhibitor, a retinoic acid receptor (RAR) agonist, or an Akt inhibitor, or any combination thereof. A composition may comprise a GSK-3 inhibitor, a transforming growth factor-beta (TGFβ) receptor inhibitor, a retinoic acid receptor (RAR) agonist, or an Akt inhibitor. A composition may comprise a glycogen synthase kinase 3 (GSK-3) inhibitor, a transforming growth factor-beta (TGFβ) receptor inhibitor, a retinoic acid receptor (RAR) agonist, and an Akt inhibitor. A composition may comprise a GSK-3 inhibitor, a transforming growth factor-beta (TGFβ) receptor inhibitor, a retinoic acid receptor (RAR) agonist, or a SET domain containing 2 (SETD2) inhibitor, or any combination thereof. A composition may comprise a GSK-3 inhibitor, a transforming growth factor-beta (TGFβ) receptor inhibitor, a retinoic acid receptor (RAR) agonist, or a SET domain containing 2 (SETD2) inhibitor. A composition may comprise a GSK-3 inhibitor, a transforming growth factor-beta (TGFβ) receptor inhibitor, a retinoic acid receptor (RAR) agonist, and a SET domain containing 2 (SETD2) inhibitor. The composition may further comprise a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor, or any combination thereof.

A composition may comprise somatic cells; and a GSK-3 inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor, or any combination thereof. A composition may comprise somatic cells; and at least 1, 2, 3, 4, 5, 6, 7, 8, 9, or 10 of a GSK-3 inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, and a Menin-MLL interaction inhibitor. A composition may comprise somatic cells; and at most 1, 2, 3, 4, 5, 6, 7, 8, 9, or 10 of a GSK-3 inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, and a Menin-MLL interaction inhibitor. A composition may comprise somatic cells; and a GSK-3 inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, and a Menin-MLL interaction inhibitor.

A composition may comprise somatic cells; a GSK-3 inhibitor and a transforming growth factor-beta (TGFβ) receptor inhibitor. A composition may comprise somatic cells; a transforming growth factor-beta (TGFβ) receptor inhibitor and a retinoic acid receptor (RAR) agonist. A composition may comprise somatic cells; a glycogen synthase kinase 3 (GSK-3) inhibitor and a retinoic acid receptor (RAR) agonist. A composition may comprise somatic cells, and a GSK-3 inhibitor. A composition may comprise somatic cells; and a transforming growth factor-beta (TGFβ) receptor inhibitor. A composition may comprise somatic cells; and a retinoic acid receptor (RAR) agonist. A composition may comprise somatic cells; and an Akt inhibitor. A composition may comprise somatic cells; and a SET domain containing 2 (SETD2) inhibitor. A composition may comprise somatic cells; and a Dot1L inhibitor. A composition may comprise somatic cells; and an agonist for the G protein-coupled receptor Smoothened. A composition may comprise somatic cells; and a Jak1/Jak2 inhibitor. A composition may comprise somatic cells; and a S-adenosyl-L-homocysteine (SAH) hydrolase inhibitor. A composition may comprise somatic cells; and a Menin-MLL interaction inhibitor.

A composition may comprise somatic cells; and a GSK-3 inhibitor, a transforming growth factor-beta (TGFβ) receptor inhibitor, or a retinoic acid receptor (RAR) agonist, or any combination thereof. A composition may comprise somatic cells; and a glycogen synthase kinase 3 (GSK-3) inhibitor, a transforming growth factor-beta (TGFβ) receptor inhibitor, or a retinoic acid receptor (RAR) agonist. A composition may comprise somatic cells; and a GSK-3 inhibitor, a transforming growth factor-beta (TGFβ) receptor inhibitor, and a retinoic acid receptor (RAR) agonist. A composition may comprise somatic cells; and a GSK-3 inhibitor, a transforming growth factor-beta (TGFβ) receptor inhibitor, a retinoic acid receptor (RAR) agonist, an Akt inhibitor, or a SET domain containing 2 (SETD2) inhibitor, or any combination thereof. A composition may comprise somatic cells; and a GSK-3 inhibitor, a transforming growth factor-beta (TGFβ) receptor inhibitor, a retinoic acid receptor (RAR) agonist, an Akt inhibitor, or a SET domain containing 2 (SETD2) inhibitor. A composition may comprise somatic cells; and a GSK-3 inhibitor, a transforming growth factor-beta (TGFβ) receptor inhibitor, a retinoic acid receptor (RAR) agonist, an Akt inhibitor, and a SET domain containing 2 (SETD2) inhibitor. A composition may comprise somatic cells; and a glycogen synthase kinase 3 (GSK-3) inhibitor, a transforming growth factor-beta (TGFβ) receptor inhibitor, a retinoic acid receptor (RAR) agonist, or an Akt inhibitor, or any combination thereof. A composition may comprise somatic cells; and a glycogen synthase kinase 3 (GSK-3) inhibitor, a transforming growth factor-beta (TGFβ) receptor inhibitor, a retinoic acid receptor (RAR) agonist, or an Akt inhibitor. A composition may comprise somatic cells; and a glycogen synthase kinase 3 (GSK-3) inhibitor, a transforming growth factor-beta (TGFβ) receptor inhibitor, a retinoic acid receptor (RAR) agonist, and an Akt inhibitor. A composition may comprise somatic cells; and a glycogen synthase kinase 3 (GSK-3) inhibitor, a transforming growth factor-beta (TGFβ) receptor inhibitor, a retinoic acid receptor (RAR) agonist, or a SET domain containing 2 (SETD2) inhibitor, or any combination thereof. A composition may comprise somatic cells; and a glycogen synthase kinase 3 (GSK-3) inhibitor, a transforming growth factor-beta (TGFβ) receptor inhibitor, a retinoic acid receptor (RAR) agonist, or a SET domain containing 2 (SETD2) inhibitor. A composition may comprise somatic cells; and a glycogen synthase kinase 3 (GSK-3) inhibitor, a transforming growth factor-beta (TGFβ) receptor inhibitor, a retinoic acid receptor (RAR) agonist, and a SET domain containing 2 (SETD2) inhibitor. The somatic cells may comprise any somatic cell described herein. For example, the somatic may comprise fibroblast or hADSCs.

A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor, or any combination thereof. A composition may comprise epithelial-like cells; and at least 1, 2, 3, 4, 5, 6, 7, 8, 9, or 10 of a glycogen synthase kinase 3 (GSK-3) inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor. A composition may comprise epithelial-like cells; and at most 1, 2, 3, 4, 5, 6, 7, 8, 9, or 10 of a glycogen synthase kinase 3 (GSK-3) inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, an Akt inhibitor, a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, and a Menin-MLL interaction inhibitor.

A composition may comprise epithelial-like cells; a glycogen synthase kinase 3 (GSK-3) inhibitor and a transforming growth factor-beta (TGFβ) receptor inhibitor. A composition may comprise epithelial-like cells; a transforming growth factor-beta (TGFβ) receptor inhibitor and a retinoic acid receptor (RAR) agonist. A composition may comprise epithelial-like cells; a glycogen synthase kinase 3 (GSK-3) inhibitor and a retinoic acid receptor (RAR) agonist. A composition may comprise epithelial-like cells; and a glycogen synthase kinase 3 (GSK-3) inhibitor. A composition may comprise epithelial-like cells; and a transforming growth factor-beta (TGFβ) receptor inhibitor. A composition may comprise epithelial-like cells; and a retinoic acid receptor (RAR) agonist. A composition may comprise epithelial-like cells; and an Akt inhibitor. A composition may comprise epithelial-like cells; and a SET domain containing 2 (SETD2) inhibitor. A composition may comprise epithelial-like cells; and a Dot1L inhibitor. A composition may comprise epithelial-like cells; and an agonist for the G protein-coupled receptor Smoothened. A composition may comprise epithelial-like cells; and a Jak1/Jak2 inhibitor. A composition may comprise epithelial-like cells; and a S-adenosyl-L-homocysteine (SAH) hydrolase inhibitor. A composition may comprise epithelial-like cells; and a Menin-MLL interaction inhibitor.

A composition may comprise epithelial-like cells; and a glycogen synthase kinase 3 (GSK-3) inhibitor, a transforming growth factor-beta (TGFβ) receptor inhibitor, or a retinoic acid receptor (RAR) agonist, or any combination thereof. A composition may comprise epithelial-like cells; and a glycogen synthase kinase 3 (GSK-3) inhibitor, a transforming growth factor-beta (TGFβ) receptor inhibitor, or a retinoic acid receptor (RAR) agonist. A composition may comprise epithelial-like cells; and a glycogen synthase kinase 3 (GSK-3) inhibitor, a transforming growth factor-beta (TGFβ) receptor inhibitor, and a retinoic acid receptor (RAR) agonist. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, an Akt inhibitor, or a SET domain containing 2 (SETD2) inhibitor, or any combination thereof. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, an Akt inhibitor, or a SET domain containing 2 (SETD2) inhibitor. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, an Akt inhibitor, and a SET domain containing 2 (SETD2) inhibitor. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, or an Akt inhibitor, or any combination thereof. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, or an Akt inhibitor. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, and an Akt inhibitor. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, or a SET domain containing 2 (SETD2) inhibitor, or any combination thereof. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, or a SET domain containing 2 (SETD2) inhibitor. A composition may comprise epithelial-like cells; and a glycogen synthase kinase 3 (GSK-3) inhibitor, a transforming growth factor-beta (TGFβ) receptor inhibitor, a retinoic acid receptor (RAR) agonist, and a SET domain containing 2 (SETD2) inhibitor. The epithelial-like cells may express LIN28A, NMYC, WNT2B, PAX8, SMAD3, GLI3, KRT18, KRT19, WT1, or TBX2, or any combination thereof. The epithelial-like cells may express LIN28A. The epithelial-like cells may also express NMYC, WNT2B, PAX8, SMAD3, or GLI3, or a combinations thereof. The epithelial-like cells may express LIN28A and NMYC. The epithelial-like cells may express LIN28A and WNT2B. The epithelial-like cells may express LIN28A and PAX8. The epithelial-like cells may express LIN28A and SMAD3. The epithelial-like cells may express LIN28A and GLI3. The epithelial-like cells may not express any one of MMP1, ZEB1, VIM, COL1A1, COL5A1, COL6A2, PRRX1, SNAI2, TWIST1, or TWIST2.

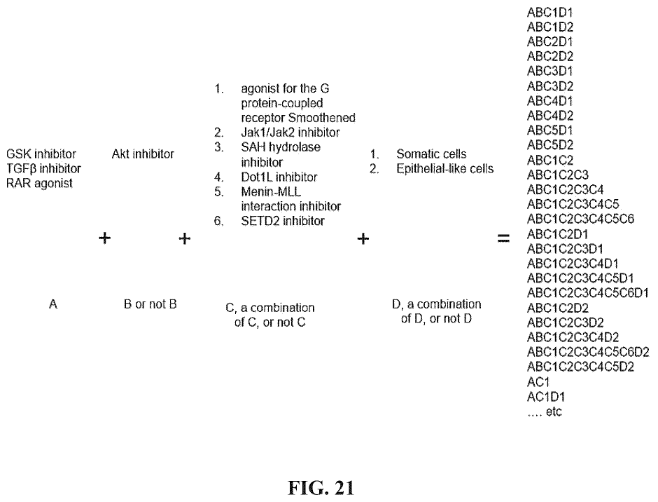

FIG. 21 depicts exemplary compositions comprising chemical reprogramming factors and optional cells in stage 1. In FIG. 21, “A” represents the combination a glycogen synthase kinase 3 (GSK-3) inhibitor, a transforming growth factor-beta (TGFβ) receptor inhibitor, and a retinoic acid receptor (RAR) agonist; “B” represents an Akt inhibitor in the composition, the composition may or may not include the Akt inhibitor; “C” represents a SET domain containing 2 (SETD2) inhibitor, a Dot1L inhibitor, an agonist for the G protein-coupled receptor Smoothened, a Jak1/Jak2 inhibitor, a S-adenosyl-L-homocysteine (SAH) hydrolase inhibitor, or a Menin-MLL interaction inhibitor, or any combination thereof, the composition may or may not have any of the compounds in group C; “D” represents somatic cells, epithelial-like cells, a combination of somatic cells and epithelial-like cells, the composition may or may not have any of these cells. For example, ABC1C2 represents a composition that includes a glycogen synthase kinase 3 (GSK-3) inhibitor, a transforming growth factor-beta (TGFβ) receptor inhibitor, a retinoic acid receptor (RAR) agonist, an Akt inhibitor, an agonist for the G protein-coupled receptor Smoothened, and a Jak1/Jak2 inhibitor.

A composition may comprise CHIR99021 or CHIR98014. A composition may comprise E-616452, A 83-01, SB431542, SB 505124, GW 788388, dorsomorphine, or SB 525334. A composition may comprise TTNPB, Ch55, or AM580. A composition may comprise SETD2-IN-1, EPZ-719, or MMSET-IN-1. A composition may comprise EPZ004777 or EPZ5676. A composition may comprise SAG, Purmorphamine, Hh-Ag1.5, or human SHH. A composition may comprise Ruxolitinib, Tofacitinib, AZD1480, Baricitinib, S-Ruxolitinib, or Fedratinib. A composition may comprise DZNep, NepA, Adox, or DZA. A composition may comprise VTP50469, MI3454, or WDR5-IN-4.

A composition may comprise CHIR99021 or CHIR98014; E-616452, A 83-01, SB431542, SB 505124, GW 788388, dorsomorphine, or SB 525334; TTNPB, Ch55, or AM580; an Akt inhibitor; SETD2-IN-1, EPZ-719, or MMSET-IN-1; EPZ004777 or EPZ5676; SAG, Purmorphamine, Hh-Ag1.5, or human SHH; Ruxolitinib, Tofacitinib, AZD1480, Baricitinib, S-Ruxolitinib, or Fedratinib; DZNep, NepA, Adox, or DZA; VTP50469, MI3454, or WDR5-IN-4; or any combination thereof. A composition may comprise CHIR99021 or CHIR98014; E-616452, A 83-01, SB431542, SB 505124, GW 788388, dorsomorphine, or SB 525334; TTNPB, Ch55, or AM580; an Akt inhibitor; SETD2-IN-1, EPZ-719, or MMSET-IN-1; EPZ004777 or EPZ5676; SAG, Purmorphamine, Hh-Ag1.5, or human SHH; Ruxolitinib, Tofacitinib, AZD1480, Baricitinib, S-Ruxolitinib, or Fedratinib; DZNep, NepA, Adox, or DZA; and VTP50469, MI3454, or WDR5-IN-4.

A composition may comprise CHIR99021 or CHIR98014; E-616452, A 83-01, SB431542, SB 505124, GW 788388, dorsomorphine, or SB 525334; TTNPB, Ch55, or AM580; or any combination thereof. A composition may comprise CHIR99021 or CHIR98014; E-616452, A 83-01, SB431542, SB 505124, GW 788388, dorsomorphine, or SB 525334; or TTNPB, Ch55, or AM580. A composition may comprise CHIR99021 or CHIR98014; E-616452, A 83-01, SB431542, SB 505124, GW 788388, dorsomorphine, or SB 525334; and TTNPB, Ch55, or AM580. A composition may comprise CHIR99021 or CHIR98014; E-616452, A 83-01, SB431542, SB 505124, GW 788388, dorsomorphine, or SB 525334; TTNPB, Ch55, or AM580; an Akt inhibitor; SETD2-IN-1, EPZ-719, or MMSET-IN-1; or any combination thereof. A composition may comprise CHIR99021 or CHIR98014; E-616452, A 83-01, SB431542, SB 505124, GW 788388, dorsomorphine, or SB 525334; TTNPB, Ch55, or AM580; an Akt inhibitor; or SETD2-IN-1, EPZ-719, or MMSET-IN-1. A composition may comprise CHIR99021 or CHIR98014; E-616452, A 83-01, SB431542, SB 505124, GW 788388, dorsomorphine, or SB 525334; TTNPB, Ch55, or AM580; an Akt inhibitor; and SETD2-IN-1, EPZ-719, or MMSET-IN-1. A composition may comprise CHIR99021 or CHIR98014; E-616452, A 83-01, SB431542, SB 505124, GW 788388, dorsomorphine, or SB 525334; TTNPB, Ch55, or AM580; or an Akt inhibitor; or any combination thereof. A composition may comprise CHIR99021 or CHIR98014; E-616452, A 83-01, SB431542, SB 505124, GW 788388, dorsomorphine, or SB 525334; TTNPB, Ch55, or AM580; or an Akt inhibitor. A composition may comprise CHIR99021 or CHIR98014; E-616452, A 83-01, SB431542, SB 505124, GW 788388, dorsomorphine, or SB 525334; TTNPB, Ch55, or AM580; and an Akt inhibitor. A composition may comprise CHIR99021 or CHIR98014; E-616452, A 83-01, SB431542, SB 505124, GW 788388, dorsomorphine, or SB 525334; TTNPB, Ch55, or AM580; or an SET domain containing 2 (SETD2) inhibitor; or any combination thereof. A composition may comprise CHIR99021 or CHIR98014; E-616452, A 83-01, SB431542, SB 505124, GW 788388, dorsomorphine, or SB 525334; TTNPB, Ch55, or AM580; or an SET domain containing 2 (SETD2) inhibitor. A composition may comprise CHIR99021 or CHIR98014; E-616452, A 83-01, SB431542, SB 505124, GW 788388, dorsomorphine, or SB 525334; TTNPB, Ch55, or AM580; and an SET domain containing 2 (SETD2) inhibitor.

A composition may comprise CHIR99021. A composition may comprise E-616452. A composition may comprise TTNPB. A composition may comprise AKTi. A composition may comprise SETD2-IN-1. A composition may comprise EPZ5676. A composition may comprise SAG. A composition may comprise Ruxolitinib. A composition may comprise DZNep. A composition may comprise VTP50469.

A composition may comprise CHIR99021, E-616452, TTNPB, AKTi, SETD2-IN-1, EPZ5676, SAG, Ruxolitinib, DZNep, or VTP50469, or any combination thereof. A composition may comprise CHIR99021, E-616452, TTNPB, AKTi, SETD2-IN-1, EPZ5676, SAG, Ruxolitinib, DZNep, and VTP50469.

A composition may comprise CHIR99021, E-616452, or TTNPB, or any combination thereof. A composition may comprise CHIR99021, E-616452, or TTNPB. A composition may comprise CHIR99021, E-616452, and TTNPB. A composition may comprise CHIR99021, E-616452, TTNPB, AKTi, or SETD2-IN-1, or any combination thereof. A composition may comprise CHIR99021, E-616452, TTNPB, AKTi, or SETD2-IN-1. A composition may comprise CHIR99021, E-616452, TTNPB, AKTi, and SETD2-IN-1. A composition may comprise CHIR99021, E-616452, TTNPB, or AKTi, or any combination thereof. A composition may comprise CHIR99021, E-616452, TTNPB, or AKTi. A composition may comprise CHIR99021, E-616452, TTNPB, and AKTi. A composition may comprise CHIR99021, E-616452, TTNPB, or SETD2-IN-1, or any combination thereof. A composition may comprise CHIR99021, E-616452, TTNPB, or SETD2-IN-1. A composition may comprise CHIR99021, E-616452, TTNPB, and SETD2-IN-1.

A composition may comprise at least about 0.1 micromolar (μM), 0.2 μM, 0.3 μM, 0.4 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 100 μM, 150 μM, 200 μM, 250 μM or more CHIR99021 within the composition. A composition may comprise at most about 0.1 μM, 0.2 μM, 0.3 μM, 0.4 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 100 μM, 150 μM, 200 μM, or 250 μM CHIR99021 within the composition. A composition may comprise about 0.5 μM CHIR99021 within the composition. A composition may comprise about 1 μM CHIR99021 within the composition. A composition may comprise about 2 μM CHIR99021 within the composition. A composition may comprise about 3 μM CHIR99021 within the composition. A composition may comprise about 4 μM CHIR99021 within the composition. A composition may comprise about 5 μM CHIR99021 within the composition. A composition may comprise about 6 μM CHIR99021 within the composition. A composition may comprise about 7 μM CHIR99021 within the composition. A composition may comprise about 8 μM CHIR99021 within the composition. A composition may comprise about 9 μM CHIR99021 within the composition. A composition may comprise about 10 μM CHIR99021 within the composition. A composition may comprise about 15 μM CHIR99021 within the composition. A composition may comprise about 20 μM CHIR99021 within the composition. A composition may comprise about 30 μM CHIR99021 within the composition. A composition may comprise about 40 μM CHIR99021 within the composition. A composition may comprise about 50 μM CHIR99021 within the composition. A composition may comprise about 0.1 μM to about 100 μM CHIR99021 within the composition. A composition may comprise about 0.2 μM to about 75 μM CHIR99021 within the composition. A composition may comprise about 0.5 μM to about 50 μM CHIR99021 within the composition. A composition may comprise about 1 μM to about 25 μM CHIR99021 within the composition. A composition may comprise about 2 μM to about 12.5 μM CHIR99021 within the composition. A composition may comprise about 4 μM to about 6.25 μM CHIR99021 within the composition.

A composition may comprise at least about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, 500 μM or more E-616452 within the composition. A composition may comprise at most about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, or 500 μM E-616452 within the composition. A composition may comprise about 1 μM E-616452 within the composition. A composition may comprise about 2 μM E-616452 within the composition. A composition may comprise about 3 μM E-616452 within the composition. A composition may comprise about 4 μM E-616452 within the composition. A composition may comprise about 5 μM E-616452 within the composition. A composition may comprise about 6 μM E-616452 within the composition. A composition may comprise about 7 μM E-616452 within the composition. A composition may comprise about 8 μM E-616452 within the composition. A composition may comprise about 9 μM E-616452 within the composition. A composition may comprise about 10 μM E-616452 within the composition. A composition may comprise about 15 μM E-616452 within the composition. A composition may comprise about 20 μM E-616452 within the composition. A composition may comprise about 30 μM E-616452 within the composition. A composition may comprise about 40 μM E-616452 within the composition. A composition may comprise about 50 μM E-616452 within the composition. A composition may comprise about 60 μM E-616452 within the composition. A composition may comprise about 70 μM E-616452 within the composition. A composition may comprise about 80 μM E-616452 within the composition. A composition may comprise about 90 μM E-616452 within the composition. A composition may comprise about 100 μM E-616452 within the composition. A composition may comprise about 1 μM to about 100 μM E-616452 within the composition. A composition may comprise about 2 μM to about 75 μM E-616452 within the composition. A composition may comprise about 3 μM to about 50 μM E-616452 within the composition. A composition may comprise about 4 μM to about 40 μM E-616452 within the composition. A composition may comprise about 5 μM to about 30 μM E-616452 within the composition. A composition may comprise about 7.5 μM to about 20 μM E-616452 within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more TTNPB within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM TTNPB within the composition. A composition may comprise about 0.2 μM TTNPB within the composition. A composition may comprise about 0.4 μM TTNPB within the composition. A composition may comprise about 0.6 μM TTNPB within the composition. A composition may comprise about 0.8 μM TTNPB within the composition. A composition may comprise about 1 μM TTNPB within the composition. A composition may comprise about 1.2 μM TTNPB within the composition. A composition may comprise about 1.4 μM TTNPB within the composition. A composition may comprise about 1.6 μM TTNPB within the composition. A composition may comprise about 1.8 μM TTNPB within the composition. A composition may comprise about 2 μM TTNPB within the composition. A composition may comprise about 4 μM TTNPB within the composition. A composition may comprise about 6 μM TTNPB within the composition. A composition may comprise about 8 μM TTNPB within the composition. A composition may comprise about 10 μM TTNPB within the composition. A composition may comprise about 12 μM TTNPB within the composition. A composition may comprise about 14 μM TTNPB within the composition. A composition may comprise about 16 μM TTNPB within the composition. A composition may comprise about 18 μM TTNPB within the composition. A composition may comprise about 20 μM TTNPB within the composition. A composition may comprise about 0.2 μM to about 20 μM TTNPB within the composition. A composition may comprise about 0.4 μM to about 15 μM TTNPB within the composition. A composition may comprise about 0.6 μM to about 10 μM TTNPB within the composition. A composition may comprise about 0.8 μM to about 8 μM TTNPB within the composition. A composition may comprise about 1 μM to about 6 μM TTNPB within the composition. A composition may comprise about 1.5 μM to about 4 μM TTNPB within the composition.

A composition may comprise at least about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, 50 μM or more AKTi within the composition. A composition may comprise at most about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, or 50 μM AKTi within the composition. A composition may comprise about 0.1 μM AKTi within the composition. A composition may comprise about 0.2 μM AKTi within the composition. A composition may comprise about 0.3 μM AKTi within the composition. A composition may comprise about 0.4 μM AKTi within the composition. A composition may comprise about 0.5 μM AKTi within the composition. A composition may comprise about 0.6 μM AKTi within the composition. A composition may comprise about 0.7 μM AKTi within the composition. A composition may comprise about 0.8 μM AKTi within the composition. A composition may comprise about 0.9 μM AKTi within the composition. A composition may comprise about 1 μM AKTi within the composition. A composition may comprise about 2 μM AKTi within the composition. A composition may comprise about 3 μM AKTi within the composition. A composition may comprise about 4 μM AKTi within the composition. A composition may comprise about 5 μM AKTi within the composition. A composition may comprise about 6 μM AKTi within the composition. A composition may comprise about 7 μM AKTi within the composition. A composition may comprise about 8 μM AKTi within the composition. A composition may comprise about 9 μM AKTi within the composition. A composition may comprise about 10 μM AKTi within the composition. A composition may comprise about 0.1 μM to about 10 μM AKTi within the composition. A composition may comprise about 0.2 μM to about 7.5 μM AKTi within the composition. A composition may comprise about 0.3 μM to about 5 μM AKTi within the composition. A composition may comprise about 0.4 μM to about 4 μM AKTi within the composition. A composition may comprise about 0.5 μM to about 3 μM AKTi within the composition. A composition may comprise about 0.75 μM to about 2 μM AKTi within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more SETD2-IN-1 within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM SETD2-IN-1 within the composition. A composition may comprise about 0.05 μM SETD2-IN-1 within the composition. A composition may comprise about 0.1 μM SETD2-IN-1 within the composition. A composition may comprise about 0.15 μM SETD2-IN-1 within the composition. A composition may comprise about 0.2 μM SETD2-IN-1 within the composition. A composition may comprise about 0.25 μM SETD2-IN-1 within the composition. A composition may comprise about 0.3 μM SETD2-IN-1 within the composition. A composition may comprise about 0.35 μM SETD2-IN-1 within the composition. A composition may comprise about 0.4 μM SETD2-IN-1 within the composition. A composition may comprise about 0.45 μM SETD2-IN-1 within the composition. A composition may comprise about 0.5 μM SETD2-IN-1 within the composition. A composition may comprise about 1 μM SETD2-IN-1 within the composition. A composition may comprise about 1.5 μM SETD2-IN-1 within the composition. A composition may comprise about 2 μM SETD2-IN-1 within the composition. A composition may comprise about 2.5 μM SETD2-IN-1 within the composition. A composition may comprise about 3 μM SETD2-IN-1 within the composition. A composition may comprise about 3.5 μM SETD2-IN-1 within the composition. A composition may comprise about 4 μM SETD2-IN-1 within the composition. A composition may comprise about 4.5 μM SETD2-IN-1 within the composition. A composition may comprise about 5 μM SETD2-IN-1 within the composition. A composition may comprise about 0.05 μM to about 2.5 μM SETD2-IN-1 within the composition. A composition may comprise about 0.1 μM to about 1.875 μM SETD2-IN-1 within the composition. A composition may comprise about 0.15 μM to about 1.25 μM SETD2-IN-1 within the composition. A composition may comprise about 0.2 μM to about 1 μM SETD2-IN-1 within the composition. A composition may comprise about 0.25 μM to about 0.75 μM SETD2-IN-1 within the composition. A composition may comprise about 0.375 μM to about 0.5 μM SETD2-IN-1 within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more SAG within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM SAG within the composition. A composition may comprise about 0.05 μM SAG within the composition. A composition may comprise about 0.1 μM SAG within the composition. A composition may comprise about 0.15 μM SAG within the composition. A composition may comprise about 0.2 μM SAG within the composition. A composition may comprise about 0.25 μM SAG within the composition. A composition may comprise about 0.3 μM SAG within the composition. A composition may comprise about 0.35 μM SAG within the composition. A composition may comprise about 0.4 μM SAG within the composition. A composition may comprise about 0.45 μM SAG within the composition. A composition may comprise about 0.5 μM SAG within the composition. A composition may comprise about 1 μM SAG within the composition. A composition may comprise about 1.5 μM SAG within the composition. A composition may comprise about 2 μM SAG within the composition. A composition may comprise about 2.5 μM SAG within the composition. A composition may comprise about 3 μM SAG within the composition. A composition may comprise about 3.5 μM SAG within the composition. A composition may comprise about 4 μM SAG within the composition. A composition may comprise about 4.5 μM SAG within the composition. A composition may comprise about 5 μM SAG within the composition. A composition may comprise about 0.05 μM to about 2.5 μM SAG within the composition. A composition may comprise about 0.1 μM to about 1.875 μM SAG within the composition. A composition may comprise about 0.15 μM to about 1.25 μM SAG within the composition. A composition may comprise about 0.2 μM to about 1 μM SAG within the composition. A composition may comprise about 0.25 μM to about 0.75 μM SAG within the composition. A composition may comprise about 0.375 μM to about 0.5 μM SAG within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more VTP50469 within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM VTP50469 within the composition. A composition may comprise about 0.05 μM VTP50469 within the composition. A composition may comprise about 0.1 μM VTP50469 within the composition. A composition may comprise about 0.15 μM VTP50469 within the composition. A composition may comprise about 0.2 μM VTP50469 within the composition. A composition may comprise about 0.25 μM VTP50469 within the composition. A composition may comprise about 0.3 μM VTP50469 within the composition. A composition may comprise about 0.35 μM VTP50469 within the composition. A composition may comprise about 0.4 μM VTP50469 within the composition. A composition may comprise about 0.45 μM VTP50469 within the composition. A composition may comprise about 0.5 μM VTP50469 within the composition. A composition may comprise about 1 μM VTP50469 within the composition. A composition may comprise about 1.5 μM VTP50469 within the composition. A composition may comprise about 2 μM VTP50469 within the composition. A composition may comprise about 2.5 μM VTP50469 within the composition. A composition may comprise about 3 μM VTP50469 within the composition. A composition may comprise about 3.5 μM VTP50469 within the composition. A composition may comprise about 4 μM VTP50469 within the composition. A composition may comprise about 4.5 μM VTP50469 within the composition. A composition may comprise about 5 μM VTP50469 within the composition. A composition may comprise about 0.05 μM to about 2.5 μM VTP50469 within the composition. A composition may comprise about 0.1 μM to about 1.875 μM VTP50469 within the composition. A composition may comprise about 0.15 μM to about 1.25 μM VTP50469 within the composition. A composition may comprise about 0.2 μM to about 1 μM VTP50469 within the composition. A composition may comprise about 0.25 μM to about 0.75 μM VTP50469 within the composition. A composition may comprise about 0.375 μM to about 0.5 μM VTP50469 within the composition.

A composition may comprise at least about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, 50 μM or more Ruxolitinib within the composition. A composition may comprise at most about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, or 50 μM Ruxolitinib within the composition. A composition may comprise about 0.1 μM Ruxolitinib within the composition. A composition may comprise about 0.2 μM Ruxolitinib within the composition. A composition may comprise about 0.3 μM Ruxolitinib within the composition. A composition may comprise about 0.4 μM Ruxolitinib within the composition. A composition may comprise about 0.5 μM Ruxolitinib within the composition. A composition may comprise about 0.6 μM Ruxolitinib within the composition. A composition may comprise about 0.7 μM Ruxolitinib within the composition. A composition may comprise about 0.8 μM Ruxolitinib within the composition. A composition may comprise about 0.9 μM Ruxolitinib within the composition. A composition may comprise about 1 μM Ruxolitinib within the composition. A composition may comprise about 2 μM Ruxolitinib within the composition. A composition may comprise about 3 μM Ruxolitinib within the composition. A composition may comprise about 4 μM Ruxolitinib within the composition. A composition may comprise about 5 μM Ruxolitinib within the composition. A composition may comprise about 6 μM Ruxolitinib within the composition. A composition may comprise about 7 μM Ruxolitinib within the composition. A composition may comprise about 8 μM Ruxolitinib within the composition. A composition may comprise about 9 μM Ruxolitinib within the composition. A composition may comprise about 10 μM Ruxolitinib within the composition. A composition may comprise about 0.1 μM to about 10 μM Ruxolitinib within the composition. A composition may comprise about 0.2 μM to about 7.5 μM Ruxolitinib within the composition. A composition may comprise about 0.3 μM to about 5 μM Ruxolitinib within the composition. A composition may comprise about 0.4 μM to about 4 μM Ruxolitinib within the composition. A composition may comprise about 0.5 μM to about 3 μM Ruxolitinib within the composition. A composition may comprise about 0.75 μM to about 2 μM Ruxolitinib within the composition.

A composition may comprise at least about 0.0004 μM, 0.0008 μM, 0.0012 μM, 0.0016 μM, 0.002 μM, 0.004 μM, 0.006 μM, 0.008 μM, 0.01 μM, 0.012 μM, 0.014 μM, 0.016 μM, 0.018 μM, 0.02 μM, 0.04 μM, 0.06 μM, 0.08 μM, 0.1 μM, 0.12 μM, 0.14 μM, 0.16 μM, 0.18 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM or more DZNep within the composition. A composition may comprise at most about 0.0004 μM, 0.0008 μM, 0.0012 μM, 0.0016 μM, 0.002 μM, 0.004 μM, 0.006 μM, 0.008 μM, 0.01 μM, 0.012 μM, 0.014 μM, 0.016 μM, 0.018 μM, 0.02 μM, 0.04 μM, 0.06 μM, 0.08 μM, 0.1 μM, 0.12 μM, 0.14 μM, 0.16 μM, 0.18 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, or 1 μM DZNep within the composition. A composition may comprise about 0.002 μM DZNep within the composition. A composition may comprise about 0.004 μM DZNep within the composition. A composition may comprise about 0.006 μM DZNep within the composition. A composition may comprise about 0.008 μM DZNep within the composition. A composition may comprise about 0.01 μM DZNep within the composition. A composition may comprise about 0.012 μM DZNep within the composition. A composition may comprise about 0.014 μM DZNep within the composition. A composition may comprise about 0.016 μM DZNep within the composition. A composition may comprise about 0.018 μM DZNep within the composition. A composition may comprise about 0.02 μM DZNep within the composition. A composition may comprise about 0.04 μM DZNep within the composition. A composition may comprise about 0.06 μM DZNep within the composition. A composition may comprise about 0.08 μM DZNep within the composition. A composition may comprise about 0.1 μM DZNep within the composition. A composition may comprise about 0.12 μM DZNep within the composition. A composition may comprise about 0.14 μM DZNep within the composition. A composition may comprise about 0.16 μM DZNep within the composition. A composition may comprise about 0.18 μM DZNep within the composition. A composition may comprise about 0.2 μM DZNep within the composition. A composition may comprise about 0.002 μM to about 0.2 μM DZNep within the composition. A composition may comprise about 0.0025 μM to about 0.15 μM DZNep within the composition. A composition may comprise about 0.005 μM to about 0.1 μM DZNep within the composition. A composition may comprise about 0.0075 μM to about 0.75 μM DZNep within the composition. A composition may comprise about 0.01 μM to about 0.5 μM DZNep within the composition. A composition may comprise about 0.015 μM to about 0.4 μM DZNep within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more EPZ5676 within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM EPZ5676 within the composition. A composition may comprise about 0.2 μM EPZ5676 within the composition. A composition may comprise about 0.4 μM EPZ5676 within the composition. A composition may comprise about 0.6 μM EPZ5676 within the composition. A composition may comprise about 0.8 μM EPZ5676 within the composition. A composition may comprise about 1 μM EPZ5676 within the composition. A composition may comprise about 1.2 μM EPZ5676 within the composition. A composition may comprise about 1.4 μM EPZ5676 within the composition. A composition may comprise about 1.6 μM EPZ5676 within the composition. A composition may comprise about 1.8 μM EPZ5676 within the composition. A composition may comprise about 2 μM EPZ5676 within the composition. A composition may comprise about 4 μM EPZ5676 within the composition. A composition may comprise about 6 μM EPZ5676 within the composition. A composition may comprise about 8 μM EPZ5676 within the composition. A composition may comprise about 10 μM EPZ5676 within the composition. A composition may comprise about 12 μM EPZ5676 within the composition. A composition may comprise about 14 μM EPZ5676 within the composition. A composition may comprise about 16 μM EPZ5676 within the composition. A composition may comprise about 18 μM EPZ5676 within the composition. A composition may comprise about 20 μM EPZ5676 within the composition. A composition may comprise about 0.2 μM to about 20 μM EPZ5676 within the composition. A composition may comprise about 0.4 μM to about 15 μM EPZ5676 within the composition. A composition may comprise about 0.6 μM to about 10 μM EPZ5676 within the composition. A composition may comprise about 0.8 μM to about 8 μM EPZ5676 within the composition. A composition may comprise about 1 μM to about 6 μM EPZ5676 within the composition. A composition may comprise about 1.5 μM to about 4 μM EPZ5676 within the composition.

Another aspect of the invention is to provide a composition that comprises reprogramming factors for stage 1 conversion, that further comprises one or more of a histone acetyltransferase inhibitor. In some embodiments, the histone acetyltransferase inhibitor disclosed herein is a KAT3A/KAT3B inhibitor or a KAT6A inhibitor. In some embodiments, the histone acetyltransferase inhibitor disclosed herein is selected from A485, ICBP112, GEN049, CBP/P300 IN 12, SGC-CBP30, CBP/P300 IN 8, WM8014, WM1119, or any combination thereof. In some embodiment, the histone acetyltransferase inhibitor disclosed herein is selected from A485, WM8014, or a combination of both.

In some embodiments, the composition disclosed herein comprises a histone acetyltransferase inhibitor at a concentration of 0.01 to 10 μM. In some embodiments, the composition disclosed herein comprises a histone acetyltransferase inhibitor at a concentration of 0.05 to 5 μM. In some embodiments, the composition disclosed herein comprises a histone acetyltransferase inhibitor at a concentration of 0.1 to 5 μM. In some embodiments, the composition disclosed herein comprises a histone acetyltransferase inhibitor at a concentration of 0.1 to 2 μM.

In some embodiments, the composition disclosed herein comprises A485 at a concentration of 0.1 to 2 μM, or WM8014 at a concentration of 0.1 to 5 μM, or a combination of both. In some embodiments, the composition disclosed herein comprises A485 at a concentration of 0.5 to 1 μM, or WM8014 at a concentration of 0.5 to 1 μM, or a combination of both. In some embodiments, the composition disclosed herein comprises A485 at a concentration of 0.5 μM, or WM8014 at a concentration of 1 μM, or a combination of both.

In some embodiments, the histone acetyltransferase inhibitor disclosed herein is selected from SGC/CBP300, A485, CBP/P300 IN 8, WM8014, GEN049, WM1119, CBP/P300 IN 12, ICBP112, or any combination thereof. In a preferred embodiment, it is SGC/CBP300. In some embodiments, the concentration of the histone acetyltransferase inhibitor disclosed herein is 0.4-10 μM. In a preferred embodiment, the concentration of the histone acetyltransferase inhibitor disclosed herein is 2 WM.

In some embodiments, the composition disclosed herein comprises 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, and 0.5 μM A-485.

In some embodiments, the composition disclosed herein comprises 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, and 1 μM WM8014.

In some embodiments, the composition disclosed herein comprises 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 1 μM WM8014, and 0.5 μM A-485.

In some embodiments, the composition disclosed herein comprises 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 0.5 μM A-485, and 0.5 μM AM095.

In some embodiments, the composition disclosed herein comprises 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 1 μM WM8014, and 0.5 μM AM095.

In some embodiments, the composition disclosed herein comprises 5 μM CHIR999021, 10 μM 616452, 2 μM TTNPB, 0.5 μM SAG, 2 μM EPZ5676, 0.02 μM DZNep, 1 μM Ruxolitinib, 0.5 μM VTP50469, 1 μM HY-10249A (CAS No. 842148-40-7), 0.2 μM SETD2IN1, 0.2 μM JNKIN8, 0.5 μM A-485, 1 μM WM8014, and 0.5 μM AM095.

The cell population with increased differentiation potential is epithelial-like cells that are positive for the pluripotency marker Lin28A, and compared with the somatic cells, it has epithelial cell markers with increased expression levels and somatic cell markers with decreased expression levels.

Subsequent to contacting any populations of stage 1 cells with any compositions described herein, the cells may be incubated in hypoxic condition. For example, the stage 1 cells may be incubated with at most 21%, 20%, 19%, 18%, 17%, 16%, 15%, 14%, 13%, 12%, 11%, 10%, 9%, 8%, 7%, 6%, 5%, 4%, 3%, 2%, 1% or lower atmospheric oxygen. The hypoxic condition may comprise about 10% atmospheric oxygen. The hypoxic condition may comprise about 9% atmospheric oxygen. The hypoxic condition may comprise about 8% atmospheric oxygen. The hypoxic condition may comprise about 7% atmospheric oxygen. The hypoxic condition may comprise about 6% atmospheric oxygen. The hypoxic condition may comprise about 5% atmospheric oxygen. The hypoxic condition may comprise about 4% atmospheric oxygen. The hypoxic condition may comprise about 3% atmospheric oxygen. The hypoxic condition may comprise about 2% atmospheric oxygen. The hypoxic condition may comprise about 1% atmospheric oxygen.

Subsequent to contacting any populations of stage 1 cells with any compositions described herein, a population of stage 1 cells may be incubated with a composition for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, or 30 days. A population of stage 1 cells may be incubated with a composition for at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, or 30 days. A population of stage 1 cells may be incubated with a composition for about 1 day. A population of stage 1 cells may be incubated with a composition for about 2 days. A population of stage 1 cells may be incubated with a composition for about 3 days. A population of stage 1 cells may be incubated with a composition for about 4 days. A population of stage 1 cells may be incubated with a composition for about 5 days. A population of stage 1 cells may be incubated with a composition for about 6 days. A population of stage 1 cells may be incubated with a composition for about 7 days. A population of stage 1 cells may be incubated with a composition for about 8 days. A population of stage 1 cells may be incubated with a composition for about 9 days. A population of stage 1 cells may be incubated with a composition for about 10 days. A population of stage 1 cells may be incubated with a composition for about 11 days. A population of stage 1 cells may be incubated with a composition for about 12 days. A population of stage 1 cells may be incubated with a composition for about 13 days. A population of stage 1 cells may be incubated with a composition for about 14 days. A population of stage 1 cells may be incubated with a composition for about 15 days. A population of stage 1 cells may be incubated with a composition for about 16 days. A population of stage 1 cells may be incubated with a composition for about 17 days. A population of stage 1 cells may be incubated with a composition for about 18 days. A population of stage 1 cells may be incubated with a composition for about 19 days. A population of stage 1 cells may be incubated with a composition for about 20 days. A population of stage 1 cells may be incubated with a composition for about 25 days.

Any of the compositions may not comprise feeder cells or serum. Any of the compositions may not comprise feeder cells and serum. Any of the compositions may not comprise feeder cells. Any of the compositions may be serum-free. Any of the compositions may comprise feeder cells. Any of the compositions may comprise serum.

### Stage 2

In some aspects, provided herein are stage 2 methods and compositions for conversion of an epithelial-like cell into a cell with a higher cell potency (e.g., less specialized cell), such as an intermediate plastic state cell—the conversion process referred herein also as “stage 2”. A stage 2 method may be part of a conversion process that reprograms somatic cells or epithelial-like cells into pluripotent stem cells. A stage 2 method may be the second stage of a conversion process that reprograms somatic cells into pluripotent stem cells.

A stage 2 method may comprise contacting a first cell population with a first composition. A stage 2 method may comprise, subsequent to or during the contacting, converting a subset of the first cell population into different cells. The cell population comprising the different cells may comprise a second cell population. A stage 2 method may comprise incubating the first cell population with the first composition for a period of time. The subset of the first cell population may be converted into the different cells prior to, during, or subsequent to the incubating. In some cases, a stage 2 method may comprise removing the first composition from the second population of cells. In other cases, a stage 2 method may comprise removing the first composition from the first population of cells.

A population of stage 2 cells may comprise at least a subset of stage 1 cells. A population of stage 2 cells may comprise at most a subset of stage 1 cells. A population of stage 2 cells may comprise epithelial-like cells or somatic cells. A population of stage 2 cells may comprise epithelial-like cells. A population of stage 2 cells may comprise somatic cells. A population of stage 2 cells may comprise epithelial-like cells and somatic cells.

A first population of stage 2 cells may comprise any populations of stage 1 cells. In some case, the first population of stage 2 cells may comprise the second population of stage 1 cells.

The second population of stage 2 cells may comprise intermediate plastic state cells. The first population of stage 2 cells may not comprise intermediate plastic state cells. An intermediate plastic state cell may not be a naturally occurring cell. An intermediate plastic state cell may express a combination of genes that are not expressed by a naturally occurring cell. An intermediate plastic state cell may express at least one gene at level at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 5-fold, 100-fold, or higher, relative to a naturally occurring cell. An intermediate plastic state cell may express at least one gene at level at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% lower, relative to a naturally occurring cell. The second population of stage 2 cells may comprise somatic cells, epithelial-like cells, or intermediate plastic state cells. The second population of stage 2 cells may comprise somatic cells, epithelial-like cells, and intermediate plastic state cells. The second population of stage 2 cells may not comprise somatic cells or epithelial-like cells. In some cases, the second population of stage 2 cells may comprise fewer somatic cells or epithelial-like cells than the first population of stage 2 cells. For example, the second population of stage 2 cells may have 0.001%, 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90% or 99% fewer somatic cells or epithelial-like cells than the first population of stage 2 cells. In some cases, the second population of stage 2 cells may comprise more intermediate plastic state cells than the first population of stage 2 cells. For example, the second population of stage 2 cells may have 0.001%, 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 5-fold, 100-fold, or more intermediate plastic state cells than the first population of stage 2 cells.

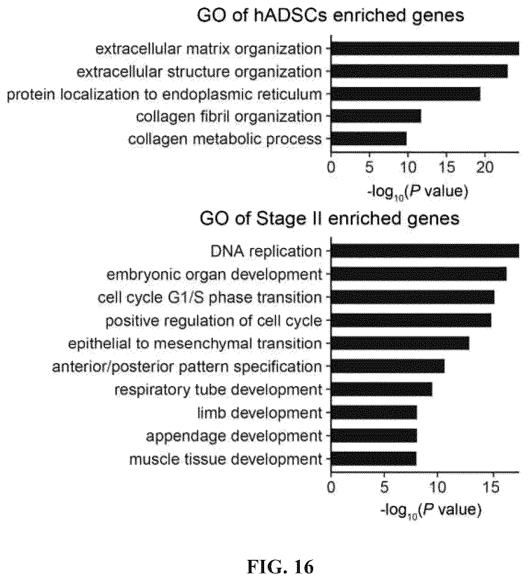

Intermediate plastic state cells may show decreased expression of genes expressed by somatic cells. Intermediate plastic state cells may show increased expressions of genes involved in embryonic development, increased cell proliferation, and decreased methylation epigenetic state. Promoter regions of genes related to embryonic development, cell cycle and stem cell proliferation can be demethylated in intermediate plastic state cells. Intermediate plastic state cells may undergo dedifferentiation, relative to somatic cells or epithelial-like cells. The upregulated genes in intermediate plastic state cells may comprise those described in FIG. 16. In some cases, genes related to limb and appendage development may be upregulated and have open chromatin structures in intermediate plastic state cells. Intermediate plastic state cells can be reprogrammed to acquire characteristics of developing human limb bud cells, similar to the situation of axolotl limb regeneration in which genes governing embryonic limb development are reactivated during dedifferentiation. However, dedifferentiation was not found in frogs and mice, according to Guan 2002, of which the limb tissue showed no notable activation of an embryonic gene expression program following injury.

An intermediate plastic state cell may express LIN28A, SALL4, MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2, or any combination thereof. An intermediate plastic state cell may express LIN28A. An intermediate plastic state cell may express SALL4. An intermediate plastic state cell may express MSX2. An intermediate plastic state cell may express NMYC. An intermediate plastic state cell may express WNT4. An intermediate plastic state cell may express FGF19. An intermediate plastic state cell may express TOP2A. An intermediate plastic state cell may express MSX1. An intermediate plastic state cell may express HOXB9. An intermediate plastic state cell may express WT1. An intermediate plastic state cell may express GATA2. An intermediate plastic state cell may express HMGA2. An intermediate plastic state cell may express LEF1. An intermediate plastic state cell may express FGF9. An intermediate plastic state cell may express HOXA9. An intermediate plastic state cell may express HOXA1. An intermediate plastic state cell may express PTCH1. An intermediate plastic state cell may express HOXA5. An intermediate plastic state cell may express CCND2. An intermediate plastic state cell may express SDC1. An intermediate plastic state cell may express TBX3. An intermediate plastic state cell may express BMP4. An intermediate plastic state cell may express IGF2. An intermediate plastic state cell may express one or more of LIN28A, SALL4, MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2.

An intermediate plastic state cell may express LIN28A or SALL4. An intermediate plastic state cell may express LIN28A and SALL4. An intermediate plastic state cell may express LIN28A and SALL4; and MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2, or any combination thereof. An intermediate plastic state cell may express LIN28A and SALL4; and MSX2. An intermediate plastic state cell may express LIN28A and SALL4; and NMYC. An intermediate plastic state cell may express LIN28A and SALL4; and WNT4. An intermediate plastic state cell may express LIN28A and SALL4; and FGF19. An intermediate plastic state cell may express LIN28A and SALL4; and TOP2A. An intermediate plastic state cell may express LIN28A and SALL4; and MSX1. An intermediate plastic state cell may express LIN28A and SALL4; and HOXB9. An intermediate plastic state cell may express LIN28A and SALL4; and WT1. An intermediate plastic state cell may express LIN28A and SALL4; and GATA2. An intermediate plastic state cell may express LIN28A and SALL4; and HMGA2. An intermediate plastic state cell may express LIN28A and SALL4; and LEF1. An intermediate plastic state cell may express LIN28A and SALL4; and FGF9. An intermediate plastic state cell may express LIN28A and SALL4; and HOXA9. An intermediate plastic state cell may express LIN28A and SALL4; and HOXA1. An intermediate plastic state cell may express LIN28A and SALL4; and PTCH1. An intermediate plastic state cell may express LIN28A and SALL4; and HOXA5. An intermediate plastic state cell may express LIN28A and SALL4; and CCND2. An intermediate plastic state cell may express LIN28A and SALL4; and SDC1. An intermediate plastic state cell may express LIN28A and SALL4; and TBX3. An intermediate plastic state cell may express LIN28A and SALL4; and BMP4. An intermediate plastic state cell may express LIN28A and SALL4; and IGF2. An intermediate plastic state cell may express LIN28A and SALL4; and one or more of MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2.

An intermediate plastic state cell may express LIN28A and SALL4; a second gene; and a third gene. An epithelial-like cell may express LIN28A and SALL4; one or more second genes; and one or more third genes. The second gene expressed by the intermediate plastic state cell may comprise MSX2, NMYC, WNT4, FGF19, or TOP2A, or any combination thereof. A somatic cell or epithelial-like cell may not express SALL4. A somatic cell or epithelial-like cell may not express both SALL4 and LIN28A. A somatic cell or epithelial-like cell may not express SALL4; a second gene; or a third gene. A somatic cell or epithelial-like cell may not express the second gene. A somatic cell or epithelial-like cell may not express the third gene. A somatic cell or epithelial-like cell may not express LIN28A or SALL4; may not express one or more second genes; and may not express one or more third genes. The second genes may comprise MSX2, NMYC, WNT4, FGF19, or TOP2A, or any combination thereof. The second genes may comprise MSX2. The second gene thereof may comprise NMYC. The second genes may comprise WNT4. The second gene thereof may comprise FGF19. The second genes may comprise TOP2A. The third genes may comprise MSX1, HOXB9, WT1, GATA2, HMGA2, or LEF1, or any combination thereof. The third genes may comprise MSX1. The third genes may comprise HOXB9. The third gene thereof may comprise WT1. The third genes may comprise GATA2. The third genes may comprise HMGA2. The third genes may comprise LEF1. An intermediate plastic state cell may express LIN28A and SALL4; a second gene; a third gene; and a fourth gene. A somatic cell or epithelial-like cell may not express the fourth gene. The fourth gene may comprise FGF9, HOXA9, HOXA1, PTCH1, HOXA5, CCND2, SDC1, TBX3, BMP4, or IGF2, or any combination thereof. The fourth gene may comprise FGF9. The fourth gene may comprise HOXA9. The fourth gene may comprise HOXA1. The fourth gene may comprise PTCH1. The fourth gene may comprise HOXA5. The fourth gene may comprise CCND2. The fourth gene may comprise SDC1. The fourth gene may comprise TBX3. The fourth gene may comprise BMP4. The fourth gene may comprise IGF2.

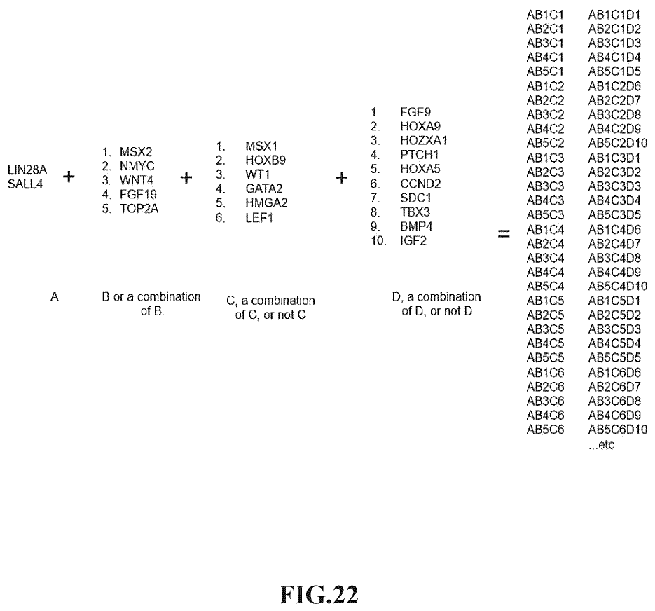

FIG. 22 depicts gene expression profile of exemplary intermediate plastic state cells in stage 2. In FIG. 22, “A” represents expression of LIN28A and SALL4; “B” represents expression of the second gene including MSX2, NMYC, WNT4, FGF19, or TOP2A, or any combination thereof; “C” represents expression the third gene including of MSX1, HOXB9, WT1, GATA2, HMGA2, or LEF1, or any combination thereof; “D” represents expression of the fourth gene including FGF9, HOXA9, HOXA1, PTCH1, HOXA5, CCND2, SDC1, TBX3, BMP4, or IGF2, or any combination thereof. For example, AB1C1D1 represents intermediate plastic state cells that express LIN28A, SALL4, MSX2, and MSX1; AB1C1 represents intermediate plastic state cells that express LIN28A, SALL4, MSX2, MSX1, and FGF9. The cells can express more than one of B, C, or D. For example, AB1B2C1C2D1D2 represents intermediate plastic state cells that express LIN28A, SALL4, MSX2, NMYC, MSX1, HOXB9, FGF9 and HOXA9.

A cell of the second population of stage 2 cells may express higher levels of LIN28A, SALL4, MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2, or any combination thereof, relative to a cell of the first population of stage 2 cells or any populations of the stage 1 cells. The higher level of expression of any one of LIN28A, SALL4, MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2 in a cell of the second population of stage 2 cells may be at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 5-fold, 100-fold, or more, relative to a cell of the first population of stage 2 cells or any populations of the stage 1 cells. A cell of the first population of stage 2 cells or any populations of the stage 1 cells may express lower levels of any one of LIN28A, SALL4, MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2, relative to a cell of the second population of stage 2 cells. The lower level of expression of any one of LIN28A, SALL4, MSX2, NMYC, WNT4, FGF19, TOP2A, MSX1, HOXB9, WT1, GATA2, HMGA2, LEF1, FGF9, HOXA9, HOXA1, PTCH1, HOXA5, CCND2, SDC1, TBX3, BMP4, or IGF2 in a cell of the first population of stage 2 cells or any populations of the stage 1 cells may be at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%, relative to a cell of the second population of stage 2 cells. The levels of expression can be measured by any methods described herein. For examples, gene expression can be measured by methods described in EXAMPLE 2. Gene expression can be measured by using any one of SEQ ID NO: 1-83 (including controls).

In some aspects, provided herein is a composition that comprises reprogramming factors for stage 2 conversion, or comprises cells of stage 2 (the first population of cells or the second population of cells), or comprises cells of stage 2 (the first population of cells or the second population of cells) and reprogramming factors for stage 2 conversion. In some cases, a composition comprises a culture medium comprising the reprogramming factors for stage 2 conversion.

In some cases, a composition comprises an isolated population of the second population of stage 2 cells. In some cases, a composition comprises an isolated population the first population of stage 2 cells. An isolated population of stage 2 cells may comprise at least about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more cells. An isolated population of stage 2 cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 cells. An isolated population of stage 2 cells may comprise at least one intermediate plastic state cell. In some cases, an isolated population of stage 2 cells may comprise at least about 1, 1×10\hat{ }1, 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more intermediate plastic states cells. An isolated population of cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 intermediate plastic states cells. In some cases, an isolated population of stage 3 cells may comprise at least about 1×10\hat{ }1, 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more intermediate plastic state cells, epithelial-like cells, or somatic cells, or any combination thereof. An isolated population of stage 3 cells may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 intermediate plastic state cells, epithelial-like cells, or somatic cells, or any combination thereof.

A composition may comprise a chemical reprogramming factor. A composition may comprise a plurality of chemical reprogramming factors. A composition may comprise at least 1, 2, 3, 4, 5, 6, 7, 8, 9, 10, 11, 12, 13, 14, 15, 16, 17, 18, 19, 20, 21, 22, 23, 24, 25 or more chemical reprogramming factors. A composition may comprise at most 1, 2, 3, 4, 5, 6, 7, 8, 9, 10, 11, 12, 13, 14, 15, 16, 17, 18, 19, 20, 21, 22, 23, 24, or 25 chemical reprogramming factors. A composition may comprise 1 chemical reprogramming factors. A composition may comprise 2 chemical reprogramming factors. A composition may comprise 3 chemical reprogramming factors. A composition may comprise 4 chemical reprogramming factors. A composition may comprise 5 chemical reprogramming factors. A composition may comprise 6 chemical reprogramming factors. A composition may comprise 7 chemical reprogramming factors. A composition may comprise 8 chemical reprogramming factors. A composition may comprise 9 chemical reprogramming factors. A composition may comprise 10 chemical reprogramming factors. A composition may comprise 11 chemical reprogramming factors. A composition may comprise 12 chemical reprogramming factors. A composition may comprise 13 chemical reprogramming factors. A composition may comprise 14 chemical reprogramming factors. A composition may comprise 15 chemical reprogramming factors. A composition may comprise 16 chemical reprogramming factors. A composition may comprise 17 chemical reprogramming factors. A composition may comprise 18 chemical reprogramming factors. A composition may comprise 19 chemical reprogramming factors. A composition may comprise 20 chemical reprogramming factors. A composition may comprise 21 chemical reprogramming factors. A composition may comprise 22 chemical reprogramming factors. A composition may comprise 23 chemical reprogramming factors. A composition may comprise 24 chemical reprogramming factors. A composition may comprise 25 chemical reprogramming factors. A chemical reprogramming factor in a composition may comprise any chemical reprogramming factors described here.

A composition may comprise a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, a BMP receptor/AMPK inhibitor, or a casein kinase 2 inhibitor, or any combination thereof. A composition may comprise at least 1, 2, 3, 4, 5, 6, 7, 8, 9, 10, 11, 12, 13, 14, or 15 of a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor. A composition may comprise at most 1, 2, 3, 4, 5, 6, 7, 8, 9, 10, 11, 12, 13, 14, or 15 of a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor. A composition may comprise a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, and a casein kinase 2 inhibitor.

A composition may comprise a glycogen synthase kinase 3 (GSK-3) inhibitor. A composition may comprise a transforming growth factor-beta (TGFβ) receptor inhibitor. A composition may comprise a retinoic acid receptor (RAR) agonist. A composition may comprise a c-Jun kinase inhibitor. A composition may comprise a CBP/p300 bromodomain inhibitor. A composition may comprise a S-adenosyl-L-homocysteine (SAH) hydrolase inhibitor. A composition may comprise an adenosine kinase inhibitor. A composition may comprise a Dot1L inhibitor. A composition may comprise a Menin-MLL interaction inhibitor. A composition may comprise a SET domain containing 2 (SETD2) inhibitor. A composition may comprise an agonist for the G protein-coupled receptor Smoothened. A composition may comprise a ROCK inhibitor. A composition may comprise a BMP receptor/AMPK inhibitor. A composition may comprise a Jak1/Jak2 inhibitor. A composition may comprise a p38 MAPK inhibitor. A composition may comprise an Akt inhibitor. A composition may comprise a casein kinase 2 inhibitor.

A composition may comprise a glycogen synthase kinase 3 (GSK-3) inhibitor, a transforming growth factor-beta (TGFβ) receptor inhibitor, or a c-Jun kinase inhibitor, or any combination thereof. A composition may comprise a glycogen synthase kinase 3 (GSK-3) inhibitor, a transforming growth factor-beta (TGFβ) receptor inhibitor, and a c-Jun kinase inhibitor. A composition may comprise a glycogen synthase kinase 3 (GSK-3) inhibitor, a transforming growth factor-beta (TGFβ) receptor inhibitor, a c-Jun kinase inhibitor, a CBP/p300 bromodomain inhibitor, a S-adenosyl-L-homocysteine (SAH) hydrolase inhibitor, or an adenosine kinase inhibitor, or any combination thereof. A composition may comprise a glycogen synthase kinase 3 (GSK-3) inhibitor, a transforming growth factor-beta (TGFβ) receptor inhibitor, a c-Jun kinase inhibitor, a CBP/p300 bromodomain inhibitor; and a S-adenosyl-L-homocysteine (SAH) hydrolase inhibitor or adenosine kinase inhibitor. A composition may comprise a glycogen synthase kinase 3 (GSK-3) inhibitor, a transforming growth factor-beta (TGFβ) receptor inhibitor, a c-Jun kinase inhibitor, a CBP/p300 bromodomain inhibitor, and a S-adenosyl-L-homocysteine (SAH) hydrolase inhibitor. A composition may comprise a glycogen synthase kinase 3 (GSK-3) inhibitor, a transforming growth factor-beta (TGFβ) receptor inhibitor, a c-Jun kinase inhibitor, a CBP/p300 bromodomain inhibitor, and an adenosine kinase inhibitor. A composition may comprise a glycogen synthase kinase 3 (GSK-3) inhibitor, a transforming growth factor-beta (TGFβ) receptor inhibitor, a c-Jun kinase inhibitor, and a S-adenosyl-L-homocysteine (SAH) hydrolase inhibitor. A composition may comprise a glycogen synthase kinase 3 (GSK-3) inhibitor, a transforming growth factor-beta (TGFβ) receptor inhibitor, a c-Jun kinase inhibitor, and an adenosine kinase inhibitor. A composition may comprise a glycogen synthase kinase 3 (GSK-3) inhibitor, a transforming growth factor-beta (TGFβ) receptor inhibitor, a c-Jun kinase inhibitor; and a S-adenosyl-L-homocysteine (SAH) hydrolase inhibitor or an adenosine kinase inhibitor. A composition may comprise a glycogen synthase kinase 3 (GSK-3) inhibitor, a transforming growth factor-beta (TGFβ) receptor inhibitor, a c-Jun kinase inhibitor, a CBP/p300 bromodomain inhibitor, a S-adenosyl-L-homocysteine (SAH) hydrolase inhibitor, and an adenosine kinase inhibitor. The composition may further comprise a retinoic acid receptor (RAR) agonist, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor.

A composition may comprise epithelial-like cells that express LIN28A; and a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor, or any combination thereof. A composition may comprise epithelial-like cells that express LIN28A; and at least 1, 2, 3, 4, 5, 6, 7, 8, 9, 10, 11, 12, 13, 14, or 15 of a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor. A composition may comprise epithelial-like cells that express LIN28A; and at most 1, 2, 3, 4, 5, 6, 7, 8, 9, 10, 11, 12, 13, 14, or 15 of a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor. A composition may comprise epithelial-like cells that express LIN28A; and a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, and a casein kinase 2 inhibitor.

A composition may comprise epithelial-like cells that express LIN28A; and a glycogen synthase kinase 3 (GSK-3) inhibitor. A composition may comprise epithelial-like cells that express LIN28A; and a transforming growth factor-beta (TGFβ) receptor inhibitor. A composition may comprise epithelial-like cells that express LIN28A; and a retinoic acid receptor (RAR) agonist. A composition may comprise epithelial-like cells that express LIN28A; and a c-Jun kinase inhibitor. A composition may comprise epithelial-like cells that express LIN28A; and a CBP/p300 bromodomain inhibitor. A composition may comprise epithelial-like cells that express LIN28A; and a S-adenosyl-L-homocysteine (SAH) hydrolase inhibitor. A composition may comprise epithelial-like cells that express LIN28A, and an adenosine kinase inhibitor. A composition may comprise epithelial-like cells that express LIN28A; and a Dot1L inhibitor. A composition may comprise epithelial-like cells that express LIN28A; and a Menin-MLL interaction inhibitor. A composition may comprise epithelial-like cells that express LIN28A; and a SET domain containing 2 (SETD2) inhibitor. A composition may comprise epithelial-like cells that express LIN28A; and an agonist for the G protein-coupled receptor Smoothened. A composition may comprise epithelial-like cells that express LIN28A; and a ROCK inhibitor. A composition may comprise epithelial-like cells that express LIN28A; and a BMP receptor/AMPK inhibitor. A composition may comprise epithelial-like cells that express LIN28A; and a Jak1/Jak2 inhibitor. A composition may comprise epithelial-like cells that express LIN28A; and a p38 MAPK inhibitor. A composition may comprise epithelial-like cells that express LIN28A; and an Akt inhibitor. A composition may comprise epithelial-like cells that express LIN28A; and a casein kinase 2 inhibitor.

A composition may comprise epithelial-like cells that express LIN28A; and a glycogen synthase kinase 3 (GSK-3) inhibitor, a transforming growth factor-beta (TGFβ) receptor inhibitor, or a c-Jun kinase inhibitor, or any combination thereof. A composition may comprise epithelial-like cells that express LIN28A; and a glycogen synthase kinase 3 (GSK-3) inhibitor, a transforming growth factor-beta (TGFβ) receptor inhibitor, and a c-Jun kinase inhibitor. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, a S-adenosyl-L-homocysteine (SAH) hydrolase inhibitor, or an adenosine kinase inhibitor, or any combination thereof. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor, a CBP/p300 bromodomain inhibitor; and a S-adenosyl-L-homocysteine (SAH) hydrolase inhibitor or adenosine kinase inhibitor. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, and a S-adenosyl-L-homocysteine (SAH) hydrolase inhibitor. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, and an adenosine kinase inhibitor. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor, and a S-adenosyl-L-homocysteine (SAH) hydrolase inhibitor. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor, and an adenosine kinase inhibitor. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor; and a S-adenosyl-L-homocysteine (SAH) hydrolase inhibitor or an adenosine kinase inhibitor. A composition may comprise epithelial-like cells that express LIN28A; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, a S-adenosyl-L-homocysteine (SAH) hydrolase inhibitor, and an adenosine kinase inhibitor. The composition may further comprise a retinoic acid receptor (RAR) agonist, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor. The epithelial-like cells may express LIN28A, NMYC, WNT2B, PAX8, SMAD3, GLI3, KRT18, KRT19, WT1, or TBX2, or any combination thereof. The epithelial-like cells may express LIN28A. The epithelial-like cells may also express NMYC, WNT2B, PAX8, SMAD3, or GLI3, or any combination thereof. The epithelial-like cells may express LIN28A and NMYC. The epithelial-like cells may express LIN28A and WNT2B. The epithelial-like cells may express LIN28A and PAX8. The epithelial-like cells may express LIN28A and SMAD3. The epithelial-like cells may express LIN28A and GLI3. The epithelial-like cells may not express any one of MMP1, ZEB1, VIM, COL1A1, COL5A1, COL6A2, PRRX1, SNAI2, TWIST1, or TWIST2.

A composition may comprise intermediate plastic state cells; and a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor, or any combination thereof. A composition may comprise intermediate plastic state cells; and at least 1, 2, 3, 4, 5, 6, 7, 8, 9, 10, 11, 12, 13, 14, or 15 of a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor. A composition may comprise intermediate plastic state cells; and at most 1, 2, 3, 4, 5, 6, 7, 8, 9, 10, 11, 12, 13, 14, or 15 of a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor. A composition may comprise intermediate plastic state cells; and a glycogen synthase kinase 3 (GSK-3) inhibitor, a transforming growth factor-beta (TGFβ) receptor inhibitor, a retinoic acid receptor (RAR) agonist, a c-Jun kinase inhibitor, a CBP/p300 bromodomain inhibitor, a S-adenosyl-L-homocysteine (SAH) hydrolase inhibitor, an adenosine kinase inhibitor, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, and a casein kinase 2 inhibitor.

A composition may comprise intermediate plastic state cells; and a glycogen synthase kinase 3 (GSK-3) inhibitor. A composition may comprise intermediate plastic state cells; and a transforming growth factor-beta (TGFβ) receptor inhibitor. A composition may comprise intermediate plastic state cells; and a retinoic acid receptor (RAR) agonist. A composition may comprise intermediate plastic state cells; and a c-Jun kinase inhibitor. A composition may comprise intermediate plastic state cells; and a CBP/p300 bromodomain inhibitor. A composition may comprise intermediate plastic state cells; and a S-adenosyl-L-homocysteine (SAH) hydrolase inhibitor. A composition may comprise intermediate plastic state cells; and an adenosine kinase inhibitor. A composition may comprise intermediate plastic state cells; and a Dot1L inhibitor. A composition may comprise intermediate plastic state cells; and a Menin-MLL interaction inhibitor. A composition may comprise intermediate plastic state cells; and a SET domain containing 2 (SETD2) inhibitor. A composition may comprise intermediate plastic state cells; and an agonist for the G protein-coupled receptor Smoothened. A composition may comprise intermediate plastic state cells; and a ROCK inhibitor. A composition may comprise intermediate plastic state cells; and a BMP receptor/AMPK inhibitor. A composition may comprise intermediate plastic state cells; and a Jak1/Jak2 inhibitor. A composition may comprise intermediate plastic state cells; and a p38 MAPK inhibitor. A composition may comprise intermediate plastic state cells; and an Akt inhibitor. A composition may comprise intermediate plastic state cells; and a casein kinase 2 inhibitor.

A composition may comprise intermediate plastic state cells; and a glycogen synthase kinase 3 (GSK-3) inhibitor, a transforming growth factor-beta (TGFβ) receptor inhibitor, or a c-Jun kinase inhibitor, or any combination thereof. A composition may comprise intermediate plastic state cells; and a glycogen synthase kinase 3 (GSK-3) inhibitor, a transforming growth factor-beta (TGFβ) receptor inhibitor, and a c-Jun kinase inhibitor. A composition may comprise intermediate plastic state cells; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, a S-adenosyl-L-homocysteine (SAH) hydrolase inhibitor, or an adenosine kinase inhibitor, or any combination thereof. A composition may comprise intermediate plastic state cells; and a glycogen synthase kinase 3 (GSK-3) inhibitor, a transforming growth factor-beta (TGFβ) receptor inhibitor, a c-Jun kinase inhibitor, a CBP/p300 bromodomain inhibitor; and a S-adenosyl-L-homocysteine (SAH) hydrolase inhibitor or adenosine kinase inhibitor. A composition may comprise intermediate plastic state cells; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, and a S-adenosyl-L-homocysteine (SAH) hydrolase inhibitor. A composition may comprise intermediate plastic state cells; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, and an adenosine kinase inhibitor. A composition may comprise intermediate plastic state cells; and a glycogen synthase kinase 3 (GSK-3) inhibitor, a transforming growth factor-beta (TGFβ) receptor inhibitor, a c-Jun kinase inhibitor, and a S-adenosyl-L-homocysteine (SAH) hydrolase inhibitor. A composition may comprise intermediate plastic state cells; and a glycogen synthase kinase 3 (GSK-3) inhibitor, a transforming growth factor-beta (TGFβ) receptor inhibitor, a c-Jun kinase inhibitor, and an adenosine kinase inhibitor. A composition may comprise intermediate plastic state cells; and a glycogen synthase kinase 3 (GSK-3) inhibitor, a transforming growth factor-beta (TGFβ) receptor inhibitor, a c-Jun kinase inhibitor; and a S-adenosyl-L-homocysteine (SAH) hydrolase inhibitor or an adenosine kinase inhibitor. A composition may comprise intermediate plastic state cells; and a glycogen synthase kinase 3 (GSK-3) inhibitor, a transforming growth factor-beta (TGFβ) receptor inhibitor, a c-Jun kinase inhibitor, a CBP/p300 bromodomain inhibitor, a S-adenosyl-L-homocysteine (SAH) hydrolase inhibitor, and an adenosine kinase inhibitor. The composition may further comprise a retinoic acid receptor (RAR) agonist, a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor.

The intermediate plastic state cells of the compositions may express LIN28A, SALL4, MSX2, NMYC, WNT4, FGF19, or TOP2A, or any combination thereof. The intermediate plastic state cells of the compositions may express LIN28A and SALL4. The intermediate plastic state cells of the compositions may also express MSX2, NMYC, WNT4, FGF19, TOP2A, or a combinations thereof. The intermediate plastic state cells of the compositions may express LIN28A, SALL4, and MSX2. The intermediate plastic state cells of the compositions may express LIN28A, SALL4, and NMYC. The intermediate plastic state cells of the compositions may express LIN28A, SALL4, and WNT4. The intermediate plastic state cells of the compositions may express LIN28A, SALL4, and FGF19. The intermediate plastic state cells of the compositions may express LIN28A, SALL4, and TOP2A. The intermediate plastic state cells of the compositions may further express any one of MSX1, HOXB9, WT1, GATA2, HMGA2, or LEF1, or any combination thereof. Additionally, the intermediate plastic state cells of the compositions may further express any one of FGF9, HOXA9, HOXA1, PTCH1, HOXA5, CCND2, SDC1, TBX3, BMP4, or IGF2, or any combination thereof.

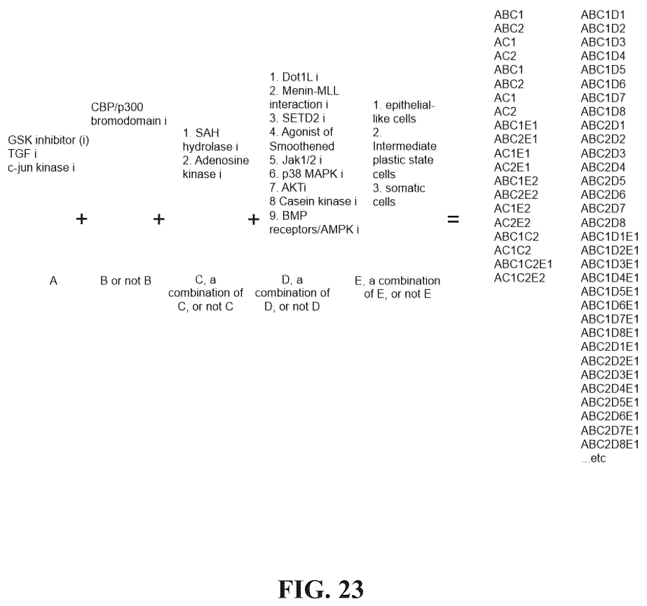

FIG. 23 depicts exemplary compositions comprising chemical reprogramming factors and optional cells in stage 2. In FIG. 23, “A” represents the combination of a glycogen synthase kinase 3 (GSK-3) inhibitor, a transforming growth factor-beta (TGFβ) receptor inhibitor, and a c-Jun kinase inhibitor; “B” represents the combination of a CBP/p300 bromodomain inhibitor or without the CBP/p300 bromodomain inhibitor; “C” represents a S-adenosyl-L-homocysteine (SAH) hydrolase inhibitor, or an adenosine kinase inhibitor, or a combination thereof, the composition may or may not have compounds in group C; “D” represents a Dot1L inhibitor, a Menin-MLL interaction inhibitor, a SET domain containing 2 (SETD2) inhibitor, an agonist for the G protein-coupled receptor Smoothened, a ROCK inhibitor, a BMP receptor/AMPK inhibitor, a Jak1/Jak2 inhibitor, a p38 MAPK inhibitor, an Akt inhibitor, or a casein kinase 2 inhibitor, or any combination thereof, the composition may or may not have compounds in group D; “E” represents epithelial-like cells, intermediate plastic state cells, or somatic cells, or any combination thereof, the composition may or may not have any of these cells. For example, ABC1D1D2E2 represents a composition that includes a glycogen synthase kinase 3 (GSK-3) inhibitor, a transforming growth factor-beta (TGFβ) receptor inhibitor, a c-Jun kinase inhibitor, a S-adenosyl-L-homocysteine (SAH) hydrolase inhibitor, a Dot1L inhibitor, a Menin-MLL interaction inhibitor, and intermediate plastic state cells.

A composition may comprise CHIR99021 or CHIR98014; E-616452, A 83-01, SB431542, SB 505124, GW 788388, dorsomorphine, or SB 525334; TTNPB, Ch55, or AM580; JNKIN8, JNKIN7, JNKIN5, or JNKIN12; SGC-CBP30, I-CBP112, GNE272, or GNE409; DZNep, NepA, Adox, or DZA; 5-ITU or ABT 702; EPZ004777 or EPZ5676; VTP50469, MI3454, or WDR5-IN-4; SETD2-IN-1, EPZ-719, or MMSET-IN-1; SAG, Purmorphamine, Hh-Ag1.5, or human SHH; Y-27632 or thiazovivin; Dorsomorphin; Ruxolitinib, Tofacitinib, AZD1480, Baricitinib, S-Ruxolitinib, or Fedratinib; BIRB796, SB203580, or SB202190; AKTi; CX-4945, TPP 22, or Ellagic acid, or any combination thereof. A composition may comprise at least 1, 2, 3, 4, 5, 6, 7, 8, 9, 10, 11, 12, 13, 14, or 15 of CHIR99021 or CHIR98014; E-616452, A 83-01, SB431542, SB 505124, GW 788388, dorsomorphine, or SB 525334; TTNPB, Ch55, or AM580; JNKIN8, JNKIN7, JNKIN5, or JNKIN12; SGC-CBP30, I-CBP112, GNE272, or GNE409; DZNep, NepA, Adox, or DZA; 5-ITU or ABT 702; EPZ004777 or EPZ5676; VTP50469, MI3454, or WDR5-IN-4; SETD2-IN-1, EPZ-719, or MMSET-IN-1; SAG, Purmorphamine, Hh-Ag1.5, or human SHH; Y-27632 or thiazovivin; Dorsomorphin; Ruxolitinib, Tofacitinib, AZD1480, Baricitinib, S-Ruxolitinib, or Fedratinib; BIRB796, SB203580, or SB202190; AKTi; or CX-4945, TPP 22, or Ellagic acid. A composition may comprise at most 1, 2, 3, 4, 5, 6, 7, 8, 9, 10, 11, 12, 13, 14, or 15 of CHIR99021 or CHIR98014; E-616452, A 83-01, SB431542, SB 505124, GW 788388, dorsomorphine, or SB 525334; TTNPB, Ch55, or AM580; JNKIN8, JNKIN7, JNKIN5, or JNKIN12; SGC-CBP30, I-CBP112, GNE272, or GNE409; DZNep, NepA, Adox, or DZA; 5-ITU or ABT 702; EPZ004777 or EPZ5676; VTP50469, MI3454, or WDR5-IN-4; SETD2-IN-1, EPZ-719, or MMSET-IN-1; SAG, Purmorphamine, Hh-Ag1.5, or human SHH; Y-27632 or thiazovivin; Dorsomorphin; Ruxolitinib, Tofacitinib, AZD1480, Baricitinib, S-Ruxolitinib, or Fedratinib; BIRB796, SB203580, or SB202190; AKTi; or CX-4945, TPP 22, or Ellagic acid. A composition may comprise CHIR99021 or CHIR98014; E-616452, A 83-01, SB431542, SB 505124, GW 788388, dorsomorphine, or SB 525334; TTNPB, Ch55, or AM580; JNKIN8, JNKIN7, JNKIN5, or JNKIN12; SGC-CBP30, I-CBP112, GNE272, or GNE409; DZNep, NepA, Adox, or DZA; 5-ITU or ABT 702; EPZ004777 or EPZ5676; VTP50469, MI3454, or WDR5-IN-4; SETD2-IN-1, EPZ-719, or MMSET-IN-1; SAG, Purmorphamine, Hh-Ag1.5, or human SHH; Y-27632 or thiazovivin; Dorsomorphin; Ruxolitinib, Tofacitinib, AZD1480, Baricitinib, S-Ruxolitinib, or Fedratinib; BIRB796, SB203580, or SB202190; AKTi; and CX-4945, TPP 22, or Ellagic acid.

A composition may comprise CHIR99021 or CHIR98014. A composition may comprise E-616452, A 83-01, SB431542, SB 505124, GW 788388, dorsomorphine, or SB 525334. A composition may comprise a TTNPB, Ch55, or AM580. A composition may comprise JNKIN8, JNKIN7, JNKIN5, or JNKIN12. A composition may comprise SGC-CBP30, I-CBP112, GNE272, or GNE409. A composition may comprise DZNep, NepA, Adox, or DZA. A composition may comprise 5-ITU or ABT 702. A composition may comprise EPZ004777 or EPZ5676. A composition may comprise VTP50469, MI3454, or WDR5-IN-4. A composition may comprise SETD2-IN-1, EPZ-719, or MMSET-IN-1. A composition may comprise SAG, Purmorphamine, Hh-Ag1.5, or human SHH. A composition may comprise Y-27632 or thiazovivin. A composition may comprise Dorsomorphin. A composition may comprise Ruxolitinib, Tofacitinib, AZD1480, Baricitinib, S-Ruxolitinib, or Fedratinib. A composition may comprise BIRB796, SB203580, or SB202190. A composition may comprise an AKTi. A composition may comprise CX-4945, TPP 22, or Ellagic acid.

A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; or any combination thereof. A composition may comprise CHIR99021 or CHIR98014; E-616452, A 83-01, SB431542, SB 505124, GW 788388, dorsomorphine, or SB 525334; and a c-Jun kinase inhibitor. A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; SGC-CBP30, I-CBP112, GNE272, or GNE409; DZNep, NepA, Adox, or DZA; 5-ITU or ABT 702; or any combination thereof. A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; a CBP/p300 bromodomain inhibitor; and a S-adenosyl-L-homocysteine (SAH) hydrolase inhibitor or adenosine kinase inhibitor. A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; SGC-CBP30, I-CBP112, GNE272, or GNE409; and a S-adenosyl-L-homocysteine (SAH) hydrolase inhibitor. A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; SGC-CBP30, I-CBP112, GNE272, or GNE409; and an adenosine kinase inhibitor. A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; and DZNep, NepA, Adox, or DZA. A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; and an adenosine kinase inhibitor. A composition may comprise CHIR99021 or CHIR98014; E-616452, A 83-01, SB431542, SB 505124, GW 788388, dorsomorphine, or SB 525334; a c-Jun kinase inhibitor; and a S-adenosyl-L-homocysteine (SAH) hydrolase inhibitor or an adenosine kinase inhibitor. A composition may comprise CHIR99021 or CHIR98014; E-616452, A 83-01, SB431542, SB 505124, GW 788388, dorsomorphine, or SB 525334; JNKIN8, JNKIN7, JNKIN5, or JNKIN12; SGC-CBP30, I-CBP112, GNE272, or GNE409; DZNep, NepA, Adox, or DZA; and an adenosine kinase inhibitor. The composition may further comprise TTNPB, Ch55, or AM580; EPZ004777 or EPZ5676; VTP50469, MI3454, or WDR5-IN-4; SETD2-IN-1, EPZ-719, or MMSET-IN-1; SAG, Purmorphamine, Hh-Ag1.5, or human SHH; Y-27632 or thiazovivin; Dorsomorphin; Ruxolitinib, Tofacitinib, AZD1480, Baricitinib, S-Ruxolitinib, or Fedratinib; BIRB796, SB203580, or SB202190; AKTi; or CX-4945, TPP 22, or Ellagic acid.

A composition may comprise CHIR99021; E-616452; TTNPB; JNKIN8; SGC-CBP30; DZNep; 5-ITU; EPZ004777; VTP50469; SETD2-IN-1; SAG; Y-27632; Dorsomorphin; Ruxolitinib; BIRB796; AKTi; or CX-4945, or any combination thereof. A composition may comprise at least 1, 2, 3, 4, 5, 6, 7, 8, 9, 10, 11, 12, 13, 14, or 15 of CHIR99021; E-616452; TTNPB; JNKIN8; SGC-CBP30; DZNep; 5-ITU; EPZ004777; VTP50469; SETD2-IN-1; SAG; Y-27632; Dorsomorphin; Ruxolitinib; BIRB796; AKTi; or CX-4945. A composition may comprise at most 1, 2, 3, 4, 5, 6, 7, 8, 9, 10, 11, 12, 13, 14, or 15 of CHIR99021; E-616452; TTNPB; JNKIN8; SGC-CBP30; DZNep; 5-ITU; EPZ004777; VTP50469; SETD2-IN-1; SAG; Y-27632; Dorsomorphin; Ruxolitinib; BIRB796; AKTi; or CX-4945. A composition may comprise CHIR99021; E-616452; TTNPB; JNKIN8; SGC-CBP30; DZNep; 5-ITU; EPZ004777; VTP50469; SETD2-IN-1; SAG; Y-27632; Dorsomorphin; Ruxolitinib; BIRB796; AKTi; and CX-4945, TPP 22, or Ellagic acid.

A composition may comprise CHIR99021. A composition may comprise E-616452. A composition may comprise a TTNPB. A composition may comprise JNKIN8. A composition may comprise SGC-CBP30. A composition may comprise DZNep. A composition may comprise 5-ITU. A composition may comprise EPZ004777. A composition may comprise VTP50469. A composition may comprise SETD2-IN-1. A composition may comprise SAG. A composition may comprise Y-27632. A composition may comprise Dorsomorphin. A composition may comprise Ruxolitinib. A composition may comprise BIRB796. A composition may comprise an AKTi. A composition may comprise CX-4945, TPP 22, or Ellagic acid.

A composition may comprise CHIR99021; E-616452; or JNKIN8; or any combination thereof. A composition may comprise CHIR99021; E-616452; and a c-Jun kinase inhibitor. A composition may comprise CHIR99021; E-616452; JNKIN8; SGC-CBP30; DZNep; or 5-ITU; or any combination thereof. A composition may comprise CHIR99021; E-616452; JNKIN8; a CBP/p300 bromodomain inhibitor; and a S-adenosyl-L-homocysteine (SAH) hydrolase inhibitor or adenosine kinase inhibitor. A composition may comprise CHIR99021; E-616452; JNKIN8; SGC-CBP30; and a S-adenosyl-L-homocysteine (SAH) hydrolase inhibitor. A composition may comprise CHIR99021; E-616452; JNKIN8; SGC-CBP30; and an adenosine kinase inhibitor. A composition may comprise CHIR99021; E-616452; JNKIN8; and a S-adenosyl-L-homocysteine (SAH) hydrolase inhibitor. A composition may comprise CHIR99021; E-616452; JNKIN8; and an adenosine kinase inhibitor. A composition may comprise CHIR99021; E-616452; a c-Jun kinase inhibitor; and a S-adenosyl-L-homocysteine (SAH) hydrolase inhibitor or an adenosine kinase inhibitor. A composition may comprise CHIR99021; E-616452; JNKIN8; SGC-CBP30; DZNep; and an adenosine kinase inhibitor. The composition may further comprise TTNPB; EPZ004777; VTP50469; SETD2-IN-1; SAG; Y-27632; Dorsomorphin; Ruxolitinib; BIRB796; AKTi; or CX-4945.

A composition may comprise at least about 0.1 μM, 0.2 μM, 0.3 μM, 0.4 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 100 μM, 150 μM, 200 μM, 250 μM or more CHIR99021 within the composition. A composition may comprise at most about 0.1 μM, 0.2 μM, 0.3 μM, 0.4 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 100 μM, 150 μM, 200 μM, or 250 μM CHIR99021 within the composition. A composition may comprise about 0.5 μM CHIR99021 within the composition. A composition may comprise about 1 μM CHIR99021 within the composition. A composition may comprise about 2 μM CHIR99021 within the composition. A composition may comprise about 3 μM CHIR99021 within the composition. A composition may comprise about 4 μM CHIR99021 within the composition. A composition may comprise about 5 μM CHIR99021 within the composition. A composition may comprise about 6 μM CHIR99021 within the composition. A composition may comprise about 7 μM CHIR99021 within the composition. A composition may comprise about 8 μM CHIR99021 within the composition. A composition may comprise about 9 μM CHIR99021 within the composition. A composition may comprise about 10 μM CHIR99021 within the composition. A composition may comprise about 15 μM CHIR99021 within the composition. A composition may comprise about 20 μM CHIR99021 within the composition. A composition may comprise about 30 μM CHIR99021 within the composition. A composition may comprise about 40 μM CHIR99021 within the composition. A composition may comprise about 50 μM CHIR99021 within the composition. A composition may comprise about 0.1 μM to about 100 μM CHIR99021 within the composition. A composition may comprise about 0.2 μM to about 75 μM CHIR99021 within the composition. A composition may comprise about 0.5 μM to about 50 μM CHIR99021 within the composition. A composition may comprise about 1 μM to about 25 μM CHIR99021 within the composition. A composition may comprise about 2 μM to about 12.5 μM CHIR99021 within the composition. A composition may comprise about 4 μM to about 6.25 μM CHIR99021 within the composition.

A composition may comprise at least about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, 500 μM or more E-616452 within the composition. A composition may comprise at most about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, or 500 μM E-616452 within the composition. A composition may comprise about 1 μM E-616452 within the composition. A composition may comprise about 2 μM E-616452 within the composition. A composition may comprise about 3 μM E-616452 within the composition. A composition may comprise about 4 μM E-616452 within the composition. A composition may comprise about 5 μM E-616452 within the composition. A composition may comprise about 6 μM E-616452 within the composition. A composition may comprise about 7 μM E-616452 within the composition. A composition may comprise about 8 μM E-616452 within the composition. A composition may comprise about 9 μM E-616452 within the composition. A composition may comprise about 10 μM E-616452 within the composition. A composition may comprise about 15 μM E-616452 within the composition. A composition may comprise about 20 μM E-616452 within the composition. A composition may comprise about 30 μM E-616452 within the composition. A composition may comprise about 40 μM E-616452 within the composition. A composition may comprise about 50 μM E-616452 within the composition. A composition may comprise about 60 μM E-616452 within the composition. A composition may comprise about 70 μM E-616452 within the composition. A composition may comprise about 80 μM E-616452 within the composition. A composition may comprise about 90 μM E-616452 within the composition. A composition may comprise about 100 μM E-616452 within the composition. A composition may comprise about 1 μM to about 100 μM E-616452 within the composition. A composition may comprise about 2 μM to about 75 μM E-616452 within the composition. A composition may comprise about 3 μM to about 50 μM E-616452 within the composition. A composition may comprise about 4 μM to about 40 μM E-616452 within the composition. A composition may comprise about 5 μM to about 30 μM E-616452 within the composition. A composition may comprise about 7.5 μM to about 20 μM E-616452 within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more JNKIN8 within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM JNKIN8 within the composition. A composition may comprise about 0.05 μM JNKIN8 within the composition. A composition may comprise about 0.1 μM JNKIN8 within the composition. A composition may comprise about 0.15 μM JNKIN8 within the composition. A composition may comprise about 0.2 μM JNKIN8 within the composition. A composition may comprise about 0.25 μM JNKIN8 within the composition. A composition may comprise about 0.3 μM JNKIN8 within the composition. A composition may comprise about 0.35 μM JNKIN8 within the composition. A composition may comprise about 0.4 μM JNKIN8 within the composition. A composition may comprise about 0.45 μM JNKIN8 within the composition. A composition may comprise about 0.5 μM JNKIN8 within the composition. A composition may comprise about 1 μM JNKIN8 within the composition. A composition may comprise about 1.5 μM JNKIN8 within the composition. A composition may comprise about 2 μM JNKIN8 within the composition. A composition may comprise about 2.5 μM JNKIN8 within the composition. A composition may comprise about 3 μM JNKIN8 within the composition. A composition may comprise about 3.5 μM JNKIN8 within the composition. A composition may comprise about 4 μM JNKIN8 within the composition. A composition may comprise about 4.5 μM JNKIN8 within the composition. A composition may comprise about 5 μM JNKIN8 within the composition. A composition may comprise about 0.05 μM to about 2.5 μM JNKIN8 within the composition. A composition may comprise about 0.1 μM to about 1.875 μM JNKIN8 within the composition. A composition may comprise about 0.15 μM to about 1.25 μM JNKIN8 within the composition. A composition may comprise about 0.2 μM to about 1 μM JNKIN8 within the composition. A composition may comprise about 0.25 μM to about 0.75 μM JNKIN8 within the composition. A composition may comprise about 0.375 μM to about 0.5 μM JNKIN8 within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more SGC-CBP30 within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM SGC-CBP30 within the composition. A composition may comprise about 0.2 μM SGC-CBP30 within the composition. A composition may comprise about 0.4 μM SGC-CBP30 within the composition. A composition may comprise about 0.6 μM SGC-CBP30 within the composition. A composition may comprise about 0.8 μM SGC-CBP30 within the composition. A composition may comprise about 1 μM SGC-CBP30 within the composition. A composition may comprise about 1.2 μM SGC-CBP30 within the composition. A composition may comprise about 1.4 μM SGC-CBP30 within the composition. A composition may comprise about 1.6 μM SGC-CBP30 within the composition. A composition may comprise about 1.8 μM SGC-CBP30 within the composition. A composition may comprise about 2 μM SGC-CBP30 within the composition. A composition may comprise about 4 μM SGC-CBP30 within the composition. A composition may comprise about 6 μM SGC-CBP30 within the composition. A composition may comprise about 8 μM SGC-CBP30 within the composition. A composition may comprise about 10 μM SGC-CBP30 within the composition. A composition may comprise about 12 μM SGC-CBP30 within the composition. A composition may comprise about 14 μM SGC-CBP30 within the composition. A composition may comprise about 16 μM SGC-CBP30 within the composition. A composition may comprise about 18 μM SGC-CBP30 within the composition. A composition may comprise about 20 μM SGC-CBP30 within the composition. A composition may comprise about 0.2 μM to about 20 μM SGC-CBP30 within the composition. A composition may comprise about 0.4 μM to about 15 μM SGC-CBP30 within the composition. A composition may comprise about 0.6 μM to about 10 μM SGC-CBP30 within the composition. A composition may comprise about 0.8 μM to about 8 μM SGC-CBP30 within the composition. A composition may comprise about 1 μM to about 6 μM SGC-CBP30 within the composition. A composition may comprise about 1.5 μM to about 4 μM SGC-CBP30 within the composition.

A composition may comprise at least about 0.0004 μM, 0.0008 μM, 0.0012 μM, 0.0016 μM, 0.002 μM, 0.004 μM, 0.006 μM, 0.008 μM, 0.01 μM, 0.012 μM, 0.014 μM, 0.016 μM, 0.018 μM, 0.02 μM, 0.04 μM, 0.06 μM, 0.08 μM, 0.1 μM, 0.12 μM, 0.14 μM, 0.16 μM, 0.18 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM or more DZNep within the composition. A composition may comprise at most about 0.0004 μM, 0.0008 μM, 0.0012 μM, 0.0016 μM, 0.002 μM, 0.004 μM, 0.006 μM, 0.008 μM, 0.01 μM, 0.012 μM, 0.014 μM, 0.016 μM, 0.018 μM, 0.02 μM, 0.04 μM, 0.06 μM, 0.08 μM, 0.1 μM, 0.12 μM, 0.14 μM, 0.16 μM, 0.18 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, or 1 μM DZNep within the composition. A composition may comprise about 0.002 μM DZNep within the composition. A composition may comprise about 0.004 μM DZNep within the composition. A composition may comprise about 0.006 μM DZNep within the composition. A composition may comprise about 0.008 μM DZNep within the composition. A composition may comprise about 0.01 μM DZNep within the composition. A composition may comprise about 0.012 μM DZNep within the composition. A composition may comprise about 0.014 μM DZNep within the composition. A composition may comprise about 0.016 μM DZNep within the composition. A composition may comprise about 0.018 μM DZNep within the composition. A composition may comprise about 0.02 μM DZNep within the composition. A composition may comprise about 0.04 μM DZNep within the composition. A composition may comprise about 0.06 μM DZNep within the composition. A composition may comprise about 0.08 μM DZNep within the composition. A composition may comprise about 0.1 μM DZNep within the composition. A composition may comprise about 0.12 μM DZNep within the composition. A composition may comprise about 0.14 μM DZNep within the composition. A composition may comprise about 0.16 μM DZNep within the composition. A composition may comprise about 0.18 μM DZNep within the composition. A composition may comprise about 0.2 μM DZNep within the composition. A composition may comprise about 0.002 μM to about 0.2 μM DZNep within the composition. A composition may comprise about 0.0025 μM to about 0.15 μM DZNep within the composition. A composition may comprise about 0.005 μM to about 0.1 μM DZNep within the composition. A composition may comprise about 0.0075 μM to about 0.75 μM DZNep within the composition. A composition may comprise about 0.01 μM to about 0.5 μM DZNep within the composition. A composition may comprise about 0.015 μM to about 0.4 μM DZNep within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more 5-ITU within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM 5-ITU within the composition. A composition may comprise about 0.05 μM 5-ITU within the composition. A composition may comprise about 0.1 μM 5-ITU within the composition. A composition may comprise about 0.15 μM 5-ITU within the composition. A composition may comprise about 0.2 μM 5-ITU within the composition. A composition may comprise about 0.25 μM 5-ITU within the composition. A composition may comprise about 0.3 μM 5-ITU within the composition. A composition may comprise about 0.35 μM 5-ITU within the composition. A composition may comprise about 0.4 μM 5-ITU within the composition. A composition may comprise about 0.45 μM 5-ITU within the composition. A composition may comprise about 0.5 μM 5-ITU within the composition. A composition may comprise about 1 μM 5-ITU within the composition. A composition may comprise about 1.5 μM 5-ITU within the composition. A composition may comprise about 2 μM 5-ITU within the composition. A composition may comprise about 2.5 μM 5-ITU within the composition. A composition may comprise about 3 μM 5-ITU within the composition. A composition may comprise about 3.5 μM 5-ITU within the composition. A composition may comprise about 4 μM 5-ITU within the composition. A composition may comprise about 4.5 μM 5-ITU within the composition. A composition may comprise about 5 μM 5-ITU within the composition. A composition may comprise about 0.05 μM to about 2.5 μM 5-ITU within the composition. A composition may comprise about 0.1 μM to about 1.875 μM 5-ITU within the composition. A composition may comprise about 0.15 μM to about 1.25 μM 5-ITU within the composition. A composition may comprise about 0.2 μM to about 1 μM 5-ITU within the composition. A composition may comprise about 0.25 μM to about 0.75 μM 5-ITU within the composition. A composition may comprise about 0.375 μM to about 0.5 μM 5-ITU within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more TTNPB within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM TTNPB within the composition. A composition may comprise about 0.2 μM TTNPB within the composition. A composition may comprise about 0.4 μM TTNPB within the composition. A composition may comprise about 0.6 μM TTNPB within the composition. A composition may comprise about 0.8 μM TTNPB within the composition. A composition may comprise about 1 μM TTNPB within the composition. A composition may comprise about 1.2 μM TTNPB within the composition. A composition may comprise about 1.4 μM TTNPB within the composition. A composition may comprise about 1.6 μM TTNPB within the composition. A composition may comprise about 1.8 μM TTNPB within the composition. A composition may comprise about 2 μM TTNPB within the composition. A composition may comprise about 4 μM TTNPB within the composition. A composition may comprise about 6 μM TTNPB within the composition. A composition may comprise about 8 μM TTNPB within the composition. A composition may comprise about 10 μM TTNPB within the composition. A composition may comprise about 12 μM TTNPB within the composition. A composition may comprise about 14 μM TTNPB within the composition. A composition may comprise about 16 μM TTNPB within the composition. A composition may comprise about 18 μM TTNPB within the composition. A composition may comprise about 20 μM TTNPB within the composition. A composition may comprise about 0.2 μM to about 20 μM TTNPB within the composition. A composition may comprise about 0.4 μM to about 15 μM TTNPB within the composition. A composition may comprise about 0.6 μM to about 10 μM TTNPB within the composition. A composition may comprise about 0.8 μM to about 8 μM TTNPB within the composition. A composition may comprise about 1 μM to about 6 μM TTNPB within the composition. A composition may comprise about 1.5 μM to about 4 μM TTNPB within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more EPZ5676 within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM EPZ5676 within the composition. A composition may comprise about 0.2 μM EPZ5676 within the composition. A composition may comprise about 0.4 μM EPZ5676 within the composition. A composition may comprise about 0.6 μM EPZ5676 within the composition. A composition may comprise about 0.8 μM EPZ5676 within the composition. A composition may comprise about 1 μM EPZ5676 within the composition. A composition may comprise about 1.2 μM EPZ5676 within the composition. A composition may comprise about 1.4 μM EPZ5676 within the composition. A composition may comprise about 1.6 μM EPZ5676 within the composition. A composition may comprise about 1.8 μM EPZ5676 within the composition. A composition may comprise about 2 μM EPZ5676 within the composition. A composition may comprise about 4 μM EPZ5676 within the composition. A composition may comprise about 6 μM EPZ5676 within the composition. A composition may comprise about 8 μM EPZ5676 within the composition. A composition may comprise about 10 μM EPZ5676 within the composition. A composition may comprise about 12 μM EPZ5676 within the composition. A composition may comprise about 14 μM EPZ5676 within the composition. A composition may comprise about 16 μM EPZ5676 within the composition. A composition may comprise about 18 μM EPZ5676 within the composition. A composition may comprise about 20 μM EPZ5676 within the composition. A composition may comprise about 0.2 μM to about 20 μM EPZ5676 within the composition. A composition may comprise about 0.4 μM to about 15 μM EPZ5676 within the composition. A composition may comprise about 0.6 μM to about 10 μM EPZ5676 within the composition. A composition may comprise about 0.8 μM to about 8 μM EPZ5676 within the composition. A composition may comprise about 1 μM to about 6 μM EPZ5676 within the composition. A composition may comprise about 1.5 μM to about 4 μM EPZ5676 within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more VTP50469 within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM VTP50469 within the composition. A composition may comprise about 0.05 μM VTP50469 within the composition. A composition may comprise about 0.1 μM VTP50469 within the composition. A composition may comprise about 0.15 μM VTP50469 within the composition. A composition may comprise about 0.2 μM VTP50469 within the composition. A composition may comprise about 0.25 μM VTP50469 within the composition. A composition may comprise about 0.3 μM VTP50469 within the composition. A composition may comprise about 0.35 μM VTP50469 within the composition. A composition may comprise about 0.4 μM VTP50469 within the composition. A composition may comprise about 0.45 μM VTP50469 within the composition. A composition may comprise about 0.5 μM VTP50469 within the composition. A composition may comprise about 1 μM VTP50469 within the composition. A composition may comprise about 1.5 μM VTP50469 within the composition. A composition may comprise about 2 μM VTP50469 within the composition. A composition may comprise about 2.5 μM VTP50469 within the composition. A composition may comprise about 3 μM VTP50469 within the composition. A composition may comprise about 3.5 μM VTP50469 within the composition. A composition may comprise about 4 μM VTP50469 within the composition. A composition may comprise about 4.5 μM VTP50469 within the composition. A composition may comprise about 5 μM VTP50469 within the composition. A composition may comprise about 0.05 μM to about 2.5 μM VTP50469 within the composition. A composition may comprise about 0.1 μM to about 1.875 μM VTP50469 within the composition. A composition may comprise about 0.15 μM to about 1.25 μM VTP50469 within the composition. A composition may comprise about 0.2 μM to about 1 μM VTP50469 within the composition. A composition may comprise about 0.25 μM to about 0.75 μM VTP50469 within the composition. A composition may comprise about 0.375 μM to about 0.5 μM VTP50469 within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more SETD2-IN-1 within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM SETD2-IN-1 within the composition. A composition may comprise about 0.05 μM SETD2-IN-1 within the composition. A composition may comprise about 0.1 μM SETD2-IN-1 within the composition. A composition may comprise about 0.15 μM SETD2-IN-1 within the composition. A composition may comprise about 0.2 μM SETD2-IN-1 within the composition. A composition may comprise about 0.25 μM SETD2-IN-1 within the composition. A composition may comprise about 0.3 μM SETD2-IN-1 within the composition. A composition may comprise about 0.35 μM SETD2-IN-1 within the composition. A composition may comprise about 0.4 μM SETD2-IN-1 within the composition. A composition may comprise about 0.45 μM SETD2-IN-1 within the composition. A composition may comprise about 0.5 μM SETD2-IN-1 within the composition. A composition may comprise about 1 μM SETD2-IN-1 within the composition. A composition may comprise about 1.5 μM SETD2-IN-1 within the composition. A composition may comprise about 2 μM SETD2-IN-1 within the composition. A composition may comprise about 2.5 μM SETD2-IN-1 within the composition. A composition may comprise about 3 μM SETD2-IN-1 within the composition. A composition may comprise about 3.5 μM SETD2-IN-1 within the composition. A composition may comprise about 4 μM SETD2-IN-1 within the composition. A composition may comprise about 4.5 μM SETD2-IN-1 within the composition. A composition may comprise about 5 μM SETD2-IN-1 within the composition. A composition may comprise about 0.05 μM to about 2.5 μM SETD2-IN-1 within the composition. A composition may comprise about 0.1 μM to about 1.875 μM SETD2-IN-1 within the composition. A composition may comprise about 0.15 μM to about 1.25 μM SETD2-IN-1 within the composition. A composition may comprise about 0.2 μM to about 1 μM SETD2-IN-1 within the composition. A composition may comprise about 0.25 μM to about 0.75 μM SETD2-IN-1 within the composition. A composition may comprise about 0.375 μM to about 0.5 μM SETD2-IN-1 within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more SAG within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM SAG within the composition. A composition may comprise about 0.05 μM SAG within the composition. A composition may comprise about 0.1 μM SAG within the composition. A composition may comprise about 0.15 μM SAG within the composition. A composition may comprise about 0.2 μM SAG within the composition. A composition may comprise about 0.25 μM SAG within the composition. A composition may comprise about 0.3 μM SAG within the composition. A composition may comprise about 0.35 μM SAG within the composition. A composition may comprise about 0.4 μM SAG within the composition. A composition may comprise about 0.45 μM SAG within the composition. A composition may comprise about 0.5 μM SAG within the composition. A composition may comprise about 1 μM SAG within the composition. A composition may comprise about 1.5 μM SAG within the composition. A composition may comprise about 2 μM SAG within the composition. A composition may comprise about 2.5 μM SAG within the composition. A composition may comprise about 3 μM SAG within the composition. A composition may comprise about 3.5 μM SAG within the composition. A composition may comprise about 4 μM SAG within the composition. A composition may comprise about 4.5 μM SAG within the composition. A composition may comprise about 5 μM SAG within the composition. A composition may comprise about 0.05 μM to about 2.5 μM SAG within the composition. A composition may comprise about 0.1 μM to about 1.875 μM SAG within the composition. A composition may comprise about 0.15 μM to about 1.25 μM SAG within the composition. A composition may comprise about 0.2 μM to about 1 μM SAG within the composition. A composition may comprise about 0.25 μM to about 0.75 μM SAG within the composition. A composition may comprise about 0.375 μM to about 0.5 μM SAG within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more Dorsomorphin within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM Dorsomorphin within the composition. A composition may comprise about 0.05 μM Dorsomorphin within the composition. A composition may comprise about 0.1 μM Dorsomorphin within the composition. A composition may comprise about 0.15 μM Dorsomorphin within the composition. A composition may comprise about 0.2 μM Dorsomorphin within the composition. A composition may comprise about 0.25 μM Dorsomorphin within the composition. A composition may comprise about 0.3 μM Dorsomorphin within the composition. A composition may comprise about 0.35 μM Dorsomorphin within the composition. A composition may comprise about 0.4 μM Dorsomorphin within the composition. A composition may comprise about 0.45 μM Dorsomorphin within the composition. A composition may comprise about 0.5 μM Dorsomorphin within the composition. A composition may comprise about 1 μM Dorsomorphin within the composition. A composition may comprise about 1.5 μM Dorsomorphin within the composition. A composition may comprise about 2 μM Dorsomorphin within the composition. A composition may comprise about 2.5 μM Dorsomorphin within the composition. A composition may comprise about 3 μM Dorsomorphin within the composition. A composition may comprise about 3.5 μM Dorsomorphin within the composition. A composition may comprise about 4 μM Dorsomorphin within the composition. A composition may comprise about 4.5 μM Dorsomorphin within the composition. A composition may comprise about 5 μM Dorsomorphin within the composition. A composition may comprise about 0.05 μM to about 2.5 μM Dorsomorphin within the composition. A composition may comprise about 0.1 μM to about 1.875 μM Dorsomorphin within the composition. A composition may comprise about 0.15 μM to about 1.25 μM Dorsomorphin within the composition. A composition may comprise about 0.2 μM to about 1 μM Dorsomorphin within the composition. A composition may comprise about 0.25 μM to about 0.75 μM Dorsomorphin within the composition. A composition may comprise about 0.375 μM to about 0.5 μM Dorsomorphin within the composition.

A composition may comprise at least about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, 500 μM or more Y-27632 within the composition. A composition may comprise at most about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, or 500 μM Y-27632 within the composition. A composition may comprise about 1 μM Y-27632 within the composition. A composition may comprise about 2 μM Y-27632 within the composition. A composition may comprise about 3 μM Y-27632 within the composition. A composition may comprise about 4 μM Y-27632 within the composition. A composition may comprise about 5 μM Y-27632 within the composition. A composition may comprise about 6 μM Y-27632 within the composition. A composition may comprise about 7 μM Y-27632 within the composition. A composition may comprise about 8 μM Y-27632 within the composition. A composition may comprise about 9 μM Y-27632 within the composition. A composition may comprise about 10 μM Y-27632 within the composition. A composition may comprise about 15 μM Y-27632 within the composition. A composition may comprise about 20 μM Y-27632 within the composition. A composition may comprise about 30 μM Y-27632 within the composition. A composition may comprise about 40 μM Y-27632 within the composition. A composition may comprise about 50 μM Y-27632 within the composition. A composition may comprise about 60 μM Y-27632 within the composition. A composition may comprise about 70 μM Y-27632 within the composition. A composition may comprise about 80 μM Y-27632 within the composition. A composition may comprise about 90 μM Y-27632 within the composition. A composition may comprise about 100 μM Y-27632 within the composition. A composition may comprise about 1 μM to about 100 μM Y-27632 within the composition. A composition may comprise about 2 μM to about 75 μM Y-27632 within the composition. A composition may comprise about 3 μM to about 50 μM Y-27632 within the composition. A composition may comprise about 4 μM to about 40 μM Y-27632 within the composition. A composition may comprise about 5 μM to about 30 μM Y-27632 within the composition. A composition may comprise about 7.5 μM to about 20 μM Y-27632 within the composition.

A composition may comprise at least about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, 50 μM or more Ruxolitinib within the composition. A composition may comprise at most about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, or 50 μM Ruxolitinib within the composition. A composition may comprise about 0.1 μM Ruxolitinib within the composition. A composition may comprise about 0.2 μM Ruxolitinib within the composition. A composition may comprise about 0.3 μM Ruxolitinib within the composition. A composition may comprise about 0.4 μM Ruxolitinib within the composition. A composition may comprise about 0.5 μM Ruxolitinib within the composition. A composition may comprise about 0.6 μM Ruxolitinib within the composition. A composition may comprise about 0.7 μM Ruxolitinib within the composition. A composition may comprise about 0.8 μM Ruxolitinib within the composition. A composition may comprise about 0.9 μM Ruxolitinib within the composition. A composition may comprise about 1 μM Ruxolitinib within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more BIRB796 within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM BIRB796 within the composition. A composition may comprise about 0.2 μM BIRB796 within the composition. A composition may comprise about 0.4 μM BIRB796 within the composition. A composition may comprise about 0.6 μM BIRB796 within the composition. A composition may comprise about 0.8 μM BIRB796 within the composition. A composition may comprise about 1 μM BIRB796 within the composition. A composition may comprise about 1.2 μM BIRB796 within the composition. A composition may comprise about 1.4 μM BIRB796 within the composition. A composition may comprise about 1.6 μM BIRB796 within the composition. A composition may comprise about 1.8 μM BIRB796 within the composition. A composition may comprise about 2 μM BIRB796 within the composition. A composition may comprise about 4 μM BIRB796 within the composition. A composition may comprise about 6 μM BIRB796 within the composition. A composition may comprise about 8 μM BIRB796 within the composition. A composition may comprise about 10 μM BIRB796 within the composition. A composition may comprise about 12 μM BIRB796 within the composition. A composition may comprise about 14 μM BIRB796 within the composition. A composition may comprise about 16 μM BIRB796 within the composition. A composition may comprise about 18 μM BIRB796 within the composition. A composition may comprise about 20 μM BIRB796 within the composition. A composition may comprise about 0.2 μM to about 20 μM BIRB796 within the composition. A composition may comprise about 0.4 μM to about 15 μM BIRB796 within the composition. A composition may comprise about 0.6 μM to about 10 μM BIRB796 within the composition. A composition may comprise about 0.8 μM to about 8 μM BIRB796 within the composition. A composition may comprise about 1 μM to about 6 μM BIRB796 within the composition. A composition may comprise about 1.5 μM to about 4 μM BIRB796 within the composition.

A composition may comprise at least about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, 50 μM or more AKTi within the composition. A composition may comprise at most about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, or 50 μM AKTi within the composition. A composition may comprise about 0.1 μM AKTi within the composition. A composition may comprise about 0.2 μM AKTi within the composition. A composition may comprise about 0.3 μM AKTi within the composition. A composition may comprise about 0.4 μM AKTi within the composition. A composition may comprise about 0.5 μM AKTi within the composition. A composition may comprise about 0.6 μM AKTi within the composition. A composition may comprise about 0.7 μM AKTi within the composition. A composition may comprise about 0.8 μM AKTi within the composition. A composition may comprise about 0.9 μM AKTi within the composition. A composition may comprise about 1 μM AKTi within the composition. A composition may comprise about 2 μM AKTi within the composition. A composition may comprise about 3 μM AKTi within the composition. A composition may comprise about 4 μM AKTi within the composition. A composition may comprise about 5 μM AKTi within the composition. A composition may comprise about 6 μM AKTi within the composition. A composition may comprise about 7 μM AKTi within the composition. A composition may comprise about 8 μM AKTi within the composition. A composition may comprise about 9 μM AKTi within the composition. A composition may comprise about 10 μM AKTi within the composition. A composition may comprise about 0.1 μM to about 10 μM AKTi within the composition. A composition may comprise about 0.2 μM to about 7.5 μM AKTi within the composition. A composition may comprise about 0.3 μM to about 5 μM AKTi within the composition. A composition may comprise about 0.4 μM to about 4 μM AKTi within the composition. A composition may comprise about 0.5 μM to about 3 μM AKTi within the composition. A composition may comprise about 0.75 μM to about 2 μM AKTi within the composition.

A composition may comprise at least about 0.016 μM, 0.018 μM, 0.02 μM, 0.022 μM, 0.024 μM, 0.026 μM, 0.028 μM, 0.03 μM, 0.032 μM, 0.034 μM, 0.036 μM, 0.038 μM, 0.04 μM, 0.05 μM, 0.06 μM, 0.07 μM, 0.08 μM, 0.09 μM, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM or more CX-4945 within the composition. A composition may comprise at most about 0.016 μM, 0.018 μM, 0.02 μM, 0.022 μM, 0.024 μM, 0.026 μM, 0.028 μM, 0.03 μM, 0.032 μM, 0.034 μM, 0.036 μM, 0.038 μM, 0.04 μM, 0.05 μM, 0.06 μM, 0.07 μM, 0.08 μM, 0.09 μM, 0.1 μM, 0.15 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM CX-4945 within the composition. A composition may comprise about 0.08 μM CX-4945 within the composition. A composition may comprise about 0.1 μM CX-4945 within the composition. A composition may comprise about 0.2 μM CX-4945 within the composition. A composition may comprise about 0.3 μM CX-4945 within the composition. A composition may comprise about 0.4 μM CX-4945 within the composition. A composition may comprise about 0.5 μM CX-4945 within the composition. A composition may comprise about 0.6 μM CX-4945 within the composition. A composition may comprise about 0.7 μM CX-4945 within the composition. A composition may comprise about 0.8 μM CX-4945 within the composition. A composition may comprise about 0.9 μM CX-4945 within the composition. A composition may comprise about 1 μM CX-4945 within the composition. A composition may comprise about 1.5 μM CX-4945 within the composition. A composition may comprise about 2 μM CX-4945 within the composition. A composition may comprise about 3 μM CX-4945 within the composition. A composition may comprise about 4 μM CX-4945 within the composition. A composition may comprise about 5 μM CX-4945 within the composition. A composition may comprise about 6 μM CX-4945 within the composition. A composition may comprise about 7 μM CX-4945 within the composition. A composition may comprise about 8 μM CX-4945 within the composition. A composition may comprise about 0.08 μM to about 8 μM CX-4945 within the composition. A composition may comprise about 0.1 μM to about 4 μM CX-4945 within the composition. A composition may comprise about 0.15 μM to about 2 μM CX-4945 within the composition. A composition may comprise about 0.25 μM to about 1.5 μM CX-4945 within the composition. A composition may comprise about 0.5 μM to about 1 μM CX-4945 within the composition.

In another aspect of the invention, another aspect of the invention is to provide a composition that comprises reprogramming factors for stage 2 conversion, which is used to further enhance the differentiation potential of the cell population with increased differentiation potential disclosed herein, making it closer to pluripotent stem cells. The composition comprises a GSK-3β inhibitor, a transforming growth factor-beta (TGFβ) receptor inhibitor, an retinoic acid receptor (RAR) agonist, a G protein-coupled receptor Smoothened agonist, a c-Jun kinase inhibitor, a S-adenosyl-L-homocysteine (SAH) hydrolase inhibitor, and a DNA methyltransferase (DNMT) inhibitor. In some embodiments, the composition comprises a GSK=3β inhibitor, a transforming growth factor-beta (TGFβ) receptor inhibitor, an retinoic acid receptor (RAR) agonist, a G protein-coupled receptor Smoothened agonist, a c-Jun kinase inhibitor, a S-adenosyl-L-homocysteine (SAH) hydrolase inhibitor, a DNMT inhibitor, and any one selected from a ROCK1 inhibitor, a Dot1L inhibitor, a Menin-MLL interaction inhibitor, a MAPK inhibitor, a Jak1/Jak2 inhibitor, a histone acetyltransferase inhibitor, a Aktt inhibitor, a casein kinase inhibitor, or any combination thereof. In some embodiments, the composition comprises a GSK-3β inhibitor, a transforming growth factor-beta (TGFβ) receptor inhibitor, an retinoic acid receptor (RAR) agonist, a G protein-coupled receptor Smoothened agonist, a c-Jun kinase inhibitor, a S-adenosyl-L-homocysteine (SAH) hydrolase inhibitor, a DNMT inhibitor, a ROCK1 inhibitor, a Dot1L inhibitor, a Menin-MLL interaction inhibitor, a MAPK inhibitor, a Jak1/Jak2 inhibitor, a histone acetyltransferase inhibitor, an Akt inhibitor, and a casein kinase inhibitor. In some embodiments, the GSK-3β inhibitor disclosed herein is selected from CHIR99021, TD114-2, CHIR98014, GSK 3I inhibitor XV, BIO, SB-216763, or any combination thereof. In some embodiments, the GSK3β inhibitor disclosed herein is CHIR99021. In some embodiments, the concentration of the GSK3β inhibitor disclosed herein is 3 to 12 μM. In some embodiments, the concentration of the GSK3β inhibitor disclosed herein is 5 μM. In some embodiments, the DNA methyltransferase (DNMT) inhibitor disclosed herein is selected from GSK3689032, 5-aza-C, Decitabine, or any combination thereof. In a preferred embodiment, the DNA methyltransferase (DNMT) inhibitor disclosed herein is GSK3689032. In some embodiments, the concentration of the DNMT inhibitor disclosed herein is 0.01-0.1 μM. In a preferred embodiment, the concentration of the DNMT inhibitor disclosed herein is 0.02 μM.

In some embodiments, the composition disclosed herein comprises 5 μM CHIR999021, 10 μM 616452, 2 μM TTNPB, 0.5 μM SAG, 10 μM Y-27632, 0.5 μM JNKIN8, 1 μM Ruxolitinib, 2 μM BIRB796, 2 μM SGC-CBP30, 2 μM EPZ5676, 0.2 μM DZNep, 0.5 μM VTP50469, 0.5 μM Iodotubercidin, 5 μM RA, 0.02 μM GSK3685032, 0.2 μM HY-10249A (CAS No. 842148-40-7), and 1 μM CX-4945.

Subsequent to contacting any populations of stage 2 cells with any compositions described herein, the cells may be incubated in normoxic condition. For example, the stage 2 cells may be incubated with at most 23%, 22%, 21%, 20%, or 19% atmospheric oxygen. The normoxic condition may comprise about 22% atmospheric oxygen. The normoxic condition may comprise about 21% atmospheric oxygen. The normoxic condition may comprise about 20% atmospheric oxygen.

Subsequent to contacting any populations of stage 2 cells with any compositions described herein, a population of stage 2 cells may be incubated with a composition for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, or 30 days. A population of stage 2 cells may be incubated with a composition for at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, or 30 days. A population of stage 2 cells may be incubated with a composition for about 1 day. A population of stage 2 cells may be incubated with a composition for about 2 days. A population of stage 2 cells may be incubated with a composition for about 3 days. A population of stage 2 cells may be incubated with a composition for about 4 days. A population of stage 2 cells may be incubated with a composition for about 5 days. A population of stage 2 cells may be incubated with a composition for about 6 days. A population of stage 2 cells may be incubated with a composition for about 7 days. A population of stage 2 cells may be incubated with a composition for about 8 days. A population of stage 2 cells may be incubated with a composition for about 9 days. A population of stage 2 cells may be incubated with a composition for about 10 days. A population of stage 2 cells may be incubated with a composition for about 11 days. A population of stage 2 cells may be incubated with a composition for about 12 days. A population of stage 2 cells may be incubated with a composition for about 13 days. A population of stage 2 cells may be incubated with a composition for about 14 days. A population of stage 2 cells may be incubated with a composition for about 15 days. A population of stage 2 cells may be incubated with a composition for about 16 days. A population of stage 2 cells may be incubated with a composition for about 17 days. A population of stage 2 cells may be incubated with a composition for about 18 days. A population of stage 2 cells may be incubated with a composition for about 19 days. A population of stage 2 cells may be incubated with a composition for about 20 days. A population of stage 2 cells may be incubated with a composition for about 25 days.

Any of the compositions may not comprise feeder cells or serum. Any of the compositions may not comprise feeder cells and serum. Any of the compositions may not comprise feeder cells. Any of the compositions may be serum-free. Any of the compositions may comprise feeder cells. Any of the compositions may comprise serum.

### Stage 3

In some aspects, provided herein are stage 3 methods and compositions for conversion of an intermediate plastic state cell into a cell with a higher cell potency (e.g., less specialized cell), such as a pluripotent stem cell—the conversion process referred herein also as “stage 3.” A stage 3 method may be part of a conversion process that reprograms intermediate plastic state cells into pluripotent stem cells. A stage 3 method may be the third stage of a conversion process that reprograms somatic plastic state cells into pluripotent stem cells.

A stage 3 method may comprise contacting a first cell population with a first composition. A stage 3 method may comprise, subsequent to or during the contacting, converting a subset of the first cell population into different cells. The cell population comprising the different cells may comprise a second cell population. A stage 3 method may comprise incubating the first cell population with the first composition for a period of time. The subset of the first cell population may be converted into the different cells prior to, during, or subsequent to the incubating. In some cases, a stage 3 method may comprise removing the first composition from the second population of cells. In other cases, a stage 3 method may comprise removing the first composition from the first population of cells.

A first population of stage 3 cells may comprise any populations of stage 1 or stage 2 cells. In some case, the first population of stage 3 cells may comprise the second population of stage 1 or stage 2 cells. In some case, the first population of stage 3 cells may comprise a composition in which the second population of stage 2 cells are isolated or removed from the stage 2 chemical reprogramming factors.

The second population of stage 3 cells may comprise pluripotent stem cells. the pluripotent stem cells obtained after contacting a population of cells with a composition may be referred to chemically induced pluripotent stem cells (CiPSCs). hCiPSCs may comprise human hCiPSCs (hCiPSCs). The first population of stage 3 cells may comprise pluripotent stem cells.

A hCiPSC is not a naturally occurring cell. A hCiPSC may express a combination of genes that are not expressed by a naturally occurring cell. In some cases, a hCiPSC may express at least one gene at level at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 5-fold, 100-fold, or higher, relative to a naturally occurring cell. In some cases, a hCiPSC may express at least one gene at level at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100% lower, relative to a naturally occurring cell. The second population of stage 3 cells may comprise somatic cells, epithelial-like cells, intermediate plastic state cells or hCiPSCs. The second population of stage 3 cells may comprise somatic cells, epithelial-like cells, intermediate plastic state cells and hCiPSCs. The second population of stage 3 cells may not comprise somatic cells, epithelial-like cells, or intermediate plastic state cells. In some cases, the second population of stage 3 cells may comprise fewer somatic cells, epithelial-like cells, or intermediate plastic state cells than the first population of stage 3 cells. For example, the second population of stage 3 cells may have 0.001%, 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90% or 99% fewer somatic cells, epithelial-like cells, or intermediate plastic state cells than the first population of stage 3 cells. In some cases, the second population of stage 3 cells may comprise more hCiPSCs than the first population of stage 3 cells. For example, the second population of stage 3 cells may have 0.001%, 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 5-fold, 100-fold, or more hCiPSCs than the first population of stage 3 cells.

A hCiPSC may express OCT4, SOX2, NANOG, FGF4, ZFP57, DPPA5, REX1, DPPA4, TDGF1, TRA-1-60, TRA-1-81, SSEA4, KLF4, KLF17, DPPA3, DNMT3L, or UTF1, or any combination thereof. A hCiPSC may express OCT4. A hCiPSC may express SOX2. A hCiPSC may express NANOG. A hCiPSC may express FGF4. A hCiPSC may express ZFP57. A hCiPSC may express DPPA5. A hCiPSC may express REX1. A hCiPSC may express DPPA4. A hCiPSC may express TDGF1. A hCiPSC may express TRA-1-60. A hCiPSC may express TRA-1-81. A hCiPSC may express SSEA4. A hCiPSC may express KLF4. A hCiPSC may express KLF17. A hCiPSC may express DPPA3. A hCiPSC may express DNMT3L. A hCiPSC may express UTF1. A hCiPSC may express one or more of OCT4, SOX2, NANOG, FGF4, ZFP57, DPPA5, REX1, DPPA4, TDGF1, TRA-1-60, TRA-1-81, SSEA4, KLF4, KLF17, DPPA3, DNMT3L, or UTF1.

A hCiPSC may express OCT4, SOX2, or NANOG. A hCiPSC may express OCT4, SOX2, and NANOG. A hCiPSC may express OCT4 or SOX2. A hCiPSC may express OCT4 or NANOG. A hCiPSC may express SOX2 or NANOG. A hCiPSC may express OCT4 and SOX2. A hCiPSC may express OCT4 and NANOG. A hCiPSC may express SOX2 and NANOG. A hCiPSC may express OCT4, SOX2, and NANOG; and FGF4, ZFP57, DPPA5, REX1, DPPA4, TDGF1, TRA-1-60, TRA-1-81, SSEA4, KLF4, KLF17, DPPA3, DNMT3L, or UTF1, or any combination thereof. A hCiPSC may express OCT4, SOX2, and NANOG; and FGF4. A hCiPSC may express OCT4, SOX2, and NANOG; and ZFP57. A hCiPSC may express OCT4, SOX2, and NANOG; and DPPA5. A hCiPSC may express OCT4, SOX2, and NANOG; and REX1. A hCiPSC may express OCT4, SOX2, and NANOG; and DPPA4. A hCiPSC may express OCT4, SOX2, and NANOG; and TDGF1. A hCiPSC may express OCT4, SOX2, and NANOG; and TRA-1-60. A hCiPSC may express OCT4, SOX2, and NANOG; and TRA-1-81. A hCiPSC may express OCT4, SOX2, and NANOG; and SSEA4. A hCiPSC may express OCT4, SOX2, and NANOG; and KLF4. A hCiPSC may express OCT4, SOX2, and NANOG; and KLF17. A hCiPSC may express OCT4, SOX2, and NANOG; and DPPA3. A hCiPSC may express OCT4, SOX2, and NANOG; and DNMT3L. A hCiPSC may express OCT4, SOX2, and NANOG; and UTF1. A hCiPSC may express OCT4, SOX2, and NANOG; and one or more of FGF4, ZFP57, REX1, DPPA4, TDGF1, TRA-1-60, TRA-1-81, SSEA4, KLF4, KLF17, DPPA3, DPPA5, DNMT3L, REX1, or UTF1.

A CPISC may express OCT4, SOX2, and NANOG; a second gene; and a third gene. An epithelial-like cell may express OCT4, SOX2, and NANOG; one or more second genes; and one or more third genes. The second gene expressed by the CPISC may comprise FGF4, ZFP57, DPPA5, or REX1, or any combination thereof. A somatic cell, epithelial-like cell, or an intermediate plastic state cell may not express OCT4. A somatic cell, epithelial-like cell, or an intermediate plastic state cell may not express SOX2. A somatic cell, epithelial-like cell, or an intermediate plastic state cell may not express NANOG. A somatic cell, epithelial-like cell, or an intermediate plastic state cell may not express OCT4, SOX2, or NANOG. A somatic cell, epithelial-like cell, or an intermediate plastic state cell may not express OCT4, SOX2, and NANOG. A somatic cell, epithelial-like cell, or an intermediate plastic state cell may not express OCT4, SOX2, or NANOG; a second gene; or a third gene. A somatic cell, epithelial-like cell, or an intermediate plastic state cell may not express the second gene. A somatic cell, epithelial-like cell, or an intermediate plastic state cell may not express the third gene. A somatic cell, epithelial-like cell, or an intermediate plastic state cell may not express OCT4, SOX2, or NANOG; one or more second genes; and one or more third genes.

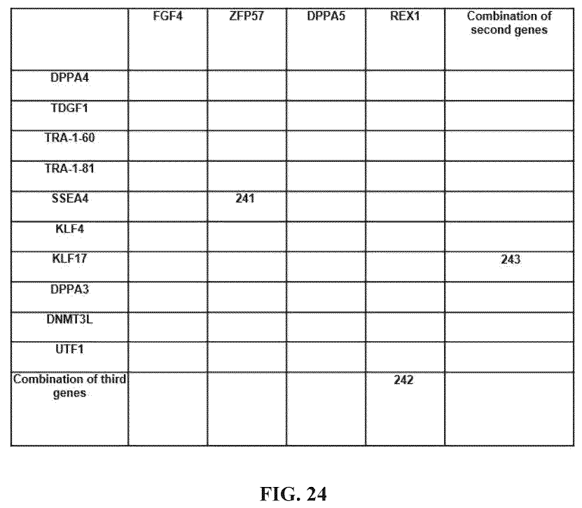

The second gene may comprise FGF4, ZFP57, DPPA5, or REX1, or any combination thereof. The second gene may comprise FGF4. The second gene may comprise ZFP57. The second gene may comprise DPPA5. The second gene may comprise REX1. The third gene may comprise DPPA4, TDGF1, TRA-1-60, TRA-1-81, SSEA4, KLF4, KLF17, DPPA3, DNMT3L, or UTF1, or any combination thereof. The third gene may comprise DPPA4. The third gene may comprise TDGF1. The third gene may comprise TRA-1-60. The third gene may comprise TRA-1-81. The third gene may comprise SSEA4. The third gene may comprise KLF4. The third gene may comprise KLF17. The third gene may comprise DPPA3. The third gene may comprise DNMT3L. The third gene may comprise UTF1. FIG. 24 depicts exemplary hCiPSCs that express OCT4, SOX2, and NANOG shown in a heatmap. The x-axis and y-axis of the heatmap show the second and the third genes, respectively. Each pixel of the heatmap represents one cell population. For example, cells 241 express OCT4, SOX2, NANOG, ZFP57, and SSEA4. Cells 242 express OCT4, SOX2, NANOG, REX1, or any combination of the third genes (e.g., TRA-1-60 and TRA-1-81). Cells 243 express OCT4, SOX2, NANOG, KLF17, or any combination of the second genes (e.g., DNMT3L and UTF1).

A cell of the second population of stage 3 cells may express higher levels of OCT4, SOX2, NANOG, FGF4, ZFP57, DPPA5, REX1, DPPA4, TDGF1, TRA-1-60, TRA-1-81, SSEA4, KLF4, KLF17, DPPA3, DNMT3L, or UTF1, or any combination thereof, relative to a cell of the first population of stage 3 cells or any populations of the stage 1 or stage 2 cells. The higher level of expression of any one of OCT4, SOX2, NANOG, FGF4, ZFP57, DPPA5, REX1, DPPA4, TDGF1, TRA-1-60, TRA-1-81, SSEA4, KLF4, KLF17, DPPA3, DNMT3L, or UTF1, or any combination thereof in a cell of the second population of stage 3 cells may be at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 5-fold, 100-fold, or more, relative to a cell of the first population of stage 3 cells or any populations of the stage 1 or stage 2 cells. A cell of the first population of stage 3 cells or any populations of the stage 1 or stage 2 cells may express lower levels of any one of OCT4, SOX2, NANOG, FGF4, ZFP57, DPPA5, REX1, DPPA4, TDGF1, TRA-1-60, TRA-1-81, SSEA4, KLF4, KLF17, DPPA3, DNMT3L, or UTF1, relative to a cell of the second population of stage 3 cells. The lower level of expression of any one of OCT4, SOX2, NANOG, FGF4, ZFP57, DPPA5, REX1, DPPA4, TDGF1, TRA-1-60, TRA-1-81, SSEA4, KLF4, KLF17, DPPA3, DNMT3L, or UTF1 in a cell of the first population of stage 3 cells or any populations of the stage 1 or stage 2 cells may be at least about 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%, relative to a cell of the second population of stage 3 cells. The levels of expression can be measured by any methods described herein. For examples, gene expression can be measured by methods described in EXAMPLE 2. Gene expression can be measured by using any one of SEQ ID NO: 1-83 (including controls).

In some aspects, provided herein is a composition that comprises reprogramming factors for stage 3 conversion, or comprises cells of stage 3 (the first population of cells or the second population of cells), or comprises cells of stage 3 (the first population of cells or the second population of cells) and reprogramming factors for stage 3 conversion. In some cases, a composition comprises a culture medium comprising the reprogramming factors for stage 3 conversion.

In some cases, a composition may comprise an isolated population of the second population of stage 3 cells. In some cases, a composition may comprise an isolated population the first population of stage 3 cells. An isolated population of stage 3 cells may comprise at least about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more cells. An isolated population of stage 3 cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 cells. An isolated population of stage 3 cells may comprise at least one hCiPSC. In some cases, an isolated population of stage 3 cells may comprise at least about 1, 1×10\hat{ }1, 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more hCiPSCs. An isolated population of cell may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 hCiPSCs. In some cases, an isolated population of stage 3 cells may comprise at least about 1×10\hat{ }1, 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10 or more hCiPSCs, intermediate plastic state cells, epithelial-like cells, or somatic cells, or any combination thereof. An isolated population of stage 3 cells may comprise at most about 1×10\hat{ }2, 1×10\hat{ }3, 1×10\hat{ }4, 1×10\hat{ }5, 1×10\hat{ }6, 1×10\hat{ }7, 1×10\hat{ }8, 1×10\hat{ }9, or 1×10\hat{ }10 hCiPSCs, intermediate plastic state cells, epithelial-like cells, or somatic cells, or any combination thereof.

A composition may comprise a chemical reprogramming factor. A composition may comprise a plurality of chemical reprogramming factors. A composition may comprise at least 1, 2, 3, 4, 5, 6, 7, 8, 9, 10, 11, 12, 13, 14, 15, or more chemical reprogramming factors. A composition may comprise at most 1, 2, 3, 4, 5, 6, 7, 8, 9, 10, 11, 12, 13, 14, or 15 chemical reprogramming factors. A composition may comprise 1 chemical reprogramming factors. A composition may comprise 2 chemical reprogramming factors. A composition may comprise 3 chemical reprogramming factors. A composition may comprise 4 chemical reprogramming factors. A composition may comprise 5 chemical reprogramming factors. A composition may comprise 6 chemical reprogramming factors. A composition may comprise 7 chemical reprogramming factors. A composition may comprise 8 chemical reprogramming factors. A composition may comprise 9 chemical reprogramming factors. A composition may comprise 10 chemical reprogramming factors. A composition may comprise 11 chemical reprogramming factors. A composition may comprise 12 chemical reprogramming factors. A composition may comprise 13 chemical reprogramming factors. A composition may comprise 14 chemical reprogramming factors. A composition may comprise 15 chemical reprogramming factors.

A composition may comprise a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor, or any combination thereof. A composition may comprise at least 1, 2, 3, 4, 5, 6, 7, or 8 of a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor. A composition may comprise at most 1, 2, 3, 4, 5, 6, 7, or 8 of a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor. A composition may comprise a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, and a S-adenosyl-L-homocysteine (SAH) hydrolase inhibitor. A composition may comprise a MEK inhibitor. A composition may comprise a B-Raf inhibitor. A composition may comprise a histone deacetylase inhibitor. A composition may comprise a Wnt inhibitor. A composition may comprise a glycogen synthase kinase 3 (GSK-3) inhibitor. A composition may comprise a ROCK inhibitor. A composition may comprise an inhibitor of histone demethylation. A composition may comprise a Dot1L inhibitor. A composition may comprise a S-adenosyl-L-homocysteine (SAH) hydrolase inhibitor.

A composition may comprise a MEK inhibitor, a B-Raf inhibitor, or a histone deacetylase inhibitor. A composition may comprise a MEK inhibitor or a B-Raf inhibitor. A composition may comprise a MEK inhibitor or a histone deacetylase inhibitor. A composition may comprise a B-Raf inhibitor or a histone deacetylase inhibitor. A composition may comprise a MEK inhibitor, a B-Raf inhibitor, and a histone deacetylase inhibitor.

A composition may comprise a MEK inhibitor, a B-Raf inhibitor, a histone deacetylase inhibitor, and a Wnt inhibitor. A composition may comprise a MEK inhibitor, a B-Raf inhibitor, a histone deacetylase inhibitor, and a glycogen synthase kinase 3 (GSK-3) inhibitor. A composition may comprise a MEK inhibitor, a B-Raf inhibitor, a histone deacetylase inhibitor, and a ROCK inhibitor. A composition may comprise a MEK inhibitor, a B-Raf inhibitor, a histone deacetylase inhibitor, and an inhibitor of histone demethylation. A composition may comprise a MEK inhibitor, a B-Raf inhibitor, a histone deacetylase inhibitor, and a Dot1L inhibitor. A composition may comprise a MEK inhibitor, a B-Raf inhibitor, a histone deacetylase inhibitor, and a S-adenosyl-L-homocysteine (SAH) hydrolase inhibitor. A composition may comprise a MEK inhibitor, a B-Raf inhibitor, a histone deacetylase inhibitor; and one or more of a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor.

A composition may comprise intermediate plastic state cells that express LIN28A and SALL4; and a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor, or any combination thereof. A composition may comprise intermediate plastic state cells that express LIN28A and SALL4; and at least 1, 2, 3, 4, 5, 6, 7, or 8 of a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor. A composition may comprise intermediate plastic state cells that express LIN28A and SALL4; and at most 1, 2, 3, 4, 5, 6, 7, or 8 of a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor. A composition may comprise intermediate plastic state cells that express LIN28A and SALL4; and a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, and a S-adenosyl-L-homocysteine (SAH) hydrolase inhibitor. A composition may comprise intermediate plastic state cells that express LIN28A and SALL4; and a MEK inhibitor. A composition may comprise intermediate plastic state cells that express LIN28A and SALL4; and a B-Raf inhibitor. A composition may comprise intermediate plastic state cells that express LIN28A and SALL4; and a histone deacetylase inhibitor. A composition may comprise intermediate plastic state cells that express LIN28A and SALL4; and a Wnt inhibitor. A composition may comprise intermediate plastic state cells that express LIN28A and SALL4; and a glycogen synthase kinase 3 (GSK-3) inhibitor. A composition may comprise intermediate plastic state cells that express LIN28A and SALL4; and a ROCK inhibitor. A composition may comprise intermediate plastic state cells that express LIN28A and SALL4; and an inhibitor of histone demethylation. A composition may comprise intermediate plastic state cells that express LIN28A and SALL4; and a Dot1L inhibitor. A composition may comprise intermediate plastic state cells that express LIN28A and SALL4; and a S-adenosyl-L-homocysteine (SAH) hydrolase inhibitor. The intermediate plastic state cells may further express one or more of MSX2, NMYC, WNT4, FGF19, or TOP2A. Additionally, the intermediate plastic state cells may further express one or more of FGF9, HOXA9, HOXA1, PTCH1, HOXA5, CCND2, SDC1, TBX3, BMP4, or IGF2.

A composition may comprise intermediate plastic state cells that express LIN28A and SALL4; and a MEK inhibitor, a B-Raf inhibitor, or a histone deacetylase inhibitor. A composition may comprise intermediate plastic state cells that express LIN28A and SALL4; and a MEK inhibitor or a B-Raf inhibitor. A composition may comprise intermediate plastic state cells that express LIN28A and SALL4; and a MEK inhibitor or a histone deacetylase inhibitor. A composition may comprise intermediate plastic state cells that express LIN28A and SALL4; and a B-Raf inhibitor or a histone deacetylase inhibitor. A composition may comprise intermediate plastic state cells that express LIN28A and SALL4; and a MEK inhibitor, a B-Raf inhibitor, and a histone deacetylase inhibitor.

A composition may comprise intermediate plastic state cells that express LIN28A and SALL4; and a MEK inhibitor, a B-Raf inhibitor, and a histone deacetylase inhibitor, and a Wnt inhibitor. A composition may comprise intermediate plastic state cells that express LIN28A and SALL4; and a MEK inhibitor, a B-Raf inhibitor, a histone deacetylase inhibitor, and a glycogen synthase kinase 3 (GSK-3) inhibitor. A composition may comprise intermediate plastic state cells that express LIN28A and SALL4; and a MEK inhibitor, a B-Raf inhibitor, a histone deacetylase inhibitor, and a ROCK inhibitor. A composition may comprise intermediate plastic state cells that express LIN28A and SALL4; and a MEK inhibitor, a B-Raf inhibitor, a histone deacetylase inhibitor, and an inhibitor of histone demethylation. A composition may comprise intermediate plastic state cells that express LIN28A and SALL4; and a MEK inhibitor, a B-Raf inhibitor, a histone deacetylase inhibitor, and a Dot1L inhibitor. A composition may comprise intermediate plastic state cells that express LIN28A and SALL4; and a MEK inhibitor, a B-Raf inhibitor, a histone deacetylase inhibitor, and a S-adenosyl-L-homocysteine (SAH) hydrolase inhibitor. A composition may comprise intermediate plastic state cells that express LIN28A and SALL4; a MEK inhibitor, a B-Raf inhibitor, and a histone deacetylase inhibitor; and one or more of a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor.

A composition may comprise hCiPSCs that express OCT4, SOX2, or NANOG; and a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor, or any combination thereof. A composition may comprise hCiPSCs that express OCT4, SOX2, or NANOG; and at least 1, 2, 3, 4, 5, 6, 7, or 8 of a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor. A composition may comprise hCiPSCs that express OCT4, SOX2, or NANOG; and at most 1, 2, 3, 4, 5, 6, 7, or 8 of a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor. A composition may comprise hCiPSCs that express OCT4, SOX2, or NANOG; and a MEK inhibitor, a B-Raf inhibitor, a histone deacetylase inhibitor, a Wnt inhibitor, a glycogen synthase kinase 3 (GSK-3) inhibitor, a ROCK inhibitor, an inhibitor of histone demethylation, a Dot1L inhibitor, and a S-adenosyl-L-homocysteine (SAH) hydrolase inhibitor. A composition may comprise hCiPSCs that express OCT4, SOX2, or NANOG; and a MEK inhibitor. A composition may comprise hCiPSCs that express OCT4, SOX2, or NANOG; and a B-Raf inhibitor. A composition may comprise hCiPSCs that express OCT4, SOX2, or NANOG; and a histone deacetylase inhibitor. A composition may comprise hCiPSCs that express OCT4, SOX2, or NANOG; and a Wnt inhibitor. A composition may comprise hCiPSCs that express OCT4, SOX2, or NANOG; and a glycogen synthase kinase 3 (GSK-3) inhibitor. A composition may comprise hCiPSCs that express OCT4, SOX2, or NANOG; and a ROCK inhibitor. A composition may comprise hCiPSCs that express OCT4, SOX2, or NANOG; and an inhibitor of histone demethylation. A composition may comprise hCiPSCs that express OCT4, SOX2, or NANOG; and a Dot1L inhibitor.

A composition may comprise hCiPSCs that express OCT4, SOX2, or NANOG; and a MEK inhibitor, a B-Raf inhibitor, or a histone deacetylase inhibitor. A composition may comprise hCiPSCs that express OCT4, SOX2, or NANOG; and a MEK inhibitor or a B-Raf inhibitor. A composition may comprise hCiPSCs that express OCT4, SOX2, or NANOG; and a MEK inhibitor or a histone deacetylase inhibitor. A composition may comprise hCiPSCs that express OCT4, SOX2, or NANOG; and a B-Raf inhibitor or a histone deacetylase inhibitor. A composition may comprise hCiPSCs that express OCT4, SOX2, or NANOG; and a MEK inhibitor, a B-Raf inhibitor, and a histone deacetylase inhibitor.

A composition may comprise hCiPSCs that express OCT4, SOX2, or NANOG; and a MEK inhibitor, a B-Raf inhibitor, a histone deacetylase inhibitor, and a Wnt inhibitor. A composition may comprise hCiPSCs that express OCT4, SOX2, or NANOG; and a MEK inhibitor, a B-Raf inhibitor, a histone deacetylase inhibitor, and a glycogen synthase kinase 3 (GSK-3) inhibitor. A composition may comprise hCiPSCs that express OCT4, SOX2, or NANOG; and a MEK inhibitor, a B-Raf inhibitor, a histone deacetylase inhibitor, and a ROCK inhibitor. A composition may comprise hCiPSCs that express OCT4, SOX2, or NANOG; and a MEK inhibitor, a B-Raf inhibitor, a histone deacetylase inhibitor, and an inhibitor of histone demethylation. A composition may comprise hCiPSCs that express OCT4, SOX2, or NANOG; and a MEK inhibitor, a B-Raf inhibitor, a histone deacetylase inhibitor; and a Dot1L inhibitor. A composition may comprise hCiPSCs that express OCT4, SOX2, or NANOG; and a MEK inhibitor, a B-Raf inhibitor, a histone deacetylase inhibitor, and a S-adenosyl-L-homocysteine (SAH) hydrolase inhibitor. A composition may comprise hCiPSCs that express OCT4, SOX2, or NANOG; and a MEK inhibitor, a B-Raf inhibitor, and a histone deacetylase inhibitor; and one or more of a Wnt inhibitor, a glycogen synthase kinase 3 (GSK-3) inhibitor, a ROCK inhibitor, an inhibitor of histone demethylation, a Dot1L inhibitor, or a S-adenosyl-L-homocysteine (SAH) hydrolase inhibitor.

The hCiPSCs of the compositions may further express one or more of FGF4, ZFP57, DPPA5, or REX1. Additionally, the hCiPSCs of the compositions also express one or more of DPPA4, TDGF1, TRA-1-60, TRA-1-81, SSEA4, KLF4, KLF17, DPPA3, DNMT3L, or UTF1.

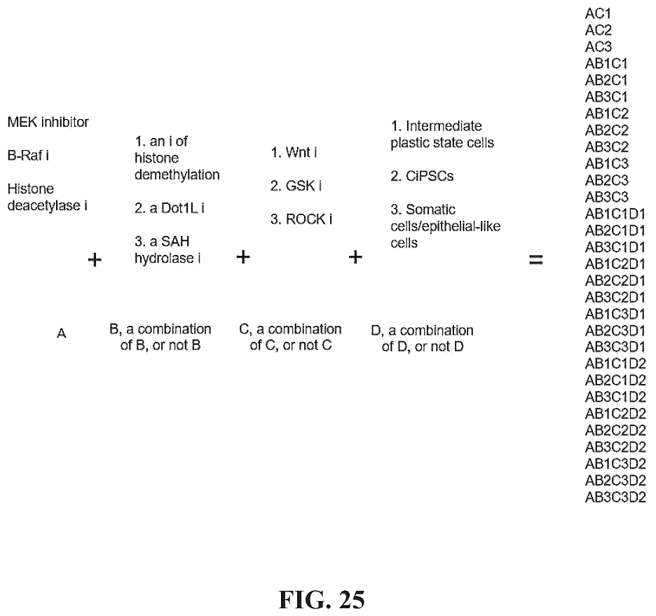

FIG. 25 depicts exemplary compositions comprising chemical reprogramming factors and optional cells in stage 3. In FIG. 25, “A” represents the combination of a MEK inhibitor, a B-Raf inhibitor, and a histone deacetylase inhibitor; “B” represents an inhibitor of histone demethylation, a Dot1L inhibitor, or a S-adenosyl-L-homocysteine (SAH) hydrolase inhibitor, or any combination thereof, the composition may or may not have any of the compounds in group B; “C” represents a Wnt inhibitor, a glycogen synthase kinase 3 (GSK-3) inhibitor, or a ROCK inhibitor, any combination thereof, the composition may or may not have any of the compounds in group C; “D” represents intermediate plastic state cells, or hCiPSCs, somatic cells/epithelial-like cells, or any combination thereof, the composition may or may not have any of these cells. For example, AB1C1D2 represents a composition that includes a MEK inhibitor, a B-Raf inhibitor, a histone deacetylase inhibitor, an inhibitor of histone demethylation, and hCiPSCs.

A composition may comprise PD0325901, AZD8330, or TAK-733; SB590885, Vemurafenib, RAF265, and PLX4720; VPA, LMK235, MS275, or HDACi IV; IWR-1 or IWP-2; CHIR99021 or CHIR98014; Y-27632 or thiazovivin; Tranylcypromine; EPZ004777 or EPZ5676; DZNep, NepA, Adox, or DZA; or any combination thereof. A composition may comprise at least 1, 2, 3, 4, 5, 6, 7, or 8 of PD0325901, AZD8330, or TAK-733; SB590885, Vemurafenib, RAF265, and PLX4720; VPA, LMK235, MS275, or HDACi IV; IWR-1 or IWP-2; CHIR99021 or CHIR98014; Y-27632 or thiazovivin; Tranylcypromine; EPZ004777 or EPZ5676; or DZNep, NepA, Adox, or DZA. A composition may comprise at most 1, 2, 3, 4, 5, 6, 7, or 8 of PD0325901, AZD8330, or TAK-733; SB590885, Vemurafenib, RAF265, and PLX4720; VPA, LMK235, MS275, or HDACi IV; IWR-1 or IWP-2; CHIR99021 or CHIR98014; Y-27632 or thiazovivin; Tranylcypromine; EPZ004777 or EPZ5676; or DZNep, NepA, Adox, or DZA. A composition may comprise PD0325901, AZD8330, or TAK-733; SB590885, Vemurafenib, RAF265, and PLX4720; VPA, LMK235, MS275, or HDACi IV; IWR-1 or IWP-2; CHIR99021 or CHIR98014; Y-27632 or thiazovivin; Tranylcypromine; EPZ004777 or EPZ5676; and DZNep, NepA, Adox, or DZA. A composition may comprise PD0325901, AZD8330, or TAK-733. A composition may comprise SB590885, Vemurafenib, RAF265, and PLX4720. A composition may comprise VPA, LMK235, MS275, or HDACi IV. A composition may comprise IWR-1 or IWP-2. A composition may comprise CHIR99021 or CHIR98014. A composition may comprise Y-27632 or thiazovivin. A composition may comprise Tranylcypromine. A composition may comprise EPZ004777 or EPZ5676. A composition may comprise DZNep, NepA, Adox, or DZA.

A composition may comprise PD0325901, AZD8330, or TAK-733, SB590885, Vemurafenib, RAF265, and PLX4720, or VPA, LMK235, MS275, or HDACi IV. A composition may comprise PD0325901, AZD8330, or TAK-733 or SB590885, Vemurafenib, RAF265, and PLX4720. A composition may comprise PD0325901, AZD8330, or TAK-733 or VPA, LMK235, MS275, or HDACi IV. A composition may comprise SB590885, Vemurafenib, RAF265, and PLX4720 or VPA, LMK235, MS275, or HDACi IV. A composition may comprise PD0325901, AZD8330, or TAK-733, SB590885, Vemurafenib, RAF265, and PLX4720, and VPA, LMK235, MS275, or HDACi IV.

A composition may comprise PD0325901, AZD8330, or TAK-733, SB590885, Vemurafenib, RAF265, and PLX4720, and VPA, LMK235, MS275, or HDACi IV; and IWR-1 or IWP-2. A composition may comprise PD0325901, AZD8330, or TAK-733, SB590885, Vemurafenib, RAF265, and PLX4720, and VPA, LMK235, MS275, or HDACi IV; and CHIR99021 or CHIR98014. A composition may comprise PD0325901, AZD8330, or TAK-733, SB590885, Vemurafenib, RAF265, and PLX4720, and VPA, LMK235, MS275, or HDACi IV; and Y-27632 or thiazovivin. A composition may comprise PD0325901, AZD8330, or TAK-733, SB590885, Vemurafenib, RAF265, and PLX4720, and VPA, LMK235, MS275, or HDACi IV; and Tranylcypromine. A composition may comprise PD0325901, AZD8330, or TAK-733, SB590885, Vemurafenib, RAF265, and PLX4720, and VPA, LMK235, MS275, or HDACi IV; and EPZ004777 or EPZ5676. A composition may comprise PD0325901, AZD8330, or TAK-733, SB590885, Vemurafenib, RAF265, and PLX4720, and VPA, LMK235, MS275, or HDACi IV; and DZNep, NepA, Adox, or DZA. A composition may comprise PD0325901, AZD8330, or TAK-733, SB590885, Vemurafenib, RAF265, and PLX4720, and VPA, LMK235, MS275, or HDACi IV; and one or more of IWR-1 or IWP-2, CHIR99021 or CHIR98014, Y-27632 or thiazovivin, Tranylcypromine, EPZ004777 or EPZ5676, or DZNep, NepA, Adox, or DZA.

A composition may comprise PD0325901; SB590885; VPA; IWP-2; CHIR99021; Y-27632; Tranylcypromine; EPZ004777; or DZNep; or any combination thereof. A composition may comprise at least 1, 2, 3, 4, 5, 6, 7, or 8 of PD0325901; SB590885; VPA; IWP-2; CHIR99021; Y-27632; Tranylcypromine; EPZ004777; or DZNep. A composition may comprise at most 1, 2, 3, 4, 5, 6, 7, or 8 of PD0325901; SB590885; VPA; IWP-2; CHIR99021; Y-27632; Tranylcypromine; EPZ004777; or DZNep. A composition may comprise PD0325901; SB590885; VPA; IWP-2; CHIR99021; Y-27632; Tranylcypromine; EPZ004777; and DZNep. A composition may comprise PD0325901. A composition may comprise SB590885. A composition may comprise VPA. A composition may comprise IWP-2. A composition may comprise CHIR99021. A composition may comprise Y-27632. A composition may comprise Tranylcypromine. A composition may comprise EPZ004777. A composition may comprise DZNep.

A composition may comprise PD0325901, SB590885, or VPA. A composition may comprise PD0325901 or SB590885. A composition may comprise PD0325901 or VPA. A composition may comprise SB590885 or VPA. A composition may comprise PD0325901, SB590885, and VPA.

A composition may comprise PD0325901, SB590885, and VPA; and 1WP-2. A composition may comprise PD0325901, SB590885, and VPA; and CHIR99021. A composition may comprise PD0325901, SB590885, and VPA; and Y-27632. A composition may comprise PD0325901, SB590885, and VPA; and Tranylcypromine. A composition may comprise PD0325901, SB590885, and VPA; and EPZ004777. A composition may comprise PD0325901, SB590885, and VPA; and DZNep. A composition may comprise PD0325901, SB590885, and VPA; and one or more of IWP-2, CHIR99021, Y-27632, Tranylcypromine, EPZ004777, or DZNep.

A composition may comprise at least about 0.02 μM, 0.04 μM, 0.06 μM, 0.08 μM, 0.1 μM, 0.12 μM, 0.14 μM, 0.16 μM, 0.18 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 45 μM, 50 μM or more PD0325901 within the composition. A composition may comprise at most about 0.02 μM, 0.04 μM, 0.06 μM, 0.08 μM, 0.1 μM, 0.12 μM, 0.14 μM, 0.16 μM, 0.18 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 25 μM, 30 μM, 45 μM, or 50 μM PD0325901 within the composition. A composition may comprise about 0.1 μM PD0325901 within the composition. A composition may comprise about 0.2 μM PD0325901 within the composition. A composition may comprise about 0.3 μM PD0325901 within the composition. A composition may comprise about 0.4 μM PD0325901 within the composition. A composition may comprise about 0.5 μM PD0325901 within the composition. A composition may comprise about 0.6 μM PD0325901 within the composition. A composition may comprise about 0.7 μM PD0325901 within the composition. A composition may comprise about 0.8 μM PD0325901 within the composition. A composition may comprise about 0.9 μM PD0325901 within the composition. A composition may comprise about 1 μM PD0325901 within the composition. A composition may comprise about 2 μM PD0325901 within the composition. A composition may comprise about 3 μM PD0325901 within the composition. A composition may comprise about 4 μM PD0325901 within the composition. A composition may comprise about 5 μM PD0325901 within the composition. A composition may comprise about 6 μM PD0325901 within the composition. A composition may comprise about 7 μM PD0325901 within the composition. A composition may comprise about 8 μM PD0325901 within the composition. A composition may comprise about 9 μM PD0325901 within the composition. A composition may comprise about 10 μM PD0325901 within the composition. A composition may comprise about 0.1 μM to about 10 μM PD0325901 within the composition. A composition may comprise about 0.2 μM to about 7.5 μM PD0325901 within the composition. A composition may comprise about 0.3 μM to about 5 μM PD0325901 within the composition. A composition may comprise about 0.4 μM to about 4 μM PD0325901 within the composition. A composition may comprise about 0.5 μM to about 3 μM PD0325901 within the composition. A composition may comprise about 0.75 μM to about 2 μM PD0325901 within the composition.

A composition may comprise at least about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, 25 μM or more SB590885 within the composition. A composition may comprise at most about 0.01 μM, 0.02 μM, 0.03 μM, 0.04 μM, 0.05 μM, 0.1 μM, 0.15 μM, 0.2 μM, 0.25 μM, 0.3 μM, 0.35 μM, 0.4 μM, 0.45 μM, 0.5 μM, 0.75 μM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 7.5 μM, 10 μM, 12.5 μM, 15 μM, 17.5 μM, 20 μM, 22.5 μM, or 25 μM SB590885 within the composition. A composition may comprise about 0.05 μM SB590885 within the composition. A composition may comprise about 0.1 μM SB590885 within the composition. A composition may comprise about 0.15 μM SB590885 within the composition. A composition may comprise about 0.2 μM SB590885 within the composition. A composition may comprise about 0.25 μM SB590885 within the composition. A composition may comprise about 0.3 μM SB590885 within the composition. A composition may comprise about 0.35 μM SB590885 within the composition. A composition may comprise about 0.4 μM SB590885 within the composition. A composition may comprise about 0.45 μM SB590885 within the composition. A composition may comprise about 0.5 μM SB590885 within the composition. A composition may comprise about 1 μM SB590885 within the composition. A composition may comprise about 1.5 μM SB590885 within the composition. A composition may comprise about 2 μM SB590885 within the composition. A composition may comprise about 2.5 μM SB590885 within the composition. A composition may comprise about 3 μM SB590885 within the composition. A composition may comprise about 3.5 μM SB590885 within the composition. A composition may comprise about 4 μM SB590885 within the composition. A composition may comprise about 4.5 μM SB590885 within the composition. A composition may comprise about 5 μM SB590885 within the composition. A composition may comprise about 0.05 μM to about 2.5 μM SB590885 within the composition. A composition may comprise about 0.1 μM to about 1.875 μM SB590885 within the composition. A composition may comprise about 0.15 μM to about 1.25 μM SB590885 within the composition. A composition may comprise about 0.2 μM to about 1 μM SB590885 within the composition. A composition may comprise about 0.25 μM to about 0.75 μM SB590885 within the composition. A composition may comprise about 0.375 μM to about 0.5 μM SB590885 within the composition.

A composition may comprise at least about 0.02 millimolar (mM), 0.04 mM, 0.06 mM, 0.08 mM, 0.1 mM, 0.12 mM, 0.14 mM, 0.16 mM, 0.18 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 45 mM, 50 mM or more VPA within the composition. A composition may comprise at most about 0.02 mM, 0.04 mM, 0.06 mM, 0.08 mM, 0.1 mM, 0.12 mM, 0.14 mM, 0.16 mM, 0.18 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 45 mM, or 50 mM VPA within the composition. A composition may comprise about 0.1 mM VPA within the composition. A composition may comprise about 0.2 mM VPA within the composition. A composition may comprise about 0.3 mM VPA within the composition. A composition may comprise about 0.4 mM VPA within the composition. A composition may comprise about 0.5 mM VPA within the composition. A composition may comprise about 0.6 mM VPA within the composition. A composition may comprise about 0.7 mM VPA within the composition. A composition may comprise about 0.8 mM VPA within the composition. A composition may comprise about 0.9 mM VPA within the composition. A composition may comprise about 1 mM VPA within the composition. A composition may comprise about 2 mM VPA within the composition. A composition may comprise about 3 mM VPA within the composition. A composition may comprise about 4 mM VPA within the composition. A composition may comprise about 5 mM VPA within the composition. A composition may comprise about 6 mM VPA within the composition. A composition may comprise about 7 mM VPA within the composition. A composition may comprise about 8 mM VPA within the composition. A composition may comprise about 9 mM VPA within the composition. A composition may comprise about 10 mM VPA within the composition. A composition may comprise about 0.1 mM to about 10 mM VPA within the composition. A composition may comprise about 0.2 mM to about 7.5 mM VPA within the composition. A composition may comprise about 0.3 mM to about 5 mM VPA within the composition. A composition may comprise about 0.4 mM to about 4 mM VPA within the composition. A composition may comprise about 0.5 mM to about 3 mM VPA within the composition. A composition may comprise about 0.75 mM to about 2 mM VPA within the composition.

A composition may comprise at least about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, 500 μM or more Tranylcypromine within the composition. A composition may comprise at most about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, or 500 μM Tranylcypromine within the composition. A composition may comprise about 1 μM Tranylcypromine within the composition. A composition may comprise about 2 μM Tranylcypromine within the composition. A composition may comprise about 3 μM Tranylcypromine within the composition. A composition may comprise about 4 μM Tranylcypromine within the composition. A composition may comprise about 5 μM Tranylcypromine within the composition. A composition may comprise about 6 μM Tranylcypromine within the composition. A composition may comprise about 7 μM Tranylcypromine within the composition. A composition may comprise about 8 μM Tranylcypromine within the composition. A composition may comprise about 9 μM Tranylcypromine within the composition. A composition may comprise about 10 μM Tranylcypromine within the composition. A composition may comprise about 15 μM Tranylcypromine within the composition. A composition may comprise about 20 μM Tranylcypromine within the composition. A composition may comprise about 30 μM Tranylcypromine within the composition. A composition may comprise about 40 μM Tranylcypromine within the composition. A composition may comprise about 50 μM Tranylcypromine within the composition. A composition may comprise about 60 μM Tranylcypromine within the composition. A composition may comprise about 70 μM Tranylcypromine within the composition. A composition may comprise about 80 μM Tranylcypromine within the composition. A composition may comprise about 90 μM Tranylcypromine within the composition. A composition may comprise about 100 μM Tranylcypromine within the composition. A composition may comprise about 1 μM to about 100 μM Tranylcypromine within the composition. A composition may comprise about 2 μM to about 75 μM Tranylcypromine within the composition. A composition may comprise about 3 μM to about 50 μM Tranylcypromine within the composition. A composition may comprise about 4 μM to about 40 μM Tranylcypromine within the composition. A composition may comprise about 5 μM to about 30 μM Tranylcypromine within the composition. A composition may comprise about 7.5 μM to about 20 μM Tranylcypromine within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more EPZ5676 within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM EPZ5676 within the composition. A composition may comprise about 0.2 μM EPZ5676 within the composition. A composition may comprise about 0.4 μM EPZ5676 within the composition. A composition may comprise about 0.6 μM EPZ5676 within the composition. A composition may comprise about 0.8 μM EPZ5676 within the composition. A composition may comprise about 1 μM EPZ5676 within the composition. A composition may comprise about 1.2 μM EPZ5676 within the composition. A composition may comprise about 1.4 μM EPZ5676 within the composition. A composition may comprise about 1.6 μM EPZ5676 within the composition. A composition may comprise about 1.8 μM EPZ5676 within the composition. A composition may comprise about 2 μM EPZ5676 within the composition. A composition may comprise about 4 μM EPZ5676 within the composition. A composition may comprise about 6 μM EPZ5676 within the composition. A composition may comprise about 8 μM EPZ5676 within the composition. A composition may comprise about 10 μM EPZ5676 within the composition. A composition may comprise about 12 μM EPZ5676 within the composition. A composition may comprise about 14 μM EPZ5676 within the composition. A composition may comprise about 16 μM EPZ5676 within the composition. A composition may comprise about 18 μM EPZ5676 within the composition. A composition may comprise about 20 μM EPZ5676 within the composition. A composition may comprise about 0.2 μM to about 20 μM EPZ5676 within the composition. A composition may comprise about 0.4 μM to about 15 μM EPZ5676 within the composition. A composition may comprise about 0.6 μM to about 10 μM EPZ5676 within the composition. A composition may comprise about 0.8 μM to about 8 μM EPZ5676 within the composition. A composition may comprise about 1 μM to about 6 μM EPZ5676 within the composition. A composition may comprise about 1.5 μM to about 4 μM EPZ5676 within the composition.

A composition may comprise at least about 0.0004 μM, 0.0008 μM, 0.0012 μM, 0.0016 μM, 0.002 μM, 0.004 μM, 0.006 μM, 0.008 μM, 0.01 μM, 0.012 μM, 0.014 μM, 0.016 μM, 0.018 μM, 0.02 μM, 0.04 μM, 0.06 μM, 0.08 μM, 0.1 μM, 0.12 μM, 0.14 μM, 0.16 μM, 0.18 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, 1 μM or more DZNep within the composition. A composition may comprise at most about 0.0004 μM, 0.0008 μM, 0.0012 μM, 0.0016 μM, 0.002 μM, 0.004 μM, 0.006 μM, 0.008 μM, 0.01 μM, 0.012 μM, 0.014 μM, 0.016 μM, 0.018 μM, 0.02 μM, 0.04 μM, 0.06 μM, 0.08 μM, 0.1 μM, 0.12 μM, 0.14 μM, 0.16 μM, 0.18 μM, 0.2 μM, 0.25 μM, 0.3 μM, 0.35 μM, 0.4 μM, 0.45 μM, 0.5 μM, 0.55 μM, 0.6 μM, 0.65 μM, 0.7 μM, 0.75 μM, 0.8 μM, 0.85 μM, 0.9 μM, 0.95 μM, or 1 μM DZNep within the composition. A composition may comprise about 0.002 μM DZNep within the composition. A composition may comprise about 0.004 μM DZNep within the composition. A composition may comprise about 0.006 μM DZNep within the composition. A composition may comprise about 0.008 μM DZNep within the composition. A composition may comprise about 0.01 μM DZNep within the composition. A composition may comprise about 0.012 μM DZNep within the composition. A composition may comprise about 0.014 μM DZNep within the composition. A composition may comprise about 0.016 μM DZNep within the composition. A composition may comprise about 0.018 μM DZNep within the composition. A composition may comprise about 0.02 μM DZNep within the composition. A composition may comprise about 0.04 μM DZNep within the composition. A composition may comprise about 0.06 μM DZNep within the composition. A composition may comprise about 0.08 μM DZNep within the composition. A composition may comprise about 0.1 μM DZNep within the composition. A composition may comprise about 0.12 μM DZNep within the composition. A composition may comprise about 0.14 μM DZNep within the composition. A composition may comprise about 0.16 μM DZNep within the composition. A composition may comprise about 0.18 μM DZNep within the composition. A composition may comprise about 0.2 μM DZNep within the composition. A composition may comprise about 0.002 μM to about 0.2 μM DZNep within the composition. A composition may comprise about 0.0025 μM to about 0.15 μM DZNep within the composition. A composition may comprise about 0.005 μM to about 0.1 μM DZNep within the composition. A composition may comprise about 0.0075 μM to about 0.75 μM DZNep within the composition. A composition may comprise about 0.01 μM to about 0.5 μM DZNep within the composition. A composition may comprise about 0.015 μM to about 0.4 μM DZNep within the composition.

A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, 100 μM or more IWP-2 within the composition. A composition may comprise at least about 0.04 μM, 0.08 μM, 0.12 μM, 0.16 μM, 0.2 μM, 0.4 μM, 0.6 μM, 0.8 μM, 1 μM, 1.2 μM, 1.4 μM, 1.6 μM, 1.8 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 30 μM, 40 μM, 50 μM, 60 μM, 70 μM, 80 μM, 90 μM, or 100 μM IWP-2 within the composition. A composition may comprise about 0.2 μM IWP-2 within the composition. A composition may comprise about 0.4 μM IWP-2 within the composition. A composition may comprise about 0.6 μM IWP-2 within the composition. A composition may comprise about 0.8 μM IWP-2 within the composition. A composition may comprise about 1 μM IWP-2 within the composition. A composition may comprise about 1.2 μM IWP-2 within the composition. A composition may comprise about 1.4 μM IWP-2 within the composition. A composition may comprise about 1.6 μM IWP-2 within the composition. A composition may comprise about 1.8 μM IWP-2 within the composition. A composition may comprise about 2 μM IWP-2 within the composition. A composition may comprise about 4 μM IWP-2 within the composition. A composition may comprise about 6 μM IWP-2 within the composition. A composition may comprise about 8 μM IWP-2 within the composition. A composition may comprise about 10 μM IWP-2 within the composition. A composition may comprise about 12 μM IWP-2 within the composition. A composition may comprise about 14 μM IWP-2 within the composition. A composition may comprise about 16 μM IWP-2 within the composition. A composition may comprise about 18 μM IWP-2 within the composition. A composition may comprise about 20 μM IWP-2 within the composition. A composition may comprise about 0.2 μM to about 20 μM IWP-2 within the composition. A composition may comprise about 0.4 μM to about 15 μM IWP-2 within the composition. A composition may comprise about 0.6 μM to about 10 μM IWP-2 within the composition. A composition may comprise about 0.8 μM to about 8 μM IWP-2 within the composition. A composition may comprise about 1 μM to about 6 μM IWP-2 within the composition. A composition may comprise about 1.5 μM to about 4 μM IWP-2 within the composition.

A composition may comprise at least about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, 50 μM or more CHIR99021 within the composition. A composition may comprise at most about 0.02 μM, 0.04 μM, 0.06 μM, 0.08 μM, 0.1 μM, 0.2 μM, 0.3 μM, 0.4 μM, 0.5 μM, 0.6 μM, 0.7 μM, 0.8 μM, 0.9 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 15 μM, 20 μM, 25 μM, 30 μM, 35 μM, 40 μM, 45 μM, or 50 μM CHIR99021 within the composition. A composition may comprise about 0.1 μM CHIR99021 within the composition. A composition may comprise about 0.2 μM CHIR99021 within the composition. A composition may comprise about 0.3 μM CHIR99021 within the composition. A composition may comprise about 0.4 μM CHIR99021 within the composition. A composition may comprise about 0.5 μM CHIR99021 within the composition. A composition may comprise about 0.6 μM CHIR99021 within the composition. A composition may comprise about 0.7 μM CHIR99021 within the composition. A composition may comprise about 0.8 μM CHIR99021 within the composition. A composition may comprise about 0.9 μM CHIR99021 within the composition. A composition may comprise about 1 μM CHIR99021 within the composition. A composition may comprise about 2 μM CHIR99021 within the composition. A composition may comprise about 3 μM CHIR99021 within the composition. A composition may comprise about 4 μM CHIR99021 within the composition. A composition may comprise about 5 μM CHIR99021 within the composition. A composition may comprise about 6 μM CHIR99021 within the composition. A composition may comprise about 7 μM CHIR99021 within the composition. A composition may comprise about 8 μM CHIR99021 within the composition. A composition may comprise about 9 μM CHIR99021 within the composition. A composition may comprise about 10 μM CHIR99021 within the composition. A composition may comprise about 0.1 μM to about 10 μM CHIR99021 within the composition. A composition may comprise about 0.2 μM to about 7.5 μM CHIR99021 within the composition. A composition may comprise about 0.3 μM to about 5 μM CHIR99021 within the composition. A composition may comprise about 0.4 μM to about 4 μM CHIR99021 within the composition. A composition may comprise about 0.5 μM to about 3 μM CHIR99021 within the composition. A composition may comprise about 0.75 μM to about 2 μM CHIR99021 within the composition.

A composition may comprise at least about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, 500 μM or more Y-27632 within the composition. A composition may comprise at most about 0.2 μM, 0.4 μM, 0.6 μM, 0.8 μM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 30 μM, 40 μM, 50 μM, 60 μM, 70 μM, 80 μM, 90 μM, 100 μM, 200 μM, 300 μM, 400 μM, or 500 μM Y-27632 within the composition. A composition may comprise about 1 μM Y-27632 within the composition. A composition may comprise about 2 μM Y-27632 within the composition. A composition may comprise about 3 μM Y-27632 within the composition. A composition may comprise about 4 μM Y-27632 within the composition. A composition may comprise about 5 μM Y-27632 within the composition. A composition may comprise about 6 μM Y-27632 within the composition. A composition may comprise about 7 μM Y-27632 within the composition. A composition may comprise about 8 μM Y-27632 within the composition. A composition may comprise about 9 μM Y-27632 within the composition. A composition may comprise about 10 μM Y-27632 within the composition. A composition may comprise about 15 μM Y-27632 within the composition. A composition may comprise about 20 μM Y-27632 within the composition. A composition may comprise about 30 μM Y-27632 within the composition. A composition may comprise about 40 μM Y-27632 within the composition. A composition may comprise about 50 μM Y-27632 within the composition. A composition may comprise about 60 μM Y-27632 within the composition. A composition may comprise about 70 μM Y-27632 within the composition. A composition may comprise about 80 μM Y-27632 within the composition. A composition may comprise about 90 μM Y-27632 within the composition. A composition may comprise about 100 μM Y-27632 within the composition. A composition may comprise about 1 μM to about 100 μM Y-27632 within the composition. A composition may comprise about 2 μM to about 75 μM Y-27632 within the composition. A composition may comprise about 3 μM to about 50 μM Y-27632 within the composition. A composition may comprise about 4 μM to about 40 μM Y-27632 within the composition. A composition may comprise about 5 μM to about 30 μM Y-27632 within the composition. A composition may comprise about 7.5 μM to about 20 μM Y-27632 within the composition.

Subsequent to contacting any populations of stage 3 cells with any compositions described herein, the cells may be incubated in normoxic condition. For example, the stage 3 cells may be incubated with at most 23%, 22%, 21%, 20%, or 19% atmospheric oxygen. The normoxic condition may comprise about 22% atmospheric oxygen. The normoxic condition may comprise about 21% atmospheric oxygen. The normoxic condition may comprise about 20% atmospheric oxygen.

Subsequent to contacting any populations of stage 3 cells with any compositions described herein, a population of stage 3 cells may be incubated with a composition for at least about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, or 30 days. A population of stage 3 cells may be incubated with a composition for at most about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, or 30 days. A population of stage 3 cells may be incubated with a composition for about 1 day. A population of stage 3 cells may be incubated with a composition for about 2 days. A population of stage 3 cells may be incubated with a composition for about 3 days. A population of stage 3 cells may be incubated with a composition for about 4 days. A population of stage 3 cells may be incubated with a composition for about 5 days. A population of stage 3 cells may be incubated with a composition for about 6 days. A population of stage 3 cells may be incubated with a composition for about 7 days. A population of stage 3 cells may be incubated with a composition for about 8 days. A population of stage 3 cells may be incubated with a composition for about 9 days. A population of stage 3 cells may be incubated with a composition for about 10 days. A population of stage 3 cells may be incubated with a composition for about 11 days. A population of stage 3 cells may be incubated with a composition for about 12 days. A population of stage 3 cells may be incubated with a composition for about 13 days. A population of stage 3 cells may be incubated with a composition for about 14 days. A population of stage 3 cells may be incubated with a composition for about 15 days. A population of stage 3 cells may be incubated with a composition for about 16 days. A population of stage 3 cells may be incubated with a composition for about 17 days. A population of stage 3 cells may be incubated with a composition for about 18 days. A population of stage 3 cells may be incubated with a composition for about 19 days. A population of stage 3 cells may be incubated with a composition for about 20 days. A population of stage 3 cells may be incubated with a composition for about 25 days.

In some cases, subsequent to incubating a population of stage 3 cells with a first composition for a first period of time, the first composition may be removed from the cells. The cells can then be contacted with a second composition. Subsequent to incubating the cells with the second composition for a second period of time, the second composition may be removed from the cells. The cells can then be contacted with a third composition and incubated with the third composition for a third period of time. The first period of time may comprise at least about 1, 2, 3, 4, 5, 6, 7, or 8 days. The first period of time may comprise at most about 1, 2, 3, 4, 5, 6, 7, or 8 days. The second period of time may comprise at least about 1, 2, 3, 4, 5, 6, 7, or 8 days. The second period of time may comprise at most about 1, 2, 3, 4, 5, 6, 7, or 8 days. The third period of time may comprise at least about 1, 2, 3, 4, 5, 6, 7, or 8 days. The third period of time may comprise at most about 1, 2, 3, 4, 5, 6, 7, or 8 days. The first composition may comprise the histone deacetylase inhibitor, the inhibitor of histone demethylation, the S-adenosyl-L-homocysteine (SAH) hydrolase inhibitor, or the Dot1L inhibitor. The second composition may comprise the histone deacetylase inhibitor with the amount that is at least about 30%, 40%, 50%, 60%, or 70% less than the first composition; the inhibitor of histone demethylation; the S-adenosyl-L-homocysteine (SAH) hydrolase inhibitor; or the Dot1L inhibitor. The second composition may comprise the histone deacetylase inhibitor with the amount that is at most about 30%, 40%, 50%, 60%, or 70% less than the first composition; the inhibitor of histone demethylation; the S-adenosyl-L-homocysteine (SAH) hydrolase inhibitor; or the Dot1L inhibitor. The third composition may not comprise the inhibitor of histone demethylation; the S-adenosyl-L-homocysteine (SAH) hydrolase inhibitor; or the Dot1L inhibitor.

Any of the compositions may not comprise feeder cells or serum. Any of the compositions may not comprise feeder cells and serum. Any of the compositions may not comprise feeder cells. Any of the compositions may be serum-free. Any of the compositions may comprise feeder cells. Any of the compositions may comprise serum.

### Chemical Reprogramming Factors

Compositions provided herein may comprise at least a cell or at least a chemical reprogramming factor. In some cases, a composition provided herein comprises a cell or a population of cells. In some cases, a composition provided herein comprises a chemical reprogramming factor. A composition may comprise at least a chemical reprogramming factor. A composition may comprise a plurality of chemical reprogramming factors. A composition may comprise at least a cell and at least a chemical reprogramming factor. A composition may comprise a population of cells and a plurality of chemical reprogramming factors.

In some cases, the compositions described herein comprise a chemical reprogramming factor. A chemical reprogramming factor may facilitate a conversion of a cell type to another cell type. A chemical reprogramming factor may facilitate a cell conversion by increasing the number of reprogrammed cells obtained from the same starting cell density cultured for the same length of time and/or improving the quality of reprogrammed cells, measured in terms of characteristics selected from the ability of the cells to express pluripotency factors such as OCT4, SOX2 and NANOG and number of passages in culture, when compared to a reprograming method that does not use the same chemical reprogramming factor.

A chemical reprogramming factor may regulate a cellular component involved in a cellular activity or biological activity. In some cases, the compositions described herein comprise a plurality of chemical reprogramming factors. A chemical reprogramming factor may comprise a chemical compound that can facilitate the conversion of a first population of cells to a second population of cells. A chemical reprogramming factor may comprise a chemical compound that can facilitate the conversion of a cell type to another cell type. A chemical reprogramming factor may not induce a genetic modification a cell. A genetic modification may comprise a change in the make-up of a genome of a cell. The genome may comprise a chromosomal deoxyribonucleic acid (DNA) or an extra-chromosomal DNA (e.g., a mitochondrial DNA). A genetic modification may comprise a mutation. The mutation may comprise a substitution, deletion, or insertion of the genome. In other cases, the mutation also comprises a DNA rearrangement of the chromosomal or mitochondrial DNA. A chemical reprogramming factor may not add exogenous genetic materials into a cell. A chemical reprogramming factor may not add exogenous genetic materials a chromosomal or extra-chromosomal DNA of the cell.

A chemical reprogramming factor may comprise a chemical compound or molecule. A chemical reprogramming factor may comprise an organic compound or an inorganic compound. A chemical reprogramming factor may comprise an organic compound. A chemical reprogramming factor may comprise an inorganic compound. A chemical reprogramming factor may be peptide-based. A chemical reprogramming factor may not be peptide-based. A chemical reprogramming factor may comprise a carbohydrate moiety. A chemical reprogramming factor may not comprise a carbohydrate moiety. A chemical reprogramming factor may comprise a lipid moiety. A chemical reprogramming factor may not comprise a lipid moiety. A chemical reprogramming factor may comprise a drug. In some cases, A chemical reprogramming factor may comprise any chemical reprogramming factors described herein. In some cases, a chemical reprogramming factor is substituted with another chemical reprogramming factor described herein. In some cases, a chemical reprogramming factor is substituted with another chemical reprogramming factor identified based on the methods described herein.

A chemical reprogramming factor may elicit a biological activity of a cell when the cell contacted the chemical reprogramming factor. A biological activity may comprise a biophysical or biochemical response of a cell. Such a biophysical response may comprise cell locomotion, cell attachment/detachment, cell polarization, cell shape change, or any combination thereof. A biochemical response may comprise gene expression, protein expression, RNA expression, post-transcriptional modification of a protein/DNA/ribonucleic acid (RNA), post-translational modification of a protein, endocytosis, exocytosis, cell proliferation, cell-cycle progression, cell differentiation, or any combination thereof. In some cases, a biological activity comprises a cellular activity described herein.

In some cases, two chemical reprogramming factors share a same biological activity. To measure a biological activity of a chemical reprogramming factor, a biological assay may be used. A biological assay may qualitatively or quantitatively reflect or report a biological activity to be measured. The specific assay used for a specific biological activity may depend on the biological activity being measure. The biological assay may be an in vitro assay. The biological assay may be an in vivo assay. The specific assay may derive a measurable value of the biological activity of a chemical reprogramming factor. In some cases, two chemical reprogramming factors are determined to share a same biological activity if the difference of their measurable values in a same biological assay is within 1%, 2%, 3%, 4%, 5%, 6%, 7%, 8%, 9%, 10%, 15%, 20%, 25%, 30%, 35%, 40%, 45%, 50%, 60%, 70%, 80%, 90%, 100%, 2-fold, 3-fold, 4-fold, or 5-fold. In some cases, two chemical reprogramming factors determined to share a same biological activity may substitute each other for converting a cell. The measurable value may be derived from various markers involved in various biological activity described herein.

A chemical reprogramming factor may have a molecular weight. The molecular weight of a chemical reprogramming factor may be at most about 10000 Daltons (Da), 9000 Da, 8000 Da, 7000 Da, 6000 Da, 5000 Da, 4500 Da, 4000 Da, 3500 Da, 3000 Da, 2500 Da, 2000 Da, 1900 Da, 1800 Da, 1700 Da, 1600 Da, 1500 Da, 1400 Da, 1300 Da, 1200 Da, 1100 Da, 1000 Da, 900 Da, 800 Da, 700 Da, 600 Da, 500 Da, 400 Da, 300 Da, 200 Da, 100 Da or less. The molecular weight of a chemical reprogramming factor may be at less about 100 Da, 200 Da, 300 Da, 400 Da, 500 Da, 600 Da, 700 Da, 800 Da, 900 Da, 1000 Da, 1100 Da, 1200 Da, 1300 Da, 1400 Da, 1500 Da, 1600 Da, 1700 Da, 1800 Da, 1900 Da, 2000 Da, 2500 Da, 3000 Da, 3500 Da, 4000 Da, 4500 Da, 5000 Da, 6000 Da, 7000 Da, 8000 Da, 9000 Da, 10000 Da or more. The molecular weight of a chemical reprogramming factor may be at least about 100 Da. The molecular weight of a chemical reprogramming factor may be at least about 200 Da. The molecular weight of a chemical reprogramming factor may be at least about 300 Da. The molecular weight of a chemical reprogramming factor may be at least about 400 Da. The molecular weight of a chemical reprogramming factor may be at least about 500 Da. The molecular weight of a chemical reprogramming factor may be at least about 600 Da. The molecular weight of a chemical reprogramming factor may be at least about 700 Da. The molecular weight of a chemical reprogramming factor may be at least about 800 Da. The molecular weight of a chemical reprogramming factor may be at least about 900 Da. The molecular weight of a chemical reprogramming factor may be at least about 1000 Da. The molecular weight of a chemical reprogramming factor may be at least about 1100 Da. The molecular weight of a chemical reprogramming factor may be at least about 1200 Da. The molecular weight of a chemical reprogramming factor may be at least about 1300 Da. The molecular weight of a chemical reprogramming factor may be at least about 1400 Da. The molecular weight of a chemical reprogramming factor may be at least about 1500 Da. The molecular weight of a chemical reprogramming factor may be at least about 1600 Da. The molecular weight of a chemical reprogramming factor may be at least about 1700 Da. The molecular weight of a chemical reprogramming factor may be at least about 1800 Da. The molecular weight of a chemical reprogramming factor may be at least about 1900 Da. The molecular weight of a chemical reprogramming factor may be at least about 2000 Da. The molecular weight of a chemical reprogramming factor may be at most about 100 Da. The molecular weight of a chemical reprogramming factor may be at most about 200 Da. The molecular weight of a chemical reprogramming factor may be at most about 300 Da. The molecular weight of a chemical reprogramming factor may be at most about 400 Da. The molecular weight of a chemical reprogramming factor may be at most about 500 Da. The molecular weight of a chemical reprogramming factor may be at most about 600 Da. The molecular weight of a chemical reprogramming factor may be at most about 700 Da. The molecular weight of a chemical reprogramming factor may be at most about 800 Da. The molecular weight of a chemical reprogramming factor may be at most about 900 Da. The molecular weight of a chemical reprogramming factor may be at most about 1000 Da. The molecular weight of a chemical reprogramming factor may be at most about 1100 Da. The molecular weight of a chemical reprogramming factor may be at most about 1200 Da. The molecular weight of a chemical reprogramming factor may be at most about 1300 Da. The molecular weight of a chemical reprogramming factor may be at most about 1400 Da. The molecular weight of a chemical reprogramming factor may be at most about 1500 Da. The molecular weight of a chemical reprogramming factor may be at most about 1600 Da. The molecular weight of a chemical reprogramming factor may be at most about 1700 Da. The molecular weight of a chemical reprogramming factor may be at most about 1800 Da. The molecular weight of a chemical reprogramming factor may be at most about 1900 Da. The molecular weight of a chemical reprogramming factor may be at most about 2000 Da.

In some cases, a plurality of chemical reprogramming factors facilitates a conversion of a first population of cells to a second population of cells. In some cases, while a plurality of chemical reprogramming factors facilitates a conversion of a first population of cells to a second population of cells, a composition without any one of the chemical reprogramming factors may not facilitate the conversion of the first population of cells to the second populations of cells. In some cases, each of the plurality of chemical reprogramming factors has a dosage range to facilitate the conversion of the first population of cells to the second populations of cells. In some cases, if any one of the chemical reprogramming factors does not have the dosage range described herein, the plurality of chemical reprogramming factors may not facilitate the conversion of the first population of cells to the second populations of cells. In some cases, the dosage range for a chemical reprogramming factor for converting a population of cells may comprises any dosage ranges described herein. In some cases, the dosage range for a chemical reprogramming factor for converting a population of cells may comprises the dosage ranges identified using the methods described herein (e.g., using the methods described in the EXAMPLEs described herein.

In some cases, two chemical reprogramming factors may substitute each other for converting a cell by testing the conversion or reprogramming efficiencies using methods described in the EXAMPLEs described herein.

Provided herein, in some aspects, are exemplary chemical reprogramming factors that may be used in a method provided herein or may be contained in a composition provided herein. These chemical reprogramming factors may increase or decrease various cellular or biological activities.

### 1. Glycogen Synthesis Kinase (GSK; or Glycogen Kinase) Inhibitors

A chemical reprogramming factor may comprise a chemical compound that can inhibit GSK. GSK may comprise a serine/threonine protein kinase that mediates phosphorylation of serine and threonine of various cellular factors. The phosphorylation of these cellular factors may control glycogen metabolism, cell signaling, or cellular transport. GSK inhibition may lead to a decrease in glycogen synthesis in the liver and muscles and/or increased blood glucose or hyperglycemia. GSK inhibition in neuroblastoma may reduce neuroendocrine marker expression (complex-like 1 (ASCL1) and chromogranin A (CgA), and/or beta-catenin) and/or suppress neuroblastoma cell growth (Carter et al., 2014; Cancer Biol Ther. 2014 May; 15(5):510-5, which is herein incorporated by reference in its entirety). GSK inhibition in cancer cells may decrease the growth of the cancer cell (Carter 2014).

A glycogen synthase kinase 3 (GSK-3) inhibitor may comprise CHIR99021 ([6-[[2-[[4-(2,4-Dichlorophenyl)-5-(5-methyl-1H-imidazol-2-yl)-2-pyrimidinyl]amino]ethyl]amino]-3-pyridinecarbonitrile]); BIO-acetoxime; GSK 3I inhibitor XV; SB-216763 ([3-(2,4-Dichlorophenyl)-4-(1-methyl-1H-indol-3-yl)-1H-pyrrole-2,5-dione]); CHIR99021 trihydrochloride (a hydrochloride salt of CHIR99021); GSK-3 Inhibitor IX ([((2Z, 3E)-6′-bromo-3-(hydroxyimino)-[2,3′-biindolinylidene]-2′-one]); GSK3 IX ([6-Bromoindirubin-3′-oxime]); GSK-3β Inhibitor XII ([3-[[6-(3-Aminophenyl)-7H-pyrrolo[2,3-d]pyrimidin-4-yl]oxy]phenol]); GSK-3 Inhibitor XVI ([6-(2-(4-(2,4-dichlorophenyl)-5-(4-methyl-1H-imidazol-2-yl)-pyrimidin-2-ylamino)ethyl-amino)-nicotinonitrile]); SB-415286 ([3-[(3-chloro-4-hydroxyphenyl)amino]-4-(2-nitrophenyl)-1H-pyrrole-2,5-dione]); Bio ([(2′Z,3′E)-6-bromoindirubin-3′-oxime]); TD114-2 ([6, 7, 9, 10, 12, 13, 15, 16, 18, 19-Decahydro-5, 29:20, 25-dimetheno-26H-dibenzo[n, t]pyrrolo[3, 4-q][1, 4, 7, 10, 13, 22]tetraoxadiazacyclote tracosine-26, 28(27H)-dione]); or CHIR98014 ([N6-[2-[[4-(2,4-Dichlorophenyl)-5-(1H-imidazol-1-yl)-2-pyrimidinyl]amino]ethyl]-3-nitro-2,6-pyridinediamine]) or any combination thereof. A glycogen synthase kinase 3 (GSK-3) inhibitor may comprise A glycogen synthase kinase 3 (GSK-3) inhibitor may comprise CHIR99021. A glycogen synthase kinase 3 (GSK-3) inhibitor may comprise BIO-acetoxime. A glycogen synthase kinase 3 (GSK-3) inhibitor may comprise GSK 3I inhibitor XV. A glycogen synthase kinase 3 (GSK-3) inhibitor may comprise SB-216763. A glycogen synthase kinase 3 (GSK-3) inhibitor may comprise CHIR99021 trihydrochloride. A glycogen synthase kinase 3 (GSK-3) inhibitor may comprise GSK-3 Inhibitor IX. A glycogen synthase kinase 3 (GSK-3) inhibitor may comprise GSK3 IX. A glycogen synthase kinase 3 (GSK-3) inhibitor may comprise GSK-3β Inhibitor XII. A glycogen synthase kinase 3 (GSK-3) inhibitor may comprise GSK-3 Inhibitor XVI. A glycogen synthase kinase 3 (GSK-3) inhibitor may comprise SB-415286. A glycogen synthase kinase 3 (GSK-3) inhibitor may comprise Bio. A glycogen synthase kinase 3 (GSK-3) inhibitor may comprise TD114-2. A glycogen synthase kinase 3 (GSK-3) inhibitor may comprise CHIR98014. aminopyrimidine. Aminopyrimidine may comprise CHIR99021, CHIR99021 trihydrochloride, or CHIR98014, or any combination thereof.

### 2. Transforming Growth Factor Beta Receptor Inhibitors (Transforming Growth Factor-Beta (TGFβ)R Inhibitors)

A chemical reprogramming factor may comprise a chemical compound that can inhibit transforming growth factor-beta (TGFβ) receptor. The type I transforming growth factor-beta (TGFβ) receptor may comprise activin receptor-like kinase (ALK) 1, ALK2, ALK3, ALK4, ALK5, ALK6, or ALK7. The type II transforming growth factor-beta (TGFβ) receptor may comprise transforming growth factor-beta (TGFβ)R2, bone morphogenetic protein receptor type 2 (BMPR2), activin receptor type-2A (ACVR2A), ACVR2B, or anti-Müllerian hormone receptor 2 (AMHR2). The type III transforming growth factor-beta (TGFβ) receptor may comprise transforming growth factor-beta (TGFβ)R3 (β-glycan). A transforming growth factor-beta (TGFβ) receptor inhibitor may inhibit type I transforming growth factor-beta (TGFβ) receptor. A transforming growth factor-beta (TGFβ) receptor inhibitor may inhibit type II transforming growth factor-beta (TGFβ) receptor. A transforming growth factor-beta (TGFβ) receptor inhibitor may inhibit type III transforming growth factor-beta (TGFβ) receptor. A transforming growth factor-beta (TGFβ) receptor inhibitor may inhibit ALK1, ALK2, ALK3, ALK4, ALK5, ALK6, ALK7, BMPR2, ACVR2A, ACVR2B, AMHR2, transforming growth factor-beta (TGFβ)R3, or any combination thereof. A transforming growth factor-beta (TGFβ) receptor inhibitor used in the subject methods or compositions may be an ALK inhibitor or a type I transforming growth factor-beta (TGFβ) receptor inhibitor, e.g., specifically inhibiting one or more type I transforming growth factor-beta (TGFβ) receptors, such as ALK1, ALK2, ALK3, ALK4, ALK5, ALK6, or ALK7. In some cases, a transforming growth factor-beta (TGFβ) receptor inhibitor used in the subject methods or compositions does not specifically inhibit transforming growth factor-beta (TGFβ) receptors other than type I transforming growth factor-beta (TGFβ) receptors, for instance, a transforming growth factor-beta (TGFβ) receptor inhibitor used in the subject methods or compositions may not inhibit any BMP receptor. In some cases, a transforming growth factor-beta (TGFβ) receptor inhibitor is an ALK5 inhibitor. In some cases, a transforming growth factor-beta (TGFβ) receptor inhibitor is an ALK5 inhibitor that does not specifically inhibit ALK2, ALK3, or ALK6.

A transforming growth factor-beta (TGFβ) receptor may be activated by growth factors or cytokines. Inhibition of the transforming growth factor-beta (TGFβ) receptor may lead to decreased cell proliferation or decreased metastasis of cancers (Derynck et al, 2003; Nature. 2003 Oct. 9; 425(6958):577-84., which is herein incorporated by reference in its entirety). Inhibition of transforming growth factor-beta (TGFβ) receptor may lead to a decreased phosphorylation of R-Smad (or Receptor-regulated Mothers against decapentaplegic; Derynck 2003). In some cases, Inhibition of transforming growth factor-beta (TGFβ) receptor may lead to a decreased transcription of genes under the regulation of Smad binding element (Derynck 2003).

A transforming growth factor-beta (TGFβ) receptor inhibitor may comprise E-616452 (or 616452; [2-(3-(6-Methylpyridin-2-yl)-1H-pyrazol-4-yl)-1,5-naphthyridine]); A 83-01 ([3-(6-Methyl-2-pyridinyl)-N-phenyl-4-(4-quinolinyl)-1H-pyrazole-1-carbothioamide]; SB 505124 ([2-[4-(1,3-Benzodioxol-5-yl)-2-(1,1-dimethylethyl)-1H-imidazol-5-yl]-6-methyl-pyridine]); GW 788388 ([4-[4-[3-(2-Pyridinyl)-1H-pyrazol-4-yl]-2-pyridinyl]-N-(tetrahydro-2H-pyran-4-yl)-benzamide]); SB 525334 ([6-[2-(1,1-Dimethylethyl)-5-(6-methyl-2-pyridinyl)-1H-imidazol-4-yl]quinoxaline]), Dorsomorphin, or SB431542, or any combination thereof. A transforming growth factor-beta (TGFβ) receptor inhibitor may comprise E-616452. A transforming growth factor-beta (TGFβ) receptor inhibitor may comprise A 83-01. A transforming growth factor-beta (TGFβ) receptor inhibitor may comprise SB 505124. A transforming growth factor-beta (TGFβ) receptor inhibitor may comprise GW 788388. A transforming growth factor-beta (TGFβ) receptor inhibitor may comprise SB 525334. A transforming growth factor-beta (TGFβ) receptor inhibitor may comprise Dorsomorphin. A transforming growth factor-beta (TGFβ) receptor inhibitor may not comprise Dorsomorphin. A transforming growth factor-beta (TGFβ) receptor inhibitor may comprise SB431542.

In some cases, a chemical reprogramming factor may comprise a BMP receptor/AMPK inhibitor. A BMP receptor/AMPK inhibitor may comprise Dorsomorphin. In some cases, Dorsomorphin may inhibit ALK2, ALK3, ALK6, or a combination thereof. In some cases, a BMP receptor/AMPK inhibitor does not specifically inhibit ALK5.

### 3. Retinoic Acid Receptor (RAR) Agonists

A chemical reprogramming factor may comprise an activator or agonist of retinoic acid receptor (RAR). Retinoic acid receptor (RAR) comprises a nuclear receptor transcription factor. Retinoic acid receptor (RAR) may comprise RAR-α or RAR-β. Retinoic acid receptor (RAR) may be activated by retinoic acids. The retinoic acid may comprise all-trans retinoic acid or 9-cis retinoic acid. Retinoic acid receptor (RAR) may form heterodimer with RXR (le Marie et al. 2019; Cells. 2019 Nov. 5; 8(11):1392., which is herein incorporated by reference in its entirety). The RAR/RXR dimer with corepressor protein may inhibit the transcription from retinoic acid response elements (RAREs) (le Marie 2019). Binding of retinoic acid receptor (RAR) agonist to retinoic acid receptor (RAR) can lead to a dissociation of the corepressor protein from the RAR/RXR complex. It can also lead to binding of RAR/RXR complex with the coactivator protein. Such binding can facilitate transcription of genes comprising the RAREs (le Marie 2019).

An RAR agonist may comprise TTNPB ([4-[(E)-2-(5,6,7,8-Tetrahydro-5,5,8,8-tetramethyl-2-naphthalenyl)-1-propenyl]benzoic acid]); Ch 55 ([4-[(1E)-3-[3,5-bis(1,1-Dimethylethyl)phenyl]-3-oxo-1-propenyl]benzoic acid]); or AM580 ([4-[(5,6,7,8-Tetrahydro-5,5,8,8-tetramethyl-2-naphthalenyl)carboxamido]benzoic acid]); or any combination thereof. An retinoic acid receptor (RAR) agonist may comprise TTNPB. An retinoic acid receptor (RAR) agonist may comprise Ch 55. Ch 55 may be a synthetic retinoid that has high affinity for RAR-α and RAR-β receptors and low affinity for cellular retinoic acid binding protein (CRABP). An retinoic acid receptor (RAR) agonist may comprise AM580. AM580 may be an analog of retinoic acid that acts as a selective RARα agonist.

### 4. S-Adenosyl-L-Homocysteine (SAH) Hydrolase Inhibitors

A chemical reprogramming factor may comprise a chemical compound that can inhibit S-adenosyl-L-homocysteine (SAH) hydrolase. S-adenosyl-L-homocysteine (SAH) hydrolase may catalyze reversible hydration of S-adenosyl-L-homocysteine (SAH) into adenosine and homocysteine (Xiao et al. 2019; Circulation. 2019 May 7; 139(19):2260-2277., which is herein incorporated by reference in its entirety). S-adenosyl-L-homocysteine (SAH) hydrolase may require NAD⁺ cofactor when catalyzing the hydration of S-adenosyl-L-homocysteine (SAH). Inhibition of S-adenosyl-L-homocysteine (SAH) hydrolase can lead to accumulation of S-adenosyl-L-homocysteine (SAH), which in turn inhibits methyltransferase that utilizes SAM (or S-Adenosyl methionine) as the methyl group donor (Xiao 2019). Accordingly, inhibition of S-adenosyl-L-homocysteine (SAH) hydrolase can lead to inhibition of methylation of various cellular factors (Xiao 2019).

A S-adenosyl-L-homocysteine (SAH) hydrolase inhibitor may comprise 3-deazaneplanocin A (DZNep, [(1S,2R,5R)-5-(4-Amino-1H-imidazo[4,5-c]pyridin-1-yl)-3-(hydroxymethyl)-3-cyclopentene-1,2-diol]); (−) Neplanocin A (NepA, [5R-(6-amino-9H-purin-9-yl)-3-(hydroxymethyl)-3-cyclopentene-1S,2R-diol]); Adenozine periodate oxidized (Adox, [(2S)-2-[(1R)-1-(6-aminopurin-9-yl)-2-oxoethoxy]-3-hydroxypropanal]); or 3-deazaadenosine (DZA, [1-β-D-ribofuranosyl-1H-imidazo[4,5-c]pyridin-4-amine]) or any combination thereof. A S-adenosyl-L-homocysteine (SAH) hydrolase inhibitor may comprise DZNep. A S-adenosyl-L-homocysteine (SAH) hydrolase inhibitor may comprise NepA. A S-adenosyl-L-homocysteine (SAH) hydrolase inhibitor may comprise Adox. A S-adenosyl-L-homocysteine (SAH) hydrolase inhibitor may comprise DZA.

### 5. Disruptor of Telomeric Silencing 1-Like (Dot1L) Inhibitors

A chemical reprogramming factor may comprise a Dot1L inhibitor. Dot1L is a histone H3 lysine 79 (H3K79) methyltransferase. Dot1L can catalyze the methylation of H3K79 (Kari et al. 2019; Clin Epigenetics. 2019 Jan. 7; 11(1):4., which is herein incorporated by reference in its entirety). Dot1L is a non-SET domain containing methyltransferase known to catalyze mono-, di-, and tri-methylation of H3K79. Inhibition of Dot1L may decrease the methylation of H3K79 (Kari 2019). Inhibition of Dot1L may decrease the phosphorylation of H2AX at serine 139 by specific DNA damage response-associated members of the phosphatidylinositol-3-kinase family induced by DNA damages (Kari 2019). Inhibition of Dot1L may homologous recombination-mediated double strand break repairs (Kari 2019).

A Dot1L inhibitor may comprise SGC 0946 ([1-[3-[[[(2R,3S,4R,5R)-5-(4-Amino-5-bromo-7H-pyrrolo[2,3-d]pyrimidin-7-yl)-3,4-dihydroxytetrahydrofuran-2-yl]methyl](isopropyl)amino]propyl]-3-[4-(2,2-dimethylethyl)phenyl]urea]); EPZ004777 ([7-[5-Deoxy-5-[[3-[[[[4-(1,1-dimethylethyl)phenyl]amino]carbonyl]amino]propyl](1-methylethyl)amino]-β-D-ribofuranosyl]-7H-pyrrolo[2,3-d]pyrimidin-4-amine]); or EPZ5676 [(2R,3R,4S,5R)-2-(6-amino-9H-purin-9-yl)-5-((((1r,3S)-3-(2-(5-(tert-butyl)-1H-benzo[d]imidazol-2-yl)ethyl)cyclobutyl)(isopropyl)amino)methyl)tetrahydrofuran-3,4-diol]); or any combination thereof. A Dot1L inhibitor may comprise SGC 0946. A Dot1L inhibitor may comprise EPZ5676. A Dot1L inhibitor may comprise EPZ004777.

### 6. Histone Deacetylase Inhibitors

A chemical reprogramming factor may comprise a histone deacetylase inhibitor. Histone deacetylase may catalyze the deacetylation of histones. For example, a histone deacetylase may remove acetyl groups from an F-N-acetyl lysine amino acid of a histone. Histone deacetylases may comprise class I, class IIA, class IIb, class III, and class IV histone deacetylase (Seto et al 2014; Cold Spring Harb Perspect Biol. 2014 Apr. 1; 6(4):a018713., which is herein incorporated by reference in its entirety). A class I histone deacetylase may comprise HDAC1, HDAC2, HDAC3, or HDAC8. A class IIA histone deacetylase may comprise HDAC4, HDAC5, HDAC7, or HDAC9. A class IIB histone deacetylase may comprise HDAC6 or HDAC10. A class 1111 histone deacetylase may comprise sirtuin family (SIRT1, SIRT2, SIRT3, SIRT4, SIRT6, and/or SIRT7) or Sir2 (in budding yeast). A class IV histone deacetylase may comprise HDAC11. A histone deacetylase inhibitor may inhibitor any one or any combinations of histone deacetylase described herein. Inhibition of a histone deacetylase may increase acetylation of histone (Seto 2014). Inhibition of a histone deacetylase may also induce cell growth arrest, cell differentiation, or apoptosis (Seto 2014). Inhibition of a histone deacetylase may also inhibit cancer cell growth (Seto 2014).

A histone deacetylase inhibitor may comprise valproic acid (VPA); apicidin ([cyclo(N—O-methyl-L-tryptophanyl-L-isoleucinyl-D-pipecolinyl-L-2-amino-8-oxodecanoyl)]; LMK235 ([N-[[6-(Hydroxyamino)-6-oxohexyl]oxy]-3,5-dimethylbenzamide]); MS275 ([(Pyridin-3-yl)methyl 4-(2-aminophenylcarbamoyl) benzylcarbamate]); CI 994 ([N-acetyldinaline4-(Acetylamino)-N-(2-aminophenyl) benzamide]); Depsipeptide; KD 5170 ([S-[2-[6-[[[4-[3-(Dimethylamino)propoxy]phenyl]sulfonyl]amino]-3-pyridinyl]-2-oxoethyl]ethanethioc acid ester]; sodium,4-pehynl butyrate; sodium butyrate ([Butanoic acid sodium salt]); or UF 010 ([4-Bromo-N′-butylbenzohydrazide)); HDACi IV; or any combination thereof. A histone deacetylase inhibitor may comprise VPA. A histone deacetylase inhibitor may comprise LMK235. A histone deacetylase inhibitor may comprise MS275. A histone deacetylase inhibitor may comprise CI 994. A histone deacetylase inhibitor may comprise KD5170. A histone deacetylase inhibitor may comprise Depsipeptide. A histone deacetylase inhibitor may comprise sodium butyrate. A histone deacetylase inhibitor may comprise UF 010.

### 7. B-Raf Inhibitors

A chemical reprogramming factor may comprise a B-Raf inhibitor. B-Raf may comprise a protooncogene. B-Raf may also be referred to as B-Raf or v-Raf murine sarcoma viral oncogene homolog B. B-Raf is a serine/threonine-protein kinase. Activated RAS or RAS-GTP may activate B-Raf (Olsen et al. 2020; Sci Rep. 2020 Nov. 18; 10(1):20113; which is herein incorporated by reference in its entirety). Activation of B-Raf may lead to increased phosphorylation of MEK, which in turn lead to increased phosphorylation of ERK (Olsen 2020). Activation of B-Raf may also lead increased cell proliferation (Olsen 2020). Inhibition of B-Raf may lead to decreased phosphorylation of MEK or ERK. Activation of B-Raf may lead decreased cell proliferation (Olsen 2020).

A B-Raf inhibitor may comprise SB590885 ([5-[2-[4-[2-(Dimethylamino) ethoxy]phenyl]-5-(4-pyridinyl)-1H-imidazol-4-yl]-2,3-dihydro-1H-inden-1-one oxime]); Vemurafenib; RAF265 (CHIR-265) (Selleckhchem catalog No. 52161); or PLX4720 (Selleckhchem catalog No. S11525); or any combination thereof. A B-Raf inhibitor may comprise SB590885. SB590885 may be a potent B-Raf inhibitor with an inhibitor constant (Ki) of 0.16 nM in a cell-free assay. SB590885 may be 11-fold greater selectivity for B-Raf over c-Raf. SB590885 may not inhibit other human kinases. A B-Raf inhibitor may comprise Vemurafenib. A B-Raf inhibitor may comprise RAF265. A B-Raf inhibitor may comprise PLX4720.

### 8. Wnt Inhibitors

A chemical reprogramming factor may comprise a Wnt inhibitor (or a Wnt signaling inhibitor). Wnt signaling pathway can be regulated by binding of a Wnt-protein ligand to a Frizzled family receptor. In canonical Wnt signaling pathway, binding of the Wnt ligand to the Frizzled family receptor can lead to a stabilization of beta-catenin, which in turn binds to Transcription factors of the T-cell family (TCF) and increases the transcription of genes under the regulation of Wnt-regulated enhancer. Wnt-signaling pathway may also comprises noncanonical planar cell polarity pathway and noncanonical Wnt/calcium pathway (Ramakrishnan et al., F1000Res. 2017 May 24; 6:746., which is herein incorporated by reference in its entirety). Inhibition of Wnt may lead to a decreased expression of beta-catenin. Activation or inhibition of Wnt signaling pathway can be assayed using TOP-flash assay, described in Molenaar et al., 1996; Cell. 1996 Aug. 9; 86(3):391-9, which is herein incorporated by reference in its entirety). Inhibition of Wnt signaling may also lead to increased expression of SPATS1 gene (see, for example, Zhai et al, Cellular Signalling. 22 (11): 1753-60, which is herein incorporated by reference in its entirety).

A Wnt inhibitor may comprise IWP-2 ([N-(6-methyl-2-benzothiazolyl)-2-[(3,4,6,7-tetrahydro-4-oxo-3-phenylthieno[3,2-d]pyrimidin-2-yl)thio]-acetamide]); WNT-C59 ([4-(2-Methyl-4-pyridinyl)-N-[4-(3-pyridinyl)phenyl]benzeneacetamide]); XAV-939 ([3,5,7,8-Tetrahydro-2-[4-(trifluoromethyl)phenyl]-4H-thiopyrano[4,3-d]pyrimidin-4-one]); or IWR-1 (Selleckchem catalog No. S7086); or any combination thereof. A Wnt inhibitor may comprise IWP-2. A Wnt inhibitor may comprise WNT-C59. A Wnt inhibitor may comprise XAV-939. A Wnt inhibitor may comprise IWR-1.

### 9. Rho-Associated, Coiled-Coil Containing Protein Kinase (ROCK) Inhibitors

A chemical reprogramming factor may comprise a ROCK inhibitor. ROCK is a kinase of family of serine-threonine specific protein kinases. ROCK may comprise ROCK1 or ROCK2. In some cases, ROCK can have a kinase domain, a coiled-coil region and a Pleckstrin homology (PH) domain (Tonges et al, 2011, Front Mol Neurosci. 2011; 4: 39., which is herein incorporated by reference in its entirety). ROCK may be a effector of RhoA-GTP. Binding of Rho-A may alleviate the autoinhibition by the PH domain to its kinase activity (Tonges 2011). Inhibition of ROCK may decrease phosphorylation of MLC (Tonges 2011). Inhibition of ROCK may increase the activity of MLC phosphatase (Tonges 2011). Inhibition of ROCK of decrease the activity of LIMK, which can lead to decreased phosphorylation of cofilin and increases actin depolymerization, and vice versa (Tonges 2011).

A ROCK inhibitor may comprise Y27632 ([(+)-(R)-trans-4-(1-aminoethyl)-N-(4-pyridyl)cyclohexanecarboxamide+++dihydrochloride)]); or Tzv (thiazovivin); or any combination thereof. A ROCK inhibitor may comprise Y27632. A ROCK inhibitor may comprise Tzv.

### 10. CBP/p300 Bromodomain Inhibitors

A chemical reprogramming factor may comprise a CBP/p300 bromodomain inhibitor. transcription coactivators CREB binding protein (CBP) and p300 are transcriptional coactivators. CBP and p300 may be acetyltransferases that mediate histone 3 lysine 27 acetylation (H3K27ac). In some cases, CBP and p300 have redundant functions (Martire et al 2020; BMC Mol Cell Biol. 2020 Jul. 20; 21(1):55., which is herein incorporated by reference in its entirety). Inhibition of CBP or p300 may lead to a decreased acetylation of H3K27 or H3K18 (Raisner et al., 2018; Cell Rep. 2018 Aug. 14; 24(7):1722-1729., which is herein incorporated by reference in its entirety). Inhibition of CBP and/or p300 may lead to decreased expression of target genes, such as Myc and others (Raiser 2018).

A CBP/p300 bromodomain inhibitor may decrease the acetyltransferase activity of CBP and/or p300. A CBP/p300 bromodomain inhibitor may comprise SGC-CBP30 ([2-[2-(3-chloro-4-methoxyphenyl)ethyl]-5-(3,5-dimethyl-4-isoxazolyl)-1-[(2S)-2-(4-morpholinyl)propyl]-1H-benzimidazole]); I-CBP112; GNE272; or GNE409; or any combination thereof. A CBP/p300 bromodomain inhibitor may comprise SGC-CBP30. A CBP/p300 bromodomain inhibitor may comprise I-CBP112. A CBP/p300 bromodomain inhibitor may comprise GNE272. A CBP/p300 bromodomain inhibitor may comprise GNE409.

### 11. Menin-MLL Interaction Inhibitors

A chemical reprogramming factor may comprise a Menin-MLL interaction inhibitor. Menin may be encoded by MEN1 in human. Menin may be a tumor suppressor (Cierpicki et al., 2014 Future Med Chem. 2014 March; 6(4):447-62.; which is herein incorporated by reference in its entirety). MLL may comprise a histone methyltransferase of the trithorax family (Cierpicki 2014). MLL may comprise MLL1 or MLL2. Binding of Menin and MLL may increase the transcriptional activity of MLL. Inhibition of Menin, MLL, and or Menin-MLL interactions may lead to decreased expressions of Hoxa9, Hoxc6, and/or Hoxc8 (Cierpicki 2014). Inhibition of Menin, MLL, and or Menin-MLL interactions may also decrease the expression of p27 and p18 cyclin-dependent kinase (CDK) inhibitors (Melne et al., 2005; Proc Natl Acad Sci USA. 2005 Jan. 18; 102(3):749-54, which is herein incorporated by reference in its entirety).

A Menin-MLL interaction inhibitor may comprise VTP50469, MI3454, or WDR5-IN-4, or any combination thereof. A Menin-MLL interaction inhibitor may comprise VTP50469. A Menin-MLL interaction inhibitor may comprise MI3454. A Menin-MLL interaction inhibitor may comprise WDR5-IN-4.

### 12. G Protein-Coupled Receptor Smoothened Agonists

A chemical reprogramming factor may comprise a G protein-coupled receptor Smoothened agonist. Smoothened may be encoded by the SMO gene (Arensdorf et al., 2016; Trends Pharmacol Sci. 2016 January; 37(1):62-72., which is herein incorporated by reference in its entirety). Smoothened may be a class F G protein-coupled receptor. Smoothened is a component of Hedgehog signaling pathway. Without the ligand Hedgehog, receptor Patched inhibits Smoothened. Once Hedgehog binds to Patched, Patched is internalized and degraded, activating Smoothened. Activated Smoothened can lead to the activation of Gli family transcription factors, which leads to increased expression of Ptch1, Ptch2, Gli1, CCND2, CCNE1, MYCN, BCL2, ABCG2, FGF4, VEGFA, PAX6, PAX7, PAX9, JAG1, or FOXM1 (Skoda et al., 2018; Bosn J Basic Med Sci. 2018 Feb. 20; 18(1):8-20., which is herein incorporated by reference in its entirety).

A G protein-coupled receptor Smoothened agonist may comprise SAG [3-chloro-N-[4-(methylamino)cyclohexyl]-N-[(3-pyridin-4-ylphenyl)methyl]-1-benzothiophene-2-carboxamide;hydrochloride,]); Purmorphamine; Hg-Ag1.5; or Sonic Hedgehog protein (Shh), or any combination thereof. A G protein-coupled receptor Smoothened agonist may comprise SGA. A G protein-coupled receptor Smoothened agonist may comprise Purmorphamine. A G protein-coupled receptor Smoothened agonist may comprise Hg-Ag1.5. A G protein-coupled receptor Smoothened agonist may comprise Shh. Shh can be a human Shh. Shh can also be a mammalian Shh.

### 13. JAK1/2 (Jak1/2) Inhibitors

A chemical reprogramming factor may comprise a Jak1/2 inhibitor. Jak may comprise Janus kinase. Jak1/2 may comprise tyrosine kinases. Jak1/2 may regulate cytokine signaling via interaction with type I/II cytokine receptors. These cytokine receptors can comprise interferon receptors, GM-CSF receptors, the gp130 receptors, single chain receptors, IL-2 receptors, IL-4 receptors, CT-1R receptors, CNTF receptors, NNT-1 receptors, Leptin receptors (Brooks et al., 2014; Science. 2014 May 16; 344(6185):1249783. and Gadina et al., 2001; Curr Opin Immunol. 2001 June; 13(3):363-73, each of which is herein incorporated by reference in its entirety). Inhibitions of Jak1/2 may lead to decreased expressions of decreased phosphorylation of STAT, which leads to decreased expressions of target genes, such as Fas, Bcl-2, or Bcl-X. Inhibitions of Jak1/2 may lead to increased apoptosis Hu et al., 2021; Signal Transduct Target Ther. 2021 Nov. 26; 6(1):402., which is herein incorporated by reference in its entirety).

A Jak1/2 inhibitor can comprise Ruxolitinib ([(3R)-3-cyclopentyl-3-[4-(7H-pyrrolo[2,3-d]pyrimidin-4-yl)pyrazol-1-yl]propanenitrile]); Tofacitinib; AZD1480; Baricitinib; S-Ruxolitinib; or Fedratinib; or any combination thereof. A Jak1/2 inhibitor can comprise Ruxolitinib. A Jak1/2 inhibitor can comprise Tofacitinib. A Jak1/2 inhibitor can comprise AZD1480. A Jak1/2 inhibitor can comprise Baricitinib. A Jak1/2 inhibitor can comprise S-Ruxolitinib. A Jak1/2 inhibitor can comprise Fedratinib.

### 14. Serine-Threonine Kinase Akt Inhibitors

A chemical reprogramming factor may comprise an Akt inhibitor. Akt activation may activate mTOR via the phosphorylation of mTOR at Ser2448 and lead to the activation of complex mTORC1 and/or inhibit TSC1/TSC2. Activated mTORC1 may increase the phosphorylation of 70S6K1 or eukaryotic translation initiation factor 4E-binding protein 1 (4EBP1). Phosphorylation of 4EBP-1 can promote the expression of hypoxia-inducible factor 1α, cyclin D1, and/or c-Myc, which leads to angiogenesis or cell cycle progression. Akt activation may lead to increased cell proliferation. Inhibition of Akt can decrease the phosphorylation of mTOR, TSC1/2, 4eBP-1, or 70S6K1. Inhibition of Akt can decrease the expression of hypoxia-inducible factor 1α, cyclin D1, and/or c-Myc (Li et al., 2020; Cell Death Dis. 2020 Sep. 24; 11(9):797.; which is herein incorporated by reference in its entirety).

An Akt inhibitor may comprise AKT Kinase Inhibitor (AKTi; [3-[2-(4-amino-1,2,5-oxadiazol-3-yl)-7-(3-aminopropoxy)-1-ethylimidazo[4,5-c]pyridin-4-yl]prop-2-yn-1-ol]).

15. c-Jun Kinase Inhibitors

A chemical reprogramming factor may comprise a c-Jun kinase inhibitor. c-Jun kinase is a MAP kinase family kinase. c-Jun kinase may phosphorylate c-Jun on Ser-63 and Ser-73 within its transcriptional activation domain. Activation of c-Jun kinase may lead to the activation of AP1 target genes including FosB, WEE1, PVR, MAP1LC3B and/or LGALS3), and vice versa (Schummer et al., 2016; Cancer Biol Ther. 2016 May 3; 17(5):486-97, which is herein incorporated by reference in its entirety.

A c-Jun kinase inhibitor may comprise JNKIN8 ([3-[[(E)-4-(dimethylamino)but-2-enoyl]amino]-N-[3-methyl-4-[(4-pyridin-3-ylpyrimidin-2-yl)amino]phenyl]benzamide]); JNKIN7; JNKIN5; or JNKIN12; or any combination thereof. A c-Jun kinase inhibitor may comprise JNKIN8. A c-Jun kinase inhibitor may comprise JNKIN5. A c-Jun kinase inhibitor may comprise JNKIN12. A c-Jun kinase inhibitor may comprise JNKIN7.

### 16. p38 MAPK Inhibitors

A chemical reprogramming factor may comprise a p38 MAPK inhibitor. As used herein, a p38 MAPK (or p38 mitogen-activated protein kinase) is a kinase of class of mitogen-activated protein kinases. p38 MPAK may comprise p38-a; p38-0; p38-7; or p38-6. Activation of p38 MPAK may increase the phosphorylation of MAPKAP kinase 2, ATF2, Mac, MEF2, or p53, and vice versa (Rawas et al., 2020; Int J Mol Sci. 2020 Jul. 8; 21(14):4833, which is herein incorporated by reference in its entirety). Inhibition of p38 MPAK may decrease the expression of Jun, Fos, Myc, Egr-1, Maff, Sox2, Runx2, or others described in Whitmarsh 2010; BMC Biol. 2010 Apr. 27; 8:47, which is herein incorporated by reference in its entirety). Inhibition of p38 MPAK may decrease cell proliferation.

A p38 MAPK inhibitor may comprise BIRB796 ([1-[5-tert-butyl-2-(4-methylphenyl)pyrazol-3-yl]-3-[4-(2-morpholin-4-ylethoxy)naphthalen-1-yl]urea]); SB203580; or SB202190; or any combination thereof. A p38 MAPK inhibitor may comprise BIRB796. A p38 MAPK inhibitor may comprise SB2033580. A p38 MAPK inhibitor may comprise SB202190.

### 17. MEK Inhibitors

A chemical reprogramming factor may comprise a MEK inhibitor. A MEK (mitogen-activated protein kinase kinase) is also known as MAP2K or MAPKK, may be a dual-specificity kinase enzyme which phosphorylates mitogen-activated protein kinase (MAPK). There can be seven genes that encode various MEKs, such as MAP2K1 (encoding MEK1), MAP2K2 (encoding MEK1), MAP2K3 (encoding MEK3), MAP2K4 (encoding MEK4), MAP2K5 (encoding MEK5), MAP2K6 (encoding MEK6), and MAP2K7 (encoding MEK7). MEKs may activate p38 MAPK (e.g., by MKK3 and MKK6), JNK (e.g., by MKK4 and MKK7), and ERK (e.g., by MEK1 and MEK2) (Dérijard B, et al. (1995) Science. 267 (5198): 682-5, which is herein incorporated by reference in its entirety). MEK may phosphorylate and activate a MAPK (e.g., a p38 MAPK). In some case, the inhibition of MEK and the inhibition of MAPK may lead to similar changes of biological activities. A MEK inhibitor described herein is a direct inhibitor of a MEK protein, and does not directly inhibit a MAPK protein (e.g., does not inhibit a MAPK protein in an in vitro cell-free kinase assay study that does not involve MEK proteins).

A MEK inhibitor may also comprise PD0325901, AZD8330, or TAK-733, or any combination thereof. A MEK inhibitor may comprise PD0325901. A MEK inhibitor may comprise AZD8330. A MEK inhibitor may comprise TAK-733.

### 18. Adenosine Kinase Inhibitors

A chemical reprogramming factor may comprise an adenosine kinase inhibitor. An adenosine kinase may phosphorylate adenosine to adenosine monophosphate using the gamma phosphate of ATP. Inhibition of adenosine kinase may lead to an increase amount of S-adenosyl-L-homocysteine (SAH). Increased amounts of S-adenosyl-L-homocysteine (SAH) may inhibit transmethylation reactions (Fox et al, 1978; Annu Rev Biochem. 1978; 47:655-86., which is herein incorporated by reference in its entirety).

An adenosine kinase inhibitor may comprise 5-Iodotubercidin (5-ITU; [(2R,3R,4S,5R)-2-(4-amino-5-iodopyrrolo[2,3-d]pyrimidin-7-yl)-5-(hydroxymethyl)oxolane-3,4-diol]); or ABT 702 dihydrochloride; or any combination thereof. An adenosine kinase inhibitor may comprise 5-ITU. An adenosine kinase inhibitor may comprise ABT 702).

### 19. SETD2 Inhibitors

A chemical reprogramming factor may comprise a SET domain containing 2 (SETD2) inhibitor. SETD2 may comprise a histone methyltransferase that methylates the lysine 36 of histone H3 (H3K36). SETD2 may mediates mono-, di-, or tri-methylation of H3. Inhibition of SETD2 may decrease the phosphorylation of H3 at K36. In some cases, inhibition of SETD2 may increase the frequency of deletion mutations induced by microhomology-mediated end joining (Pfister et al., 2014; Cell Rep. 2014 Jun. 26; 7(6):2006-18., which is herein incorporated by reference in its entirety). Inhibition of SETD2 may increase the frequency of DNA repair by microhomology-mediated end joining. Inhibition of SETD2 may decrease homologous recombination repair.

A SET domain containing 2 (SETD2) inhibitor may comprise SETD2-IN-1 ([N-[(1R,3S)-3-(4-acetylpiperazin-1-yl)cyclohexyl]-4-fluoro-7-methyl-1H-indole-2-carboxamide;2,2,2-trifluoroacetic acid]); EPZ-719; or MMSET-IN-1; or any combination thereof. A SET domain containing 2 (SETD2) inhibitor may comprise SETD2-IN-1. A SET domain containing 2 (SETD2) inhibitor may comprise EPZ-719. A SET domain containing 2 (SETD2) inhibitor may comprise MMSET-IN-1.

### 20. Casein Kinase 2 Inhibitors

A chemical reprogramming factor may comprise a casein kinase 2 inhibitor. Casein kinase 2 (CK2) may be a serine/threonine protein kinase. CK2 may use ATP or CTP as phosphate sources. CK2 may phosphorylate AKT, STAT, beta-catenin, or androgen receptors (Borgo et al., 2021; Signal Transduct Target Ther. 2021 May 17; 6(1):183; Signal Transduct Target Ther. 2021 May 17; 6(1):183). Inhibition of CK2 may lead to decreased phosphorylation of AKT, STAT, beta-catenin, or androgen receptors. Inhibition of CK2 may lead to decreased expression of genes regulated by AKT beta-catenin as described elsewhere in this disclosure.

A casein kinase 2 inhibitor may comprise CX-4945 [5-(3-chloroanilino)benzo[c][2,6]naphthyridine-8-carboxylic acid]).

### 21. Inhibitors of Histone Demethylation

A chemical reprogramming factor may comprise an inhibitor of histone demethylation. Histone demethylation may lead to global activation of gene expression. Inhibition of histone demethylation may result in an increased level of histone methylation. An inhibitor of histone demethylation may comprise Tranylcypromine.

### Cell Culture

In some cases, a composition may comprise a culture medium. Methods provided herein may comprise culturing a population of cells. Culturing a population of cells may comprise contacting the population of cells with a culture medium. Culturing a population of cells may comprise incubating the population of cells with the culture medium for a period of time. During or subsequent to the culturing, at least a subset of the population of cells may undergo cell proliferation. The subset of the population of cells may increase in cell numbers and/or cell mass. In some cases, during or subsequent to the culturing, a subset of the population of cells may give rise to a progeny or progenies.

During a conversion of a cell population, at least a subset of the population of cell may be converted to a different cell type. In some case, the subset of the population of cells may comprise at least a progeny of the subset of the population of cells.

The culture medium may comprise a chemical reprogramming factor. In some cases, the culture medium may not comprise A chemical reprogramming factor. In some cases, the culture medium may comprise at least a molecule for supporting the viability of a cell. In some cases, the culture medium may comprise at least a molecule for supporting the proliferation of a cell. A culture medium may comprise at least a molecule for supporting growth of a cell in vitro or ex vivo. A culture medium may comprise a peptide, a polypeptide, a growth factor, a carbon source, a nitrogen source, a mineral source, a vitamin source, water, salt, oxygen, or carbon dioxide, or any combination thereof.

The stage 1 methods provided herein may comprise plating a first population of cells or somatic cells onto a cell growth substrate. The cell growth substrate may comprise a culture plate. The culture plate may have a growth area of about 0.1 square centimeter (cm\hat{ }2), 0.2 cm\hat{ }2, 0.3 cm\hat{ }2, 0.4 cm\hat{ }2, 0.5 cm\hat{ }2, 0.6 cm\hat{ }2, 0.7 cm\hat{ }2, 0.8 cm\hat{ }2, 0.9 cm\hat{ }2, 1 cm\hat{ }2, 1.1 cm\hat{ }2, 1.2 cm\hat{ }2, 1.3 cm\hat{ }2, 1.4 cm\hat{ }2, 1.5 cm\hat{ }2, 1.6 cm\hat{ }2, 1.7 cm\hat{ }2, 1.8 cm\hat{ }2, 1.9 cm\hat{ }2, 2 cm\hat{ }2, 2.1 cm\hat{ }2, 2.2 cm\hat{ }2, 2.3 cm\hat{ }2, 2.4 cm\hat{ }2, 2.5 cm\hat{ }2, 2.6 cm\hat{ }2, 2.7 cm\hat{ }2, 2.8 cm\hat{ }2, 2.9 cm\hat{ }2, 3 cm\hat{ }2, 3.1 cm\hat{ }2, 3.2 cm\hat{ }2, 3.3 cm\hat{ }2, 3.4 cm\hat{ }2, 3.5 cm\hat{ }2, 3.6 cm\hat{ }2, 3.7 cm\hat{ }2, 3.8 cm\hat{ }2, 3.9 cm\hat{ }2, 4 cm\hat{ }2, 4.1 cm\hat{ }2, 4.2 cm\hat{ }2, 4.3 cm\hat{ }2, 4.4 cm\hat{ }2, 4.5 cm\hat{ }2, 4.6 cm\hat{ }2, 4.7 cm\hat{ }2, 4.8 cm\hat{ }2, 4.9 cm\hat{ }2, 5 cm\hat{ }2, 6 cm\hat{ }2, 7 cm\hat{ }2, 8 cm\hat{ }2, 9 cm\hat{ }2, 10 cm\hat{ }2, 20 cm\hat{ }2, 30 cm\hat{ }2, 40 cm\hat{ }2, 50 cm\hat{ }2 or more.

The first population of stage 1 cells or somatic cells may be plated on the cell growth substrate at a density. Such a density may comprise be at least about 1×10\hat{ }3 cells per cm\hat{ }2 cell growth area, 2.5×10\hat{ }3 cells per cm\hat{ }2 cell growth area, 5×10\hat{ }3 cells per cm\hat{ }2 cell growth area, 1×10\hat{ }4 cells per cm\hat{ }2 cell growth area, 2.5×10\hat{ }4 cells per cm\hat{ }2 cell growth area, 5×10\hat{ }4 cells per cm\hat{ }2 cell growth area, 1×10\hat{ }5 cells per cm\hat{ }2 cell growth area, 2.5×10≡cells per cm\hat{ }2 cell growth area, 5×10\hat{ }5 cells per cm\hat{ }2 cell growth area, 1×10\hat{ }6 cells per cm\hat{ }2 cell growth area, 2.5×10\hat{ }6 cells per cm\hat{ }2 cell growth area, 5×10\hat{ }6 cells per cm\hat{ }2 cell growth area, 1×10\hat{ }7 cells per cm\hat{ }2 cell growth area, 2.5×10\hat{ }7 cells per cm\hat{ }2 cell growth area, 5×10\hat{ }7 cells per cm\hat{ }2 cell growth area, 1×10\hat{ }8 cells per cm\hat{ }2 cell growth area, 2.5×10\hat{ }8 cells per cm\hat{ }2 cell growth area, 5×10\hat{ }8 cells per cm\hat{ }2 cell growth area or more. Such a density may comprise be at most about 1×10\hat{ }3 cells per cm\hat{ }2 cell growth area, 2.5×10\hat{ }3 cells per cm\hat{ }2 cell growth area, 5×10\hat{ }3 cells per cm\hat{ }2 cell growth area, 1×10\hat{ }4 cells per cm\hat{ }2 cell growth area, 2.5×10\hat{ }4 cells per cm\hat{ }2 cell growth area, 5×10\hat{ }4 cells per cm\hat{ }2 cell growth area, 1×10\hat{ }5 cells per cm\hat{ }2 cell growth area, 2.5×10\hat{ }5 cells per cm\hat{ }2 cell growth area, 5×10\hat{ }5 cells per cm\hat{ }2 cell growth area, 1×10\hat{ }6 cells per cm\hat{ }2 cell growth area, 2.5×10\hat{ }6 cells per cm\hat{ }2 cell growth area, 5×10\hat{ }6 cells per cm\hat{ }2 cell growth area, 1×10\hat{ }7 cells per cm\hat{ }2 cell growth area, 2.5×10\hat{ }7 cells per cm\hat{ }2 cell growth area, 5×10\hat{ }7 cells per cm\hat{ }2 cell growth area, 1×10\hat{ }8 cells per cm\hat{ }2 cell growth area, 2.5×10\hat{ }8 cells per cm\hat{ }2 cell growth area, 5×10\hat{ }8 cells per cm\hat{ }2 cell growth area or more.

When culturing the cells provided herein, the methods may comprise passaging the cells. In some cases, passaging the cells may comprise removing a first composition from a population of cells, splitting the populations of cells into a subset of the population of cells, and incubating the split population of cells with a second composition. A passage my comprise the process of passaging the cells once. The first and second compositions for passaging may be comprise the same chemical make-up. The second composition may be new or haven't contacted a cell prior to contacting the split population of cells. Hence, passaging may comprise replacing a culture medium with a new culture medium. In some cases, the split population of cells may be about 1%, 2%, 3%, 4%, 5%, 6%, 7%, 8%, 9%, 10%, 15%, 20%, 25%, 30%, 35%, 40%, 45%, 50%, 60%, 70%, 80% or the unsplit population of cells. The methods described herein may comprise at most 2, 3, 4, 5, or 6 passages. The methods may comprise at most 5 passages. The methods may comprise at most 4 passages. The methods may comprise at most 3 passages.

### Equivilant Amounts for Substituting Chemical Reprogramming Factors

In some cases, two different chemical reprogramming factors may substitute each other in any of the compositions or methods described herein when converting, contacting, or incubating cells. In some cases, two different chemical reprogramming factors may substitute each other if they increase or decrease a same biological activity. In some cases, two different chemical reprogramming factors may substitute each other if they inhibit a same enzyme, a same protein-protein binding domain, or a same molecular interaction, or any combination thereof. When a first chemical reprogramming factor substitute a second chemical reprogramming factor, an amount of the first chemical reprogramming factor may be used. Such an amount of the first chemical reprogramming factor may comprise an equivalent amount of the first chemical reprogramming factor.

To identify the equivalent amount of the first chemical reprogramming factor, one or more assays may be carried out. For example, the first and second chemical reprogramming factors may activate or inhibit a same target (e.g., an enzyme or a protein-protein binding interaction. The equivalent concentration of the first chemical reprogramming factor may be derived using a biological assay that assay the same target. A measurable value may be derived individual biological assay, each assaying the same target using the first or second chemical reprogramming factor. The equivalent amount of the first chemical reprogramming factor may be one that has a measurable value that is within 1%, 2%, 3%, 4%, 5%, 6%, 7%, 8%, 9%, 10%, 15%, 20%, 25%, 30%, 35%, 40%, 45%, or 50% of that of the second chemical reprogramming factor. In other cases, the equivalent concentration of the first chemical reprogramming factor may be derived using the methods described herein to convert cells. For example, the methods may assay the number of converted cells generated from a starting population of cells using one of the first and second chemical reprogramming factors (i.e., each of the first and second chemical reprogramming factors is assayed for how many converted cells are generated from a substantially same number of the starting population of cells (e.g., the numbers of cells are within at least 90% with each assay). The equivalent amount of the first chemical reprogramming factor may be one that has a number of converted cells that is within 1%, 2%, 3%, 4%, 5%, 6%, 7%, 8%, 9%, 10%, 15%, 20%, 25%, 30%, 35%, 40%, 45%, or 50% of that of the second chemical reprogramming factor.

### Effective Amounts of Chemical Reprogramming Factors for Cell Conversion

The methods or compositions provided herein may comprise an effective amount of a chemical reprogramming factor for cell conversion. An effective amount of a chemical reprogramming factor for cell conversions may comprise the amount of the chemical reprogramming factor that can facilitate the conversion of one cell type to another cell type. An effective amount of a chemical reprogramming factor for cell conversions comprises the amount of the chemical reprogramming factor (e.g., any amounts described herein) relative to a number of cells (e.g., any numbers of cells in a cell population before contacting one composition described herein, such that the contacting may converts at least a cell type from the cell population to another cell type).

In some cases, a chemical reprogramming factor may have an effective range for converting cells. The effective range may comprise a lower amount and a higher amount of the chemical reprogramming factors. Within the effective range, the number of converted cells generated from a starting population of cells, using the methods or compositions described herein, may be substantially the same. This substantially the same number of cells may mean that the numbers of converted cells generated from the lower and the higher amounts of the chemical reprogramming factors are within 1%, 2%, 3%, 4%, 5%, 6%, 7%, 8%, 9%, 10%, 15%, 20%, 25%, 30%, 35%, 40%, 45%, or 50%.

### Genetic Modification

A cell provided herein or a progeny thereof may comprise a genome modification. The first cell population of stage 1 may comprise one or more cells with the genetic modification. The second cell population of stage 1 may comprise one or more cells with the genetic modification. The first cell population of stage 2 may comprise one or more cells with the genetic modification. The second cell population of stage 2 may comprise one or more cells with the genetic modification. The first cell population of stage 3 may comprise one or more cells with the genetic modification. The second cell population of stage 3 may comprise one or more cells with the genetic modification. The cell with the genetic modification may comprise a somatic cell, epithelial-like cell, intermediate plastic state cell, pluripotent stem cell, or a progeny thereof, or a combination thereof. The cell with the genetic modification may comprise a somatic cell or the progeny thereof. The cell with the genetic modification may comprise an epithelial-like cell or a progeny thereof. The cell with the genetic modification may comprise an intermediate plastic state cell or a progeny thereof. The cell with the genetic modification may comprise a pluripotent stem cell or a progeny thereof.

In the methods described herein, at least a subset of a population of cells may comprise a genetic modification. During the cell conversion and/or culturing a converted cell derived from the subset of the population of cells or any progenies thereof may comprise the same genetic modification.

A genetic modification of a cell may comprise a change in the genetic material of the cell. A genetic modification may comprise a change in the make-up of a genome of a cell. The genetic material may comprise a genome of a cell. The genetic material may comprise chromosomal DNA or extra-chromosomal DNA. The extra-chromosomal DNA may comprise a mitochondrial DNA. A genetic modification may comprise a mutation. The mutation may comprise a substitution, deletion, or insertion of the genome. In other cases, the mutation also comprises a DNA rearrangement of the chromosomal or mitochondrial DNA. In some cases, a genetic modification may comprise insertions of exogenous genetic materials into a cell. In other cases, a genetic modification may comprise insertions of exogenous genetic materials into a genome of a cell.

A genetic modification may comprise random mutagenesis of a genome of the cell. A genetic modification may comprise homologous or non-homologous recombination within the genome of a cell. A genetic modification may comprising utilizing a nuclease to generate the genetic modification. The nuclease may comprise an exonuclease, an endonuclease, or a combination thereof. The nuclease may comprise an exonuclease. The nuclease may comprise an endonuclease. The endonuclease may comprise zinc finger nuclease (ZFN), transcription activator like effector nuclease (TALEN), homing endonuclease (HE), meganuclease, MegaTAL, a clustered regularly interspaced short palindromic repeats (CRISPR)-associated endonuclease, or a combination thereof. The genetic modification may also comprise a reverse transcriptase. In some embodiments, an endonuclease may introduce one or more single-stranded breaks (SSBs) and/or one or more double-stranded breaks (DSBs).

A genetic modification may comprise a cell carrying an exogenous nucleic acid. The exogenous nucleic acid may comprise DNA or ribonucleic acid (RNA). The exogenous nucleic acid may comprise a virus. The exogenous nucleic acid may not comprise a virus. The exogenous nucleic acid may comprise a viral-based vector, a non-viral-based vector, or a combination thereof. The exogenous nucleic acid may comprise a viral-based vector. The exogenous nucleic acid may comprise a non-viral-based vector. The exogenous nucleic acid may comprise a viral-based vector and non-viral-based vector.

The exogenous nucleic acid may comprise a sequence that encodes a polypeptide, a non-coding nucleic acid molecule, or a combination thereof. The exogenous nucleic acid may comprise a sequence that encodes a polypeptide. The exogenous nucleic acid may comprise a sequence that encodes non-coding nucleic acid molecule. The exogenous nucleic acid may comprise a sequence that encodes a polypeptide and a non-coding nucleic acid molecule. The non-coding nucleic acid molecule may comprise a micro ribonucleic acid (miRNA), a long non-coding RNA (lncRNA), a ribosomal ribonucleic acid (rRNA), a silencing ribonucleic acid (siRNA), a short hairpin RNA (shRNA), a ribozyme, or any combinations thereof.

A cell comprising a genetic modification may have reduced immunogenicity. In some cases, the cell comprising a genetic modification may elicit reduced immune response of a subject against the cell, when the cell is administer to the subject, relative to a comparable cell that does not comprise the genetic modification. In some cases, the reduction of the immunogenicity of a cell comprising the genetic modification may be at least about 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%, relative to a comparable cell that does not comprise the genetic modification. In some cases, the reduction of the immunogenicity of a cell comprising the genetic modification may be at most about 0.01%, 0.1%, 0.5%, 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%, relative to a comparable cell that does not comprise the genetic modification. When comparing immunogenicity, a comparable cell to a cell may comprise a cell that with the same cell type as the cell. The comparable cell may not comprise the same genetic modification of the cell comprising the genetic modification.

### Methods of Uses

Identification of a readily available source of stem cells, progenitor cells, dedifferentiated cells or cells with potency that can give rise to a desired cell type or morphology can be beneficial for therapeutic treatments or tissue engineering.

The cells obtained by methods of provided can comprise hCiPSCs, intermediate plastic state cells, and epithelia-like cells that are readily available source of stem cells, progenitor cells, dedifferentiated cells, or cells with potency. In some cases, stem cells, progenitor cells, dedifferentiated cells, or cells with potency obtained by a method provided herein express at least one of stem cell related markers such as LIN28A, SALL4, OCT4 or NANOG. Intermediate state plastic cells or epithelia-like cells obtained by methods of this application may also be used similarly as source of stem cells, progenitor cells, dedifferentiated cells or cells with potency. The availability of stem cells, progenitor cells, dedifferentiated cells or cells with potency and regenerative potentials can be beneficial in transplantation, tissue engineering, and regulation of angiogenesis, vasculogenesis, and cell replacement or cell therapies, as well as the prevention of certain diseases. Such stem cells or progenitor cells can also be used to introduce a gene into a subject as part of a gene therapy regimen. In addition, the cells obtained by a method of this application comprising one or more of stages 1, 2, or 3. For example, any of the pluripotent stem cells, epithelia-like cells, or intermediate plastic state cells may be directly induced to a desired cell type and implanted and delivered to the subject.

### Providing Differentiated Somatic Cells (Re-Differentiated Cells)

Once established, a culture of stem cells may be used to produce progeny cells, for example, fibroblasts capable of producing new tissue. The hCiPSCs (e.g., human CiPSCs or hCiPSCs) can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells, blood cells, retinal cells (and other cells involved in sensory perception, such as those that form hair cells in the ear or taste buds on the tongue), and nervous tissue including nerves.

In one embodiment, the hCiPSCs can be induced to differentiate into cells of ectodermal origin by exposing the cells to an “ectodermal differentiating” media. In another embodiment the hCiPSCs can be induced to differentiate into cells of mesodermal origin by exposing the cells to “mesodermal differentiating media”. In still another embodiment, the hCiPSCs can be induced to differentiate into cells of endodermal origin by exposing the cells to “endodermal media”. Components of “endodermal”, “mesodermal” and “ectodermal” media are known to one of skill in the art. Known cell surface markers can be used to verify that the cells are indeed differentiating into cells of the lineage of the corresponding cell culture medium. The most commonly accepted markers to confirm differentiation of the three germ layers are the expression of alpha fetal protein for endodermal cells, alpha smooth muscle actin for mesoderm, and Beta-III tubulin for ectoderm, all of which are normally expressed very early in the development of these tissues.

Differentiation of stem cells to fibroblasts or other cell types, followed by the production of tissue therefrom, can be triggered by specific exogenous growth factors or by changing the culture conditions (e.g., the density) of a stem cell culture. Methods for inducing differentiation of cells into a cell of a desired cell type can comprise, for example, hCiPSCs can be induced to differentiate by adding a substance (e.g., a growth factor, enzyme, hormone, or other signaling molecule) to the cell's environment. The differentiated cells can be expanded in culture and stored for later retrieval and use.

The intermediate plasticity state cells, and epithelia-like cells are readily available source for generating to other cell types that can be triggered by specific exogenous growth factor, small molecules, over expression genes or by changing the culture conditions (e.g., the density) of a stem cell culture. The cells induced from the intermediate plasticity state cells, and epithelia-like cells can be different cell types including but not limited to: somatic cells of hematological origin, skin derived cells, adipose cells, epithelial cells, endothelial cells, cells of mesenchymal origin, parenchymal cells (for example, hepatocytes, 3 cells), neurological cells, and connective tissue cells.

### 1. Cell Therapy

Therapeutic uses of the induced pluripotent stem cells can comprise transplanting the induced pluripotent stem cells, stem cell populations, or progeny thereof into individuals to treat a variety of pathological states including diseases and disorders resulting from cancers, wounds, neoplasms, injury, viral infections, diabetes and the like. Treatment may entail the use of the cells to produce new tissue, and the use of the tissue thus produced. The cells may be implanted, injected or otherwise administered directly to the site of tissue damage so that they will produce new tissue in vivo. In one embodiment, administration includes the administration of genetically modified hCiPSCs or their progeny. In one embodiment, the hCiPSCs are obtained from autologous cells i.e., the donor cells are autologous. However, the cells can be obtained from heterologous cells. In one embodiment, the donor cells are obtained from a donor genetically related to the recipient. In another embodiment, donor cells are obtained from a donor genetically un-related to the recipient.

If the hCiPSCs are derived from a heterologous (non-autologous/allogenic) source compared to the recipient subject, concomitant immunosuppression therapy is typically administered, e.g., administration of the immunosuppressive agent cyclosporine or FK506. However, due to the immature state of the human induced pluripotent stem cells such immunosuppressive therapy may not be required. Accordingly, in one embodiment, the human induced pluripotent stem cells can be administered to a recipient in the absence of immunomodulatory (e.g., immunosuppressive) therapy. Alternatively, the cells can be encapsulated in a membrane, which permits exchange of fluids but prevents cell/cell contact. Transplantation of microencapsulated cells is described in Balladur et al., Surgery, 117:189-94, 1995; and Dixit et al., Cell Transplantation 1:275-79 (1992), each of which is herein incorporated by reference in its entirety.

The hCiPSCs can be induced to differentiate into cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells (e.g., alpha cells, delta cells, PP cells, and beta cells), blood cells (e.g., leukocytes, erythrocytes, macrophages, and lymphocytes), retinal cells (and other cells involved in sensory perception, such as those that form hair cells in the ear or taste buds on the tongue), and nervous tissue including nerves.

### (i) Diabetes

Diabetes mellitus (DM) is a group of metabolic diseases where the subject has high blood sugar, either because the pancreas does not produce enough insulin, or, because cells do not respond to insulin that is produced.

A replacement for insulin therapy is provision of islet cells to the patient in need of insulin. Shapiro et al., N Engl J Med., 343(4):230-8 (2000), which is herein incorporated by reference in its entirety, have demonstrated that transplantation of beta cells/islets provides therapy for patients with diabetes. Although numerous insulin types are commercially available, these formulations are provided as injectables. The human induced pluripotent stem cells can provide an alternative source of islet cells to prevent or treat diabetes. For example, induced pluripotent stem cells can be isolated and differentiated to a pancreatic cell type and delivered to a subject. Alternatively, the induced pluripotent stem cells can be delivered to the pancreas of the subject and differentiated to islet cells in vivo. Accordingly, the cells can be beneficial for transplantation in order to prevent or treat the occurrence of diabetes. Methods for reducing inflammation after cytokine exposure without affecting the viability and potency of pancreatic islet cells are disclosed for example in U.S. Pat. No. 8,637,494 to Naziruddin, et al, which is herein incorporated by reference in its entirety.

### (ii) Neurological Disorders

Neurological disorders and conditions can be characterized by conditions involving the dysfunction and/or deterioration of neurons and/or glial cells, as a result of disease, hereditary conditions or injury, such as traumatic or ischemic spinal cord or brain injury. Neurological disorders and conditions can comprise any disease or disorder or symptoms or causes or effects thereof involving dysfunction, damage, or deterioration of neurons and/or glial cells. Neurological disorders and conditions, to which the cells provided herein are applicable, can include, but are not limited to, neurodegenerative diseases and conditions.

The methods disclosed herein can comprise transplanting into a subject in need thereof NSCs, neural progenitors, neural precursors, or glial cells that have been expanded in vitro such that the cells can ameliorate the neurological disorders and conditions. Transplantation of the expanded cells (e.g., NSCs, neural progenitors, neural precursors, or glial cells) can be used to improve ambulatory function in a subject suffering from various forms of myelopathy with symptoms of spasticity, rigidity, seizures, paralysis or any other hyperactivity of muscles. Methods for expanding and transplanting neural cells and neural progenitor cells for the treatment of different neurodegenerative conditions is disclosed for example, in U.S. Pat. No. 8,236,299 to Johe, et. al, which is herein incorporated by reference in its entirety.

(iii) Cancer Therapy

Therapeutic uses of the hCiPSCs and their progeny can comprise transplanting the induced pluripotent stem cells, stem cell populations, or progeny thereof into individuals to treat and/or ameliorate the symptoms associated with cancer. For example, in one embodiment, the hCiPSCs can be administered to cancer patients who have undergone chemotherapy that has killed, reduced, or damaged cells of a subject. In a typical stem cell transplant for cancer, high doses of chemotherapy are used, often along with radiation therapy, to aim to destroy all the cancer cells. This treatment also kills the stem cells in the bone marrow. Soon after treatment, stem cells are given to replace those that were destroyed.

In some cases, the hCiPSCs can be transfected or transformed (in addition to the de-differentiation factors) with at least one additional therapeutic factor. For example, once hCiPSCs are isolated, the cells may be transformed with a polynucleotide encoding a therapeutic polypeptide and then implanted or administered to a subject, or may be differentiated to a desired cell type and implanted and delivered to the subject. Under such conditions the polynucleotide is expressed within the subject for delivery of the polypeptide product.

### 2. Tissue Engineering

CiPSCs and their progeny can be used to make tissue engineered constructions. Tissue engineered constructs may be used for a variety of purposes including as prosthetic devices for the repair or replacement of damaged organs or tissues. They may also serve as in vivo delivery systems for proteins or other molecules secreted by the cells of the construct or as drug delivery systems in general. Tissue engineered constructs also find use as in vitro models of tissue function or as models for testing the effects of various treatments or pharmaceuticals. The biomaterial scaffolds for transplantation of stem cells are described in Willerth, S. M. and Sakiyama-Elbert, S. E., Combining stem cells and biomaterial scaffolds for constructing tissues and cell delivery (Jul. 9, 2008), StemBook, ed. The Stem Cell Research Community, StemBook, each of which is herein incorporated by reference in its entirety. Tissue engineering technology frequently may involve selection of an appropriate culture substrate to sustain and promote tissue growth. These substrates can be three-dimensional and processable to form scaffolds of a desired shape for the tissue of interest.

Methods for producing tissue engineered constructs and engineered native tissue are described in U.S. Pat. No. 6,962,814, which is herein incorporated by reference in its entirety. Methods for using or manufacturing tissue engineered ligaments and tendons are described in U.S. Pat. No. 7,914,579, which is herein incorporated by reference in its entirety. U.S. Pat. No. 5,716,404 discloses methods and compositions for reconstruction or augmentation of breast tissue using dissociated muscle cells implanted in combination with a polymeric matrix, which is herein incorporated by reference in its entirety. U.S. Pat. No. 8,728,495 discloses repair of cartilage using autologous dermal fibroblasts, which is herein incorporated by reference in its entirety. U.S. Published application No. 20090029322 discloses the use of stem cells to form dental tissue for use in making tooth substitute, which is herein incorporated by reference in its entirety. U.S. Published application No. 2006/0019326 discloses cell-seed tissue-engineered polymers for treatment of intracranial aneurysms, which is herein incorporated by reference in its entirety. U.S. Published application No. 2007/0059293 discloses the tissue-engineered constructs (and method for making such constructs) that can be used to replace damaged organs for example kidney, heart, liver, spleen, pancreas, bladder, ureter and urethra, which is herein incorporated by reference in its entirety.

### 3. Therapeutic Compositions

The hCiPSCs can be formulated for administration, delivery or contacting with a subject, tissue or cell to promote de-differentiation in vivo or in vitro/ex vivo. Additional factors, such as growth factors, other factors that induce differentiation or dedifferentiation, secretion products, immunomodulators, anti-inflammatory agents, regression factors, biologically active compounds that promote innervation, vascularization or enhance the lymphatic network, and drugs, can be incorporated.

The induced pluripotent cells can be administered to a patient by way of a composition that includes a population of hCiPSCs or hCiPSC progenies alone or on or in a carrier or support structure. In many embodiments, no carrier will be required. The cells can be administered by injection onto or into the site where the cells are required. In these cases, the cells will typically have been washed to remove cell culture media and will be suspended in a physiological buffer. In other embodiments, the cells are provided with or incorporated onto or into a support structure. Support structures may be meshing, solid supports, scaffolds, tubes, porous structures, and/or a hydrogel.

### Use of Small Reprogramming Factors

The compositions comprising chemical reprogramming factors can be used for tissue regeneration, tissue remodeling and repair, rejuvenation or reversing aging, and inhibiting or reversing fibrosis in vitro and in vivo.

In some cases, chemical reprogramming factors of stage 1, 2 or 3 described herein are formulated for administration, delivery or contacting with a subject, tissue or cell to promote de-differentiation, regeneration, repair and rejuvenation in vivo or in vitro/ex vivo. Additional factors, such as growth factors, other factors that induce dedifferentiation or regeneration, secretion products, immunomodulators, anti-inflammatory agents, regression factors, biologically active compounds that promote innervation, vascularization or enhance the lymphatic network, and drugs, can be incorporated.

In one embodiment, the chemical reprogramming factors are administered to a patient by way of a composition that includes all or part of the chemical reprogramming factors for stage 1, 2, or 3 described herein. The chemical reprogramming factor compositions can be administered systemically or by injection onto or into the site where the cells are lost or tissues are damaged to boost the endogenous repair ability. In some embodiments, no carrier is required. In other embodiments, the compositions can include a pharmaceutically acceptable carrier. The chemical reprogramming factors can also be formulated for sustained release, for example, using microencapsulation. The compositions comprising chemical reprogramming factors can be administered to a patient in a single dose, in multiple doses, in a continuous or intermittent manner to obtain the desired physiological effect, depending on the recipient's physiological conditions. In some embodiments chemical reprogramming factors for stage 1,2, or 3 described herein are formulated for administration, delivery or contacting with a subject, tissue or cell to promote rejuvenation. These chemical reprogramming factors can be formulated to prevent the age-associated histological changes and maintain the cells in a younger state in tissues. The rejuvenating effects can be detected by reversion of the epigenetic clock, or metabolic changes, or transcriptomic changes, such as changes in senescence, stress, or inflammation pathways. The compositions comprising chemical reprogramming factors can be administered to a patient in a single dose, in multiple doses, in a continuous or intermittent manner to obtain the desired physiological effect, depending on the recipient's physiological conditions. In some embodiments chemical reprogramming factors for stage 1,2, or 3 described herein can be formulated for administration, delivery or contacting with a subject, tissue or cell to inhibit or revise the fibrosis. Fibrosis can be detected by the changes of morphology, epigenome, or metabolic changes, or transcriptomic changes induced by the disease, stress, or inflammations. The compositions comprising chemical reprogramming factors can be administered to a patient in a single dose, in multiple doses, in a continuous or intermittent manner to inhibit or revise the fibrosis, depending on the recipient's physiological conditions

### NUMBERED EMBODIMENTS

Embodiment 1. A composition comprising:
a) a glycogen synthase kinase 3 (GSK-3) inhibitor, optionally wherein the GSK-3 inhibitor comprises CHIR99021, CHIR98014, TD114-2, GSK-3 Inhibitor XV, SB-216763 or SB-415286,
b) a transforming growth factor-beta (transforming growth factor-beta (TGFβ)) receptor inhibitor, optionally wherein the transforming growth factor-beta (TGFβ) receptor inhibitor comprises E-616452, A 83-01, SB431542, SB 505124, GW 788388, or SB 525334,
c) a retinoic acid receptor (RAR) agonist, optionally wherein the retinoic acid receptor (RAR) agonist comprises TTNPB, Ch55, or AM580, and
d) one or more of a Akt (protein kinase B) inhibitor or an SETD2 (SET Domain Containing 2) inhibitor, optionally wherein the Akt inhibitor comprises AKT Kinase Inhibitor (AKTi) and optionally wherein the SET domain containing 2 (SETD2) inhibitor comprises SETD2-IN-1, EPZ-719, or MMSET-IN-1.

Embodiment 2. The composition of embodiment 1, further comprising one or more of:
e) a disruptor of telomeric silencing 1-like (Dot1L) inhibitor, optionally wherein the Dot1L inhibitor comprises EPZ004777, EPZ5676 or SGC0946,
f) an agonist for G protein-coupled receptor Smoothened, optionally wherein the agonist for G protein-coupled receptor Smoothened comprises SAG, Purmorphamine, Hh-Ag1.5, or human Sonic Hedgehog (SHH) protein,
g) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
h) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenozine periodate oxidized (Adox), or 3-deazaadenosine (DZA),
i) a Menin-MLL interaction inhibitor, optionally wherein the Menin-MLL interaction inhibitor comprises VTP50469, MI3454, or WDR5-IN-4, or
j) a c-Jun kinase inhibitor, optionally wherein the c-Jun kinase inhibitor comprises JNKIN8, JNKIN7, JNKIN5, or JNKIN12.

Embodiment 3. The composition of embodiment 1, further comprising one or more of a KAT3A/KAT3B inhibitor or a KAT6A inhibitor, optionally wherein the KAT3A/KAT3B inhibitor is A-485, CBP/P300 IN 8, GEN049, CBP/P300 IN 12, SGCCBP30 or ICBP112 and optionally wherein the KAT6A inhibitor is WM-8014 or WM1119.

Embodiment 4. A method for reprogramming cells, comprising the steps of:
(I) contacting a first population of cells comprising the cells with a first composition comprising:
   a) a GSK-3 inhibitor;
   b) a transforming growth factor-beta (TGFβ) receptor inhibitor,
   c) an retinoic acid receptor (RAR) agonist, and
   d) one or more of an Akt inhibitor or an SET domain containing 2 (SETD2) inhibitor;
thereby obtaining a second population of cells.

Embodiment 5. The method of embodiment 4, wherein the second population of cells obtained from step (I) comprises epithelial-like cells that express LIN28A, optionally the epithelial-like cells further express one or more of KRT18, KRT19, WT1, NMYC, WNT2B, PAX8, SMAD3, GLI3, or TBX2.

Embodiment 6. The method of embodiment 4, further comprising:
(II) contacting at least a subset of the second population of cells obtained from step (I) with a second composition, thereby generating a third population of cells, wherein the third population of cells comprises intermediate plastic state cells, optionally the intermediate plastic state cells express LIN28A and SALL4, and one or more of MSX2, NMYC, SDC1, WNT4, FGF19, or TOP2A.

Embodiment 7. The method of embodiment 6, further comprising:
(III) contacting at least a subset of the third population of cells obtained from step (II) with a third composition, thereby generating a fourth population of cells, wherein the fourth population of cells comprises pluripotent stem cells, optionally the pluripotent stem cells are human chemically induced pluripotent stem cells (hCiPSCs).

Embodiment 8. The method of embodiment 4, wherein
a) the GSK-3 inhibitor comprises CHIR99021, CHIR98014, TD114-2, GSK-3 Inhibitor XV, SB-216763 or SB-415286,
b) the transforming growth factor-beta (TGFβ) receptor inhibitor comprises E-616452, A 83-01, SB431542, SB 505124, GW 788388, or SB 525334,
c) the retinoic acid receptor (RAR) agonist comprises TTNPB, Ch55, or AM580,
d) the Akt inhibitor comprises AKT Kinase Inhibitor (AKTi), or the SET domain containing 2 (SETD2) inhibitor comprises SETD2-IN-1, EPZ-719, or MMSET-IN-1.

Embodiment 9. The method of embodiment 4, wherein the first composition further comprises one or more of:
e) a disruptor of telomeric silencing 1-like (Dot1L) inhibitor, optionally wherein the Dot1L inhibitor comprises EPZ004777, EPZ5676 or SGC0946,
f) an agonist for G protein-coupled receptor Smoothened, optionally wherein the agonist for G protein-coupled receptor Smoothened comprises SAG, Purmorphamine, Hh-Ag1.5, or human Sonic Hedgehog (SHH) protein,
g) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
h) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenozine periodate oxidized (Adox), or 3-deazaadenosine (DZA),
i) a Menin-MLL interaction inhibitor, optionally wherein the Menin-MLL interaction inhibitor comprises VTP50469, MI3454, or WDR5-IN-4, or
j) a c-Jun kinase inhibitor, optionally wherein the c-Jun kinase inhibitor comprises JNKIN8, JNKIN7, JNKIN5, or JNKIN12.

Embodiment 10. The method of embodiment 4, wherein the first composition further comprises one or more of a KAT3A/KAT3B inhibitor or a KAT6A inhibitor, optionally wherein the KAT3A/KAT3B inhibitor is A-485, CBP/P300 IN 8, GEN049, CBP/P300 IN 12, SGCCBP30 or ICBP112 and optionally wherein the KAT6A inhibitor is WM-8014 or WM1119.

Embodiment 11. The method of embodiment 6, wherein the second composition comprises
a) a GSK-3 inhibitor, optionally wherein the GSK-3 inhibitor comprises CHIR99021, CHIR98014, TD114-2, GSK-3 Inhibitor XV, SB-216763 or SB-415286,
b) a transforming growth factor-beta (TGFβ) receptor inhibitor, optionally wherein the transforming growth factor-beta (TGFβ) receptor inhibitor comprises E-616452, A 83-01, SB431542, SB 505124, GW 788388, or SB 525334,
c) an retinoic acid receptor (RAR) agonist, optionally wherein the retinoic acid receptor (RAR) agonist comprises TTNPB, Ch55, or AM580,
d) a c-Jun kinase inhibitor, optionally wherein the c-Jun kinase inhibitor comprises JNKIN8, JNKIN7, JNKIN5, or JNKIN12, and
e) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenozine periodate oxidized (Adox), or 3-deazaadenosine (DZA).

Embodiment 12. The method of embodiment 11, wherein the second composition further comprises one or more of:
f) a CBP/p300 bromodomain inhibitor, optionally wherein the CBP/p300 bromodomain inhibitor comprises SGC-CBP30, I-CBP112, or GNE272,
g) an adenosine kinase inhibitor, optionally wherein the adenosine kinase inhibitor comprises 5-Iodotubercidin (5-ITU) or ABT 702,
h) a casein kinase 2 inhibitor, optionally wherein the casein kinase 2 inhibitor comprises CX-4945, TPP22, or Ellagic acid,
i) a Menin-MLL interaction inhibitor, optionally wherein the Menin-MLL interaction inhibitor comprises VTP50469, MI3454, or WDR5-IN-4,
j) an agonist for the G protein-coupled receptor Smoothened, optionally wherein the agonist for the G protein-coupled receptor Smoothened comprises SAG, Purmorphamine, Hh-Ag1.5, or human SHH,
k) a ROCK inhibitor, optionally wherein the ROCK inhibitor comprises Y-27632 or thiazovivin,
l) a BMP receptor/AMPK inhibitor, optionally wherein the BMP receptor/AMPK inhibitor comprises Dorsomorphin,
m) a Dot1L inhibitor, optionally wherein the Dot1L inhibitor comprises EPZ004777, EPZ5676 or SGC0946,
n) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
o) a p38 MAPK inhibitor, optionally wherein the p38 MAPK inhibitor comprises BIRB796, SB203580, or SB202190,
p) an SET domain containing 2 (SETD2) inhibitor, optionally wherein the SET domain containing 2 (SETD2) inhibitor comprises SETD2-IN-1, EPZ-719, or MMSET-IN-1,
q) an Akt inhibitor, optionally wherein the Akt inhibitor comprises AKT Kinase Inhibitor (AKTi),
r) an retinoic acid receptor (RAR) agonist comprising retinoic acid, or
s) a DNA methyltransferase inhibitor comprising GSK-3685032.

Embodiment 13. The method of embodiment 7, wherein the third composition comprises:
a) an MEK inhibitor, optionally wherein the MEK inhibitor comprises PD0325901, AZD8330, or TAK-733,
b) a B-Raf inhibitor, optionally wherein the B-Raf inhibitor comprises SB590885, Vemurafenib, RAF265, or PLX4720, and
c) a histone deacetylase (HDAC) inhibitor, optionally wherein the HDAC inhibitor comprises valproic acid (VPA), LMK235, MS275, or HDACi IV.

Embodiment 14. A second composition comprising:
a) a GSK-3 inhibitor, optionally wherein the GSK-3 inhibitor comprises CHIR99021, CHIR98014, TD114-2, GSK-3 Inhibitor XV, SB-216763 or SB-415286,
b) a transforming growth factor-beta (TGFβ) receptor inhibitor, optionally wherein the transforming growth factor-beta (TGFβ) receptor inhibitor comprises E-616452, A 83-01, SB431542, SB 505124, GW 788388, or SB 525334,
c) an retinoic acid receptor (RAR) agonist, optionally wherein the retinoic acid receptor (RAR) agonist comprises TTNPB, Ch55, or AM580,
d) a c-Jun kinase inhibitor, optionally wherein the c-Jun kinase inhibitor comprises JNKIN8, JNKIN7, JNKIN5, or JNKIN12, and
e) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenozine periodate oxidized (Adox), or 3-deazaadenosine (DZA).

Embodiment 15. The second composition of embodiment 14, wherein the second composition further comprises one or more of:
f) a CBP/p300 bromodomain inhibitor, optionally wherein the CBP/p300 bromodomain inhibitor comprises SGC-CBP30, I-CBP112, or GNE272,
g) an adenosine kinase inhibitor, optionally wherein the adenosine kinase inhibitor comprises 5-Iodotubercidin (5-ITU) or ABT 702,
h) a casein kinase 2 inhibitor, optionally wherein the casein kinase 2 inhibitor comprises CX-4945, TPP22, or Ellagic acid,
i) a Menin-MLL interaction inhibitor, optionally wherein the Menin-MLL interaction inhibitor comprises VTP50469, MI3454, or WDR5-IN-4,
j) an agonist for the G protein-coupled receptor Smoothened, optionally wherein the agonist for the G protein-coupled receptor Smoothened comprises SAG, Purmorphamine, Hh-Ag1.5, or human SHH,
k) a ROCK inhibitor, optionally wherein the ROCK inhibitor comprises Y-27632 or thiazovivin,
l) a BMP receptor/AMPK inhibitor, optionally wherein the BMP receptor/AMPK inhibitor comprises Dorsomorphin,
m) a Dot1L inhibitor, optionally wherein the Dot1L inhibitor comprises EPZ004777, EPZ5676, or SGC0946,
n) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
o) a p38 MAPK inhibitor, optionally wherein the p38 MAPK inhibitor comprises BIRB796, SB203580, or SB202190,
p) an SET domain containing 2 (SETD2) inhibitor, optionally wherein the SET domain containing 2 (SETD2) inhibitor comprises SETD2-IN-1, EPZ-719, or MMSET-IN-1,
q) an Akt inhibitor, optionally wherein the Akt inhibitor comprises AKT Kinase Inhibitor (AKTi),
r) an retinoic acid receptor (RAR) agonist comprising retinoic acid, or
s) a DNA methyltransferase inhibitor comprising GSK-3685032.

Embodiment 16. A third composition comprising:
a) an MEK inhibitor, optionally wherein the MEK inhibitor comprises PD0325901, AZD8330, or TAK-733,
b) a B-Raf inhibitor, optionally wherein the B-Raf inhibitor comprises SB590885, Vemurafenib, RAF265, or PLX4720, and
c) a histone deacetylase (HDAC) inhibitor, optionally wherein the HDAC inhibitor comprises valproic acid (VPA), LMK235, MS275, or HDACi IV.

Embodiment 17. The third composition of embodiment 16, wherein the third composition further comprises one or more of:
d) a Wnt inhibitor, optionally wherein the Wnt inhibitor comprises IWR-1 or IWP-2,
e) a GSK-3 inhibitor, optionally wherein the GSK-3 inhibitor comprises CHIR99021, CHIR98014, TD114-2, GSK-3 Inhibitor XV, SB-216763 or SB-415286,
f) a ROCK inhibitor, optionally wherein the ROCK inhibitor comprises Y-27632 or thiazovivin,
g) an inhibitor of histone demethylation, optionally wherein the inhibitor of histone demethylation comprises Tranylcypromine,
h) a Dot1L inhibitor, optionally wherein the Dot1L inhibitor comprises EPZ004777 or EPZ5676, or
i) a S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenozine periodate oxidized (Adox), or 3-deazaadenosine (DZA).
j) A specific activator of YAP transcriptional activity that targets Annexin A2 (ANXA2) comprises PY-60,

Embodiment 18. An isolated population of cells comprising intermediate plastic state cells that express:
a) LIN28A and SALL4;
b) one or more of MSX2, NMYC, WNT4, FGF19, or TOP2A;
c) one or more of MSX1, HOXB9, WT1, GATA2, HMGA2, or LEF1 and
d) one or more of FGF9, HOXA9, HOXA1, PTCH1, HOXA5, CCND2, SDC1, TBX3, BMP4, or IGF2.

Embodiment 19. An isolated population of cells comprising epithelial-like cells that express:
a) LIN28A,
b) one or more of NMYC, WNT2B, PAX8, SMAD3, or GLI3, and
c) one or more of KRT18, KRT19, WT1, or TBX2.

Embodiment 20. A composition comprising:
a) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
b) a transforming growth factor-beta (TGFβ) receptor inhibitor, optionally wherein the transforming growth factor-beta (TGFβ) receptor inhibitor comprises E-616452, A 83-01, SB431542, SB 505124, GW 788388, or SB 525334,
c) c) a retinoic acid receptor (RAR) agonist, optionally wherein the retinoic acid receptor (RAR) agonist comprises TTNPB, Ch55, or AM580, and
d) one or more of a Akt (protein kinase B) inhibitor or an SETD2 (SET Domain Containing 2) inhibitor, optionally wherein the Akt inhibitor comprises AKT Kinase Inhibitor (AKTi) and optionally wherein the SET domain containing 2 (SETD2) inhibitor comprises SETD2-IN-1, EPZ-719, or MMSET-IN-1.

Embodiment 21. A second composition comprising:
a) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
b) a transforming growth factor-beta (TGFβ) receptor inhibitor, optionally wherein the transforming growth factor-beta (TGFβ) receptor inhibitor comprises E-616452, A 83-01, SB431542, SB 505124, GW 788388, or SB 525334, and
c) a retinoic acid receptor (RAR) agonist, optionally wherein the retinoic acid receptor (RAR) agonist comprises TTNPB, Ch55, or AM580.

Embodiment 22. A third composition comprising:
a) an MEK inhibitor, optionally wherein the MEK inhibitor comprises PD0325901, AZD8330, or TAK-733, and
b) a histone deacetylase (HDAC) inhibitor, optionally wherein the HDAC inhibitor comprises valproic acid (VPA), LMK235, MS275, or HDACi IV.

Embodiment 23. A method for reprogramming cells, comprising the steps of:
(I) contacting a first population of cells comprising the cells with the composition of embodiment 20 thereby generating epithelial-like cells that express LIN28, optionally the epithelial-like cells further express one or more of KRT18, KRT19, WT1, NMYC, WNT2B, PAX8, SMAD3, GLI3, or TBX2.

Embodiment 24. The method of embodiment 23, further comprising:
(II) contacting at least a subset of the second population of cells obtained from step (I) with the second composition of embodiment 21 thereby generating a third population of cells, wherein the third population of cells comprises intermediate plastic state cells that express LIN28A and SALL4, and one or more of MSX2, NMYC, SDC1, WNT4, FGF19, or TOP2A.

Embodiment 25. The method of embodiment 24, further comprising:
(III) contacting at least a subset of the third population of cells obtained from step (II) with the third composition of embodiment 22, thereby generating a fourth population of cells, wherein the fourth population of cells comprises pluripotent stem cells that express OCT4, SOX2, NANOG, FGF4, ZFP57, REX1, DPPA4, TDGF1, TRA-1-60, TRA-1-81, SSEA4, KLF4, KLF17, DPPA3, DPPA5, DNMT3L, REX1, UTF1.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Methods to Convert Cells into Pluripotent Stem Cells

This example illustrates methods for converting human somatic cells into pluripotent stem cells according to some embodiments of the present disclosure.

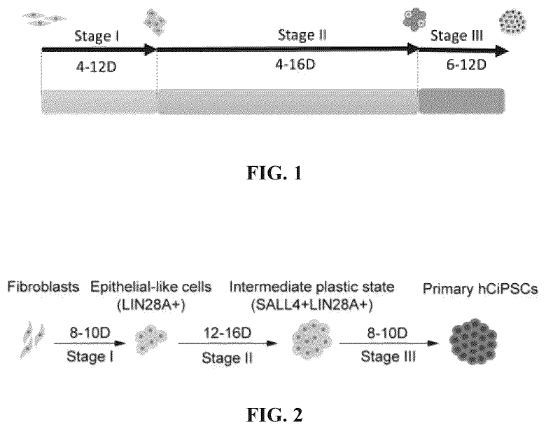

FIG. 1 illustrates a schematic of an exemplary method for converting human somatic cells into pluripotent stem cells. The method has 3 stages. During any of the 3 stages, any cell can proliferate and give rise to progeny cells. In stage 1, a first population of cells comprising somatic cells are contacted with a first composition for about 4-12 days. The first population of cells can comprise somatic cells (e.g., fibroblasts or primary human adult adipose derived mesenchymal stromal cells (hADSCs)). During and/or subsequent to contacting the first composition, at least a subset of the cells of the first population of cells or the progenies thereof are converted into a second population of cells. The second population of cells can comprise epithelial-like cells. In stage 2, the second population of cells or the progenies thereof are then contacted with a second composition for about 4-16 days. During and/or subsequent to contacting the second composition, at least a subset of the cells of the second population of cells or the progenies thereof are converted into a third population of cells. The third population of cells can comprise intermediate plastic state cells. In stage 3, the third population of cells or the progenies thereof are contacted with a third composition for about 6-12 days. During and/or subsequent to contacting the third composition, at least a subset of the cells of the third population of cells or the progenies thereof are converted into a fourth population of cells. The fourth population of cells can comprise pluripotent stem cells (e.g., human chemically induced pluripotent stem cells (hCiPSCs).

FIG. 2 illustrates a schematic of an exemplary method for converting human fibroblasts into pluripotent stem cells. The method can comprise 3 stages. During any of the 3 stages, any cell can proliferate and give rise to progeny cells. In stage 1, a first population of cells comprising fibroblasts is contacted with a first composition for about 8-10 days. During and/or subsequent to contacting the first composition, at least a subset of the cells of the first population of cells or the progenies thereof are converted into a second population of cells. The second population of cells can comprise epithelial-like cells. The epithelial-like cells express an increased amount of LIN28A, relative to the first population of cells or their progenies. In stage 2, the second population of cells or the progenies thereof are then contacted with a second composition for about 12-16 days. During and/or subsequent to contacting the second composition, at least a subset of the cells of the second population of cells or the progenies thereof are converted into a third population of cells. The third population of cells can comprise intermediate plastic state cells. The intermediate plastic state cells express both SALL4 and LIN28A. In stage 3, the third population of cells or the progenies thereof are contacted with a third composition for about 8-10 days. During and/or subsequent to contacting the third composition, at least a subset of the cells of the third population of cells or the progenies thereof are converted into a fourth population of cells. The fourth population of cells can comprise pluripotent stem cells (e.g., human chemically induced pluripotent stem cells (hCiPSCs). For stage 3 conversion, in some cases-when the stage 3 conversion medium comprises a histone deacetylase inhibitor, a S-adenosyl-L-homocysteine (SAH) hydrolase inhibitor, and a Dot1L inhibitor—the cells may first be contacted with the conversion medium for a first period of time. The conversion medium can then be removed from the cells. The cells can then be contacted with a second stage 3 conversion medium that comprises about 50% of the histone deacetylase inhibitor and without the S-adenosyl-L-homocysteine (SAH) hydrolase inhibitor and the Dot1L inhibitor for a second period of time. The second conversion medium can then be removed from the cells. The cells can then be contacted with a third stage 3 medium that does not comprise the histone deacetylase inhibitor, the S-adenosyl-L-homocysteine (SAH) hydrolase inhibitor, and the Dot1L inhibitor for a third period of time. The third stage 3 medium may allow the pluripotent stem cells or the progenies thereof to proliferate.

In one experiment, when converting hCiPSCs from somatic cell (e.g., hADSCs), hypoxia with 5% O₂ was used in stage 1 conversion. After stage 1 conversion, cells were incubated with 21% O₂. The induction medium was changed every 3-4 days. hADSCs were seeded at a density of 1×10\hat{ }4 cells per well of a 12-well plate in 15% FBS-DMEM medium and would be changed into stage 1 conversion medium the next day. For stage 1 conversion, single layer epithelial-like cells induced from hADSCs would emerge at day 4-6 and approach 100% confluence at day 8-12. Then the medium was changed into stage 2 conversion medium. For stage 2 conversion, multi-layered cell colonies appeared after 8-12 days of treatment of the cells with the conversion medium, and these cell colonies would continually grow increasingly large. The medium was changed into stage 3 conversion medium after 16 days of treatment with the stage 2 conversion medium. During the stage 3 conversion, VPA (1000 μM), Tranylcypromine (10 μM), DZNep (0.2 μM), and EPZ5676 (2 μM) were included in the stage 3 conversion medium in the first 4 days. Subsequently, the cells were contacted with a second stage 3 conversion medium with VPA (500 μM) included in the next 4 days while Tranylcypromine, DZNep, and EPZ5676 were removed. The stage 3 induction medium without VPA, Tranylcypromine, DZNep, or EPZ5676 could be applied for additional 2-4 days to allow the primary hCiPSC colonies to grow larger subsequent to the removal of the second stage 3 conversion medium.

### Example 2: Methods for Culturing and Analysis of Cells

Provided in this example are methods for culturing and analyzing the cells according to some embodiments of the present disclosure.

### Cell Culture

For culturing Primary human adult adipose derived mesenchymal stromal cells (hADSCs) and human hCiPS cells and human ES cells (H1 and H9) were cultured using the methods described in Nature, 2022, which is herein incorporated by reference in its entirety. Briefly, primary hADSCs were seeded at a density of 1.5×10\hat{ }6 cells per 100-mm dish and cultured in Mesenchymal Stem Cell Growth Medium 2 under 21% 02, 5% CO₂ at 37° C. The medium was changed every 2 days and cells were passaged by 0.25% Trypsin-EDTA before reached confluence. Primary hADSCs within 4 passages can be used for the induction of hCiPS cells. Human CiPS cells and human ES cells (H1 and H9) were maintained in mTeSR™ Plus Medium on Matrigel-coated plates (Corning, 354248) under 21% 02, 5% CO₂ at 37° C. The medium was changed every day and cells were passaged by ReLeSR™ with split ratios of around 1:10 to 1:20 when they reached ˜85% confluence. For passaging, detached cell aggregates were plated in mTeSR™ Plus Medium supplemented with Y-27632 (10 μM). After 24 hours, the medium was replaced with fresh mTeSR™ Plus Medium without Y-2:7632.

Isolation and Culture of hADSCs

hADSCs were isolated from adult adipose tissue that was obtained with informed written consent and approval by the Institute of Ethics Committee Review Board in Peking University (IRB 00001052-19070). The procedure was conducted according to the principles of the Declaration of Helsinki. Briefly: Tissues were dissociated by collagenase IV and the obtained cells were plated in a 100-mm dish in Mesenchymal Stem Cell Growth Medium 2 followed by incubation in 21% O₂, 5% CO₂ at 37° C. For hCiPSCs induction, hADSCs were seeded at a density of 1×10\hat{ }4 cells per well of a 12-well plate with 15% FBS-DMEM medium the day before stage 1 induction.

### Derivation and Culture of Human CiPS Cell Lines

Cells were dissociated by Accutase (Millipore, SCR005) after 8-12 days' induction of stage 3 condition, centrifuged at 400 g for 3 min to remove Accutase, and then re-plated at a ratio from 1:3 to 1:12 on feeder layers in the modified stage 3 medium: Knockout DMEM supplemented with 1% N2 supplement, 2% B27 supplement, 1% GlutaMAX™, 1% NEAA, 1% Penicillin-Streptomycin, 50 μg/ml Vc2p, 2 mg/mL AlbuMAX™_II and the small molecules CHIR99021 (1 μM), PD0325901 (0.5 μM), IWP-2 (2 μM), Y-27632 (10 μM), HRG (20 ng/mL), and bFGF (100 ng/mL, Origene).

### Immunofluorescence

After fixation with 4% paraformaldehyde at room temperature for 30 min, cells were blocked by PBS containing 0.1% Triton™ X-100 and 2% donkey serum at 37° C. for 1 hour. Primary antibodies incubation with appropriate dilutions were performed at 4° C. overnight in the same buffer. The secondary antibodies were incubated in PBS containing 2% donkey serum at 4° C. overnight. DNA was stained with DAPI solution. For surface marker analysis, PE Anti-Syndecan-1 (CD138) antibodies (Cat #552026; Cat #ab209584) were used in live-cell immunostaining. The antibodies (1:200) were added to the growth medium and then incubated for 2 hours. The cells were gently washed for three times with prewarmed PBS. Then the cells were fixed in situ for staining other antibodies.

### Flow Cytometry and Cell Sorting

Cells were dissociated by using 0.25% Trypsin-EDTA and then the cell suspensions were filtrated through 40 μm cell strainers. FACS analyses were performed with the BD Cytofix/Cytopenm™ Fixation/Permeablization Kit (BD Biosciences) according to the manufacturer's instructions. Flow cytometry was performed using CytoFlEX S (BeckMan Coulter). A list of antibodies for the method described herein (e.g., immunofluorescence or flow cytometry/cell sorting) can be found in TABLE 1 below.

TABLE 1
List of antibodies for methods described herein

| REAGENT or RESOURCE | SOURCE | IDENTIFIER |
|---|---|---|
| Rabbit monoclonal anti- | Abcam | Cat# ab124765; |
| Lin28A | | RRID: AB_10975201 |
| Mouse monoclonal anti- | Abcam | Cat# ab57577; |
| SALL4 | | RRID: AB_2183366 |
| Mouse monoclonal anti- | BD | Cat# 611203; |
| OCT4 | Biosciences | RRID: AB_398737 |
| Rabbit monoclonal anti- | Invitrogen | Cat# MA5-14845; |
| OCT4 | | RRID: AB_10979606 |
| Goat polyclonal anti- | R&D | Cat# AF2018; |
| SOX2 | | RRID: AB_355110 |
| Rabbit polyclonal anti- | Abcam | Cat# ab21624; |
| NANOG | | RRID: AB_446437 |
| Mouse monoclonal anti- | Millipore | Cat# MAB4360; |
| TRA-1-60 | | RRID: AB_2119183 |
| Mouse monoclonal anti- | Millipore | Cat# MAB4381; |
| TRA-1-81 | | RRID: AB_177638 |
| Mouse polyclonal anti- | Santa Cruz | Cat# sc-21704; |
| SSEA-4 | | RRID: AB_628289 |
| Anti-Histone H3 (tri | Abcam | Cat# ab6002; |
| methyl K27) | | RRID: AB_305237 |
| Anti-Histone H3 (acetyl | Abcam | Cat# ab4729; |
| K27) | | RRID: AB_2118291 |
| Anti-Histone H3 (mono | Abcam | Cat# ab8895; |
| methyl K4) | | RRID: AB_306847 |
| Anti-Histone H3 (tri | Abcam | Cat# ab8580; |
| methyl K4) | | RRID: AB_306649 |
| Alexa Fluor 488-AffiniPure | Jackson | Cat# 715-545-150; |
| Donkey Anti-Mouse IgG | Immuno | RRID: AB_2340846 |
| (H + L) | Research | |
| Alexa Fluor 488-AffiniPure | Jackson | Cat# 711-545-152; |
| Donkey Anti-Rabbit IgG | Immuno | RRID: AB_2313584 |
| (H + L) | Research | |
| Alexa Fluor 488-AffiniPure | Jackson | Cat# 705-545-147; |
| Donkey Anti-Goat IgG | Immuno | RRID: AB_2336933 |
| (H + L) | Research | |
| Cy3-AffiniPure Donkey | Jackson | Cat# 711-165-152; |
| Anti-Rabbit IgG (H + L) | Immuno | RRID: AB_2307443 |
| | Research | |
| Cy3-AffiniPure Donkey | Jackson | Cat# 715-545-150; |
| Anti-Mouse IgG (H + L) | Immuno | RRID: AB_2340846 |
| | Research | |
| Cy3-AffiniPure Donkey | Jackson | Cat# 705-165-147; |
| Anti-Goat IgG (H + L) | Immuno | RRID: AB_2307351 |
| | Research | |
| Alexa Fluor 647 Donkey | Jackson | Cat# 705-605-147; |
| Anti-Goat IgG (H + L) | Immuno | RRID: AB_2340437 |
| | Research | |
| Alexa Fluor 647 Donkey | Jackson | Cat# 715-605-150; |
| Anti-Mouse IgG (H + L) | Immuno | RRID: AB_2340862 |
| | Research | |
| Alexa Fluor 647 Donkey | Jackson | Cat# 711-605-152; |
| Anti-Rabbit IgG (H + L) | Immuno | RRID: AB_2340624 |
| | Research | |
| Mouse monoclonal Anti- | BD | Cat# 552026; |
| Human CD138 | Biosciences | RRID: AB_394323 |
| Rabbit monoclonal Anti- | Abcam | Cat# ab209584; |
| Syndecan-1 (CD138) | | |
| Rabbit monoclonal anti | Cell Signaling | Cat# 27967 |
| HOXB9 | Technology | |
| Rabbit Polyclonal Anti- | Invitrogen | PA5-105270; |
| NMYC | | RRID: AB_2853875 |
| Rabbit polyclonal Anti- | Abcam | Cat# ab29112; |
| SALL4 | | RRID: AB_777810 |
| Valproic acid sodium salt | Sigma- | Cat#P4543 |
| (VPA) | Aldrich | |
| CHIR99021 (CHIR, C) | WUXI APPTEC | N/A |
| 616452 (6) | WUXI APPTEC | N/A |
| Tranylcypromine (Tranyl, T) | Enzo | Cat#BML-EI217-0005 |
| 3-deazaneplanocin A | WUXI APPTEC | N/A |
| (DZNep) | | |
| PD0325901 | WUXI APPTEC | N/A |
| EPZ5676 | MCE | Cat# HY-15593 |
| TTNPB (N) | WUXI APPTEC | N/A |
| Y-27632 | WUXI APPTEC | N/A |
| SAG | WUXI APPTEC | N/A |
| Recombinant Human FGF2 | Origene | Cat# TP750002 |
| JNKIN8 (J) | Selleckchem | Cat# S4901 |
| Recombinant Human | PEPROTECH | Cat# 100-03 |
| Heregulin β-1 (HRG) | | |
| IWP-2 | Selleckchem | Cat# S7085 |
| SB590885 | Selleckchem | Cat# S2220 |
| Ruxolitinib (Ruxo) | Selleckchem | Cat# S1378 |
| BIRB796 (BIRB) | Selleckchem | Cat# S1574 |
| SGC-CBP30 (CBP30) | Selleckchem | Cat# S7256 |
| Dorsomorphin (DM) | MCE | Cat# HY-13418A |
| VTP50469 | Selleckchem | Cat# S8934 |
| L-Ascorbic acid 2- | Sigma-Aldrich | Cat# A8960 |
| phosphate (Vc2P) | | |
| LiCl | Sigma-Aldrich | Cat# L4408 |
| Nicotinamide (NAM) | Sigma-Aldrich | Cat# 72340 |
| AKT Kinase Inhibitor | MCE | Cat# HY-10249A |
| 5-Iodotubercidin (5ITU) | MCE | Cat# HY-15424 |
| Recombinant Human TNF-α | PEPROTECH | Cat#300-01A |
| DAPI | Roche | Cat# 10236276001 |
| 4% paraformaldehyde | DingGuo | Cat# AR-0211 |
| Normal donkey serum | Jackson | Cat# 017-000-121 |
| | Immuno | |
| | Research | |
| Triton ™ X-100 | Sigma-Aldrich | Cat# 9036-19-5 |
| PBS | CORNING | Cat# 05418005 |
| DMSO (Dimethyl sulfoxide) | Sigma-Aldrich | Cat# D2650 |
| Trizol (TRI) Reagent | Sigma-Aldrich | Cat# T9424 |
| DMEM | Gibco | Cat# C11965500BT |
| KnockOut ™ DMEM | Gibco | Cat# 10829018 |
| N2 supplement | Gibco | Cat# 17502-048 |
| B27 supplement | Gibco | Cat# 17504-044 |
| Fetal Bovine Serum (FBS) | Vistech | Cat# VIS93526487 |
| Knockout Serum | Gibco | Cat# 10828028 |
| Replacement (KSR) | | |
| Penicillin-Streptomycin | Gibco | Cat# 15140-122 |
| Collagenase IV | Gibco | Cat# 1963347 |
| 0.25% Trypsin-EDTA | Gibco | Cat# 25200-056 |
| GlutaMAX ™ | Gibco | Cat# 35050-061 |
| MEM Non-Essential Amino | Gibco | Cat# 11140050 |
| Acids Solution (NEAA) | | |
| Mesenchymal Stem Cell | PromoCell | Cat# C-28009 |
| Growth Medium 2 | | |
| AlbuMAX ™-II | Gibco | Cat# 11021-045 |
| Matrigel | Corning | Cat# 354248 |
| mTeSR ™ Plus Medium | STEMCELL | Cat# 100-0276 |
| Accutase | Millipore | Cat# SCR005 |
| ReLeSR ™ | STEMCELL | Cat# 05872 |
| Critical Commercial Assays | | |
| Cytofix/Cytoperm ™ | BD | Cat# 554714 |
| Fixation/Permeablization | Biosciences | |
| Kit | | |
| Direct-zol RNA MiniPrep Kit | Zymo Research | Cat# R2053 |
| TransScript First-Strand | TransGen | Cat# AT311-03 |
| cDNA Synthesis SuperMix | Biotech | |
| KAPA SYBR FAST qPCR | KAPA | Cat# KM4101 |
| Kit Master Mix | Biosystems | |
| Direct-zol RNA MiniPrep Kit | Zymo Research | Cat# R2072 |
| Hyperactive Universal | Vazyme | Cat# TD903-02 |
| CUT&Tag Assay Kit for | | |
| Illumina | | |
| QIAGEN Plasmid Plus Midi | QIAGEN | Cat. No./ID: 12945 |
| Kits | | |

### Teratoma Formation

CiPS cells were harvested by ReLeSR™ (STEMCELL, Cat #05872). Approximately 2×10\hat{ }6 cells were resuspended in Matrigel and then sub-cutaneously injected to the immunodeficient NPG mice. After 6-7 weeks, the teratomas were obtained and then embedded in paraffin. The paraffin sections were stained with haematoxylin and eosin. All of the mouse experiments were approved by the Institutional Animal Care and Use Committee of Peking University.

Reverse Transcription-Quantitative PCR (qRT-PCR)

Total RNA was isolated using Direct-zol RNA MiniPrep Kit (Zymo Research, R2053). cDNA was synthesized from 0.5-1 μg of total RNA using TransScript First-Strand cDNA Synthesis SuperMix (TransGen Biotech, AT311-03). qPCR was performed by using KAPA SYBR FAST qPCR Kit Master Mix (KAPA Biosystems, KM4101) on a CFX Connect™ Real-Time System (Bio-Rad). The data were analyzed using the delta-delta Ct method. GAPDH was used as a control to normalize the expression of target genes. Primer sequences for qPCR in this study are listed in TABLE 2 below.

TABLE 2
Primers used for qRT-PCR

| Gene | Forward sequence (5′→3′) | Reverse sequence (5′→3′) |
|---|---|---|
| GAPDH | TGACATCAAGAAGGTGGTGAA | GCGTCAAAGGTGGAGGAGTGG |
| | GCAGG (SEQ ID NO: 1) | GT (SEQ ID NO: 2) |
| OCT4 | CTGGGTTGATCCTCGGACCT | CCATCGGAGTTGCTCTCCA |
| | (SEQ ID NO: 3) | (SEQ ID NO: 4) |
| NANOG | GATTTGTGGGCCTGAAGAAA | CAGATCCATGGAGGAAGGAA |
| | (SEQ ID NO: 5) | (SEQ ID NO: 6) |
| SOX2 | TTGCGTGAGTGTGGATGGGATT | GGGAAATGGGAGGGGTGCAA |
| | GGTG (SEQ ID NO: 7) | AAGAGG (SEQ ID NO: 8) |
| DNMT3B | CGAAGAAGAGCCGGCCTGTAC | CGGAAGCCCATGCAACGATCT |
| | C (SEQ ID NO: 9) | C (SEQ ID NO: 10) |
| DPPA4 | GCTAACATCTGCCACCCCACCA | GGATTCTGCGGTGCTGCTGAC |
| | (SEQ ID NO: 11) | A (SEQ ID NO: 12) |
| UTF1 | CGCCGCTACAAGTTCCTTAAA | GGATCTGCTCGTCGAAGGG |
| | (SEQ ID NO: 13) | (SEQ ID NO: 14) |
| ZFP42 | AGAAACGGGCAAAGACAAGAC | GCTGACAGGTTCTATTTCCGC |
| | (SEQ ID NO: 15) | (SEQ ID NO: 16) |
| PRDM14 | TTGAGGAAGAGAATCAGATCC | CGTTCTGTACGGGGTCACTC |
| | AG (SEQ ID NO: 17) | (SEQ ID NO: 18) |
| ZIC3 | CAGGAGCTGTCGTGCAAGT | AGTAGCAGACGTGGTTGTTCT |
| | (SEQ ID NO: 19) | (SEQ ID NO: 20) |
| LIN28A | TTTCCCTCATTCCTGAACTGC | CAGCAAAATCAACCATCAAAT |
| | (SEQ ID NO: 21) | AAAC (SEQ ID NO: 22) |
| SALL4 | AGCACATCAACTCGGAGGAG | CATTCCCTGGGTGGTTCACTG |
| | (SEQ ID NO: 23) | (SEQ ID NO: 24) |
| KRT8 | CAGAAGTCCTACAAGGTGTCCA | CTCTGGTTGACCGTAACTGCG |
| | (SEQ ID NO: 25) | (SEQ ID NO: 26) |
| KRT18 | TCGCAAATACTGTGGACAATGC | GCAGTCGTGTGATATTGGTGT |
| | (SEQ ID NO: 27) | (SEQ ID NO: 28) |
| KRT19 | ACCAAGTTTGAGACGGAACAG | CCCTCAGCGTACTGATTTCCT |
| | (SEQ ID NO: 29) | (SEQ ID NO: 30) |
| SOX17 | AGGAAATCCTCAGACTCCTGGG | CCCAAACTGTTCAAGTGGCAG |
| | TT (SEQ ID NO: 31) | ACA (SEQ ID NO: 32) |
| APOAI | CCCTGGGATCGAGTGAAGGA | CTGGGACACATAGTCTCTGCC |
| | (SEQ ID NO: 33) | (SEQ ID NO: 34) |
| APOA2 | CTGTGCTACTCCTCACCATCT | CTCTCCACACATGGCTCCTTT |
| | (SEQ ID NO: 35) | (SEQ ID NO: 36) |
| FOXA2 | CCTACGAACAGGTGATGCACT | GATTTCTTCTCCCTTGCGTCT |
| | (SEQ ID NO: 37) | (SEQ ID NO: 38) |
| GATA6 | CTCAGTTCCTACGCTTCGCAT | GTCGAGGTCAGTGAACAGCA |
| | (SEQ ID NO: 39) | (SEQ ID NO: 40) |
| HNF1B | GCACCTCTCCCAGCATCTCA | GTCGGAGGATCTCTCGTTGC |
| | (SEQ ID NO: 41) | (SEQ ID NO: 42) |
| MMP1 | AAAATTACACGCCAGATTTGCC | GGTGTGACATTACTCCAGAGT |
| | (SEQ ID NO: 43) | TG (SEQ ID NO: 44) |
| PRRX1 | AAGTAGCCATGGCGCTGTA | GAGACGTGACTGCTGTGGAG |
| | (SEQ ID NO: 45) | (SEQ ID NO: 46) |
| SNAI2 | CTGAGGATCTCTGGTTGTGGT | CGAACTGGACACACATACAGT |
| | (SEQ ID NO: 47) | G (SEQ ID NO: 48) |
| TWIST1 | CGGGAGTCCGCAGTCTTA | GCTTGAGGGTCTGAATCTTG |
| | (SEQ ID NO: 50) | (SEQ ID NO: 51) |
| TWIST2 | CTTATGTTTGGGGGGAGGTT | TAGCCAAGCAATCACGGAGA |
| | (SEQ ID NO: 52) | (SEQ ID NO: 53) |
| VIM | CTTTGTCGTTGGTTAGCTGGT | GACGCCATCAACACCGAGTT |
| | (SEQ ID NO: 54) | (SEQ ID NO: 55) |
| ZEB1 | ATCAGCCTAACCATACAACTCT | CAGGAACAAATTGGCACAA |
| | (SEQ ID NO: 56) | (SEQ ID NO: 57) |
| ZEB2 | GGGTTCTTTCATTTGTTTTGGT | ACTCCAAACAGCTTCTCTTCTG |
| | (SEQ ID NO: 58) | A (SEQ ID NO: 59) |
| COL1A1 | CAGATCACGTCATCGCACAAC | GAGGGCCAAGACGAAGACATC |
| | (SEQ ID NO: 60) | (SEQ ID NO: 61) |
| COL5A1 | GCCCGGATGTCGCTTACAG | AAATGCAGACGCAGGGTACAG |
| | (SEQ ID NO: 62) | (SEQ ID NO: 63) |
| COL6A2 | GACTCCACCGAGATCGACCA | CTTGTAGCACTCTCCGTAGGC |
| | (SEQ ID NO: 64) | (SEQ ID NO: 65) |
| ALB | GCACAGAATCCTTGGTGAACAG | ATGGAAGGTGAATGTTTCAGC |
| | (SEQ ID NO: 66) | A (SEQ ID NO: 67) |
| ASL | CAGTGGACCCCATCATGGAGA | GGCTTTGCTGCCTTGAACATC |
| | (SEQ ID NO: 68) | (SEQ ID NO: 69) |
| ASS | CTTGGGGCCAAAAAGGTGTTC | GAGGTAGCGGTCCTCATACAG |
| | (SEQ ID NO: 70) | (SEQ ID NO: 71) |
| CPS1 | AATGAGGTGGGCTTAAAGCAA | AGTTCCACTCCACAGTTCAGA |
| | G (SEQ ID NO: 72) | (SEQ ID NO: 73) |
| OTC | CGGCCCGTGTATTGTCTAGC | TAGCCAGGGTGTCCAAATCTG |
| | (SEQ ID NO: 74) | (SEQ ID NO: 75) |
| CYP3A4 | GGTGGTGAATGAAACGCTCAG | ACCCCTTTGGGAATGAACATC |
| | (SEQ ID NO: 76) | (SEQ ID NO: 77) |
| F10 | CACTGGTCGCCATCTTTGTA | AGTGCATGGAAGAGACCTGC |
| | (SEQ ID NO: 78) | (SEQ ID NO: 79) |
| FROX1 | ACAGGGCTCTGAACATGCAC | GGCATTGAAAAACTCCCGTA |
| | (SEQ ID NO: 80) | (SEQ ID NO: 81) |
| HNF4A | ACTACATCAACGACCGCCAGT | ATCTGCTCGATCATCTGCCAG |
| | (SEQ ID NO: 82) | (SEQ ID NO: 83) |

### RNA Sequencing

Total RNA was isolated by using Direct-zol RNA MiniPrep Kit. The NEBNext Ultra RNA Library Prep Kit (NEB England BioLabs, E7775) was used for RNA sequencing library construction. Fragmented and randomly primed 2×150 bp paired-end libraries were sequenced by using the Illumina HiSeq X Ten system.

Single-Cell RNA Sequencing (scRNA-Seq)

Briefly, cells at different time points were harvested and resuspended at 1×10\hat{ }6 cells per milliliter in 1×PBS with 0.04% BSA. Then, the cells were loaded onto the Chromium single cell controller (10× Genomics) to generate single-cell gel beads in the emulsion according to the manufacturer's protocol. scRNA-seq libraries were constructed using the Single Cell 3′ Library and Gel Bead Kit V3.1 (10× Genomics, 1000075). The libraries were finally sequenced using the Illumina NovaSeq 6000 sequencer (performed by CapitalBio Technology).

### Whole-Genome Bisulfite Sequencing (WGBS)

Bisulfite conversion of the extracted genome DNA was performed as previously reported in Guan et al. 2022 (Nature. 2022 May; 605(7909):325-331), which is herein incorporated by reference in its entirety. The recovered bisulfite-converted DNAs were constructed into sequencing libraries. For each library, 90 gigabases (Gb) raw data was obtained by Illumina HiSeq X Ten sequencing system.

### Example 3: Stage 1 Conversion of Cells

Provided in this example are methods for stage 1 conversion of cells according to some embodiments of the present disclosure.

In stage 1, a first population of cells (e.g., somatic cells) can be converted to a second population of cells (e.g., epithelial-like cells) using a stage 1 conversion medium. The stage 1 conversion medium may comprise a glycogen synthase kinase 3 (GSK-3), a transforming growth factor-beta (TGFβ) receptor inhibitor, a retinoic acid receptor (RAR) agonist, or a serine-threonine kinase Akt inhibitor, or any combination thereof. The stage 1 conversion medium may additionally comprise a coiled-coil containing protein kinase (ROCK) inhibitor. In some cases, the stage 1 conversion medium may further comprise a Dot1L inhibitor, an agonist for the G protein-coupled receptor Smoothened, a Jak1/Jak2 inhibitor, S-adenosyl-L-homocysteine (SAH) hydrolase inhibitor, a Menin-MLL interaction inhibitor, or a SET domain containing 2 (SETD2) inhibitor, or any combination thereof.

For example, in the stage 1 conversion, hADSCs were contacted with a stage 1 conversion medium: Knockout™ DMEM supplemented with 1% N2 supplement, 2% B27 supplement, 1% GlutaMAX™, 1% NEAA, 1% Penicillin-Streptomycin, 50 μg/mlVc2p, 5 mM LiCl, 1 mM NAM, 20 ng/mL BMP4 (StemImmune LLC, HST-B4-0100), 2 mg/mL AlbuMAX™-II (Gibco, 11021045) and the small molecules CHIR999021 (5 μM), 616452 (10 μM), TTNPB (2 μM), SAG (0.5 μM), EPZ5676 (2 μM), DZNep (0.02 uM), Ruxolitinib (1 μM), VTP50469 (0.5 μM), and AKT Kinase Inhibitor (1 μM). AlbuMAX™-II could be replaced by 2% Knockout Serum Replacement (KSR).

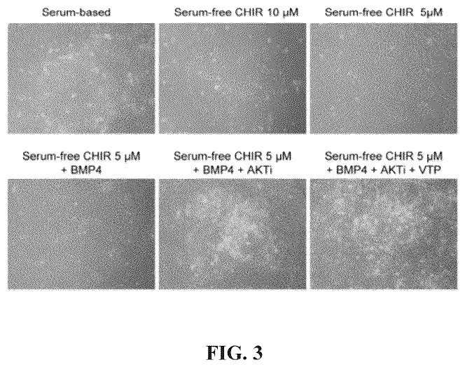

Various combinations of growth factors and chemical reprogramming factors for stage 1 conversion were tested. When converting cells in Stage 1, serum in the cell culture medium was replaced with N2B27 nutrient supplement and BMP4, a combination that was previously identified to eliminate serum from methods for converting mouse cells. N2B27+BMP4 largely facilitated cells to an epithelial-like morphology (FIG. 3). A screen of a small-molecule library also identified that the Menin-MLL interaction inhibitor VTP50469 (VTP) and ATK Kinase Inhibitor (AKTi) further promoted the emergence epithelial-like colonies (FIG. 3). In FIG. 3, CHIR indicates CHIR99021 (a glycogen synthase kinase 3 (GSK-3)).

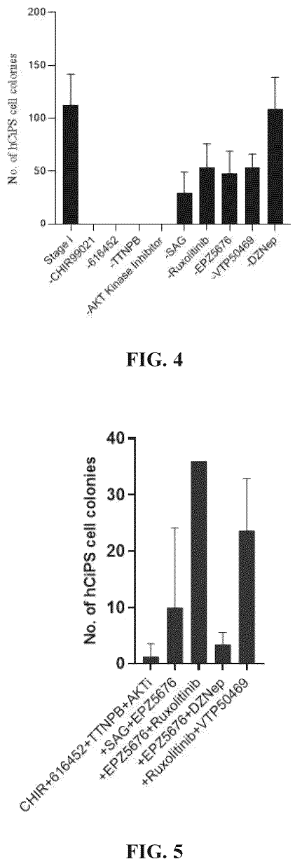

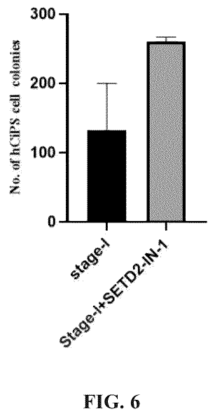

FIG. 4 shows the effects of removing various chemical reprogramming factors in the stage 1 conversion medium (“−” on the x-axis indicates removal of a particular chemical reprogramming factor in the stage 1 conversion medium) using the methods described in EXAMPLE 1. Stage 1 conversion media without any one of CHIR99021, 616452, TTNPB, and AKT Kinase Inhibitor showed minimal numbers of converted hCiPSCs. FIG. 5 shows that while the various combinations of SAG, EPZ5676, Ruxolithnib, DZNep, or VTP50469 could significantly increase the numbers of converted hCiPSCs when supplemented to the stage 1 conversion medium comprising CHIR99021, 616452, TTNPB, and AKT Kinase Inhibitor, using the methods described in EXAMPLE 1 (x-axis of FIG. 5 shows various combinations of SAG, EPZ5676, Ruxolithnib, DZNep, or VTP50469 supplemented to the stage 1 conversion medium comprising CHIR99021 (CHIR), 616452, TTNPB, and AKT Kinase Inhibitor (AKTi) shown on the far left-hand side). FIG. 6 shows that Addition of SET domain containing 2 (SETD2) inhibitor (e.g., SETD2-IN-1) significantly increased the numbers of converted hCiPSCs when supplemented to the stage 1 conversion medium using the methods described in EXAMPLE 1.

In stage 1, the conversion medium was changed every 3-4 days. hADSCs were seeded at a density of 1×104 cells per well of a 12-well plate in 15% FBS-DMEM medium and would be changed into stage I induction medium the next day. For stage I induction, single layer epithelial-like cells induced from hADSCs would emerge at day 4-6 and approach 100% confluence at day 8-12. The culture was then changed into stage 2 conversion medium. Conversion into epithelial-like cells can be measured by upregulation of and epithelial cell-related genes. Additionally, cells at the end of stage I can also show increased expression of LIN28A.

### Example 4: Stage 2 Conversion of Cells

Provided in this example are methods for stage 2 conversion of cells according to some embodiments of the present disclosure.

In stage 2, a first population of cells (e.g., epithelial-like cells) can be converted to a second population of cells (e.g., intermediate plastic state cells) using a stage 2 conversion medium. The stage 2 conversion medium may comprise a glycogen synthase kinase 3 (GSK-3), a transforming growth factor-beta (TGFβ) receptor inhibitor, a c-Jun kinase inhibitor, a S-adenosyl-L-homocysteine (SAH) hydrolase inhibitor, or an adenosine kinase inhibitor, or any combination thereof. The stage 2 conversion medium may additionally comprise a CBP/p300 bromodomain inhibitor, or a retinoic acid receptor (RAR) agonist, or any combination thereof. In some cases, the stage 2 conversion medium may further comprise a Dot1L inhibitor, a BMP receptor/AMPK inhibitor, a ROCK inhibitor, a Jak1/Jak2 inhibitor, a p38 MAPK inhibitor, a SET domain containing 2 (SETD2) inhibitor, a serine-threonine kinase, an Akt inhibitor, a Menin-MLL interaction inhibitor, or a casein kinase 2 inhibitor, or any combination thereof.

For example, in the stage 2 conversion, the population of cells comprising epithelial-like cells were contacted with a stage 2 conversion medium: KnockOut™ DMEM supplemented with 1% N2 supplement, 2% B27 supplement, 1% GlutaMAX™, 1% NEAA, 1% Penicillin-Streptomycin, 50 μg/ml Vc2p, 5 mM LiCl, 1 mM NAM, 200 ng/ml bFGF (Origene), 2 mg/mL AlbuMAX™-II and the small molecules CHIR99021 (5 μM), 616452 (10 μM), TTNPB (2 μM), SAG (0.5 μM), Y-27632 (10 μM), JNKIN8 (0.5 μM), EPZ5676 (2 μM), DZNep (0.2 uM), Ruxolitinib (1 μM), BIRB796 (2 μM), SGC-CBP30 (2 μM), Dorsormorphin (0.5 μM), VTP50469 (0.5 μM), and 5-Iodotubercidin (0.5 μM). AlbuMAX™-II could be replaced by 2% Knockout Serum Replacement (KSR).

Various combinations of chemical reprogramming factors for stage 2 conversion were tested.

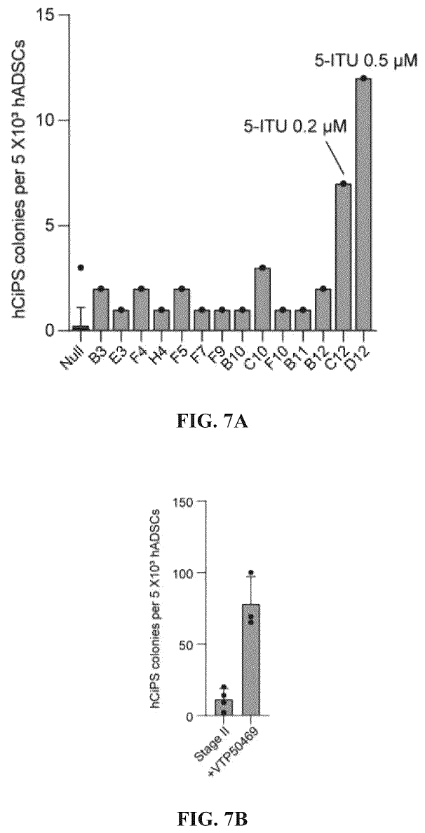

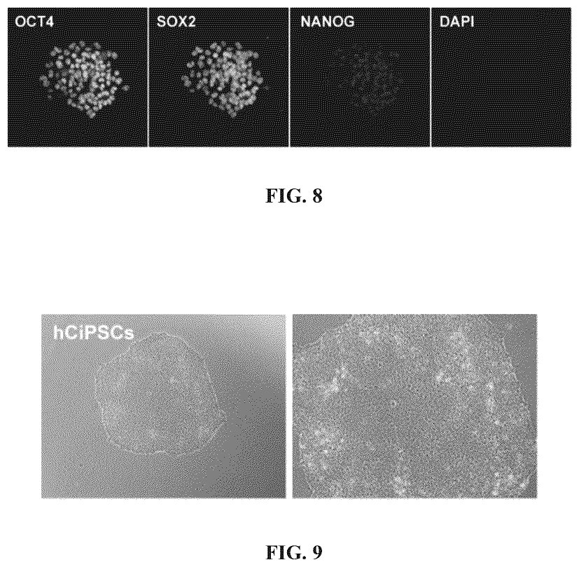

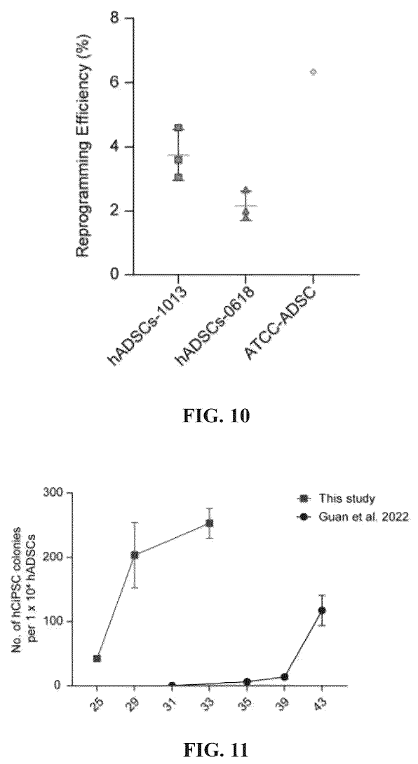

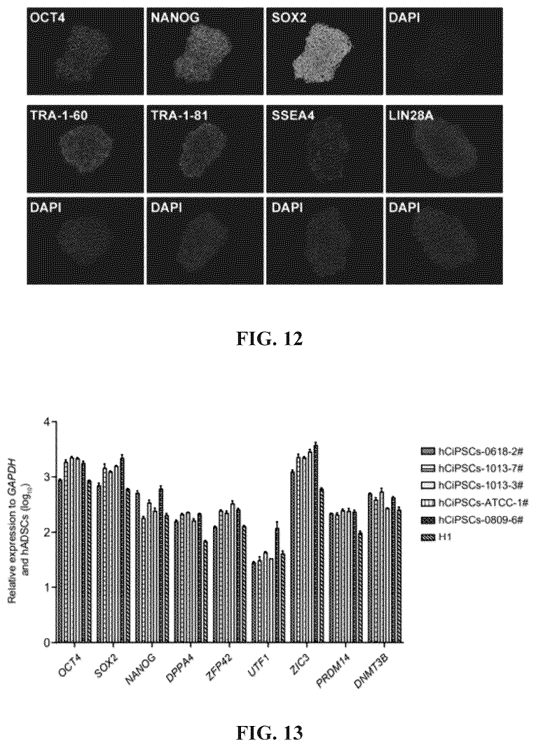

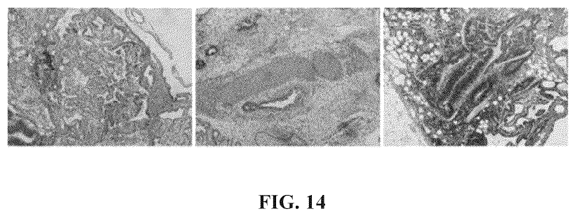

FIGS. 7A-7B show that including adenosine kinase inhibitor and/or Menin-MLL interaction inhibitor significantly increases the numbers of converted hCiPSCs when supplemented to the stage 2 conversion medium using the methods described in EXAMPLE 1. FIG. 7A shows that addition of 5-Iodotubercidin (5-ITU) in the stage 2 conversion medium (in reactions C12 and D12) increased the numbers of converted hCiPSCs by about more than 5-fold, relative to the conversion using stage 2 conversion medium without any 5-ITU (indicated by “Null”). FIG. 7B shows that addition of VPT50469 in the stage 2 conversion medium increased the numbers of converted hCiPSCs by about 7-fold. FIGs. Using the stage 2 medium comprising adenosine kinase inhibitor and Menin-MLL interaction inhibitor in the methods described in EXAMPLE 1, FIGS. 8-11 show that somatic cells could be converted to hCiPSCs within 24 days, and by day 32, at least 200 hCiPSC colonies were induced from a starting population of 10,000 somatic cells. FIG. 8 shows that hCiPSCs derived from hADSCs expressed pluripotent stem cell markers OCT4, SOX2, and/or NANOG. FIG. 9 shows that the morphology of that these hCiPSCs resembled that of the pluripotent stem cells. FIG. 10 shows that hCiPSCs could be generated from multiple donors (hADSCs-1013, hADSCs-0618, and ATCC-ADSC), with similar reprogramming efficiencies (the reprogramming efficiency was calculated by dividing the number of hCiPSCs generated and the number of hADSCs in the start somatic cells). FIG. 11 shows that the number of hCiPSC colonies generated by this EXAMPLE was significantly more than the methods described in Guan et al. 2022. As shown in FIGS. 12-14, standard pluripotency characterization confirmed that the hCiPSCs generated by method in this EXAMPLE are fully reprogrammed pluripotent stem cells. FIG. 12 shows that these hCiPSCs expressed various pluripotency markers. FIG. 13 shows the RT-qPCR analysis of pluripotency markers in the hCiPSCs generated by from somatic cells of various donors and hESCs (H1, as a control). FIG. 14 shows that single hCiPSCs from a single teratoma could generate endoderm (respiratory epithelium), mesoderm (cartilage) and ectoderm (pigmented retinal epithelium and neural tissue).

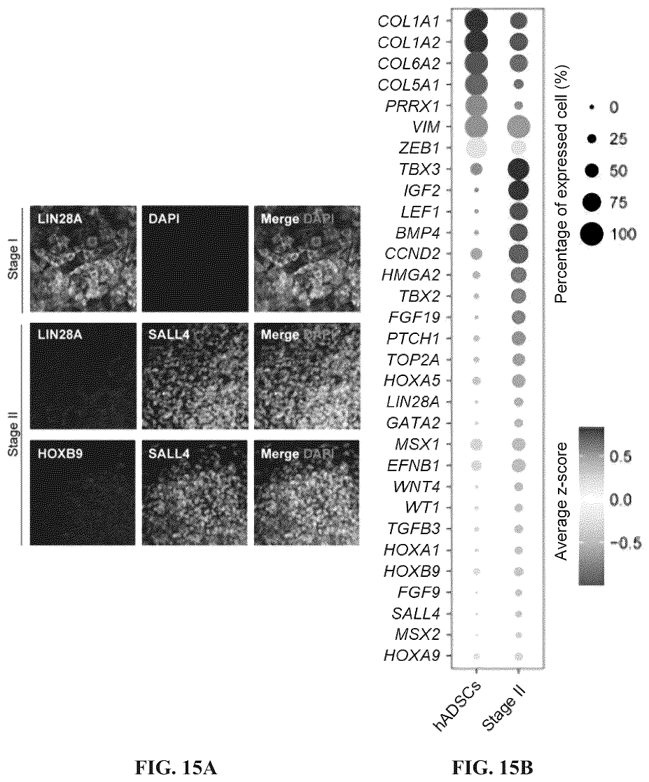

Additionally, presences of intermediate plastic state cells were confirmed by the end of stage 2. Developmental genes LIN28A, SALL4, MSX1, MSX2 and HOXB9 of the regeneration-like program were found to be expressed by the end of stage 2, as shown in FIGS. 15A-16.

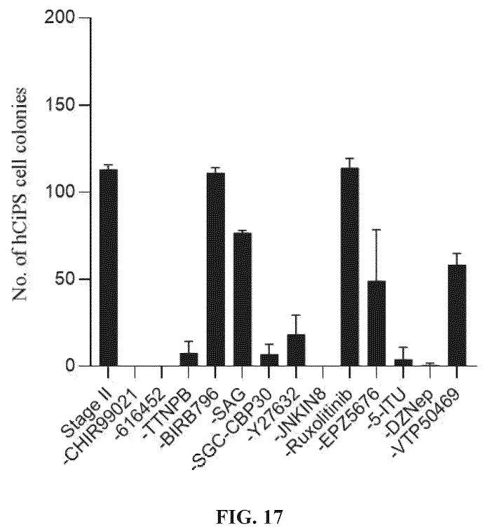

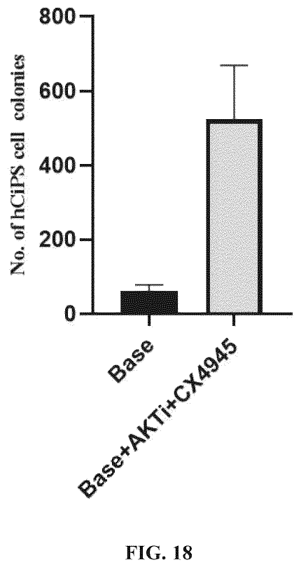

Furthermore, FIG. 17 shows the effects of removing various chemical reprogramming factors in the stage 2 conversion medium (“−” on the x-axis indicates removal of a particular chemical reprogramming factor in the stage 2 conversion medium) using the methods described in EXAMPLE 1. Stage 2 conversion media without any one of CHIR99021, 616452, TTNPB, JNKIN8, SGC-CBP300, DZNep, and 5-ITU showed minimal numbers of converted hCiPSCs. Removing EPZ5676 or VTP50469 from the Stage 2 conversion medium also reduced the numbers of converted hCiPSCs. FIG. 18 shows that including AKT Kinase Inhibitor (AKTi) and the CK2 inhibitor CX4945 in the stage 2 conversion medium (labeled as “Base”) significantly improved the reprogramming efficiency. Collectively, these data suggest that the feeder-free and serum-free system described in this EXAMPLE has high reprogramming efficiencies.

In stage 2, multi-layered cell colonies appeared after 8-12 days treatment. These cell colonies would continually grow larger. Additionally, cells at the end of stage 2 can show increased expression of SALL4 and LIN28A. The medium was changed into stage 3 conversion medium after 16 days' treatment of stage II conversion medium.

### Example 5: Stage 3 Conversion of Cells

Provided in this example are methods for stage 3 conversion of cells according to some embodiments of the present disclosure.

In stage 2, a first population of cells (e.g., intermediate plastic state cells) can be converted to a second population of cells (e.g., pluripotent stem cells) using a stage 3 conversion medium. The stage 3 conversion medium may comprise a B-Raf inhibitor, a histone deacetylase inhibitor, or a MAPK inhibitor, or any combination thereof. The stage 3 conversion medium may additionally comprise a Wnt inhibitor, a glycogen synthase kinase 3 (GSK-3), a ROCK inhibitor, an inhibitor of histone demethylation, a Dot1L inhibitor, or a S-adenosyl-L-homocysteine (SAH) hydrolase inhibitor, or any combination thereof.

For example, in stage 3, a population of cells comprising intermediate plastic state cells were contacted with a stage 3 conversion medium: Knockout™ DMEM supplemented with 1% N2 supplement, 2% B27 supplement, 1% GlutaMAX™, 1% NEAA, 1% Penicillin-Streptomycin, 50 μg/ml Vc2p, 20 ng/mL HRG and the small molecules CHIR99021 (1 μM), Y-27632 (10 μM), PD0325901 (1 μM), IWP-2 (2 μM), SB590885 (0.5 μM). During the induction process of stage 3, VPA (1000 μM), Tranylcypromine (10 μM), DZNep (0.2 μM), and EPZ5676 (2 μM) were included in the first 4 days. Subsequently, VPA (500 μM) was included in the next 4 days while Tranylcypromine, DZNep, and EPZ5676 were removed. The stage 3 conversion medium without VPA, Tranylcypromine, DZNep, or EPZ5676 could be applied for additional 2-4 days to allow the primary hCiPSCs colonies to grow larger.

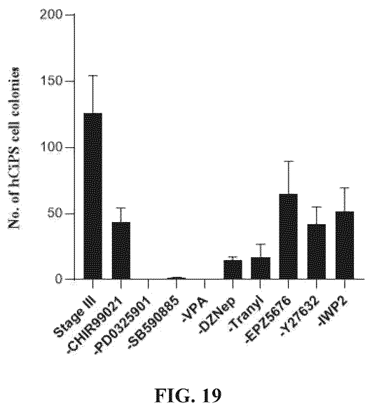

FIG. 19 shows the effects of removing various chemical reprogramming factors in the stage 3 conversion medium (“−” on the x-axis indicates removal of a particular chemical reprogramming factor in the stage 3 conversion medium) using the methods described in EXAMPLE 1. Stage 3 conversion media without any one of PD0325901, SB590885 and VPA showed minimal numbers of converted hCiPSCs.

### Derivation and Culture of Human CiPS Cell Lines

Provided herein, in some aspects, are methods to culture and maintain hCiPS cell lines. Derivation and culture of human CiPS cell lines Cells were dissociated by Accutase (Millipore, SCR005) after 8-12 days' induction of stage III condition, centrifuged at 400 g for 3 min to remove Accutase, and then re-plated at a ratio from 1:3 to 1:12 on feeder layers in the modified stage III medium: Knockout DMEM supplemented with 1% N2 supplement, 2% B27 supplement, 1% GlutaMAX™, 1% NEAA, 1% Penicillin-Streptomycin, 50 μg/ml Vc2p, 2 mg/mL AlbuMAX™-II and the small molecules CHIR99021 (1 μM), PD0325901 (0.5 μM), IWP-2 (2 μM), Y-27632 (10 μM), HRG (20 ng/mL), and bFGF (100 ng/mL, Origene). Cells were incubated under 21% O₂, 5% CO₂ at 37° C. and the medium was changed every day. Compact hCiPS cell colonies appeared after 3-7 days, and these colonies were manually picked up after 10-12 days, and mechanically dissociated into small clamps and transferred onto Matrigel coated plates (Corning, 354248) in mTeSR™ Plus Medium supplemented with Y-27632 (10 μM). After 24 hours, the culture was replaced by fresh mTeSR™ Plus Medium without Y-27632. were dissociated by Accutase (Millipore, SCR005) after 8-12 days' induction of stage III condition, centrifuged at 400 g for 3 min to remove Accutase, and then re-plated at a ratio from 1:3 to 1:12 on feeder layers in the modified stage III medium: Knockout DMEM supplemented with 1% N2 supplement, 2% B27 supplement, 1% GlutaMAX™, 1% NEAA, 1% Penicillin-Streptomycin, 50 μg/ml Vc2p, 2 mg/mL AlbuMAX™-II and the small molecules CHIR99021 (1 μM), PD0325901 (0.5 μM), IWP-2 (2 μM), Y-27632 (10 μM), HRG (20 ng/mL), and bFGF (100 ng/mL, Origene). Cells were incubated under 21% 02, 5% CO2 at 37° C. and the medium was changed every day. Compact hCiPS cell colonies appeared after 3-7 days, and these colonies were manually picked up after 10-12 days, and mechanically dissociated into small clamps and transferred onto Matrigel coated plates (Corning, 354248) in mTeSR™ Plus Medium supplemented with Y-27632 (10 μM). After 24 hours, the culture was replaced by fresh mTeSR™ Plus Medium without Y-27632.

### Example 6: Use of Histone Acetyltransferase Inhibitors

### Example 6A

This example describes the epigenetic analysis of chemically reprogrammed cells.

### (1) Chemical Induction of Human Fibroblasts

According to the method described by Liuyang S et al. (2023), chemically induced pluripotent stem cells (CiPSC) are formed from human adipose mesenchymal stem cells (hADSC) and human skin fibroblasts (hASF) (see Liuyang S et al., Cell Stem Cell. 2023 Apr. 6; 30(4):450-459.e9).

In short, hADSC or hASF are seeded at 1×10\hat{ }4 cells/well in a 12-well plate, or at 0.5-0.6×10\hat{ }4 cells/well in a 24-well plate, and cultured overnight. The next day, the medium is replaced with Stage I induction medium (Knockout DMEM supplemented with 2% B27 supplement, 1% KSR, 1% GlutaMAX, 1% NEAA, 1% penicillin-streptomycin, 50 g/mL Vc2p, 5 mM LiCl, 1 mM NAM, 20 ng/mL BMP4, 5 μM CHIR999021, 10 μM 616452, 2 μM TTNPB, 0.5 μM SAG, 2 μM EPZ5676, 0.05 μM DZNep, 1 μM ruxolitinib, 0.5 μM VTP50469, 1 μM HY-10249A (CAS No. 842148-40-7), 0.2 μM JNKIN8, and 0.2 μM SETD2-IN-1), and cultured under hypoxic conditions (5% 02) until a monolayer of epithelial-like cells appears and reaches approximately 100% confluence. hADSC cells typically require 8-10 days, and hASFs typically require 9-16 days. Immunofluorescent staining is performed on the formed monolayer epithelial-like cells to confirm positive staining for LIN28A; RT-PCR detection shows increased expression of epithelial cell markers KRTs, while fibroblast markers are reduced.

Then, the medium is replaced with Stage II induction medium (Knockout DMEM supplemented with 2% B27 supplement, 1% GlutaMAX, 1% NEAA, 1% penicillin-streptomycin, 50 g/mL Vc2p, 200 ng/mL bFGF, 5 μM CHIR99021, 10 μM 616452, 2 μM TTNPB, 0.5 μM SAG, 0.5 μM JNKIN8, 2 μM EPZ5676, 0.2 μM DZNep, 1 μM ruxolitinib, 2 μM BIRB796, 2 μM SGC-CBP30, 0.5 μM Dorsormorphin, 0.5 μM VTP50469, 0.5 μM 5ITU, 2 μM 5-Azacytidine, and 0.2 μM HY-10249A (CAS No. 842148-40-7)), and cultured under normoxic conditions (21% 02) for 8 to 12 days until the monolayer cells become multilayered, raised clones appear and gradually increase in size. Then, the medium is replaced with Stage III induction medium (Knockout DMEM supplemented with 2% B27 supplement, 1% GlutaMAX, 1% NEAA, 1% penicillin-streptomycin, 5% KSR, 50 g/mL Vc2p, 20 ng/mL recombinant human activin 3-1 (HRG), 1 μM CHIR99021, 10 μM Y-27632, 1 μM PD0325901, and 0.5 μM SB590885), and cultured under normoxic conditions for 8 to 12 days to form hCiPSC clones, with the addition of VPA (1000 μM), tranylcypromine (10 μM), DZNep (0.2 μM), and EPZ5676 (2 μM) in the medium for the first 4 days of this stage; from the 5th to 8th day, tranylcypromine, DZNep, and EPZ5676 are no longer added, and the VPA concentration is halved to 500 μM.

OCT4 immunofluorescent staining is performed on the obtained hCiPSC clones, with OCT4 positivity serving as an indicator of hCiPSC clones. The reprogramming efficiency of the method is calculated by dividing the number of OCT4-positive clones by the number of initial hADSC or hASFs plated.

### (2) Biological Property Analysis (Go Analysis)

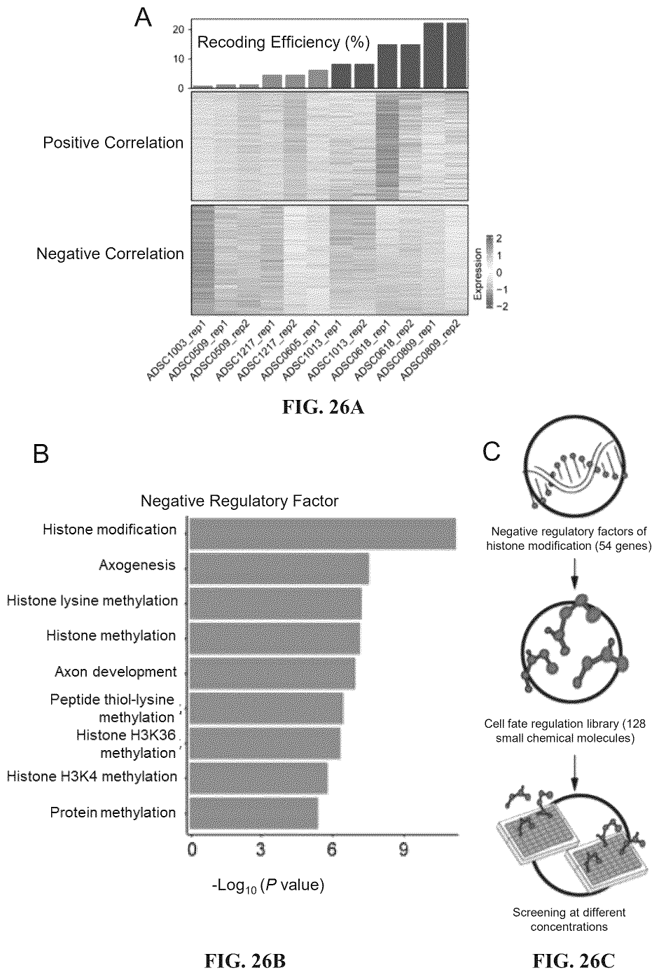

Using the above method, hCiPS clones were successfully induced in all tested somatic cells, but the reprogramming efficiency varied significantly among different cell lines, ranging from about 1% to about 20%. Therefore, hADSCs with different hCiPSC transformation efficiencies were selected and Gene Ontology (GO) analysis on their transcriptomes was performed to identify genes positively or negatively correlated with the transformation efficiency of somatic cell reprogramming, as shown in FIG. 26.

As can be seen from FIG. 26, a series of regulatory enzyme genes related to histone modification are highly expressed in cell strains with low transformation efficiency (FIG. 1b), suggesting that these epigenetic regulatory factors may be important barriers to low reprogramming transformation efficiency. Based on this, a chemical small molecule candidate library with histone modification activity was constructed.

### Example 6B

This example describes how histone acetyltransferase inhibitors can improve the transformation efficiency of somatic cell chemical reprogramming and also shorten the total transformation time of somatic cell chemical reprogramming.

Chemical reprogramming of hADSC cells is performed using a method essentially the same as in Example 6B, with the only difference being: in Method I, the time for culturing cells with Stage I induction medium is shortened to 8 days, with Stage II induction medium to 4 days, and with Stage III induction medium to 8 days, for a total culture of 20 days as the control (“8+4+8”); while in Method II, the time for culturing cells with Stage I induction medium is further shortened to 6 days, with Stage II induction medium to 4 days, and with Stage III induction medium to 8 days, for a total culture of 18 days as the control group (“6+4+8”).

In the test groups, chemical small molecules from the candidate library are added to the stage I induction medium, and the number of OCT4-positive hCiPSC clones in the control and each test group is measured on the last day of culture to screen for small molecule compounds that can significantly improve the efficiency of somatic cell reprogramming. It was found that in both the control and test groups, a monolayer of epithelial-like cells positive for LIN28A could be observed during the culture with stage I induction medium (results not shown), and if continued to be cultured until reaching approximately 100% confluence, it usually takes 5-8 days for the hADSC cell test group and 6-10 days for the hASF cell test group, significantly shortening the induction time compared to the control group.

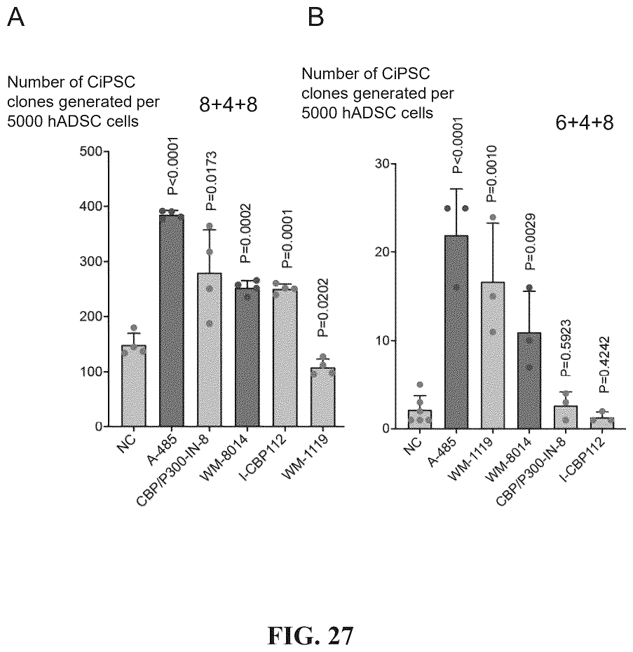

In addition, in several test groups adding histone acetyltransferase inhibitors (HATi), a significant increase in reprogramming efficiency was observed, with exemplary results shown in FIGS. 27A and 27B. In the figures, A485, CBP/P300IN8, and ICBP112 belong to CBP/P300 complex (KAT3A/B) inhibitors, while WM8014 and WM1119 belong to KAT6A (KAT6A) inhibitors. As can be seen from FIG. 27A, using the “8+4+8” culture method, the control group produces about 150 hCiPSC clones per 5000 somatic cells, with a transformation efficiency of about 3%. In the test groups with added histone acetyltransferase inhibitors (HATi), except for the KAT6A inhibitor WM1119 with a slightly lower transformation efficiency (about 2.4%), the transformation efficiency of the other four HATi is above 5%, achieving a statistically significant improvement. As can be seen from FIG. 27B, when the induction time, especially the initial induction period for somatic cells to start reprogramming, is further shortened (“6+4+8”), the transformation efficiency of the control group drops sharply, producing only about 1 hCiPSC clone per 5000 somatic cells, with a transformation efficiency of about 0.02%. At the same time, most of the test groups with added HATi can still produce hCiPSC clones with a transformation efficiency at least one order of magnitude higher than the control. Unbound by theory, this may be due to the addition of histone acetyltransferase inhibitors, which makes the chromatin of somatic cells, originally in a relatively closed state, more relaxed, thus facilitating the gene expression of early regulatory factors for reprogramming.

### Example 6C

This example describes how combinations of different types of histone acetyltransferase inhibitors can synergistically improve the reprogramming efficiency of somatic cells.

Chemical reprogramming of hADSC cells is performed using a method essentially the same as in Example 6A, with the only difference being: the time for culturing cells with Stage I induction medium is shortened to 6 days, with Stage II induction medium to 8 days, and with Stage III induction medium to 8 days, for a total culture of 22 days as the control (“6+8+8”).

The following reagents or combinations of reagents are added to the Stage I induction medium as test groups:
Test Group 1: KAT6A inhibitor WM8014 is added alone at a concentration of 1 μM;
Test Group 2: KAT3A/B inhibitor A485 is added alone at a concentration of 0.5 μM;
Test Group 3: A combination of WM8014 and A485 is added, with WM8014 at a concentration of 1 μM and A485 at a concentration of 0.5 μM.

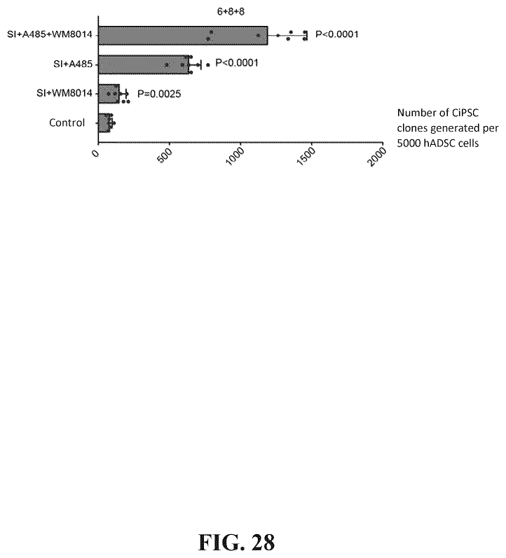

The number of OCT4-positive hCiPSC clones in the control and each test group is measured on the last day of culture to calculate the reprogramming efficiency of somatic cells. The results are shown in FIG. 28. It can be seen that when the induction time of Stage I is shortened to 6 days, the addition of the inhibitor combination A485 and WM8014 can still successfully induce a monolayer of epithelial-like cells positive for LIN28A, and ultimately achieve a transformation efficiency of up to about 24%, which is about 15 times higher than the control group, indicating that the addition of the inhibitor combination A485 and WM8014 in the early stage of reprogramming can achieve a synergistic improvement effect.

### Example 6D

This example describes how optimizing the combination of compound small molecules can improve the efficiency of generating induced pluripotent stem cells from LIN28A-positive monolayer epithelial-like cells.

Chemical reprogramming of hADSC cells is performed using a method essentially the same as in Example 6A, with the only difference being: two histone acetyltransferase inhibitors are added to the Stage I induction medium (1 μM WM8014 and 0.5 μM A485), the time for culturing cells with Stage I induction medium is shortened to 6 days, with Stage II induction medium to 4 days, and with Stage III induction medium to 8 days, for a total culture of 18 days as the control (“6+4+8”). The following reagents or combinations of reagents are added to the Stage II induction medium as test groups:
Test Group 1: RA is added alone at a concentration of 5 μM;
Test Group 2: GSK3685032 is added alone at a concentration of 0.02 μM;
Test Group 3: A combination of RA and GSK3685032 is added, with RA at a concentration of 5 μM and GSK3685032 at a concentration of 0.02 μM.

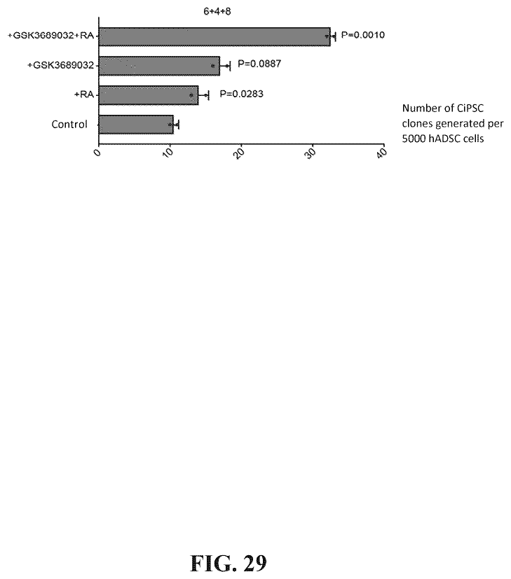

The number of OCT4-positive hCiPSC clones in the control and each test group is measured on the last day of culture to calculate the reprogramming efficiency of somatic cells. The results are shown in FIG. 29. It can be seen that under the condition of shortened induction time, the addition of RA receptor activator or DNMT inhibitor GSK3685032 alone can significantly improve the transformation of LIN28A-positive monolayer epithelial-like cells into pluripotent stem cells compared to the control group, and the combination of the two can also significantly enhance the transformation efficiency of hCiPSCs.

### Example 6E

This example describes how optimizing the combination of compound small molecules can further improve the efficiency of generating induced pluripotent stem cells from LIN28A-positive monolayer epithelial-like cells.

Chemical reprogramming of hADSC cells is performed using a method essentially the same as in Example 6A, with the only difference being: a combination of two histone acetyltransferase inhibitors is added to the Stage I induction medium (1 μM WM8014 and 0.5 μM A485), and the culture time is shortened to 8 days, a combination of RA receptor activator (5 μM RA) and DNMT inhibitor (0.02 μM GSK3685032) is added to the Stage II induction medium, and the culture time is shortened to 5 days, and the time for culturing cells with Stage III induction medium is shortened to 8 days, for a total culture of 21 days as the control (“8+5+8”).

The following reagents or combinations of reagents are added to the Stage III induction medium respectively as test groups:
Test Group 1: YAP activator PY60 is added alone at a concentration of 10 μM;
Test Group 2: YAP activator PY60 is added to a concentration of 10 μM, and the concentration of the histone deacetylase inhibitor VPA is reduced from 1000 μM to 200 μM in the first three days.

Test Group 3: PY60 is added at a concentration of 2 μM, and the concentration of VPA is reduced to 200 μM for the first six days.

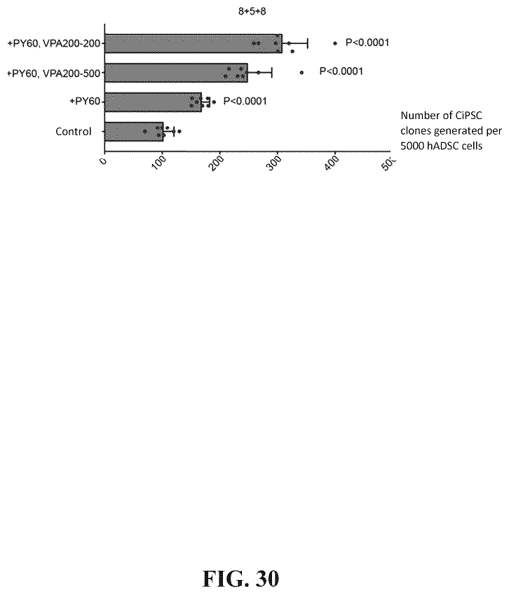

The number of OCT4-positive hCiPSC clones in the control and each test group is measured on the last day of culture to calculate the reprogramming efficiency of somatic cells. The results are shown in FIG. 30. It can be seen that the addition of YAP activator to the Stage III induction medium and further reducing the concentration of histone deacetylase inhibitors based on this, can significantly improve the transformation of LIN28A-positive monolayer epithelial-like cells into pluripotent stem cells compared to the control group, with the transformation efficiency increased up to about 3 times that of the control.

### Example 6F

This example describes several exemplary induction reprogramming methods of the present invention and compares the transformation efficiency under different induction times to determine the shortest duration for effectively obtaining hCiPSC clones through the reprogramming methods disclosed in this text.

hADSC or hASF are seeded at 0.5 to 0.7×10\hat{ }4 cells/well in a 24-well plate and cultured overnight. The next day, the medium is replaced with Stage I induction medium (Knockout DMEM supplemented with 2% B27 supplement, 1% KSR, 1% GlutaMAX, 1% NEAA, 1% penicillin-streptomycin, 50 g/mL Vc2p, 5 mM LiCl, 1 mM NAM, 20 ng/mL BMP4, 5 μM CHIR999021, 10 μM 616452, 2 μM TTNPB, 0.5 μM SAG, 2 μM EPZ5676, 0.05 μM DZNep, 1 μM ruxolitinib, 0.5 μM VTP50469, 1 μM HY-10249A (CAS No. 842148-40-7), 0.2 μM JNKIN8, 0.2 μM SETD2-IN-1, 0.5 μM AM095, 0.5 μM A485, and 1 μM WM8014), and cultured under hypoxic conditions (5% 02) until a monolayer of epithelial-like cells appears (which can also be continued to be cultured until the confluence of monolayer epithelial-like cells reaches about 100%).

Then, the medium is replaced with Stage II induction medium (Knockout DMEM supplemented with 2% B27 supplement, 1% GlutaMAX, 1% NEAA, 1% penicillin-streptomycin, 50 g/mL Vc2p, 200 ng/mL bFGF, 5 μM CHIR99021, 10 μM 616452, 2 μM TTNPB, 0.5 μM SAG, 0.5 μM JNKIN8, 2 μM EPZ5676, 0.2 μM DZNep, 1 μM ruxolitinib, 2 μM BIRB796, 2 μM SGC-CBP30, 0.5 μM Dorsormorphin, 0.5 μM VTP50469, 0.5 μM 5ITU, 2 μM 5-Azacytidine, 0.2 μM HY-10249A (CAS No. 842148-40-7), 0.02 μM GSK3685032, 5 μM RA), and cultured under normoxic conditions (21% 02) until the monolayer cells become multilayered, raised clones appear and gradually increase in size.

Then, the medium is replaced with Stage III induction medium (Knockout DMEM supplemented with 2% B27 supplement, 1% GlutaMAX, 1% NEAA, 1% penicillin-streptomycin, 5% KSR, 50 g/mL Vc2p, 20 ng/mL recombinant human activin β-1 (HRG), 1 μM CHIR99021, 10 μM Y-27632, 1 μM PD0325901, 0.5 μM SB590885), and first add a combination of 200 μM VPA, 10 μM tranylcypromine, 0.2 μM DZNep, 2 μM EPZ5676, and 10 μM PY60 (VTZEP), then replace with the addition of 200 μM VPA and 2 μM PY60, and culture under normoxic conditions until hCiPSC clones are formed.

It should be noted that the Stage III medium can also be used for further culture to enlarge the hCiPSC clones, but at this point, the number of clones hardly increases.

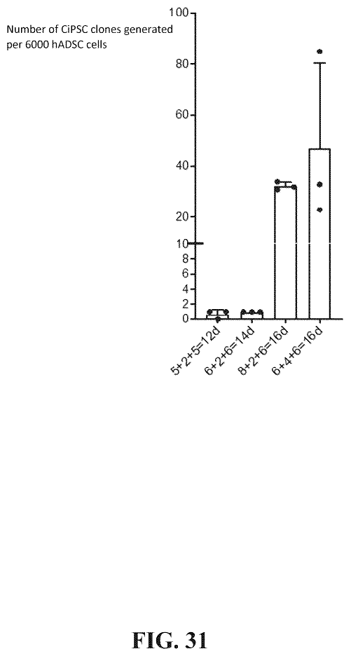

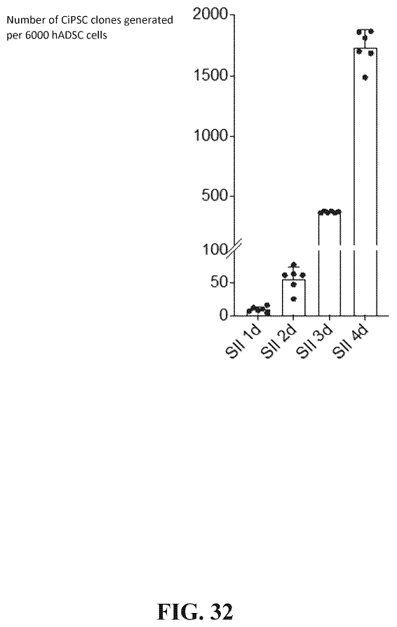

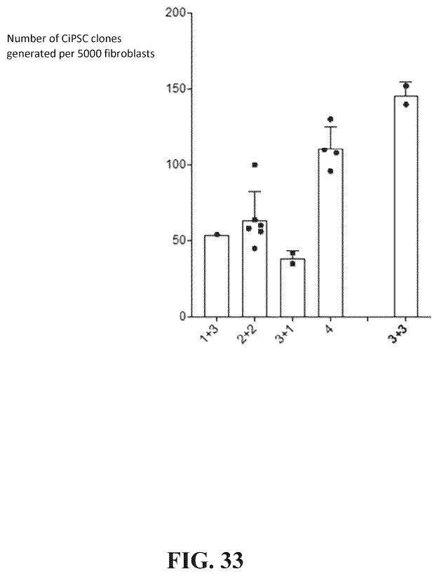

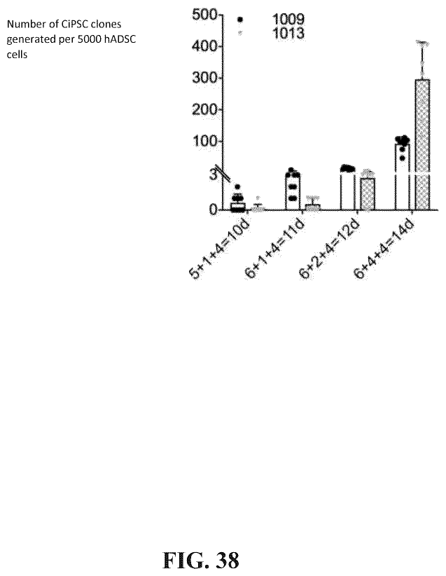

TABLE 3
Specific culture plans for each test group

| | | | | | | | | | Average |
|---|---|---|---|---|---|---|---|---|---|
| | | | | | | | | Average | Number of |
| | Stage I | Stage II | | Stage III Medium | | | | Transformation | hCiPSC |
| Group | Medium | Medium | | +VTZEP | | +V | | Efficiency | Clones |
| FIG. 31-1 | 5 days | 2 | days | 2 | days | 3 | days | 0.0111% | 0.7 |
| FIG. 31-2 | 6 days | 2 | days | 3 | days | 3 | days | 0.0167% | 1 |
| FIG. 31-3 | 8 days | 2 | days | 3 | days | 3 | days | 0.5388% | 32.3 |
| FIG. 31-4 | 6 days | 4 | days | 3 | days | 3 | days | 0.7833% | 47 |
| FIG. 32-1 | 8 days | 1 | day | 3 | days | 3 | days | 0.1583% | 9.5 |
| FIG. 32-2 | 8 days | 2 | days | 3 | days | 3 | days | 0.9417% | 56.5 |
| FIG. 32-3 | 8 days | 3 | days | 3 | days | 3 | days | 6.205% | 372.3 |
| FIG. 32-4 | 8 days | 4 | days | 3 | days | 3 | days | 28.91% | 1734.8 |
| FIG. 33-1 | 6 days | 4 | days | 1 | day | 3 | days | 1.080% | 54 |
| FIG. 33-2 | 6 days | 4 | days | 2 | days | 2 | days | 1.276% | 63.8 |
| FIG. 33-3 | 6 days | 4 | days | 3 | days | 1 | day | 0.7700% | 38.5 |
| FIG. 33-4 | 6 days | 4 | days | 4 | days | 0 | days | 2.220% | 111 |
| FIG. 33-5 | 6 days | 4 | days | 3 | days | 3 | days | 2.920% | 146 |
| FIG. 38-1-1 | 5 days | 1 | day | 2 | days | 2 | days | 0.01% | 0.5 |
| FIG. 38-2-1 | 6 days | 1 | day | 2 | days | 2 | days | 0.06% | 3.1 |
| FIG. 38-3-1 | 6 days | 2 | days | 2 | days | 2 | days | 0.29% | 14.25 |
| FIG. 38-4-1 | 6 days | 4 | days | 2 | days | 2 | days | 1.85% | 92.25 |
| FIG. 38-1-2 | 5 days | 1 | day | 2 | days | 2 | days | 0.0025% | 0.125 |
| FIG. 38-2-2 | 6 days | 1 | day | 2 | days | 2 | days | 0.01% | 0.5 |
| FIG. 38-3-2 | 6 days | 2 | days | 2 | days | 2 | days | 0.055% | 2.75 |
| FIG. 38-4-2 | 6 days | 4 | days | 2 | days | 2 | days | 5.94% | 297 |

*The above groups are the average values of more than 3 repetitions.

OCT4 and SOX2 double positivity are used as indicators of hCiPSC clones. The reprogramming efficiency of the method is calculated by dividing the number of double-positive clones in each well by the number of initial hADSCs or hASFs, and the results are shown in Table 3, FIGS. 31-33 and 38. It can be seen that by adding histone acetyltransferase inhibitors in the early stage of reprogramming, the entire duration of somatic cell chemical reprogramming to form pluripotent stem cells can be significantly shortened, with the minimum time of only 10 days to obtain hCiPSC clones. This can greatly save culture costs, improve production efficiency, and reduce the risk of contamination in the industrial production process of hCiPSCs, which is extremely beneficial. In addition, when the induction culture time is appropriately extended, the transformation efficiency of this invention method can also be stably above 1%, even achieving an efficient transformation of about 30%.

### Example 6G

This example describes the establishment of an hCiPSC line using the exemplary reprogramming method of the present invention. The method for establishing the cell line is as follows:

Preparation of hCiPSC Establishment Medium

Add 2% B27 supplement, 1% GlutaMAX, 1% NEAA, 1% penicillin-streptomycin, 2 mg/mL AlbuMAX™-II or 4% KSR, 50 g/mL Vc2p, 20 ng/mL HRG, 100 ng/mL bFGF, 1 μM CHIR99021, 0.5 μM PD0325901, and 10 μM Y-27632 to Knockout DMEM. AlbuMAX™-JJ can also be replaced with 4% KSR.

hCiPSC Establishment

Induce the culture of somatic cells (e.g., hADSC or hASF) by the same method as in Example 6F. Then, digest the cells with Accutase, centrifuge at 400 g for 3 minutes, resuspend the cells in the above establishment medium, dilute at a ratio of 1:3 to 1:12, and plate on a culture plate with Laminin-521 matrix.

Cultivate the cells under conditions of 21% O₂, 5% CO₂, and 37° C., changing the medium daily. After 10 to 12 days of culture, well-defined hCiPSC colonies are observed. After digestion with ReLeSR™ for 5 minutes, cut the hCiPSC colonies into small pieces with a glass needle, and transfer to a 48-well culture plate coated with Matrigel matrix, and culture with mTeSR™ Plus medium supplemented with 10 μM Y-27632. 24 hours later, change the medium with fresh mTeSR™ Plus medium, which does not contain Y-27632. Thereafter, change the medium every 2 days.

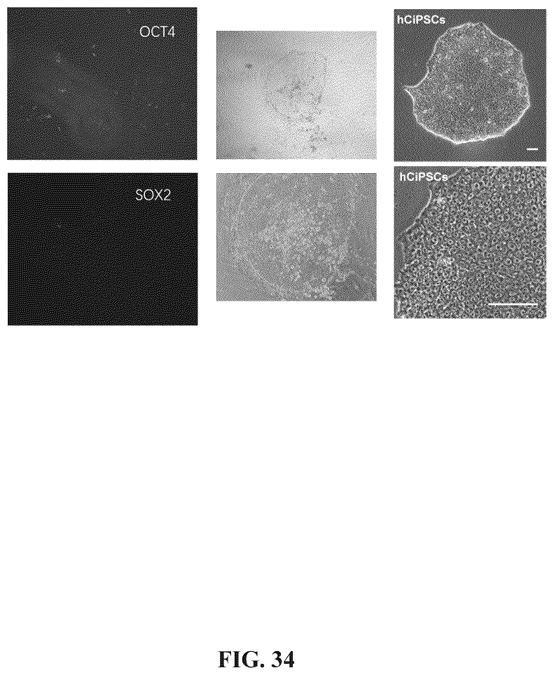

Immunofluorescent staining of OCT4 and SOX2 on the cell population at the end of culture is shown in FIG. 34. FIG. 34 indicates that the pluripotent stem cells obtained by the chemical reprogramming method disclosed in this text can be used to establish an hCiPSC line.

### Example 6H

This example compares the effects of using the medium scheme described in Example 6A (original scheme) and the medium scheme described in Example 6F (rapid scheme) on the production of hCiPSCs from human adipose-derived mesenchymal cell line (hADSC) and human skin-derived fibroblast cell line (hASF).

(1) In various cell lines, the exemplary scheme of the present invention can achieve a higher hCiPSC cell transformation efficiency.

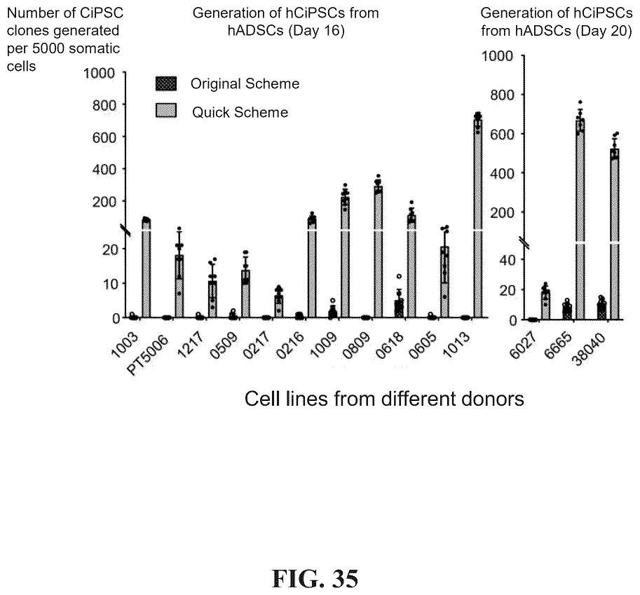

On the last day of 16 or 20 days of induction culture using the original scheme and rapid scheme, the number of hCiPSC colonies obtained was determined by OCT4 immunofluorescent staining, and the specific results are shown in Table 4 and FIG. 35. The comparison results show that the exemplary method of this text can stably obtain a large number of iPSC colonies between different batches; however, similar control methods cannot or are hardly able to produce colonies in the same period of time.

TABLE 4

| Cell | | | | | | | | | | | | | | | | |
|---|---|---|---|---|---|---|---|---|---|---|---|---|---|---|---|---|
| Line | Original Scheme (Control) | | | | | | | | Rapid Scheme | | | | | | | |
| 1003 | 1 | 0 | 0 | 0 | 0 | 0 | 0 | 0 | 94 | 90 | 77 | 86 | 89 | 82 | 77 | 73 |
| PT5006 | 0 | 0 | 0 | 0 | 0 | 0 | 0 | 0 | 30 | 21 | 20 | 17 | 17 | 12 | 7 | 21 |
| 1217 | 1 | 0 | 0 | 0 | 0 | 0 | 0 | 0 | 5 | 3 | 10 | 17 | 10 | 12 | 12 | 16 |
| 0509 | 1 | 2 | 0 | 0 | 0 | 0 | 0 | 0 | 10 | 11 | 15 | 10 | 19 | 11 | 15 | 19 |
| 0217 | 0 | 0 | 0 | 0 | 0 | 0 | 0 | 0 | 7 | 2 | 8 | 6 | 8 | 9 | 5 | 6 |
| 0216 | 1 | 1 | 1 | 0 | 0 | 0 | 0 | 0 | 68 | 100 | 72 | 90 | 92 | 61 | 89 | 124 |
| 1009 | 3 | 0 | 1 | 2 | 1 | 1 | 5 | 2 | 202 | 244 | 249 | 260 | 299 | 146 | 213 | 179 |
| 0809 | 1 | 0 | 0 | 0 | 0 | 0 | 0 | 0 | 307 | 257 | 316 | 250 | 356 | 260 | 260 | 320 |
| 0618 | 3 | 4 | 3 | 4 | 7 | 2 | 5 | 12 | 117 | 148 | 76 | 69 | 76 | 87 | 126 | 193 |
| 0605 | 0 | 0 | 0 | 0 | 0 | 1 | 0 | 0 | 15 | 13 | 29 | 25 | 19 | 40 | 18 | 6 |
| 1013 | 0 | 0 | 0 | 0 | 0 | 0 | 0 | 0 | 725 | 744 | 625 | 654 | 711 | 725 | 741 | 715 |
| HDF-6027 | 0 | 0 | 0 | 0 | 0 | 0 | 0 | 0 | 10 | 19 | 20 | 18 | 21 | 14 | 24 | 18 |
| HDF-6665 | 10 | 7 | 5 | 13 | 6 | 10 | 9 | | 645 | 614 | 682 | 671 | 704 | 762 | 599 | |
| HDF-38040 | 14 | 15 | 12 | 10 | 7 | 7 | 11 | 10 | 592 | 602 | 472 | 512 | 504 | 477 | 482 | 541 |

* The starting cell lines HDF-6027, 6665, 38040 are hASFs, and the rest are hADSCs; each starting cell line was repeated 8 times for reprogramming induction.

(2) The exemplary medium of the present invention can achieve a faster transformation curve.

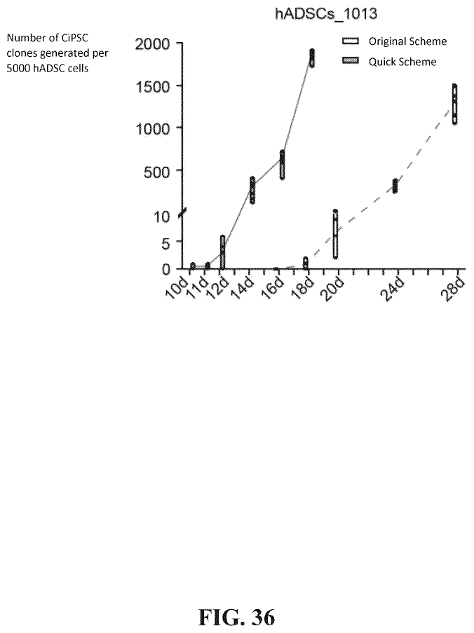

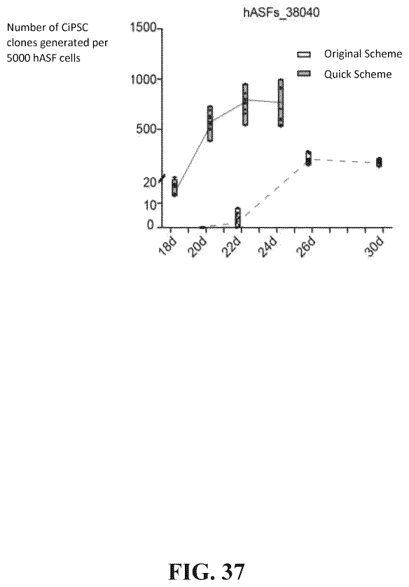

For the hADSCs, culture for 10, 11, 12, 14, 16, 18, or 20 days using the rapid scheme, or culture for 10, 11, 12, 14, 16, 18, 20, 24, 28 days using the scheme original; for the hASFs, culture for 18, 20, 22, or 24 days using the rapid scheme, or culture for 18, 20, 22, 26, or 30 days using the original scheme. On the last day of each culture scheme, the number of hCiPSC colonies induced by the rapid scheme and the control original scheme was determined by OCT4 immunofluore scent staining, and the specific results are shown in Tables 5 and 6 and FIGS. 36 and 37. The figures show that the exemplary method disclosed in this text not only enables somatic cells to transform into pluripotent stem cells more quickly but also achieves a higher transformation rate in a shorter period of time.

TABLE 5
Reprogramming effect of hASDC cells at different culture days

| | Original Scheme (Control) | | | | | | | | Rapid Scheme | | | | | | | |
|---|---|---|---|---|---|---|---|---|---|---|---|---|---|---|---|---|
| 10d | 0 | 0 | 0 | 0 | 0 | 0 | 0 | 0 | 0 | 0 | 0 | 0 | 0 | 0 | 0 | 0 |
| 11d | 0 | 0 | 0 | 0 | 0 | 0 | 0 | 0 | 1 | 2 | 0 | 0 | 0 | 0 | 0 | 0 |
| 12d | 0 | 0 | 0 | 0 | 0 | 0 | 0 | 0 | 1 | 3 | 3 | 3 | 3 | 3 | 4 | 0 |
| 14d | 0 | 0 | 0 | 0 | 0 | 0 | 0 | 0 | 66 | 74 | 79 | 38 | 13 | 34 | 38 | 40 |
| 16D | 0 | 0 | 0 | 0 | 0 | 0 | 0 | 0 | 25 | 30 | 23 | 25 | 137 | 38 | 43 | 46 |
| 18D | 1 | 2 | 3 | 1 | 2 | 3 | 1 | 1 | 368 | 200 | 145 | 277 | 193 | 239 | 255 | 245 |
| 20D | 44 | 67 | | 53 | 29 | 33 | 34 | 35 | 827 | 516 | 813 | 843 | 542 | 707 | 827 | 731 |
| 24D | 729 | 722 | 482 | 880 | 777 | 688 | 688 | 967 | — | — | — | — | — | — | — | — |
| 28D | 403 | 291 | 530 | 484 | 567 | 545 | 499 | 375 | — | — | — | — | — | — | — | — |

* “—” indicates not determined; both control and test groups were repeated eight times.

TABLE 6
Reprogramming effect of hASFs at different culture days

| | Original Scheme (Control) | | | | | | | | Rapid Scheme | | | | | | | |
|---|---|---|---|---|---|---|---|---|---|---|---|---|---|---|---|---|
| 18d | 0 | 0 | 0 | 0 | 0 | 0 | 0 | 0 | 13 | 20 | 14 | 21 | 25 | 18 | 14 | 17 |
| 20d | 0 | 0 | 0 | 0 | 0 | 0 | 0 | 0 | 692 | 502 | 554 | 612 | 497 | 731 | 383 | 632 |
| 22d | 1 | 4 | 8 | 2 | 5 | 6 | 0 | 3 | 540 | 952 | 804 | 698 | 857 | 759 | 656 | 794 |
| 24d | — | — | — | — | — | — | — | — | 592 | 915 | 542 | 543 | 997 | 530 | 905 | 604 |
| 26d | 180 | 196 | 174 | 145 | 279 | 264 | 204 | 194 | — | — | — | — | — | — | — | — |
| 30d | 204 | 170 | 213 | 184 | 130 | 157 | 164 | 174 | — | — | — | — | — | — | — | — |

* “—” indicates not determined; both control and test groups were repeated eight times.

Unbound by theory, it is believed that this is mainly due to the addition of histone acetyltransferase inhibitors in the critical early stage of reprogramming, enabling the induced somatic cells to more quickly and comprehensively be in a state that is receptive to reprogramming induction, thereby greatly shortening the time required for chemical reprogramming and significantly improving the transformation efficiency of iPSC colonies.

### Example 7: Chemical Reprogramming with KAT3A/KAT3B and KAT6A Inhibitors

### Methods

### Animals

All animal experiments were approved by the Institutional Animal Care and Use Committee of Peking University and were performed according to the Animal Protection Guidelines of Peking University. Immunodeficient NPG mice were purchased from Beijing Vitalstar Biotechnology Company and housed under a 12-h light/12-h dark cycle between 0600 and 1800 h in a temperature-controlled room (22+1° C.) with 40-60% humidity. Water and food were accessible at all times.

### Cell Culture

hADSCs, hASFs, hCiPSCs, hES cells (H1 and H9), mouse embryonic fibroblasts (MEFs) and 293T cells were cultivated according to previously reported protocols 7. In summary, 1.5×10⁶ hADSCs or hASFs were plated in 100-mm dishes and grown in Mesenchymal Stem Cell Growth Medium 2 (PromoCell) under 21% O₂ and 5% CO₂ at 37° C. The medium was replaced every 2 days. Before reaching confluence, the cells were dissociated for passaging using 0.25% trypsin-EDTA (Gibco). For hCiPSC induction, it was recommend using primary hADSCs and hASFs within four passages. hCiPSCs and hES cells (H1 and H9) were cultured in mTeSR Plus medium (STEMCELL) on plates coated with Matrigel (Corning) under 21% 02 and 5% CO₂ at 37° C. The medium was replaced daily. For passaging, when the cells reached approximately 85% confluence, they were dissociated with ReLeSR (STEMCELL), and detached cell aggregates were replated in mTeSR Plus medium supplemented with Y-27632 (10 μM) at a splitting ratio of 1:10 to 1:20. After 1 day, the medium was replaced with fresh mTeSR Plus medium without Y-27632. MEFs were cultured in high-glucose DMEM (Gibco) supplemented with 15% fetal bovine serum (FBS; Vistech) and 2% penicillin-streptomycin under 21% O₂ and 5% CO₂ at 37° C. and were dissociated for passaging using 0.25% trypsin-EDTA before reaching confluence. H1 (NIH: hESC-10-0043) and H9 (NIH: hESC-10-0062) cells were gifts from WiCell. Commercial hADSCs were purchased from Lonza (PT5006) and ATCC (PCS-500-011). Commercial hASFs were purchased from Lonza (CC2511). The HEK293T cell line was a gift from the laboratory of R. Mu (Peking University Third Hospital). Other cell lines were derived in-house. Cell lines were authenticated by STR analysis, along with immunofluorescence, RT-qPCR or RNA-seq. No cross-contamination or misidentification was identified. No known misidentified cell lines (maintained by the International Cell Line Authentication Committee) were used.

### Isolation and Culture of Primary Cells

Primary hADSCs and hASFs

Human adult adipose tissues and dermis tissues were obtained with informed written consent and were approved by the Institute of Ethics Committee Review Board at Peking University (IRB 00001052-19070). Donor information is shown in Supplementary Table 3. This study was conducted according to the principles of the Declaration of Helsinki.

hADSCs and hASFs were isolated as previously described6. For isolating hADSCs, after washing twice with PBS (Corning) supplemented with 2% penicillin-streptomycin (Gibco), adipose tissue (2-4 cm²) was minced into 1- to 2-mm² pieces and dissociated with 2 mg ml-1 collagenase IV (Gibco) at 37° C. for 1 h. The cells were then dissociated by adding high-glucose DMEM (Gibco) supplemented with 15% FBS (Vistech) and 2% penicillin-streptomycin using a pipette. After being vigorously shaken for 1 to 2 min, the suspension was transferred to 50-ml tubes, diluted with high-glucose DMEM supplemented with 15% FBS and 2% penicillin-streptomycin and centrifuged at 400 g for 5 min. After centrifugation, the precipitated cell pellets were resuspended and cultured in Mesenchymal Stem Cell Growth Medium 2.

For isolating hASFs, the dermis tissues were minced to 0.5- to 1-mm² pieces after washing twice with PBS containing 2% penicillin-streptomycin and were placed in 100-mm culture dishes, each with one drop of high-glucose DMEM supplemented with 15% FBS. The pieces were incubated at 37° C. for 4-12 h, and 3-5 ml of Mesenchymal Stem Cell Growth Medium 2 was then added to the dish gently. Outgrowths of fibroblasts were generated within 4-7 days.

For hCiPSC induction, hADSCs and hASFs were seeded 1 day before stage 1 induction in high-glucose DMEM supplemented with 15% FBS at a density of 1.2-1.4×104 cells per well in 12-well plates or 0.6-0.7×10⁴ cells per well in 24-well plates.

Commercial Primary hADSCs and hASFs

The same protocol was followed for the expansion and reprogramming of commercial primary hADSCs (Lonza-PT5006 and ATCC-PCS-500-011) and commercial primary hASFs (Lonza-CC2511-6665, Lonza-CC2511-6027 and Lonza-CC2511-38040), which were also cultured in Mesenchymal Stem Cell Growth Medium 2.

### MEFs

MEFs were isolated from day 13.5 embryos. The head, limbs, visceral tissues and gonads were removed, and the remaining embryos were washed twice with PBS containing 2% penicillin-streptomycin, minced with scissors and dissociated with 0.25% trypsin-EDTA (1-2 ml per embryo) at 37° C. After 5-10 min, MEF medium (high-glucose DMEM supplemented with 15% FBS and 2% penicillin-streptomycin) was added, and cells were pipetted several times for dissociation. After centrifuging at 400 g for 5 min, cells were collected and resuspended in MEF medium. The next day, the medium was changed to fresh MEF medium to remove nonadherent cells. MEFs usually became confluent in 2 days and were ready to passage for reprogramming.

Generation of hCiPSCs Using the Fast-Reprogramming Protocol
Medium Preparation for hCiPSC Induction

The stage 1 induction medium contained Knockout DMEM (Gibco) supplemented with 2% B27 supplement (Gibco), 1% Knockout Serum Replacement (KSR; Gibco), 1% GlutaMAX (Gibco), 1% nonessential amino acids (NEAA; Gibco), 1% penicillin-streptomycin, 50 μg ml-1 Vc2p, 5 mM LiCl, 1 mM nicotinamide, 40 ng ml⁻¹ BMP4 and the small molecules CHIR-99021 (5 μM), 616452 (10 μM), TTNPB (2 μM), SAG (0.5 μM), EPZ-5676 (2 μM), DZNep (0.05 μM), ruxolitinib (1 μM), VTP50469 (0.5 μM), AKT kinase inhibitor (1 μM), JNK-IN-8 (0.2 μM), SETD2-IN-1 (0.2 μM), AM095 free acid (0.5 μM), WM-8014 (1 μM) and A-485 (0.5 μM).

Stage 2 induction medium contained Knockout DMEM supplemented with 2% B27 supplement, 1% GlutaMAX, 1% NEAA, 1% penicillin-streptomycin, 50 μg ml⁻¹ Vc2p, 200 ng ml⁻¹ bFGF and the small molecules CHIR-99021 (5 μM), 616452 (10 μM), TTNPB (2 μM), SAG (0.5 μM), JNK-IN-8 (0.5 μM), EPZ-5676 (2 μM), DZNep (0.2 μM), ruxolitinib (1 μM), BIRB 796 (2 μM), SGC-CBP30 (2 μM), VTP50469 (0.5 μM), 5-Iodotubercidin (0.5 μM), AKT kinase inhibitor (0.2 μM), CX-4945 (1 μM), RA (5 μM) and GSK-3685032 (0.02 μM).

Stage 3 induction medium contained Knockout DMEM supplemented with 2% B27 supplement, 1% GlutaMAX, 1% NEAA, 1% penicillin-streptomycin, 5% KSR, 50 μg ml⁻¹ Vc2p, 20 ng ml⁻¹ recombinant human heregulinβ-1 (HRG) and the small molecules CHIR-99021 (1 μM), Y-27632 (10 μM), PD0325901 (1 μM) and SB-590885 (0.5 μM). Knockout DMEM containing supplements and the small molecules mentioned above served as the basal medium for stage 3. For the first 2-3 days of stage 3 induction, valproic acid sodium salt (VPA; 200 μM), tranylcypromine (10 μM), DZNep (0.2 μM), EPZ-5676 (2 μM) and PY-60 (10 μM) were added to the basal medium. For the next 2-3 days, VPA (200 μM) and PY-60 (2 μM) were added to the basal medium. Information on the small molecules, cytokines and other reagents is available in Supplementary Table 4.

Procedures for the Induction of hCiPSCs from hADSCs and hASFs

Hypoxia with 5% O₂ was used for stage 1 induction. After stage 1 induction, the culture was changed to 21% O₂.

(1) hADSCs and hASFs were seeded in high-glucose DMEM supplemented with 15% FBS at a density of 1.2-1.4×10⁴ cells per well in 12-well plates or 0.6-0.7×10⁴ cells per well in 24-well plates. The medium was changed to stage 1 induction medium the next day.

(2) For stage 1 induction, single-layer epithelium-like cells emerged on day 4. When the epithelium-like cells reached ˜100% confluence (usually after 6-8 days of stage 1 induction for hADSCs and 8-10 days for hASFs), the medium was changed to stage 2 induction medium. For the induction of hADSCs in 24-well plates, 1 ml of stage 1 medium was added to each well (2 ml per well for 12-well plates), and there was no need to change the medium during stage 1. For the induction of hASFs, stage 1 medium was replaced with fresh medium on day 6.

(3) For stage 2 induction, multilayered cell colonies appeared after 4 days of treatment, and these cell colonies continually grew larger. The medium was changed to stage 3 induction medium after 2-6 days of treatment. The recommended volume of medium for stage 2 was 1 ml per well for 24-well plates (2 ml per well for 12-well plates), and fresh medium was changed every 4 days. Four days of stage 2 induction was sufficient for all 15 tested cell lines, and extending this stage to 6 days obviously increased the reprogramming efficiency.

(4) For stage 3 induction, VPA (200 μM), tranylcypromine (10 μM), DZNep (0.2 μM), EPZ-5676 (2 μM) and PY-60 (10 μM) were added to the basal medium for the first 2-3 days. Subsequently, VPA (200 μM) and PY-60 (2 μM) were added to the basal medium for the next 2-3 days. To allow the primary hCiPSC colonies to grow larger (if necessary), basal medium was applied for an additional 2-4 days.

(5) At the end of stage 3, all the cells in the well were fixed with 4% paraformaldehyde at room temperature for subsequent immunofluorescence staining. OCT4 staining was performed, and OCT4+ colonies were considered primary hCiPSC colonies. The number of OCT4+ colonies divided by the number of inputs hADSCs or hASFs was used as a measure of reprogramming efficiency.

Generation of hCiPSCs Using the Original Reprogramming Protocol

The original reprogramming protocol for generating hCiPSCs from hADSCs and hASFs was previously described7. In brief, hADSCs and hASFs were seeded in high-glucose DMEM supplemented with 15% FBS at a density of 1×10⁴ cells per well in 12-well plates or 0.5-0.6×10⁴ cells per well in 24-well plates. The next day, the medium was changed to the original stage 1 induction medium for 8-10 days. This was followed by 8-12 days in the original stage 2 medium and then 8-10 days in the original stage 3 medium.

### Results

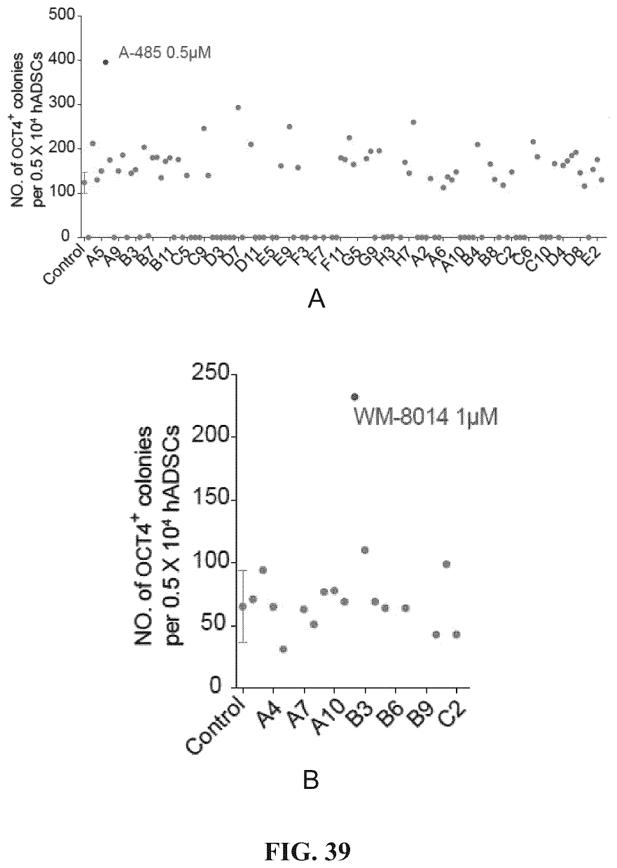

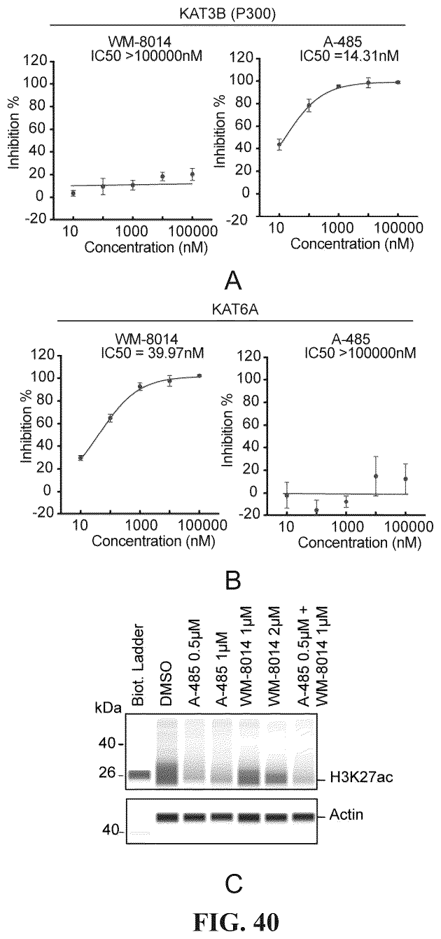

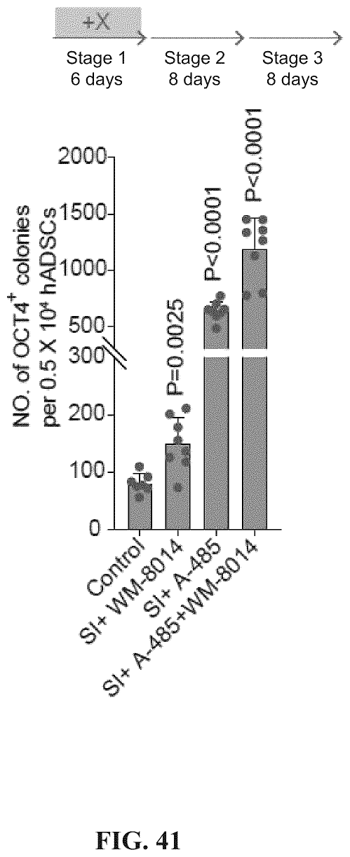

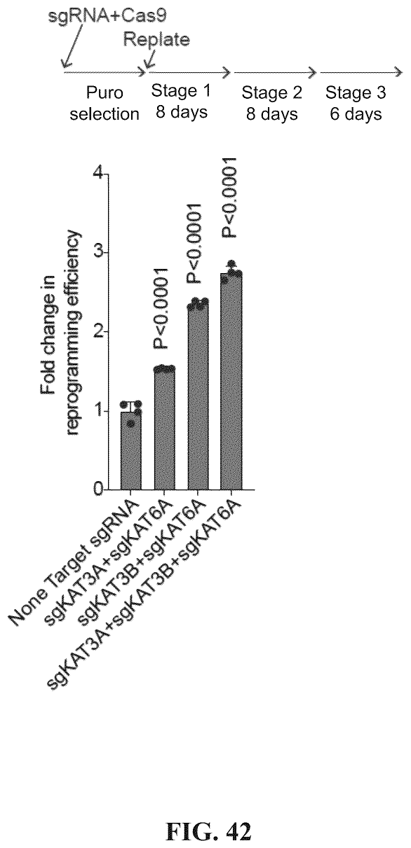

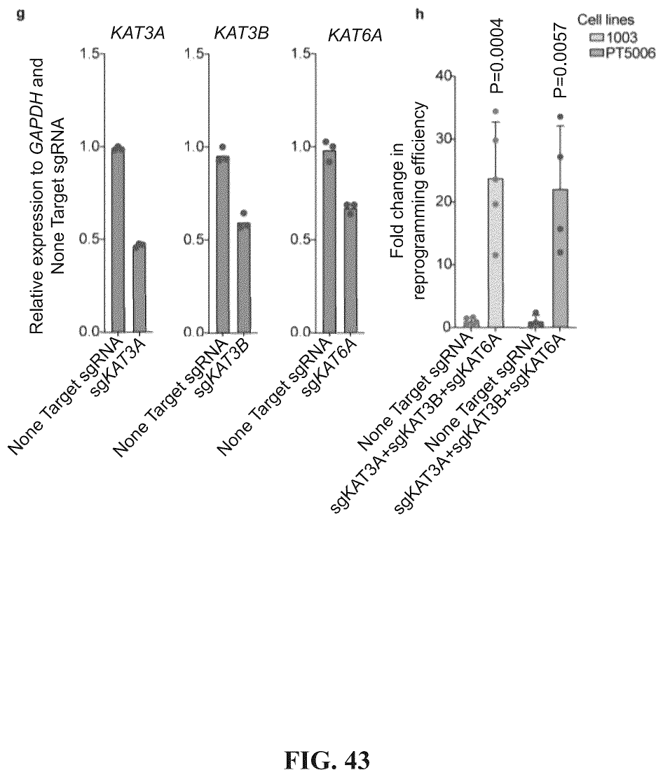

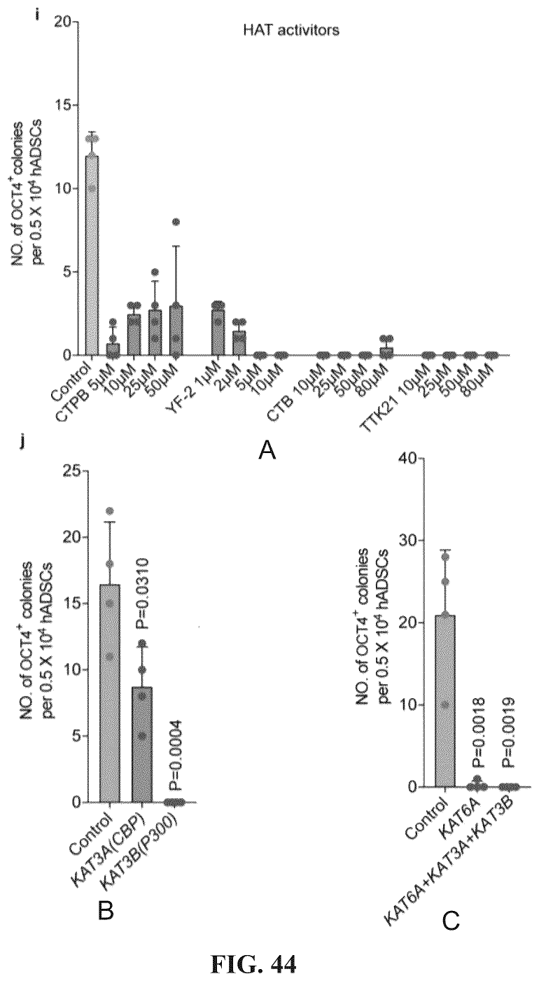

The KAT3A/KAT3B catalytic inhibitor A-485 and the KAT6A inhibitor WM-8014 were found to be most effective in improving reprogramming efficiency (FIG. 39A-B and FIG. 40A-C). KAT3A/KAT3B and KAT6A are both histone acetyltransferases (HATs) that acetylate lysine residues in histones, act as transcriptional coactivators and play important roles in maintaining cell identity. Importantly, the combination of A-485 and WM-8014 significantly improved the chemical reprogramming efficiency by more than 12-fold when the duration of stage 1 induction was reduced to only 6 days (FIG. 41), a phase when the gradual loss of somatic cell identity occurred. Moreover, the inventors further confirmed that knockdown of KAT3A/KAT3B together with KAT6A significantly promoted the generation of hCiPSCs (FIG. 42 and FIG. 43). In addition, it was found that overexpression of these genes or adding small molecular activators of KAT3A/KAT3B and KAT6A both inhibited the generation of hCiPSCs during chemical reprogramming (FIG. 44). Together, these results strongly suggest that KAT3A/KAT3B and KAT6A impede chemical reprogramming and that their combined inhibition can greatly facilitate cell fate conversion from somatic cells to hCiPSCs.

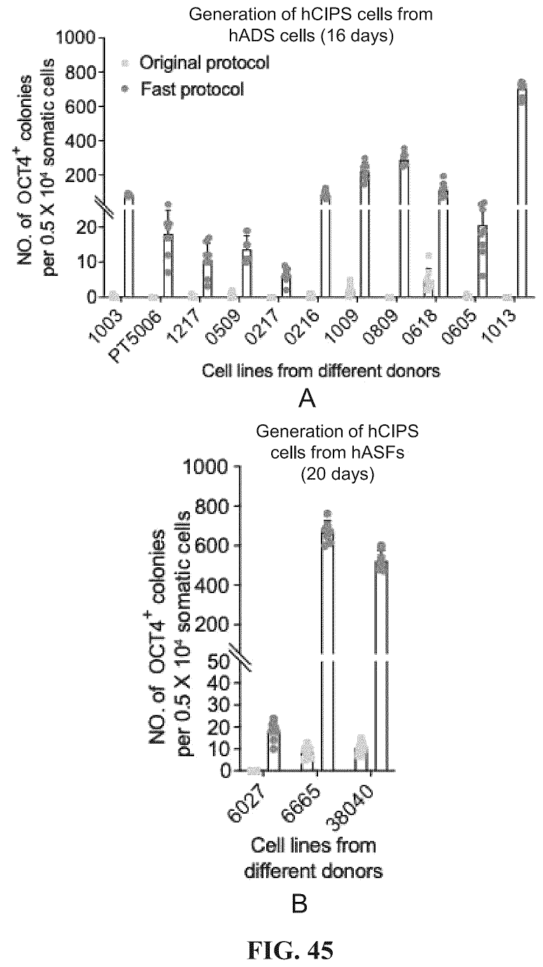

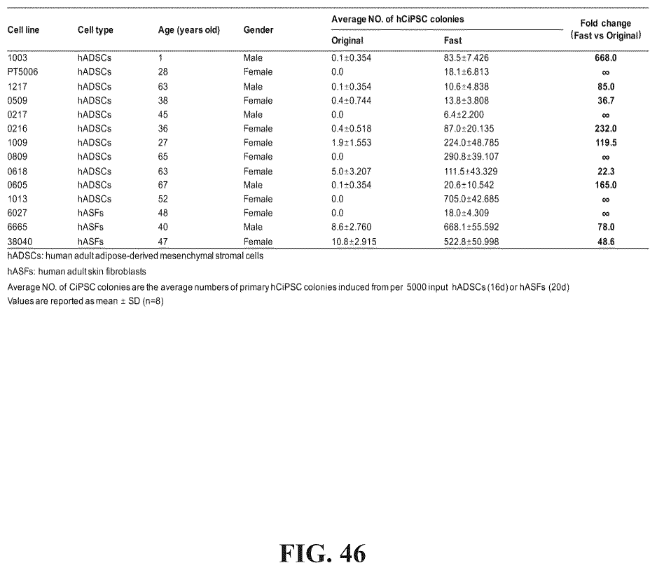

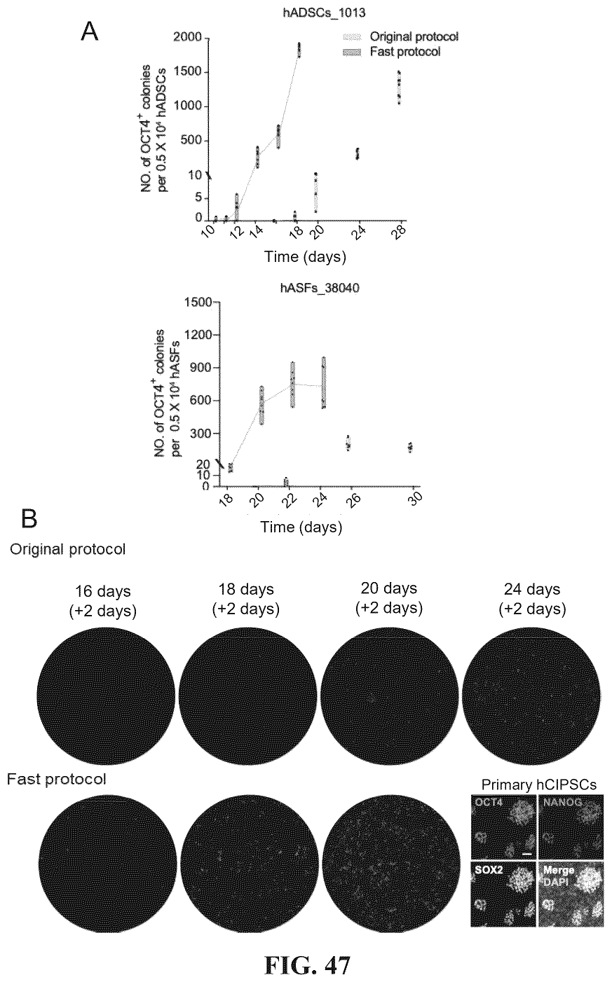

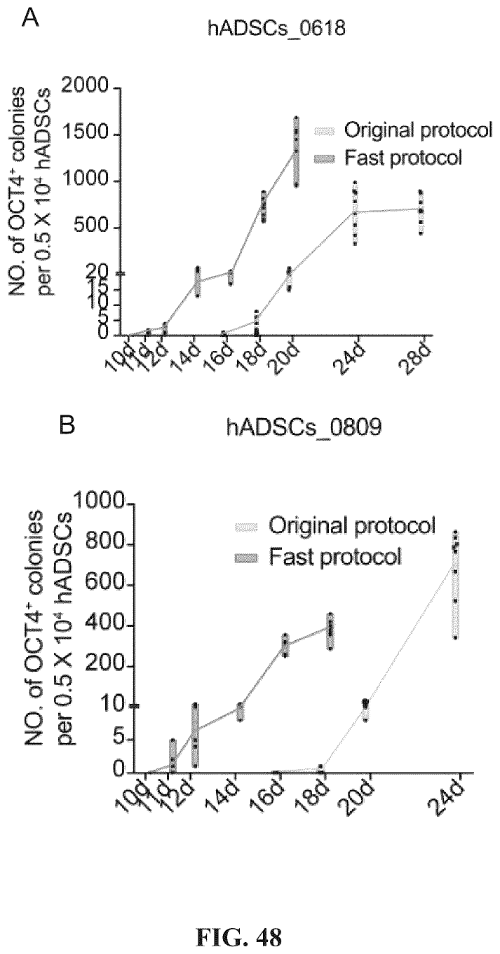

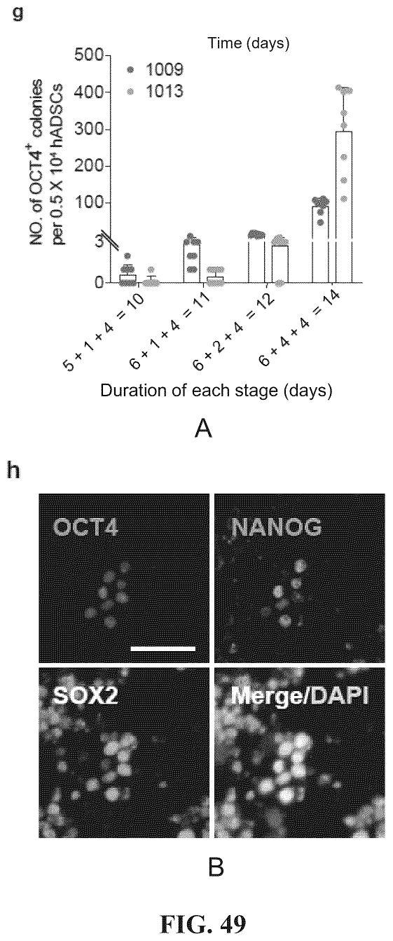

With the optimized reprogramming system, hADSCs could be robustly induced into hCiPSCs in only 16 days at efficiencies reaching 14.8%, marking, on average, an over 20-fold increase (FIGS. 45A-B and FIG. 46). This is in stark contrast to the previous chemical reprogramming protocol, which rarely generated hCiPSC colonies in such short periods (0-0.1%; FIGS. 45A-B and FIG. 46). For example, for the refractory cell line hADSC-1003, the new protocol induced almost 100 hCiPSC colonies in 16 days, whereas the original protocol induced only 0-1 colony in this time period (FIG. 45A and FIG. 46). This new protocol also improved the efficiency of hCiPSC induction from hASFs in 20 days (FIG. 45B and FIG. 46). On average, the optimized reprogramming protocol shortened induction time by approximately 10 days, accounting for about one-third of the original induction time (FIGS. 47 and 48). Notably, hCiPSC colonies emerged as early as 10 days under these optimized reprogramming conditions (FIG. 49).

### Example 8: Exemplary Chemical Reprogramming Factors

Provided in this example are exemplary chemical reprogramming factors, growth factors, and/or reagents used in the foregoing Examples. A list of these chemical reprogramming factors, growth factors, and/or reagents is shown in TABLE 3 below. Other chemicals with similar cellular activities of those described in TABLE 3 can also be used in the methods described herein.

TABLE 3
List of chemical reprogramming factors, growth factors,
or reagents for methods described herein

| REAGENT or RESOURCE | SOURCE | IDENTIFIER |
|---|---|---|
| Chemicals or Growth Factors | | |
| Valproic acid sodium salt | Sigma-Aldrich | Cat#P4543 |
| (VPA) | | |
| CHIR99021 (CHIR, C) | WUXI APPTEC | N/A |
| 616452 (6) | WUXI APPTEC | N/A |
| Tranylcypromine (Tranyl, T) | Enzo | Cat#BML-EI217- |
| | | 0005 |
| 3-deazaneplanocin A | WUXI APPTEC | N/A |
| (DZNep) | | |
| PD0325901 | WUXI APPTEC | N/A |
| EPZ5676 | MCE | Cat# HY-15593 |
| TTNPB (N) | WUXI APPTEC | N/A |
| Y-27632 | WUXI APPTEC | N/A |
| SAG | WUXI APPTEC | N/A |
| Recombinant Human FGF2 | Origene | Cat# TP750002 |
| JNKIN8 (J) | Selleckchem | Cat# S4901 |
| Recombinant Human | PEPROTECH | Cat# 100-03 |
| Heregulin β-1 (HRG) | | |
| IWP-2 | Selleckchem | Cat# S7085 |
| SB590885 | Selleckchem | Cat# S2220 |
| Ruxolitinib (Ruxo) | Selleckchem | Cat# S1378 |
| BIRB796 (BIRB) | Selleckchem | Cat# S1574 |
| SGC-CBP30 (CBP30) | Selleckchem | Cat# S7256 |
| Dorsomorphin (DM) | MCE | Cat# HY-13418A |
| VTP50469 | Selleckchem | Cat# S8934 |
| L-Ascorbic acid 2-phosphate | Sigma-Aldrich | Cat# A8960 |
| (Vc2P) | | |
| LiCl | Sigma-Aldrich | Cat# L4408 |
| Nicotinamide (NAM) | Sigma-Aldrich | Cat# 72340 |
| AKT Kinase Inhibitor | MCE | Cat# HY-10249A |
| 5-Iodotubercidin (5ITU) | MCE | Cat# HY-15424 |
| Recombinant Human TNF-α | PEPROTECH | Cat#300-01A |
| DAPI | Roche | Cat# 10236276001 |
| 4% paraformaldehyde | DingGuo | Cat# AR-0211 |
| Normal donkey serum | Jackson Immuno | Cat# 017-000-121 |
| | Research | |
| Triton ™ X-100 | Sigma-Aldrich | Cat# 9036-19-5 |
| PBS | CORNING | Cat# 05418005 |
| DMSO (Dimethyl sulfoxide) | Sigma-Aldrich | Cat# D2650 |
| Trizol (TRI) Reagent | Sigma-Aldrich | Cat# T9424 |
| DMEM | Gibco | Cat# C11965500BT |
| KnockOut ™ DMEM | Gibco | Cat# 10829018 |
| N2 supplement | Gibco | Cat# 17502-048 |
| B27 supplement | Gibco | Cat# 17504-044 |
| Fetal Bovine Serum (FBS) | Vistech | Cat# VIS93526487 |
| Knockout Serum | Gibco | Cat# 10828028 |
| Replacement (KSR) | | |
| Penicillin-Streptomycin | Gibco | Cat# 15140-122 |
| Collagenase IV | Gibco | Cat# 1963347 |
| 0.25% Trypsin-EDTA | Gibco | Cat# 25200-056 |
| GlutaMAX ™ | Gibco | Cat# 35050-061 |
| MEM Non-Essential Amino | Gibco | Cat# 11140050 |
| Acids Solution (NEAA) | | |
| Mesenchymal Stem Cell | PromoCell | Cat# C-28009 |
| Growth Medium 2 | | |
| AlbuMAX ™-II | Gibco | Cat# 11021-045 |
| Matrigel | Corning | Cat# 354248 |
| mTeSR ™ Plus Medium | STEMCELL | Cat# 100-0276 |
| Accutase | Millipore | Cat# SCR005 |
| ReLeSR ™ | STEMCELL | Cat# 05872 |
| Reagents for Various Assays | | |
| Cytofix/Cytoperm ™ | BD Biosciences | Cat# 554714 |
| Fixation/Permeablization Kit | | |
| Direct-zol RNA MiniPrep Kit | Zymo Research | Cat# R2053 |
| TransScript First-Strand | TransGen | Cat# AT311-03 |
| cDNA Synthesis SuperMix | Biotech | |
| KAPA SYBR FAST qPCR | KAPA Biosystems | Cat# KM4101 |
| Kit Master Mix | | |
| Direct-zol RNA MiniPrep Kit | Zymo Research | Cat# R2072 |
| Hyperactive Universal | Vazyme | Cat# TD903-02 |
| CUT&Tag Assay Kit for | | |
| Illumina | | |
| QIAGEN Plasmid Plus Midi | QIAGEN | Cat. No./ID: 12945 |
| Kits | | |

While preferred embodiments of the present disclosure have been shown and described herein, it may be obvious to those skilled in the art that such embodiments are provided by way of example only. It is not intended that the disclosure be limited by the specific examples provided within the specification. While the invention has been described with reference to the aforementioned specification, the descriptions and illustrations of the embodiments herein are not meant to be construed in a limiting sense. Numerous variations, changes, and substitutions may now occur to those skilled in the art without departing from the disclosure. Furthermore, it shall be understood that all aspects of the disclosure are not limited to the specific depictions, configurations or relative proportions set forth herein which depend upon a variety of conditions and variables. It may be understood that various alternatives to the embodiments described herein may be employed in practicing the present disclosure. It is therefore contemplated that the disclosure shall also cover any such alternatives, modifications, variations or equivalents. It is intended that the following claims define the scope of the invention and that methods and structures within the scope of these claims and their equivalents be covered thereby.

What is claimed is:

1. A composition comprising:
a) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
b) a transforming growth factor-beta (TGFβ) receptor inhibitor, optionally wherein the transforming growth factor-beta (TGFβ) receptor inhibitor comprises E-616452, A 83-01, SB431542, SB 505124, GW 788388, or SB 525334,
c) a retinoic acid receptor (RAR) agonist, optionally wherein the retinoic acid receptor (RAR) agonist comprises TTNPB, Ch55, or AM580, and
d) one or more of a Akt (protein kinase B) inhibitor or an SET domain containing 2 (SETD2) inhibitor, optionally wherein the Akt (protein kinase B) inhibitor comprises AKT Kinase Inhibitor (AKTi) and optionally wherein the SET domain containing 2 (SETD2) inhibitor comprises SETD2-IN-1, EPZ-719, or MMSET-IN-1.

2. The composition of claim 1, further comprising one or more of:
e) a disruptor of telomeric silencing 1-like (Dot1L) inhibitor, optionally wherein the disruptor of telomeric silencing 1-like (Dot1L) inhibitor comprises EPZ004777, EPZ5676 or SGC0946,
f) an agonist for G protein-coupled receptor Smoothened, optionally wherein the agonist for G protein-coupled receptor Smoothened comprises SAG, Purmorphamine, Hh-Ag1.5, or human Sonic Hedgehog (SHH) protein,
g) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
h) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenosine periodate oxidized (Adox), or 3-deazaadenosine (DZA),
i) a Menin-MLL interaction inhibitor, optionally wherein the Menin-MLL interaction inhibitor comprises VTP50469, MI3454, or WDR5-IN-4, or
j) a c-Jun kinase inhibitor, optionally wherein the c-Jun kinase inhibitor comprises JNKIN8, JNKIN7, JNKIN5, or JNKIN12.

3. The composition of claim 1, further comprising one or more of a KAT3A/KAT3B inhibitor or a KAT6A inhibitor, optionally wherein the KAT3A/KAT3B inhibitor is A-485, CBP/P300 IN 8, GEN049, CBP/P300 IN 12, SGCCBP30 or ICBP112 and optionally wherein the KAT6A inhibitor is WM-8014 or WM1119.

4. A method for reprogramming cells, comprising the steps of:
(I) contacting a first population of cells comprising the cells with a first composition comprising:
a) a glycogen synthase kinase 3 (GSK-3) inhibitor;
b) a transforming growth factor-beta (TGFβ) receptor inhibitor,
c) a retinoic acid receptor (RAR) agonist, and
d) one or more of an Akt (protein kinase B) inhibitor or an SET domain containing 2 (SETD2) inhibitor;
thereby obtaining a second population of cells.

5. The method of claim 4, wherein the second population of cells obtained from step (I) comprises epithelial-like cells that express LIN28A, optionally the epithelial-like cells further express one or more of KRT18, KRT19, WT1, NMYC, WNT2B, PAX8, SMAD3, GLI3, or TBX2.

6. The method of claim 4, further comprising:
(II) contacting at least a subset of the second population of cells obtained from step (I) with a second composition, thereby generating a third population of cells, wherein the third population of cells comprises intermediate plastic state cells, optionally the intermediate plastic state cells express LIN28A and SALL4, and one or more of MSX2, NMYC, SDC1, WNT4, FGF19, or TOP2A.

7. The method of claim 6, further comprising:
(III) contacting at least a subset of the third population of cells obtained from step (II) with a third composition, thereby generating a fourth population of cells, wherein the fourth population of cells comprises pluripotent stem cells, optionally the pluripotent stem cells are human chemically induced pluripotent stem cells (hCiPSCs).

8. The method of claim 4, wherein
a) the glycogen synthase kinase 3 (GSK-3) inhibitor comprises CHIR99021, CHIR98014, TD114-2, GSK-3 Inhibitor XV, SB-216763 or SB-415286,
b) the transforming growth factor-beta (TGFβ) receptor inhibitor comprises E-616452, A 83-01, SB431542, SB 505124, GW 788388, or SB 525334,
c) the retinoic acid receptor (RAR) agonist comprises TTNPB, Ch55, or AM580,
d) the Akt (protein kinase B) inhibitor comprises AKT Kinase Inhibitor (AKTi), or the SET domain containing 2 (SETD2) inhibitor comprises SETD2-IN-1, EPZ-719, or MMSET-IN-1.

9. The method of claim 4, wherein the first composition further comprises one or more of:
e) a disruptor of telomeric silencing 1-like (Dot1L) inhibitor, optionally wherein the disruptor of telomeric silencing 1-like (Dot1L) inhibitor comprises EPZ004777, EPZ5676 or SGC0946,
f) an agonist for G protein-coupled receptor Smoothened, optionally wherein the agonist for G protein-coupled receptor Smoothened comprises SAG, Purmorphamine, Hh-Ag1.5, or human Sonic Hedgehog (SHH) protein,
g) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
h) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenosine periodate oxidized (Adox), or 3-deazaadenosine (DZA),
i) a Menin-MLL interaction inhibitor, optionally wherein the Menin-MLL interaction inhibitor comprises VTP50469, MI3454, or WDR5-IN-4, or
j) a c-Jun kinase inhibitor, optionally wherein the c-Jun kinase inhibitor comprises JNKIN8, JNKIN7, JNKIN5, or JNKIN12.

10. The method of claim 4, wherein the first composition further comprises one or more of a KAT3A/KAT3B inhibitor or a KAT6A inhibitor, optionally wherein the KAT3A/KAT3B inhibitor is A-485, CBP/P300 IN 8, GEN049, CBP/P300 IN 12, SGCCBP30 or ICBP112 and optionally wherein the KAT6A inhibitor is WM-8014 or WM1119.

11. The method of claim 6, wherein the second composition comprises
a) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
b) a Transforming growth factor-beta (TGFβ) receptor inhibitor, optionally wherein the Transforming growth factor-beta (TGFβ) receptor inhibitor comprises E-616452, A 83-01, SB431542, SB 505124, GW 788388, or SB 525334,
c) a retinoic acid receptor (RAR) agonist, optionally wherein the retinoic acid receptor (RAR) agonist comprises TTNPB, Ch55, or AM580,
d) a c-Jun kinase inhibitor, optionally wherein the c-Jun kinase inhibitor comprises JNKIN8, JNKIN7, JNKIN5, or JNKIN12, and
e) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenosine periodate oxidized (Adox), or 3-deazaadenosine (DZA).

12. The method of claim 11, wherein the second composition further comprises one or more of:
f) a CBP/p300 bromodomain inhibitor, optionally wherein the CBP/p300 bromodomain inhibitor comprises SGC-CBP30, I-CBP112, or GNE272,
g) an adenosine kinase inhibitor, optionally wherein the adenosine kinase inhibitor comprises 5-Iodotubercidin (5-ITU) or ABT 702,
h) a casein kinase 2 inhibitor, optionally wherein the casein kinase 2 inhibitor comprises CX-4945, TPP22, or Ellagic acid,
i) a Menin-MLL interaction inhibitor, optionally wherein the Menin-MLL interaction inhibitor comprises VTP50469, MI3454, or WDR5-IN-4,
j) an agonist for the G protein-coupled receptor Smoothened, optionally wherein the agonist for the G protein-coupled receptor Smoothened comprises SAG, Purmorphamine, Hh-Ag1.5, or human Sonic Hedgehog (SHH),
k) a ROCK inhibitor, optionally wherein the ROCK inhibitor comprises Y-27632 or thiazovivin,
l) a BMP receptor/AMPK inhibitor, optionally wherein the BMP receptor/AMPK inhibitor comprises Dorsomorphin,
m) a disruptor of telomeric silencing 1-like (Dot1L) inhibitor, optionally wherein the disruptor of telomeric silencing 1-like (Dot1L) inhibitor comprises EPZ004777, EPZ5676 or SGC0946,
n) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
o) a p38 MAPK inhibitor, optionally wherein the p38 MAPK inhibitor comprises BIRB796, SB203580, or SB202190,
p) an SET domain containing 2 (SETD2) inhibitor, optionally wherein the SET domain containing 2 (SETD2) inhibitor comprises SETD2-IN-1, EPZ-719, or MMSET-IN-1,
q) an Akt (protein kinase B) inhibitor, optionally wherein the Akt (protein kinase B) inhibitor comprises AKT Kinase Inhibitor (AKTi),
r) a retinoic acid receptor (RAR) agonist comprising retinoic acid, or
s) a DNA methyltransferase inhibitor comprising GSK-3685032.

13. The method of claim 7, wherein the third composition comprises:
a) an MEK inhibitor, optionally wherein the MEK inhibitor comprises PD0325901, AZD8330, or TAK-733,
b) a B-Raf inhibitor, optionally wherein the B-Raf inhibitor comprises SB590885, Vemurafenib, RAF265, or PLX4720, and
c) a histone deacetylase (HDAC) inhibitor, optionally wherein the HDAC inhibitor comprises valproic acid (VPA), LMK235, MS275, or HDACi IV.

14. A second composition comprising:
a) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
b) a Transforming growth factor-beta (TGFβ) receptor inhibitor, optionally wherein the Transforming growth factor-beta (TGFβ) receptor inhibitor comprises E-616452, A 83-01, SB431542, SB 505124, GW 788388, or SB 525334,
c) a retinoic acid receptor (RAR) agonist, optionally wherein the retinoic acid receptor (RAR) agonist comprises TTNPB, Ch55, or AM580,
d) a c-Jun kinase inhibitor, optionally wherein the c-Jun kinase inhibitor comprises JNKIN8, JNKIN7, JNKIN5, or JNKIN12, and
e) an S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenosine periodate oxidized (Adox), or 3-deazaadenosine (DZA).

15. The second composition of claim 14, wherein the second composition further comprises one or more of:
f) a CBP/p300 bromodomain inhibitor, optionally wherein the CBP/p300 bromodomain inhibitor comprises SGC-CBP30, I-CBP112, or GNE272,
g) an adenosine kinase inhibitor, optionally wherein the adenosine kinase inhibitor comprises 5-Iodotubercidin (5-ITU) or ABT 702,
h) a casein kinase 2 inhibitor, optionally wherein the casein kinase 2 inhibitor comprises CX-4945, TPP22, or Ellagic acid,
i) a Menin-MLL interaction inhibitor, optionally wherein the Menin-MLL interaction inhibitor comprises VTP50469, MI3454, or WDR5-IN-4,
j) an agonist for the G protein-coupled receptor Smoothened, optionally wherein the agonist for the G protein-coupled receptor Smoothened comprises SAG, Purmorphamine, Hh-Ag1.5, or human Sonic Hedgehog (SHH),
k) a ROCK inhibitor, optionally wherein the ROCK inhibitor comprises Y-27632 or thiazovivin,
l) a BMP receptor/AMPK inhibitor, optionally wherein the BMP receptor/AMPK inhibitor comprises Dorsomorphin,
m) a disruptor of telomeric silencing 1-like (Dot1L) inhibitor, optionally wherein the disruptor of telomeric silencing 1-like (Dot1L) inhibitor comprises EPZ004777, EPZ5676, or SGC0946,
n) a Jak1/Jak2 inhibitor, optionally wherein the Jak1/Jak2 inhibitor comprises Ruxolitinib, Tofacitinib, AZD1480, Baricitinib, S-Ruxolitinib, or Fedratinib,
o) a p38 MAPK inhibitor, optionally wherein the p38 MAPK inhibitor comprises BIRB796, SB203580, or SB202190,
p) an SET domain containing 2 (SETD2) inhibitor, optionally wherein the SET domain containing 2 (SETD2) inhibitor comprises SETD2-IN-1, EPZ-719, or MMSET-IN-1,
q) an Akt inhibitor, optionally wherein the Akt inhibitor comprises AKT Kinase Inhibitor (AKTi),
r) a retinoic acid receptor (RAR) agonist, optionally wherein the retinoic acid receptor (RAR) agonist comprising retinoic acid, or
s) a DNA methyltransferase inhibitor, optionally wherein the DNA methyltransferase inhibitor comprising GSK-3685032.

16. A third composition comprising:
a) an MEK inhibitor, optionally wherein the MEK inhibitor comprises PD0325901, AZD8330, or TAK-733,
b) a B-Raf inhibitor, optionally wherein the B-Raf inhibitor comprises SB590885, Vemurafenib, RAF265, or PLX4720, and
c) a histone deacetylase (HDAC) inhibitor, optionally wherein the histone deacetylase (HDAC) inhibitor comprises valproic acid (VPA), LMK235, MS275, or HDACi IV.

17. The third composition of claim 16, wherein the third composition further comprises one or more of:
d) a Wnt inhibitor, optionally wherein the Wnt inhibitor comprises IWR-1 or IWP-2,
e) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
f) a ROCK inhibitor, optionally wherein the ROCK inhibitor comprises Y-27632 or thiazovivin,
g) an inhibitor of histone demethylation, optionally wherein the inhibitor of histone demethylation comprises Tranylcypromine,
h) a disruptor of telomeric silencing 1-like (Dot1L) inhibitor, optionally wherein the disruptor of telomeric silencing 1-like (Dot1L) inhibitor comprises EPZ004777 or EPZ5676,
i) a S-adenosyl-L-homocysteine (SAH) hydrolase inhibitor, optionally wherein the S-adenosyl-L-homocysteine (SAH) hydrolase inhibitor comprises deazaneplanocin A (DZNep), Neplanocin A (NepA), Adenosine periodate oxidized (Adox), or 3-deazaadenosine (DZA), or
j) a specific activator of YAP transcriptional activity that targets Annexin A2 (ANXA2), optionally wherein the specific activator of YAP transcriptional activity that targets Annexin A2 (ANXA2) comprises PY-60.

18. An isolated population of cells comprising intermediate plastic state cells that express:
a) LIN28A and SALL4;
b) one or more of MSX2, NMYC, WNT4, FGF19, or TOP2A;
c) one or more of MSX1, HOXB9, WT1, GATA2, HMGA2, or LEF1 and
d) one or more of FGF9, HOXA9, HOXA1, PTCH1, HOXA5, CCND2, SDC1, TBX3, BMP4, or IGF2.

19. An isolated population of cells comprising epithelial-like cells that express:
a) LIN28A,
b) one or more of NMYC, WNT2B, PAX8, SMAD3, or GLI3, and
c) one or more of KRT18, KRT19, WT1, or TBX2.

20. A composition comprising:
a) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
b) a transforming growth factor-beta (TGFβ) receptor inhibitor, optionally wherein the transforming growth factor-beta (TGFβ) receptor inhibitor comprises E-616452, A 83-01, SB431542, SB 505124, GW 788388, or SB 525334,
c) a retinoic acid receptor (RAR) agonist, optionally wherein the retinoic acid receptor (RAR) agonist comprises TTNPB, Ch55, or AM580, and
d) one or more of a Akt (protein kinase B) inhibitor or an SET domain containing 2) inhibitor, optionally wherein the Akt inhibitor comprises AKT Kinase Inhibitor (AKTi) and optionally wherein the SET domain containing 2 (SETD2) inhibitor comprises SETD2-IN-1, EPZ-719, or MMSET-IN-1.

21. A second composition comprising:
a) a glycogen synthase kinase 3 (GSK-3) inhibitor, optionally wherein the glycogen synthase kinase 3 (GSK-3) inhibitor comprises CHIR99021, CHIR98014, TD114-2, GSK-3 Inhibitor XV, SB-216763 or SB-415286,
b) a transforming growth factor-beta (TGFβ) receptor inhibitor, optionally wherein the transforming growth factor-beta (TGFβ) receptor inhibitor comprises E-616452, A 83-01, SB431542, SB 505124, GW 788388, or SB 525334, and
c) a retinoic acid receptor (RAR) agonist, optionally wherein the retinoic acid receptor (RAR) agonist comprises TTNPB, Ch55, or AM580.

22. A third composition comprising:
a) an MEK inhibitor, optionally wherein the MEK inhibitor comprises PD0325901, AZD8330, and TAK-733, and
b) a histone deacetylase (HDAC) inhibitor, optionally wherein the histone deacetylase (HDAC) inhibitor comprises valproic acid (VPA), LMK235, MS275, and HDACi IV.

23. A method for reprogramming cells, comprising the steps of:
(I) contacting a first population of cells comprising the cells with the composition of claim 20 thereby generating epithelial-like cells that express LIN28, optionally the epithelial-like cells further express one or more of KRT18, KRT19, WT1, NMYC, WNT2B, PAX8, SMAD3, GLI3, or TBX2.

24. The method of claim 23, further comprising:
(II) contacting at least a subset of the second population of cells obtained from step (I) with the second composition of claim 21 thereby generating a third population of cells, wherein the third population of cells comprises intermediate plastic state cells that express LIN28A and SALL4, and one or more of MSX2, NMYC, SDC1, WNT4, FGF19, or TOP2A.

25. The method of claim 24, further comprising:
(III) contacting at least a subset of the third population of cells obtained from step (II) with the third composition of claim 22, thereby generating a fourth population of cells, wherein the fourth population of cells comprises pluripotent stem cells that express OCT4, SOX2, NANOG, FGF4, ZFP57, REX1, DPPA4, TDGF1, TRA-1-60, TRA-1-81, SSEA4, KLF4, KLF17, DPPA3, DPPA5, DNMT3L, REX1, UTF1.
